(19) United States
(12) Patent Application Publication
(10) Pub. No.: US 2023/0158159 A1
(43) Pub. Date: May 25, 2023

(54) CHIMERIC DEGRADERS OF CYCLIN-DEPENDENT KINASE 9 AND USES THEREOF

(71) Applicant: Massachusetts Institute of Technology, Cambridge, MA (US)

(72) Inventors: Angela N. Koehler, Belmont, MA (US); Andre Richters, Boston, MA (US); Julie Urgiles, Boston, MA (US); Florian Kabinger, Vienna (AT); Mohammed Abraham Toure, Cambridge, MA (US)

(73) Assignee: Massachusetts Institute of Technology, Cambridge, MA (US)

(21) Appl. No.: 17/920,574

(22) PCT Filed: Apr. 22, 2021
(86) PCT No.: PCT/US2021/028573

§ 371 Date: Oct. 21, 2022

Related U.S. Application Data
(60) Provisional application No. 63/014,861, filed on Apr. 24, 2020.

(51) Int. Cl. A61K 47/55; A61K 47/54; A61P 35/02

(52) U.S. Cl. CPC A61K 47/55; A61K 47/545; A61P 35/02

(57) ABSTRACT

Provided herein are bifunctional compounds that bind cyclin-dependent kinase 9 (CDK9) and/or promote targeted ubiquitination for the degradation of CDK9. In particular, provided are compounds that can bind CDK9, a protein whose dysregulation is implicated in a variety of cancers, and can promote CDK9′s degradation by recruiting an E3 ubiquitin ligase (e.g., Cereblon, VHL). The E3 ubiquitin ligase can ubiquitinate CDK9, marking it for proteasomal degradation. Also provided are pharmaceutical compositions comprising the bifunctional compounds. Also provided are methods of treating cancer, and methods of promoting the degradation of CDK9 protein by E3 ubiquitin ligase activity in a subject or biological sample by administering a compound or composition described herein.

### RELATED APPLICATIONS

This application claims priority under 35 U.S.C. § 119(e) to U.S. Provisional Application, U.S. Ser. No. 63/014,861, filed Apr. 24, 2020, which is incorporated herein by reference in its entirety.

### BACKGROUND

The serine/threonine kinase cyclin-dependent kinase 9 (CDK9) facilitates the phosphorylation of specific protein substrates and thereby modulates their stability and/or activation state. CDK9 and its regulatory cyclin T1 assemble the functional positive transcription elongation factor b (P-TEFb) complex, which phosphorylates the C-terminal domain (CTD) of the largest domain of the multiprotein complex RNA polymerase II (Pol II) RPB1/POLR2A. In turn, Pol II transitions from abortive to productive elongation. Therefore, CDK9 is heavily involved in the regulation of transcription. Other CDK9/cyclin T1 phosphorylation targets include EP300, MYOD1, RPB1/POLR2A, and AR as well as the negative elongation factors DSIF and NELF.

Due to its central role transcriptional regulation, which is frequently dysregulated in cancer, CDK9 has become the target of several drug development efforts. Dysregulation of CDK9 has been observed in a number of solid tumors, including prostate cancer, neuroblastoma, hepatocellular carcinoma, and lymphoma. Moreover, osteosarcoma patients with high CDK9 tumor-expression levels have significantly shorter survival than patients with low CDK9 expression. CDK9 pathway dysregulation has likewise been observed in liquid tumors, such as acute myeloid leukemia (AML) and acute lymphoblastic leukemia (ALL). Even though several CDK9 inhibitors are available, CDK9 is difficult to therapeutically inhibit with small molecules since the structure of its catalytic ATP-binding cleft is similar to many other kinases. Therefore, selective inhibition of CDK9 is challenging.

Recently, a new therapeutic strategy to reduce and/or eliminate proteins associated with certain pathological states, PROTAC (proteolysis targeting chimeras; e.g., see U.S. Patent Application, U.S. Ser. No. 14/792,414, filed Jul. 6, 2015), was developed. PROTACs are heterobifunctional molecules containing two small molecule binding moieties, joined together by a linker. One of the small molecule ligands is designed to bind with high affinity to a target protein in the cell while the other ligand is able to bind with high affinity to an E3 ligase. In the cell, the PROTAC selectively binds to the target protein of interest. The PROTAC then recruits a specific E3 ligase to the target protein to form a ternary complex with both the target protein and the E3 ligase held in close proximity. The E3 ligase then recruits an E2 conjugating enzyme to the ternary complex. The E2 is then able to ubiquitinate the target protein, labelling an available lysine residue on the protein, and then the E2 dissociates from the ternary complex. The E3 ligase can then recruit additional E2 molecules resulting in poly-ubiquitination of the target protein, labelling the target protein for degradation by the cell's proteasome machinery. The PROTAC can then dissociate from the target protein and initiate another catalytic cycle. The poly-ubiquitinated target protein is recognized and degraded by the proteasome.

### SUMMARY

Because kinases such as CDK9 are difficult to target via traditional small molecule inhibition, compounds that can take advantage of cellular machinery involved in protein homeostasis (e.g., ubiquitination and proteasome degradation via PROTAC) may be advantageous therapeutic agents in targeting CDK9. The present disclosure describes the conjugation of a CDK9 binding moiety with an E3 ubiquitin ligase binding moiety (e.g., pomalidomide) to provide compounds that can induce the ubiquitination of CDK9 and promote its degradation in cells. Accordingly, the present disclosure provides new compounds, compositions, kits, uses, and methods for the treatment of cancer (e.g., prostate cancer).

In one aspect, provided herein are compounds of Formula (I):

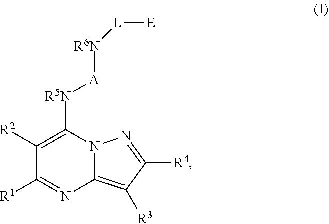

or a pharmaceutically acceptable salt, solvate, hydrate, polymorph, co-crystal, tautomer, stereoisomer, isotopically labeled derivative, or prodrug thereof, wherein:

each of R¹, R², R³, and R⁴ is independently hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, substituted or unsubstituted heteroalkyl, —N(R^{A})₂, —OR^{A}, —SR^{A}, —C(═O)OR^{A}, —C(═O)N(R^{A})₂, —NR^{A}C(═O)R^{A}, —C(═O)R^{A}, —NR^{A}C(═O)OR^{A}, —NR^{A}C(═O)N(R^{A})₂, —OC(═O)R^{A}, —OC(═O)OR^{A}, —OC(═O)N(R^{A})₂, —S(O)₂N(R^{A})₂, or —NR^{A}S(O)₂R^{A};

each of R⁵ and R⁶ is independently hydrogen, substituted or unsubstituted alkyl, —C(═O)R^{A}, or a nitrogen protecting group;

A is substituted or unsubstituted carbocyclylene, or substituted or unsubstituted heterocyclylene;

E is an E3 ligase binding moiety;

L is a bond, substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, —S(O)₂NR^{A}—, —NR^{A}S(O)₂—, or a combination thereof; and

each occurrence of R^{A} is, independently, hydrogen, substituted or unsubstituted acyl, substituted or unsubstituted alkyl, substituted or unsubstituted alkenyl, substituted or unsubstituted alkynyl, substituted or unsubstituted heteroalkyl, substituted or unsubstituted carbocyclyl, substituted or unsubstituted heterocyclyl, substituted or unsubstituted aryl, substituted or unsubstituted heteroaryl, an oxygen protecting group when attached to an oxygen atom, or a nitrogen protecting group when attached to a nitrogen atom, or two R^{A} groups are joined to form a substituted or unsubstituted heterocyclic ring.

In certain embodiments, the compound of Formula (I) is of Formula (I-a), (I-b), (I-c), (I-d), (I-e), (I-f), (I-g), (I-h), (I-i), (I-j), or (I-k):

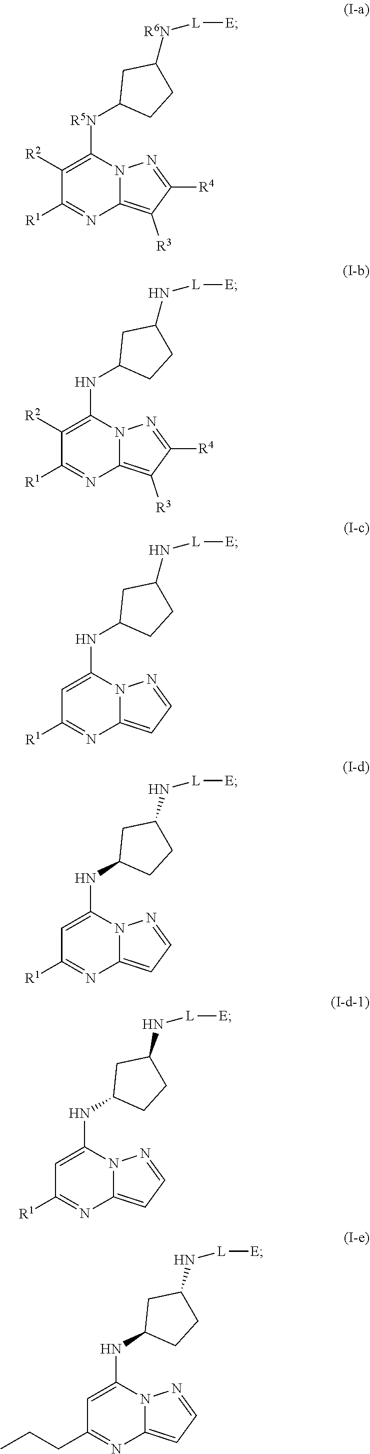

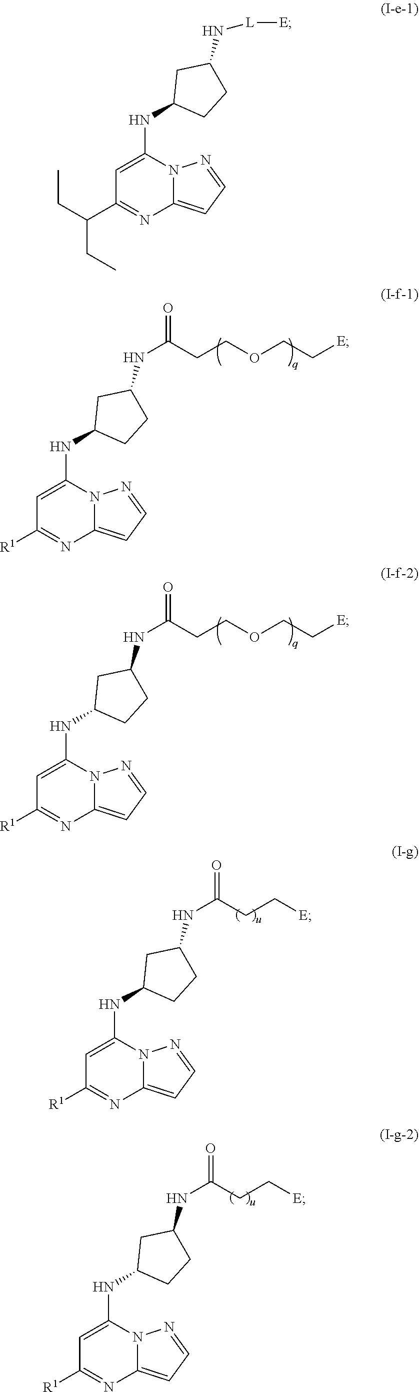

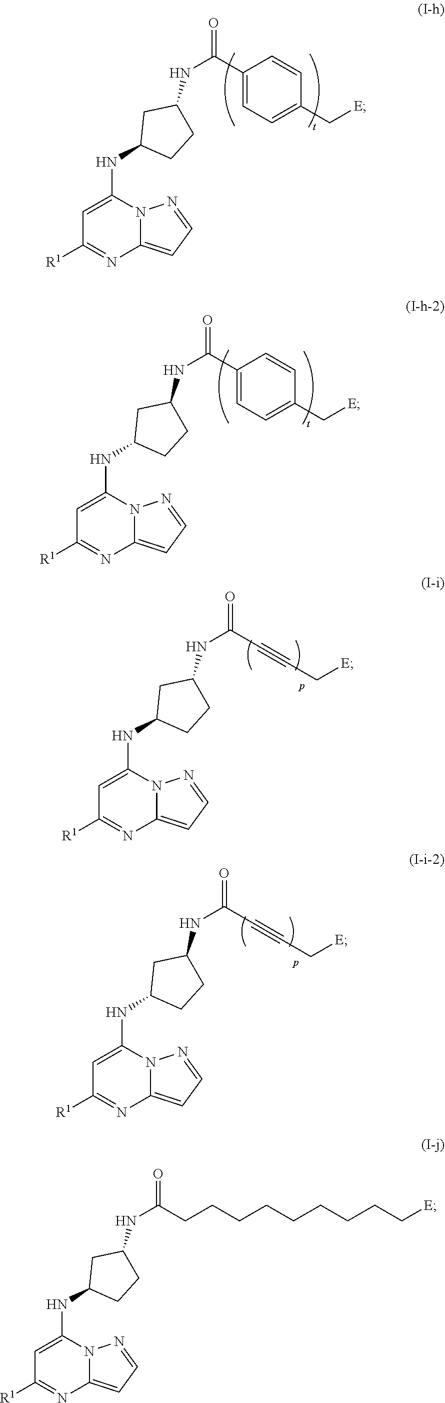

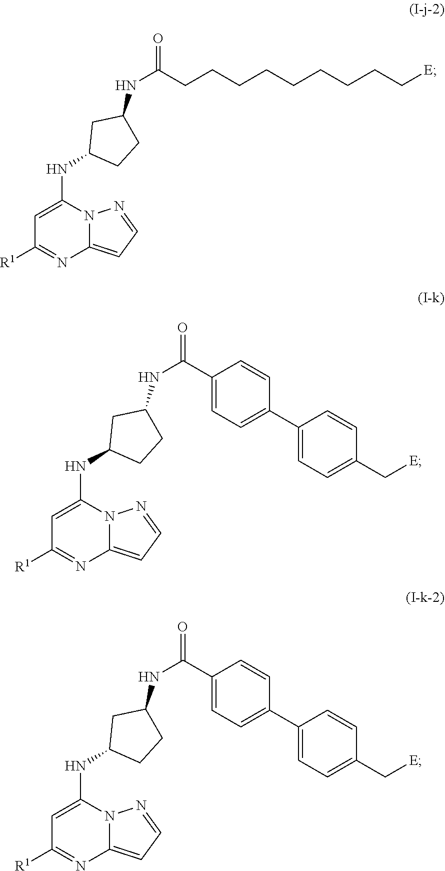

or a pharmaceutically acceptable salt, solvate, hydrate, polymorph, co-crystal, tautomer, stereoisomer, isotopically labeled derivative, or prodrug thereof.

Exemplary compounds of Formula (I) include, but are not limited to:

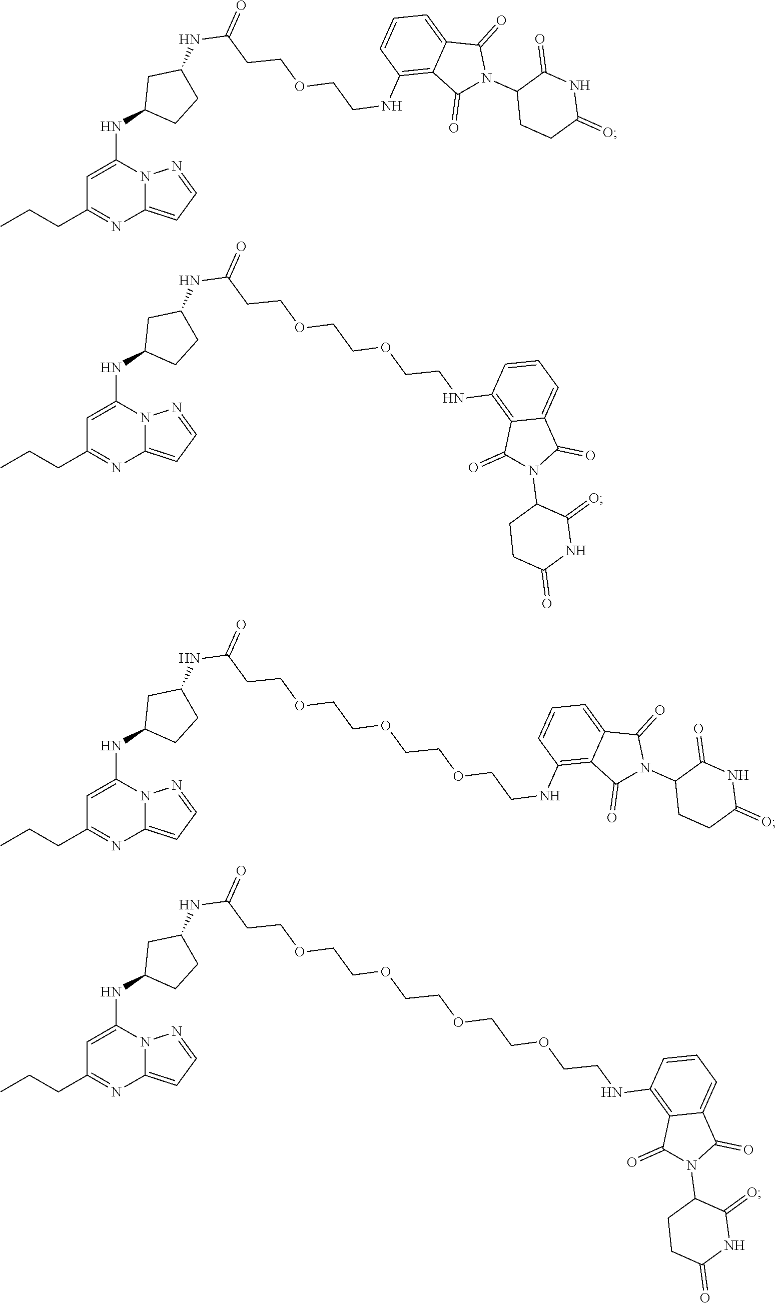 [SMILES: CCCc1cc(N[C@@H]2CC[C@@H](NC(=O)CCOCCNc3cccc4c3C(=O)N(C3CCC(=O)NC3=O)C4=O)C2)n2nccc2n1 ; CCCc1cc(N[C@@H]2CC[C@@H](NC(=O)CCOCCOCCNc3cccc4c3C(=O)N(C3CCC(=O)NC3=O)C4=O)C2)n2nccc2n1 ; CCCc1cc(N[C@@H]2CC[C@@H](NC(=O)CCOCCOCCOCCNc3cccc4c3C(=O)N(C3CCC(=O)NC3=O)C4=O)C2)n2nccc2n1 ; CCCc1cc(N[C@@H]2CC[C@@H](NC(=O)CCOCCOCCOCCOCCNc3cccc4c3C(=O)N(C3CCC(=O)NC3=O)C4=O)C2)n2nccc2n1]

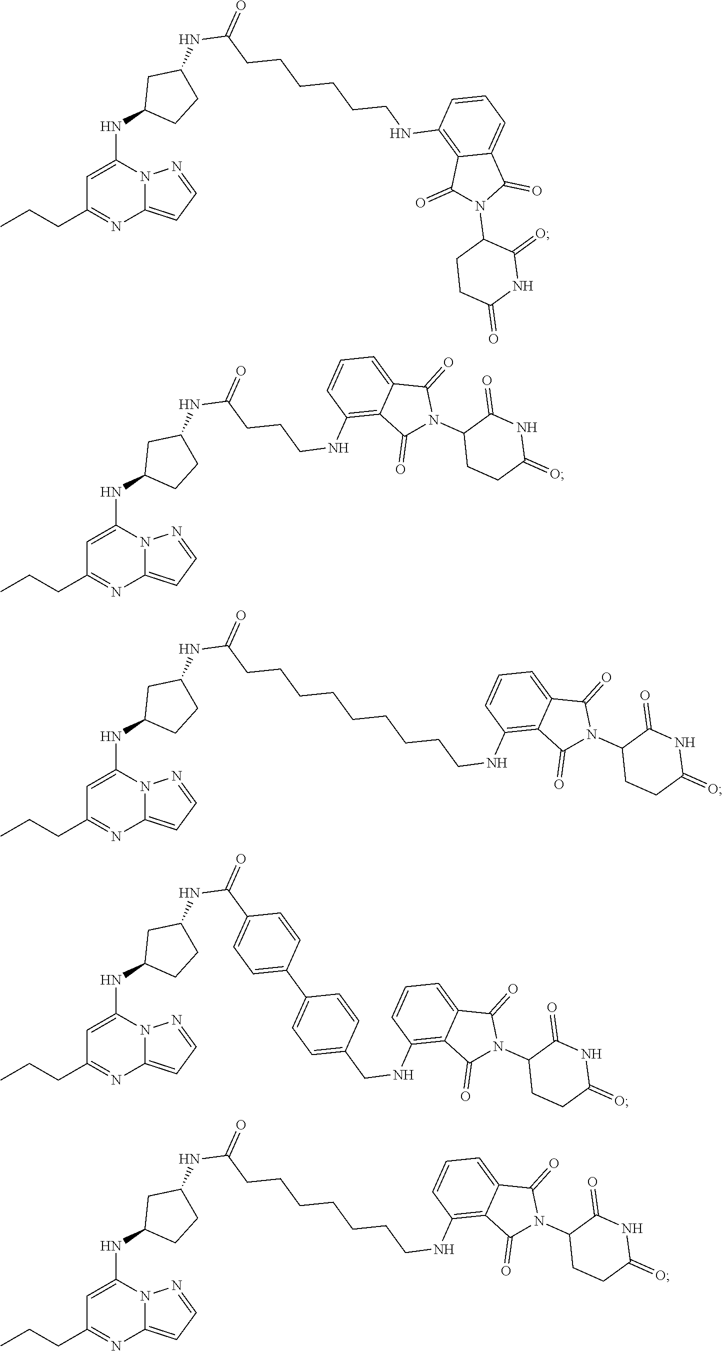

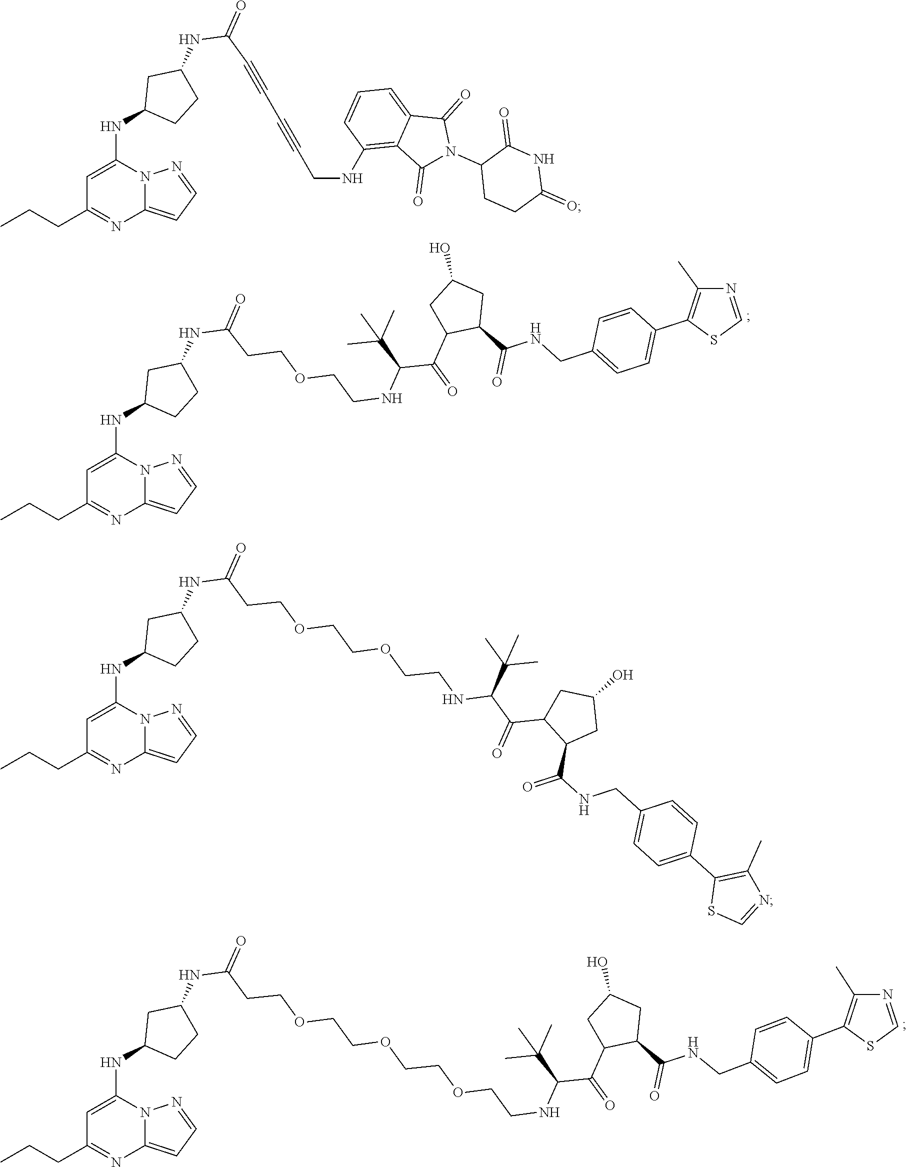 [SMILES: CCCc1cc(N[C@@H]2CC[C@@H](NC(=O)C#CC#CCNc3cccc4c3C(=O)N(C3CCC(=O)NC3=O)C4=O)C2)n2nccc2n1 ; CCCc1cc(N[C@@H]2CC[C@@H](NC(=O)CCOCCN[C@H](C(=O)C3C[C@H](O)C[C@H]3C(=O)NCc3ccc(-c4scnc4C)cc3)C(C)(C)C)C2)n2nccc2n1 ; CCCc1cc(N[C@@H]2CC[C@@H](NC(=O)CCOCCOCCN[C@H](C(=O)C3C[C@H](O)C[C@H]3C(=O)NCc3ccc(-c4scnc4C)cc3)C(C)(C)C)C2)n2nccc2n1 ; CCCc1cc(N[C@@H]2CC[C@@H](NC(=O)CCOCCOCCOCCN[C@H](C(=O)C3C[C@H](O)C[C@H]3C(=O)NCc3ccc(-c4scnc4C)cc3)C(C)(C)C)C2)n2nccc2n1]

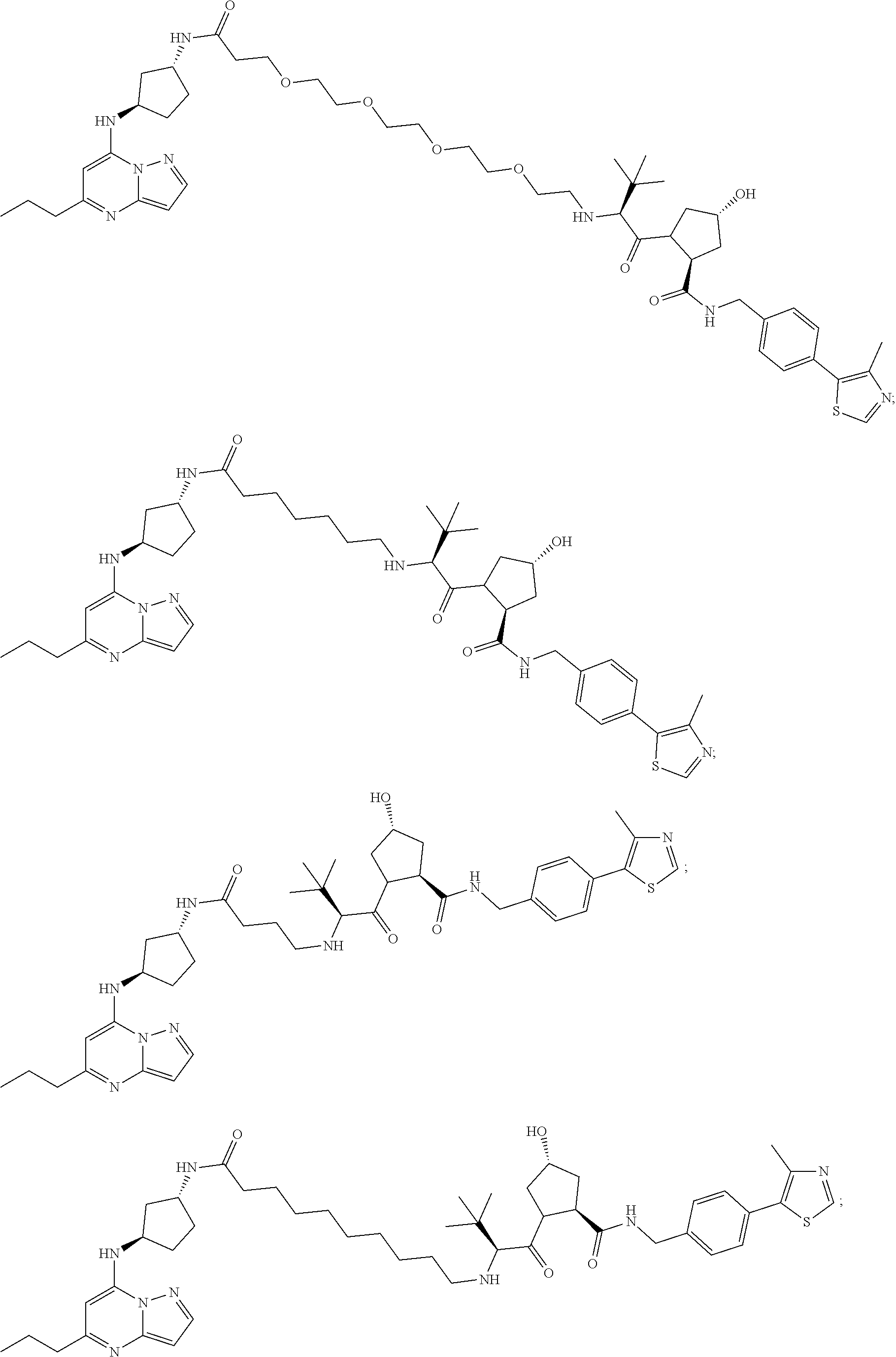 [SMILES: CCCc1cc(N[C@@H]2CC[C@@H](NC(=O)CCOCCOCCOCCOCCN[C@H](C(=O)C3C[C@H](O)C[C@H]3C(=O)NCc3ccc(-c4scnc4C)cc3)C(C)(C)C)C2)n2nccc2n1 ; CCCc1cc(N[C@@H]2CC[C@@H](NC(=O)CCCCCCN[C@H](C(=O)C3C[C@H](O)C[C@H]3C(=O)NCc3ccc(-c4scnc4C)cc3)C(C)(C)C)C2)n2nccc2n1 ; CCCc1cc(N[C@@H]2CC[C@@H](NC(=O)CCCN[C@H](C(=O)C3C[C@H](O)C[C@H]3C(=O)NCc3ccc(-c4scnc4C)cc3)C(C)(C)C)C2)n2nccc2n1 ; CCCc1cc(N[C@@H]2CC[C@@H](NC(=O)CCCCCCCCCN[C@H](C(=O)C3C[C@H](O)C[C@H]3C(=O)NCc3ccc(-c4scnc4C)cc3)C(C)(C)C)C2)n2nccc2n1]

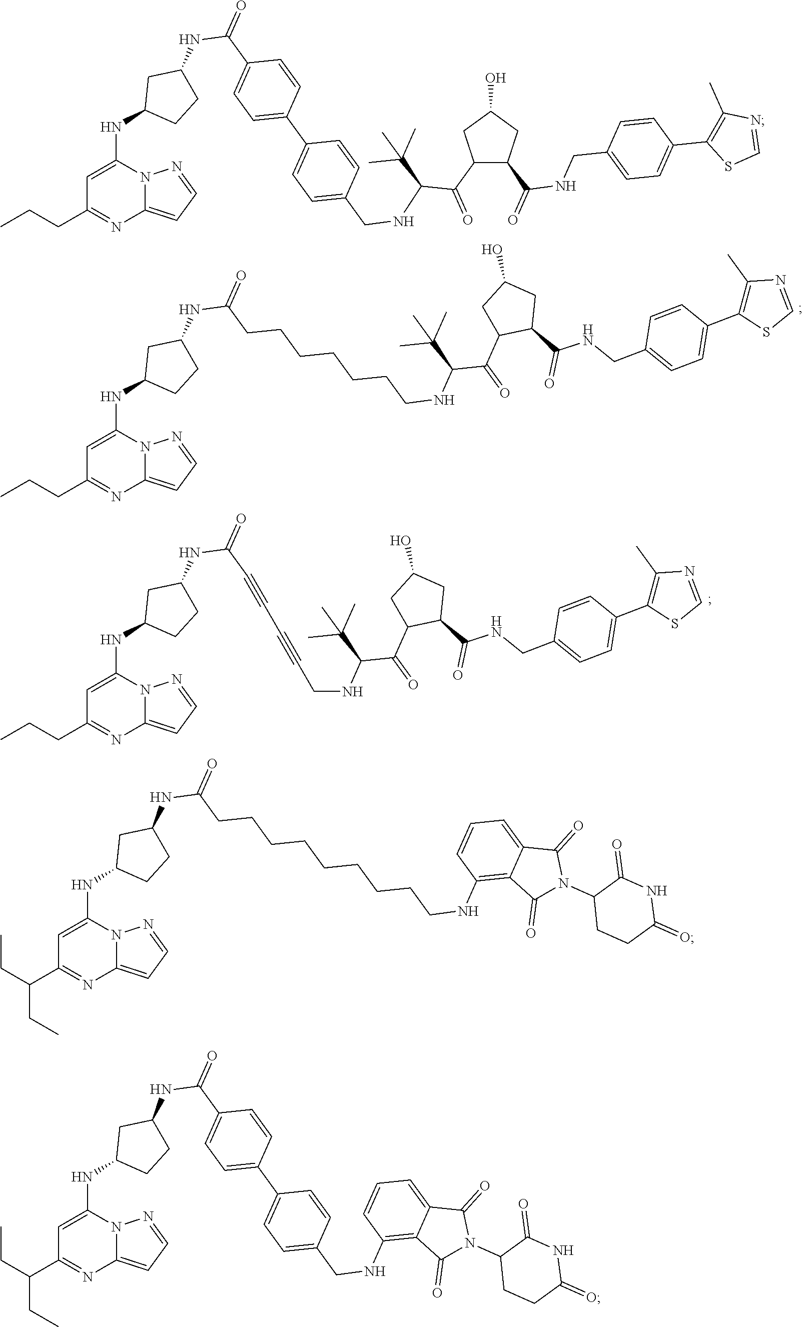

and pharmaceutically acceptable salts, solvates, hydrates, polymorphs, co-crystals, tautomers, stereoisomers, isotopically labeled derivatives, and prodrugs thereof.

In another aspect, provided are compounds of the formula:

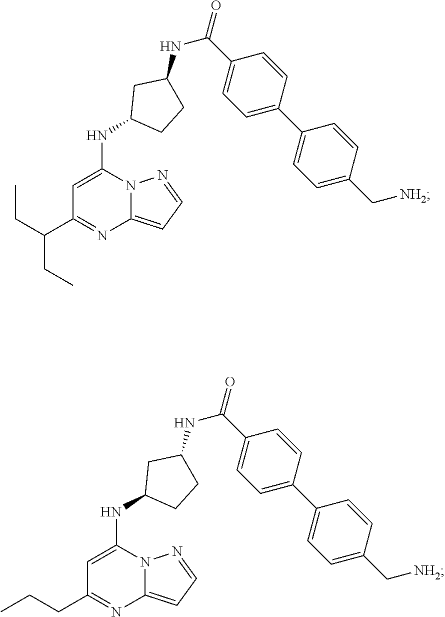 [SMILES: CCC(CC)c1cc(N[C@H]2CC[C@H](NC(=O)c3ccc(-c4ccc(CN)cc4)cc3)C2)n2nccc2n1 ; CCCc1cc(N[C@@H]2CC[C@@H](NC(=O)c3ccc(-c4ccc(CN)cc4)cc3)C2)n2nccc2n1]

and pharmaceutically acceptable salts, solvates, hydrates, polymorphs, co-crystals, tautomers, stereoisomers, isotopically labeled derivatives, and prodrugs thereof.

In another aspect, provided are pharmaceutical compositions comprising a compound of Formula (I), or a pharmaceutically acceptable salt thereof, and a pharmaceutically acceptable excipient.

In another aspect, provided are methods of treating cancer in a subject in need thereof, the method comprising administering a compound of Formula (I), or a pharmaceutically acceptable salt thereof, or a pharmaceutical composition comprising a compound of Formula (I), to the subject. In certain embodiments, the cancer is a solid tumor or a hematological cancer. In certain embodiments, the cancer is a leukemia or a lymphoma. In certain embodiments, the cancer is acute myeloid leukemia (AML) or acute lymphoblastic leukemia (ALL).

In another aspect, provided are methods of promoting the degradation of cyclin-dependent kinase 9 (CDK9) in a subject in need thereof, the method comprising administering a compound of Formula (I), or a pharmaceutically acceptable salt thereof, or a pharmaceutical composition comprising a compound of Formula (I), to the subject.

In another aspect, provided are methods of promoting the ubiquitination of cyclin-dependent kinase 9 (CDK9) by an E3 ubiquitin ligase in a subject in need thereof, the method comprising contacting a compound of Formula (I), or a pharmaceutically acceptable salt thereof, or a pharmaceutical composition comprising a compound of Formula (I), with a tissue. In certain embodiments, the E3 ubiquitin ligase is Cereblon or von Hippel-Lindau tumor suppressor (VHL).

In another aspect, provided are kits comprising a compound of Formula (I), or a pharmaceutically acceptable salt thereof, or a pharmaceutical composition comprising a compound of Formula (I). In certain embodiments, the kit further comprises instructions for administration (e.g., human administration) and/or use.

The details of certain embodiments of the invention are set forth in the Detailed Description of Certain Embodiments, as described below. Other features, objects, and advantages of the invention will be apparent from the Definitions, Examples, Figures, and Claims.

### BRIEF DESCRIPTION OF THE DRAWINGS

FIGS. 1A-C show a kinase selectivity panel with IC₅₀ determination in presence of varying concentrations of ATP as well target engagement studies. FIG. 1A shows the chemical structure of KI-ARv3 and its analysis in a kinase selectivity panel (Eurofins) using 10 μM of compound tested against 413 individual kinases with ATP concentrations at their K_{M} app. FIG. 1B is a graph showing dose-dependent assessment of the inhibitory activity of KI-ARv3 towards CDK9 in the presence of different ATP concentrations. IC₅₀ shifts indicate that KI-ARv3 is an ATP-competitive inhibitor. FIG. 1C shows AffiGel bead-based target engagement studies of KI-ARv3 in LNCaP cell lysates analyzed by western blot. Lane 1: Ladder; Lane 2: 30 μg LNCaP lysate; Lane 3: blocked beads; Lane 4: KI-ARv3 modified beads; Lane 5: 4+10 μM KI-ARv3; Lane 6: 4+20 μM KI-ARv3; Lane 6: 4+40 μM KI-ARv3.

FIG. 2 is a western blot showing the ability of compounds KI-ARv3-D01 through -D08 to degrade CDK9 in MOLT4 cells (human T lymphoblast; ALL) at 1 μM compound concentration.

FIG. 3 is a western blot showing the ability of compounds KI-ARv3-D02, KI-ARv3-D07, and KI-ARv3-D08 to degrade CDK9 in MOLT4 cells after 6 hours and 24 hours treatment with low dose (1 μM) and high dose (5 μM) of each compound.

FIG. 4 is a western blot showing the ability of compounds KI-ARv3-D07 and KI-ARv3-D08 to degrade CDK9 in MOLT4 cells after 6 hours treatment with a range of doses (10 nM, 100 nM, 500 nM, 1 μM, 10 μM). Rescue experiments with proteasome inhibitor MG132 and parent compound KI-ARv3 were performed at 1 μM compound concentration.

FIG. 5 is a western blot showing the ability of compounds KI-ARv3-D07 and KI-ARv3-D08 to degrade CDK9 in MOLT4 cells after 24 hours treatment with a range of doses (10 nM, 100 nM, 500 nM, 1 μM, 10 μM). Rescue experiments with parent compound KI-ARv3 were performed at 1 μM compound concentration.

FIG. 6 is a western blot showing the ability of compounds KI-ARv3-D07 and KI-ARv3-D08 to degrade CDK9 in 22Rv1 cells after 6 hours treatment with a range of doses (10 nM, 100 nM, 500 nM, 1 μM, 10 μM). Rescue experiments with proteasome inhibitor MG132 and parent compound KI-ARv3 were performed at 1 μM compound concentration.

FIG. 7 is a western blot showing the ability of compounds KI-ARv3-D07 and KI-ARv3-D08 to degrade CDK9 in 22Rv1 cells after 24 hours treatment with a range of doses (10 nM, 100 nM, 500 nM, 1 μM, 10 μM). Rescue experiments with parent compound KI-ARv3 were performed at 1 μM compound concentration.

FIGS. 8A-C show evaluation of KI-ARv3-D07 and -08 in MV-4-11 (FIG. 8A) and Rh-30 (FIG. 8C) cells as well as side by side comparison of both cell lines (FIG. 8B). FIGS. 8A and 8C show single treatments: 10 nM, 100 nM, 500 nM, 1 μM, and 10 μM; and co-treatments with MG132 or KI-ARv3 at 500 nM degrader concentration and KI-ARv3 at 5 μM (single treatment and co-treatment), MG132 at 5 μM (co-treatment only), treatment time: 6 hr. FIG. 8B treatment time is 8 hr.

FIGS. 9A-D show evaluation of KI-ARv3-D11 and -D18 in 22Rv1 (FIG. 9A, 16 h treatment time), MOLT-4 (FIG. 9B, 7 h treatment time), and Rh-30 (FIGS. 9C and 9D, 16 h treatment time) cell lines.

FIG. 10 shows evaluation of KI-ARv3-D10 and KB-0742-D02 in MOLT-4 cell line (8 h treatment time).

### DEFINITIONS

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H., Tables of Resolving Agents and Optical Resolutions p. 268 (E.L. Eliel, Ed., Univ. of Notre Dame Press, Notre Dame, IN 1972). The invention additionally encompasses compounds as individual isomers substantially free of other isomers, and alternatively, as mixtures of various isomers.

In a formula, [symbol] is a single bond where the stereochemistry of the moieties immediately attached thereto is not specified, [symbol] is absent or a single bond, and [symbol] or [symbol] is a single or double bond.

Unless otherwise stated, structures depicted herein are also meant to include compounds that differ only in the presence of one or more isotopically enriched atoms. For example, compounds having the present structures except for the replacement of hydrogen by deuterium or tritium, replacement of ¹⁹F with ¹⁸F, or the replacement of ¹²C with ¹³C or ¹⁴C are within the scope of the disclosure. Such compounds are useful, for example, as analytical tools or probes in biological assays.

When a range of values is listed, it is intended to encompass each value and sub-range within the range. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of a straight-chain or branched saturated hydrocarbon group having from 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, iso-butyl), pentyl (C₅) (e.g., n-pentyl, 3-pentanyl, amyl, neopentyl, 3-methyl-2-butanyl, tertiary amyl), and hexyl (C₆) (e.g., n-hexyl). Additional examples of alkyl groups include n-heptyl (C₇), n-octyl (C₈), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₀ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu), unsubstituted isobutyl (i-Bu)). In certain embodiments, the alkyl group is a substituted C₁₋₁₀ alkyl (such as substituted C₁₋₆ alkyl, e.g., —CF₃, Bn).

The term “haloalkyl” is a substituted alkyl group, wherein one or more of the hydrogen atoms are independently replaced by a halogen, e.g., fluoro, bromo, chloro, or iodo. In some embodiments, the haloalkyl moiety has 1 to 8 carbon atoms (“C₁₋₈ haloalkyl”). In some embodiments, the haloalkyl moiety has 1 to 6 carbon atoms (“C₁₋₆ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). Examples of haloalkyl groups include —CHF₂, —CH₂F, —CF₃, —CH₂CF₃, —CF₂CF₃, —CF₂CF₂CF₃, —CCl₃, —CFCl₂, —CF₂Cl, and the like.

The term “heteroalkyl” refers to an alkyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkyl group refers to a saturated group having from 1 to 20 carbon atoms and 1 or more heteroatoms within the parent chain (“heteroC₁₋₂₀ alkyl”). In some embodiments, a heteroalkyl group is a saturated group having 1 to 18 carbon atoms and 1 or more heteroatoms within the parent chain (“heteroC₁₋₁₈ alkyl”). In some embodiments, a heteroalkyl group is a saturated group having 1 to 16 carbon atoms and 1 or more heteroatoms within the parent chain (“heteroC₁₋₁₆ alkyl”). In some embodiments, a heteroalkyl group is a saturated group having 1 to 14 carbon atoms and 1 or more heteroatoms within the parent chain (“heteroC₁₋₁₄ alkyl”). In some embodiments, a heteroalkyl group is a saturated group having 1 to 12 carbon atoms and 1 or more heteroatoms within the parent chain (“heteroC₁₋₁₂ alkyl”). In some embodiments, a heteroalkyl group is a saturated group having 1 to 10 carbon atoms and 1 or more heteroatoms within the parent chain (“heteroC₁₋₁₀ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 4 carbon atoms and 1 or 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, the heteroalkyl group defined herein is a partially unsaturated group having 1 or more heteroatoms within the parent chain and at least one unsaturated carbon, such as a carbonyl group. For example, a heteroalkyl group may comprise an amide or ester functionality in its parent chain such that one or more carbon atoms are unsaturated carbonyl groups. Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₂₀ alkyl. In certain embodiments, the heteroalkyl group is an unsubstituted heteroC₁₋₁₀ alkyl. In certain embodiments, the heteroalkyl group is a substituted heteroC₁₋₂₀ alkyl. In certain embodiments, the heteroalkyl group is an unsubstituted heteroC₁₋₁₀ alkyl.

The term “alkenyl” refers to a radical of a straight-chain or branched hydrocarbon group having from 2 to 10 carbon atoms and one or more carbon-carbon double bonds (e.g., 1, 2, 3, or 4 double bonds). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₂₋₁₀ alkenyl. In certain embodiments, the alkenyl group is a substituted C₂₋₁₀ alkenyl. In an alkenyl group, a C═C double bond for which the stereochemistry is not specified (e.g., —CH═CHCH₃ or

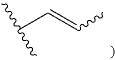

may be an (E)- or (Z)- double bond.

The term “heteroalkenyl” refers to an alkenyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkenyl group refers to a group having from 2 to 10 carbon atoms, at least one double bond, and 1 or more heteroatoms within the parent chain (“heteroC₂₋₁₀ alkenyl”). In some embodiments, a heteroalkenyl group has 2 to 9 carbon atoms at least one double bond, and 1 or more heteroatoms within the parent chain (“heteroC₂₋₉ alkenyl”). In some embodiments, a heteroalkenyl group has 2 to 8 carbon atoms, at least one double bond, and 1 or more heteroatoms within the parent chain (“heteroC₂₋₈ alkenyl”). In some embodiments, a heteroalkenyl group has 2 to 7 carbon atoms, at least one double bond, and 1 or more heteroatoms within the parent chain (“heteroC₂₋₇ alkenyl”). In some embodiments, a heteroalkenyl group has 2 to 6 carbon atoms, at least one double bond, and 1 or more heteroatoms within the parent chain (“heteroC₂₋₆ alkenyl”). In some embodiments, a heteroalkenyl group has 2 to 5 carbon atoms, at least one double bond, and 1 or 2 heteroatoms within the parent chain (“heteroC₂₋₅ alkenyl”). In some embodiments, a heteroalkenyl group has 2 to 4 carbon atoms, at least one double bond, and 1 or 2 heteroatoms within the parent chain (“heteroC₂₋₄ alkenyl”). In some embodiments, a heteroalkenyl group has 2 to 3 carbon atoms, at least one double bond, and 1 heteroatom within the parent chain (“heteroC₂₋₃ alkenyl”). In some embodiments, a heteroalkenyl group has 2 to 6 carbon atoms, at least one double bond, and 1 or 2 heteroatoms within the parent chain (“heteroC₂₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₂₋₁₀ alkenyl. In certain embodiments, the heteroalkenyl group is a substituted heteroC₂₋₁₀ alkenyl.

The term “alkynyl” refers to a radical of a straight-chain or branched hydrocarbon group having from 2 to 10 carbon atoms and one or more carbon-carbon triple bonds (e.g., 1, 2, 3, or 4 triple bonds) (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₂₋₁₀ alkynyl. In certain embodiments, the alkynyl group is a substituted C₂₋₁₀ alkynyl.

The term “heteroalkynyl” refers to an alkynyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkynyl group refers to a group having from 2 to 10 carbon atoms, at least one triple bond, and 1 or more heteroatoms within the parent chain (“heteroC₂₋₁₀ alkynyl”). In some embodiments, a heteroalkynyl group has 2 to 9 carbon atoms, at least one triple bond, and 1 or more heteroatoms within the parent chain (“heteroC₂₋₉ alkynyl”). In some embodiments, a heteroalkynyl group has 2 to 8 carbon atoms, at least one triple bond, and 1 or more heteroatoms within the parent chain (“heteroC₂₋₈ alkynyl”). In some embodiments, a heteroalkynyl group has 2 to 7 carbon atoms, at least one triple bond, and 1 or more heteroatoms within the parent chain (“heteroC₂₋₇ alkynyl”). In some embodiments, a heteroalkynyl group has 2 to 6 carbon atoms, at least one triple bond, and 1 or more heteroatoms within the parent chain (“heteroC₂₋₆ alkynyl”). In some embodiments, a heteroalkynyl group has 2 to 5 carbon atoms, at least one triple bond, and 1 or 2 heteroatoms within the parent chain (“heteroC₂₋₅ alkynyl”). In some embodiments, a heteroalkynyl group has 2 to 4 carbon atoms, at least one triple bond, and 1 or 2 heteroatoms within the parent chain (“heteroC₂₋₄ alkynyl”). In some embodiments, a heteroalkynyl group has 2 to 3 carbon atoms, at least one triple bond, and 1 heteroatom within the parent chain (“heteroC₂₋₃ alkynyl”). In some embodiments, a heteroalkynyl group has 2 to 6 carbon atoms, at least one triple bond, and 1 or 2 heteroatoms within the parent chain (“heteroC₂₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₂₋₁₀ alkynyl. In certain embodiments, the heteroalkynyl group is a substituted heteroC₂₋₁₀ alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include, without limitation, azirdinyl, oxiranyl, and thiiranyl. Exemplary 4-membered heterocyclyl groups containing 1 heteroatom include, without limitation, azetidinyl, oxetanyl, and thietanyl. Exemplary 5-membered heterocyclyl groups containing 1 heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include, without limitation, dioxolanyl, oxathiolanyl and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered heterocyclyl groups containing 2 heteroatoms include, without limitation, piperazinyl, morpholinyl, dithianyl, and dioxanyl. Exemplary 6-membered heterocyclyl groups containing 3 heteroatoms include, without limitation, triazinyl. Exemplary 7-membered heterocyclyl groups containing 1 heteroatom include, without limitation, azepanyl, oxepanyl and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

“Aralkyl” is a subset of “alkyl” and refers to an alkyl group substituted by an aryl group, wherein the point of attachment is on the alkyl moiety.

The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include, without limitation, pyrrolyl, furanyl, and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl, and phenazinyl.

“Heteroaralkyl” is a subset of “alkyl” and refers to an alkyl group substituted by a heteroaryl group, wherein the point of attachment is on the alkyl moiety.

The term “unsaturated bond” refers to a double or triple bond.

The term “unsaturated” or “partially unsaturated” refers to a moiety that includes at least one double or triple bond.

The term “saturated” refers to a moiety that does not contain a double or triple bond, i.e., the moiety only contains single bonds.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which may be substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, and includes any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety. The invention is not intended to be limited in any manner by the exemplary substituents described herein.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₃, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;

or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};

each instance of R^{aa} is, independently, selected from C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;

each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S; wherein X⁻ is a counterion;

each instance of R^{ee} is, independently, selected from C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;

each instance of F^{ff} is, independently, selected from hydrogen, C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, 3-10 membered heterocyclyl, C₆₋₁₀ aryl and 5-10 membered heteroaryl, or two e groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and

each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), -00O2(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(═NH)NH(C₁₋₆ alkyl), —OC(═NH)NH₂, —NHC(═NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂(C₁₋₆ alkyl), —SO₂O(C₁₋₆ alkyl), —OSO₂(C₁₋₆ alkyl), —SO(C₁₋₆ alkyl), —Si(C₁₋₆ alkyl)₃, —OSi(C₁₋₆ alkyl)₃ —C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion.

The term “halo” or “halogen” refers to fluorine (fluoro, —F), chlorine (chloro, —Cl), bromine (bromo, —Br), or iodine (iodo, —I).

The term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R^{cc})₃⁺X⁻, —OP(OR^{cc})₂, —OP(OR^{cc})₃⁺X⁻, —OP(═O)(R^{aa})₂, —OP(═O)(OR^{cc})₂, and —OP(═O)(N(R^{bb})₂)₂, wherein X⁻, R^{aa}, R^{bb}, and R^{cc} are as defined herein.

The term “amino” refers to the group —NH₂. The term “substituted amino,” by extension, refers to a monosubstituted amino, a disubstituted amino, or a trisubstituted amino. In certain embodiments, the “substituted amino” is a monosubstituted amino or a disubstituted amino group.

The term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, and —NHP(═O)(N(R^{bb})₂)₂, wherein R^{aa}, R^{bb} and R^{cc} are as defined herein, and wherein R^{bb} of the group —NH(R^{bb}) is not hydrogen.

The term “disubstituted amino” refers to an amino group wherein the nitrogen atom directly attached to the parent molecule is substituted with two groups other than hydrogen, and includes groups selected from —N(R^{bb})₂, —NR^{bb}C(═O)R^{aa}, —NR^{bb}CO₂R^{aa}, —NR^{bb}C(═O)N(R^{bb})₂, —NR^{bb}C(═NR^{bb})N(R^{bb})₂, —NR^{bb}SO₂R^{aa}, —NR^{bb}P(═O)(OR^{cc})₂, and —NR^{bb}P(═O)(N(R^{bb})₂)₂, wherein R^{aa}, R^{bb}, and R^{cc} are as defined herein, with the proviso that the nitrogen atom directly attached to the parent molecule is not substituted with hydrogen.

The term “trisubstituted amino” refers to an amino group wherein the nitrogen atom directly attached to the parent molecule is substituted with three groups, and includes groups selected from —N(R^{bb})₃ and —N(R^{bb})₃⁺X⁻, wherein R^{bb} and X⁻ are as defined herein.

The term “acyl” refers to a group having the general formula —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, —C(═S)O(R^{X1}), —C(═S)S(R^{X1}), —C(═NR^{X1})R^{X1}, —C(═NR^{X1})OR^{X1}, —C(═NR^{X1})SR^{X1}, and —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “carbonyl” refers a group wherein the carbon directly attached to the parent molecule is sp² hybridized, and is substituted with an oxygen, nitrogen or sulfur atom, e.g., a group selected from ketones (e.g., —C(═O)R^{aa}), carboxylic acids (e.g., —CO₂H), aldehydes (—CHO), esters (e.g., —CO₂R^{aa}, —C(═O)SR^{aa}, —C(═S)SR^{aa}), amides (e.g., —C(═O)N(R^{bb})₂, —C(═O)NR^{bb}SO₂R^{aa}, —C(═S)N(R^{bb})₂), and imines (e.g., —C(═NR^{bb})R^{aa}, —C(═NR^{bb})OR^{aa}), —C(═NR^{bb})N(R^{bb})₂), wherein R^{aa} and R^{bb} are as defined herein.

The term “silyl” refers to the group —Si(R^{aa})₃, wherein R^{aa} is as defined herein.

The term “oxo” refers to the group ═O, and the term “thiooxo” refers to the group ═S.

Nitrogen atoms can be substituted or unsubstituted as valency permits, and include primary, secondary, tertiary, and quaternary nitrogen atoms. Exemplary nitrogen atom substituents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —S OR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc}, and R^{dd} are as defined herein.

In certain embodiments, the substituent present on the nitrogen atom is an nitrogen protecting group (also referred to herein as an “amino protecting group”). Nitrogen protecting groups include, but are not limited to, —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, nitrogen protecting groups such as amide groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, and o-(benzoyloxymethyl)benzamide.

Nitrogen protecting groups such as carbamate groups (e.g., —C(═O)OR^{aa}) include, but are not limited to, methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Nitrogen protecting groups such as sulfonamide groups (e.g., —S(═O)₂R^{aa}) include, but are not limited to, p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STAB ASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N[2-(trimethylsilyl)ethoxylmethylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N-(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N4phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys). In certain embodiments, a nitrogen protecting group is benzyl (Bn), tert-butyloxycarbonyl (BOC), carbobenzyloxy (Cbz), 9-flurenylmethyloxycarbonyl (Fmoc), trifluoroacetyl, triphenylmethyl, acetyl (Ac), benzoyl (Bz), p-methoxybenzyl (PMB), 3,4-dimethoxybenzyl (DMPM), p-methoxyphenyl (PMP), 2,2,2-trichloroethyloxycarbonyl (Troc), triphenylmethyl (Tr), tosyl (Ts), brosyl (Bs), nosyl (Ns), mesyl (Ms), triflyl (Tf), or dansyl (Ds).

In certain embodiments, the substituent present on an oxygen atom is an oxygen protecting group (also referred to herein as an “hydroxyl protecting group”). Oxygen protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})R^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include, but are not limited to, methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TB MPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro (1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). In certain embodiments, an oxygen protecting group is silyl. In certain embodiments, an oxygen protecting group is t-butyldiphenylsilyl (TBDPS), t-butyldimethylsilyl (TBDMS), triisoproylsilyl (TIPS), triphenylsilyl (TPS), triethylsilyl (TES), trimethylsilyl (TMS), triisopropylsiloxymethyl (TOM), acetyl (Ac), benzoyl (Bz), allyl carbonate, 2,2,2-trichloroethyl carbonate (Troc), 2-trimethylsilylethyl carbonate, methoxymethyl (MOM), 1-ethoxyethyl (EE), 2-methyoxy-2-propyl (MOP), 2,2,2-trichloroethoxyethyl, 2-methoxyethoxymethyl (MEM), 2-trimethylsilylethoxymethyl (SEM), methylthiomethyl (MTM), tetrahydropyranyl (THP), tetrahydrofuranyl (THF), p-methoxyphenyl (PMP), triphenylmethyl (Tr), methoxytrityl (MMT), dimethoxytrityl (DMT), allyl, p-methoxybenzyl (PMB), t-butyl, benzyl (Bn), allyl, or pivaloyl (Piv).

In certain embodiments, the substituent present on a sulfur atom is a sulfur protecting group (also referred to as a “thiol protecting group”). Sulfur protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference. In certain embodiments, a sulfur protecting group is acetamidomethyl, t-Bu, 3-nitro-2-pyridine sulfenyl, 2-pyridine-sulfenyl, or triphenylmethyl.

A “counterion” or “anionic counterion” is a negatively charged group associated with a positively charged group in order to maintain electronic neutrality. An anionic counterion may be monovalent (i.e., including one formal negative charge). An anionic counterion may also be multivalent (i.e., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methansulfonate, trifluoromethanesulfonate, p—toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄⁻, BPh₄⁻, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

The term “leaving group” is given its ordinary meaning in the art of synthetic organic chemistry and refers to an atom or a group capable of being displaced by a nucleophile. See, for example, Smith, March's Advanced Organic Chemistry 6th ed. (501-502). Examples of suitable leaving groups include, but are not limited to, halogen (such as F, Cl, Br, or I (iodine)), alkoxycarbonyloxy, aryloxycarbonyloxy, alkanesulfonyloxy, arenesulfonyloxy, alkyl-carbonyloxy (e.g., acetoxy), arylcarbonyloxy, aryloxy, methoxy, N,O-dimethylhydroxylamino, pixyl, and haloformates. In some cases, the leaving group is a sulfonic acid ester, such as toluenesulfonate (tosylate, —OTs), methanesulfonate (mesylate, —OMs), p-bromobenzenesulfonyloxy (brosylate, —OBs), —OS(═O)₂(CF₂)₃CF₃ (nonaflate, —ONf), or trifluoromethanesulfonate (triflate, —OTf). In some cases, the leaving group is a brosylate, such as p-bromobenzenesulfonyloxy. In some cases, the leaving group is a nosylate, such as 2-nitrobenzenesulfonyloxy. The leaving group may also be a phosphineoxide (e.g., formed during a Mitsunobu reaction) or an internal leaving group such as an epoxide or cyclic sulfate. Other non-limiting examples of leaving groups are water, ammonia, alcohols, ether moieties, thioether moieties, zinc halides, magnesium moieties, diazonium salts, and copper moieties. Further exemplary leaving groups include, but are not limited to, halo (e.g., chloro, bromo, iodo) and activated substituted hydroxyl groups (e.g., —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OP(R^{cc})₂, —OP(R^{cc})₃, —OP(═O)₂R^{aa}, —OP(═O)(R^{aa})₂, —OP(═O)(OR^{cc})₂, —OP(═O)₂N(R^{bb})₂, and —OP(═O)(NR^{bb})₂, wherein R^{aa}, R^{bb}, and R^{cc} are as defined herein).

As used herein, use of the phrase “at least one instance” refers to 1, 2, 3, 4, or more instances, but also encompasses a range, e.g., for example, from 1 to 4, from 1 to 3, from 1 to 2, from 2 to 4, from 2 to 3, or from 3 to 4 instances, inclusive.

A “non-hydrogen group” refers to any group that is defined for a particular variable that is not hydrogen.

These and other exemplary substituents are described in more detail in the Detailed Description, Examples, and claims. The invention is not intended to be limited in any manner by the above exemplary listing of substituents.

### Other Definitions

The following definitions are more general terms used throughout the present application.

As used herein, the term “salt” refers to any and all salts, and encompasses pharmaceutically acceptable salts.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions, such as halide, hydroxide, carboxylate, sulfate, phosphate, nitrate, lower alkyl sulfonate, and aryl sulfonate.

The term “solvate” refers to forms of the compound, or a salt thereof, that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates, ethanolates, and methanolates.

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula R.x H₂O, wherein R is the compound, and x is a number greater than 0. A given compound may form more than one type of hydrate, including, e.g., monohydrates (x is 1), lower hydrates (x is a number greater than 0 and smaller than 1, e.g., hemihydrates (R.0.5 H₂O)), and polyhydrates (x is a number greater than 1, e.g., dihydrates (R.2 H₂O) and hexahydrates (R.6 H₂O)).

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The term “polymorph” refers to a crystalline form of a compound (or a salt, hydrate, or solvate thereof). All polymorphs have the same elemental composition. Different crystalline forms usually have different X-ray diffraction patterns, infrared spectra, melting points, density, hardness, crystal shape, optical and electrical properties, stability, and solubility. Recrystallization solvent, rate of crystallization, storage temperature, and other factors may cause one crystal form to dominate. Various polymorphs of a compound can be prepared by crystallization under different conditions.

The term “prodrugs” refers to compounds that have cleavable groups and become by solvolysis or under physiological conditions the compounds described herein, which are pharmaceutically active in vivo. Such examples include, but are not limited to, choline ester derivatives and the like, N-alkylmorpholine esters and the like. Other derivatives of the compounds described herein have activity in both their acid and acid derivative forms, but in the acid sensitive form often offer advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides, and anhydrides derived from acidic groups pendant on the compounds described herein are particular prodrugs. In some cases it is desirable to prepare double ester type prodrugs such as (acyloxy)alkyl esters or ((alkoxycarbonyl)oxy)alkylesters. C₁₋₈ alkyl, C₂₋₈ alkenyl, C₂₋₈ alkynyl, aryl, C₇₋₁₂ substituted aryl, and C₇₋₁₂ arylalkyl esters of the compounds described herein may be preferred.

The terms “composition” and “formulation” are used interchangeably.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease. The subject may also be a plant. In certain embodiments, the plant is a land plant. In certain embodiments, the plant is a non-vascular land plant. In certain embodiments, the plant is a vascular land plant. In certain embodiments, the plant is a seed plant. In certain embodiments, the plant is a cultivated plant. In certain embodiments, the plant is a dicot. In certain embodiments, the plant is a monocot. In certain embodiments, the plant is a flowering plant. In some embodiments, the plant is a cereal plant, e.g., maize, corn, wheat, rice, oat, barley, rye, or millet. In some embodiments, the plant is a legume, e.g., a bean plant, e.g., soybean plant. In some embodiments, the plant is a tree or shrub.

The term “biological sample” refers to any sample including tissue samples (such as tissue sections and needle biopsies of a tissue); cell samples (e.g., cytological smears (such as Pap or blood smears) or samples of cells obtained by microdis section); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

The term “tissue” refers to any biological tissue of a subject (including a group of cells, a body part, or an organ) or a part thereof, including blood and/or lymph vessels, which is the object to which a compound, particle, and/or composition of the invention is delivered. A tissue may be an abnormal or unhealthy tissue, which may need to be treated. A tissue may also be a normal or healthy tissue that is under a higher than normal risk of becoming abnormal or unhealthy, which may need to be prevented. In certain embodiments, the tissue is the central nervous system. In certain embodiments, the tissue is the brain.

The term “administer,” “administering,” or “administration” refers to implanting, absorbing, ingesting, injecting, inhaling, or otherwise introducing a compound described herein, or a composition thereof, in or on a subject.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms). Treatment may also be continued after symptoms have resolved, for example, to delay or prevent recurrence.

The terms “condition,” “disease,” and “disorder” are used interchangeably.

An “effective amount” of a compound described herein refers to an amount sufficient to elicit the desired biological response. An effective amount of a compound described herein may vary depending on such factors as the desired biological endpoint, the pharmacokinetics of the compound, the condition being treated, the mode of administration, and the age and health of the subject. In certain embodiments, an effective amount is a therapeutically effective amount. In certain embodiments, an effective amount is a prophylactic treatment. In certain embodiments, an effective amount is the amount of a compound described herein in a single dose. In certain embodiments, an effective amount is the combined amounts of a compound described herein in multiple doses.

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces, or avoids symptoms, signs, or causes of the condition, and/or enhances the therapeutic efficacy of another therapeutic agent. In certain embodiments, a therapeutically effective amount is an amount sufficient for CDK binding and/or promoting the degradation of CDK9. In certain embodiments, a therapeutically effective amount is an amount sufficient for treating a cancer.

A “prophylactically effective amount” of a compound described herein is an amount sufficient to prevent a condition, or one or more signs or symptoms associated with the condition, or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent.

A “proliferative disease” refers to a disease that occurs due to abnormal growth or extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); or 4) the pathological angiogenesis as in proliferative retinopathy and tumor metastasis. Exemplary proliferative diseases include cancers (i.e., “malignant neoplasms”), benign neoplasms, angiogenesis, inflammatory diseases, and autoimmune diseases.

The term “angiogenesis” refers to the physiological process through which new blood vessels form from pre-existing vessels. Angiogenesis is distinct from vasculogenesis, which is the de novo formation of endothelial cells from mesoderm cell precursors. The first vessels in a developing embryo form through vasculogenesis, after which angiogenesis is responsible for most blood vessel growth during normal or abnormal development. Angiogenesis is a vital process in growth and development, as well as in wound healing and in the formation of granulation tissue. However, angiogenesis is also a fundamental step in the transition of tumors from a benign state to a malignant one, leading to the use of angiogenesis inhibitors in the treatment of cancer. Angiogenesis may be chemically stimulated by angiogenic proteins, such as growth factors (e.g., VEGF). “Pathological angiogenesis” refers to abnormal (e.g., excessive or insufficient) angiogenesis that amounts to and/or is associated with a disease.

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An exemplary pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites. The term “metastasis,” “metastatic,” or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is located. For example, a prostate cancer that has migrated to bone is said to be metastasized prostate cancer and includes cancerous prostate cancer cells growing in bone tissue.

The term “cancer” refers to a class of diseases characterized by the development of abnormal cells that proliferate uncontrollably and have the ability to infiltrate and destroy normal body tissues. See, e.g., Stedman's Medical Dictionary, 25th ed.; Hensyl ed.; Williams & Wilkins: Philadelphia, 1990. Exemplary cancers include, but are not limited to, hematological malignancies. The term “hematological malignancy” refers to tumors that affect blood, bone marrow, and/or lymph nodes. Exemplary hematological malignancies include, but are not limited to, leukemia, such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma, such as Hodgkin lymphoma (HL) (e.g., B-cell HL, T-cell HL) and non-Hodgkin lymphoma (NHL) (e.g., B-cell NHL, such as diffuse large cell lymphoma (DLCL) (e.g., diffuse large B-cell lymphoma (DLBCL, e.g., activated B-cell (ABC) DLBCL (ABC-DLBCL))), follicular lymphoma, chronic lymphocytic leukemia/small lymphocytic lymphoma (CLL/SLL), mantle cell lymphoma (MCL), marginal zone B-cell lymphoma (e.g., mucosa-associated lymphoid tissue (MALT) lymphoma, nodal marginal zone B-cell lymphoma, splenic marginal zone B-cell lymphoma), primary mediastinal B-cell lymphoma, Burkitt lymphoma, Waldenstrom's macroglobulinemia (WM, lymphoplasmacytic lymphoma), hairy cell leukemia (HCL), immunoblastic large cell lymphoma, precursor B-lymphoblastic lymphoma, central nervous system (CNS) lymphoma (e.g., primary CNS lymphoma and secondary CNS lymphoma); and T-cell NHL, such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); lymphoma of an immune privileged site (e.g., cerebral lymphoma, ocular lymphoma, lymphoma of the placenta, lymphoma of the fetus, testicular lymphoma); a mixture of one or more leukemia/lymphoma as described above; myelodysplasia; and multiple myeloma (MM). Additional exemplary cancers include, but are not limited to, lung cancer (e.g., bronchogenic carcinoma, small cell lung cancer (SCLC), non-small cell lung cancer (NSCLC), adenocarcinoma of the lung); kidney cancer (e.g., nephroblastoma, a.k.a. Wilms' tumor, renal cell carcinoma); acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); heavy chain disease (e.g., alpha chain disease, gamma chain disease, mu chain disease; hemangioblastoma; hypopharynx cancer; inflammatory myofibroblastic tumors; immunocytic amyloidosis; liver cancer (e.g., hepatocellular cancer (HCC), malignant hepatoma); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g.,bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

The term “carcinoma” refers to a malignant new growth made up of epithelial cells tending to infiltrate the surrounding tissues and give rise to metastases. Exemplary carcinomas include, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiennoid carcinoma, carcinoma epitheliale adenoides, exophytic carcinoma, carcinoma ex ulcere, carcinoma fibrosum, gelatiniform carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma villosum.

The term “hematological cancer” refers to cancer that begins in blood-forming tissue, such as the bone marrow, or in the cells of the immune system. Examples of hematologic cancer are leukemia, lymphoma, and multiple myeloma. Hematological cancer is also called blood cancer.

The term “leukemia” refers to broadly progressive, malignant diseases of the blood-forming organs and is generally characterized by a distorted proliferation and development of leukocytes and their precursors in the blood and bone marrow. Leukemia diseases include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, Rieder cell leukemia, Schilling's leukemia, stem cell leukemia, subleukemic leukemia, and undifferentiated cell leukemia.

The term “lymphoma” refers to a group of blood cancers that develop from lymphocytes. Lymphoma disease includes diffuse large B-cell lymphoma (DLBCL), B-cell immunoblastic lymphoma, small non-cleaved cell lymphoma, human lymphotropic virus-type 1 (HTLV-1) leukemia/lymphoma, adult T-cell lymphoma, peripheral T-cell lymphoma (PTCL), cutaneous T-cell lymphoma (CTCL), mantle cell lymphoma (MCL), Hodgkin's lymphoma (HL), non-Hodgkin's lymphoma (NHL), AIDS-related lymphoma, follicular lymphoma, small lymphocytic lymphoma, T-cell/histiocyte rich large B-cell lymphoma, transformed lymphoma, primary mediastinal (thymic) large B-cell lymphoma, splenic marginal zone lymphoma, Richter's transformation, nodal marginal zone lymphoma, or ALK-positive large B-cell lymphoma.

The term “sarcoma” generally refers to a tumor which arises from transformed cells of mesenchymal origin. Sarcomas are malignant tumors of the connective tissue and are generally composed of closely packed cells embedded in a fibrillar or homogeneous substance. Sarcomas include, for example, chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilns' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphomas (e.g., Non-Hodgkin Lymphoma), immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

The term “melanoma” is taken to mean a tumor arising from the melanocytic system of the skin and other organs. Melanomas include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma subungal melanoma, and superficial spreading melanoma.

The terms “biologic,” “biologic drug,” and “biological product” refer to a wide range of products such as vaccines, blood and blood components, allergenics, somatic cells, gene therapy, tissues, nucleic acids, and proteins. Biologics may include sugars, proteins, or nucleic acids, or complex combinations of these substances, or may be living entities, such as cells and tissues. Biologics may be isolated from a variety of natural sources (e.g., human, animal, microorganism) and may be produced by biotechnological methods and other technologies.

The term “small molecule” or “small molecule therapeutic” refers to molecules, whether naturally occurring or artificially created (e.g., via chemical synthesis) that have a relatively low molecular weight. Typically, a small molecule is an organic compound (i.e., it contains carbon). The small molecule may contain multiple carbon-carbon bonds, stereocenters, and other functional groups (e.g., amines, hydroxyl, carbonyls, and heterocyclic rings, etc.). In certain embodiments, the molecular weight of a small molecule is not more than about 1,000 g/mol, not more than about 900 g/mol, not more than about 800 g/mol, not more than about 700 g/mol, not more than about 600 g/mol, not more than about 500 g/mol, not more than about 400 g/mol, not more than about 300 g/mol, not more than about 200 g/mol, or not more than about 100 g/mol. In certain embodiments, the molecular weight of a small molecule is at least about 100 g/mol, at least about 200 g/mol, at least about 300 g/mol, at least about 400 g/mol, at least about 500 g/mol, at least about 600 g/mol, at least about 700 g/mol, at least about 800 g/mol, or at least about 900 g/mol, or at least about 1,000 g/mol. Combinations of the above ranges (e.g., at least about 200 g/mol and not more than about 500 g/mol) are also possible. In certain embodiments, the small molecule is a therapeutically active agent such as a drug (e.g., a molecule approved by the U.S. Food and Drug Administration as provided in the Code of Federal Regulations (C.F.R.)). The small molecule may also be complexed with one or more metal atoms and/or metal ions. In this instance, the small molecule is also referred to as a “small organometallic molecule.” Preferred small molecules are biologically active in that they produce a biological effect in animals, preferably mammals, more preferably humans. Small molecules include, but are not limited to, radionuclides and imaging agents. In certain embodiments, the small molecule is a drug. Preferably, though not necessarily, the drug is one that has already been deemed safe and effective for use in humans or animals by the appropriate governmental agency or regulatory body. For example, drugs approved for human use are listed by the FDA under 21 C.F.R. §§ 330.5, 331 through 361, and 440 through 460, incorporated herein by reference; drugs for veterinary use are listed by the FDA under 21 C.F.R. §§ 500 through 589, incorporated herein by reference. All listed drugs are considered acceptable for use in accordance with the present invention.

The term “therapeutic agent” refers to any substance having therapeutic properties that produce a desired, usually beneficial, effect. For example, therapeutic agents may treat, ameliorate, and/or prevent disease. Therapeutic agents, as disclosed herein, may be biologics or small molecule therapeutics.

The term “E3 ubiquitin ligase” or “E3 ligase” refers to any protein that recruits an E2 ubiquitin-conjugating enzyme that has been loaded with ubiquitin, recognizes a protein substrate, and assists or directly catalyzes the transfer of ubiquitin from the E2 protein to the protein substrate.

### DETAILED DESCRIPTION OF CERTAIN EMBODIMENTS

Provided herein are bifunctional compounds that bind CDK9 and recruit an E3 ligase (e.g., Cereblon, VHL) to promote the degradation of CDK9. In one aspect, the disclosure provides compounds of Formula (I), and pharmaceutically acceptable salts, solvates, hydrates, polymorphs, co-crystals, tautomers, stereoisomers, isotopically labeled derivatives, prodrugs, and pharmaceutical compositions thereof. The compounds are useful for the treatment of diseases associated with CDK9 (e.g., cancer) in a subject in need thereof.

### Compounds

In one aspect, disclosed is a compound of Formula (I):

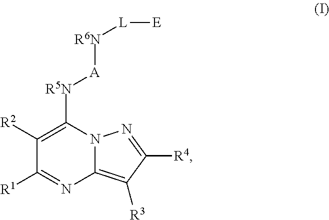

or a pharmaceutically acceptable salt, co-crystal, tautomer, stereoisomer, solvate, hydrate, polymorph, isotopically enriched derivative, or prodrug thereof, wherein:

each of R¹, R², R³, and R⁴ is independently hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, substituted or unsubstituted heteroalkyl, —N(R^{A})₂, —OR^{A}, —SR^{A}, —C(═O)OR^{A}, —C(═O)N(R^{A})₂, —NR^{A}C(═O)R^{A}, —C(═O)R^{A}, —NR^{A}C(═O)OR^{A}, —NR^{A}C(═O)N(R^{A})₂, —OC(═O)R^{A}, —OC(═O)OR^{A}, —OC(═O)N(R^{A})₂, —S(O)₂N(R^{A})₂, or —NR^{A}S(O)₂R^{A};

each of R⁵ and R⁶ is independently hydrogen, substituted or unsubstituted alkyl, —C(═O)R^{A}, or a nitrogen protecting group;

A is substituted or unsubstituted carbocyclylene, or substituted or unsubstituted heterocyclylene;

E is an E3 ligase binding moiety;

L is a bond, substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, —S(O)₂NR^{A}—, —NR^{A}S(O)₂—, or a combination thereof; and

each occurrence of R^{A} is, independently, hydrogen, substituted or unsubstituted acyl, substituted or unsubstituted alkyl, substituted or unsubstituted alkenyl, substituted or unsubstituted alkynyl, substituted or unsubstituted heteroalkyl, substituted or unsubstituted carbocyclyl, substituted or unsubstituted heterocyclyl, substituted or unsubstituted aryl, substituted or unsubstituted heteroaryl, an oxygen protecting group when attached to an oxygen atom, or a nitrogen protecting group when attached to a nitrogen atom, or two R^{A} groups are joined to form a substituted or unsubstituted heterocyclic ring.

R¹, R², R³, and R⁴

As described herein, each of R¹, R², R³, and R⁴ is independently hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, substituted or unsubstituted heteroalkyl, —N(R^{A})₂, —OR^{A}, —SR^{A}, —C(═O)OR^{A}, —C(═O)N(R^{A})₂, —NR^{A}C(═O)R^{A}, —C(═O)R^{A}, —NR^{A}C(═O)OR^{A}, —NR^{A}C(═O)N(R^{A})₂, —OC(═O)R^{A}, —OC(═O)OR^{A}, —OC(═O)N(R^{A})₂, —S(O)₂N(R^{A})₂, or —NR^{A}S(O)₂R^{A}.

In certain embodiments, each of R¹, R², R³, and R⁴ is independently hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, or substituted or unsubstituted heteroalkyl.

In certain embodiments, each of R¹, R², R³, and R⁴ is independently hydrogen, halogen, or substituted or unsubstituted alkyl. In certain embodiments, each of R¹, R², R³, and R⁴ is independently hydrogen, halogen, or substituted or unsubstituted C₁₋₅ alkyl. In certain embodiments, each of R¹, R², R³, and R⁴ is independently hydrogen, halogen, or substituted or unsubstituted C₁₋₄ alkyl. In certain embodiments, each of R¹, R², R³, and R⁴ is independently hydrogen, halogen, or unsubstituted alkyl. In certain embodiments, each of R¹, R², R³, and R⁴ is independently hydrogen, halogen, or unsubstituted C₁₋₅ alkyl. In certain embodiments, each of R¹, R², R³, and R⁴ is independently hydrogen, halogen, or unsubstituted C₁₋₄ alkyl. In certain embodiments, each of R¹, R², R³, and R⁴ is independently hydrogen or unsubstituted alkyl. In certain embodiments, each of R¹, R², R³, and R⁴ is independently hydrogen or unsubstituted C₁₋₅ alkyl. In certain embodiments, each of R¹, R², R³, and R⁴ is independently hydrogen or unsubstituted C₁₋₄ alkyl. In certain embodiments, each of R¹, R², R³, and R⁴ is independently hydrogen or unsubstituted pentanyl. In certain embodiments, each of R¹, R², R³, and R⁴ is independently hydrogen or unsubstituted 3-pentanyl. In certain embodiments, each of R¹, R², R³, and R⁴ is independently hydrogen or unsubstituted propyl. In certain embodiments, each of R¹, R², R³, and R⁴ is independently hydrogen or unsubstituted n-propyl. In certain embodiments, each of R¹, R², R³, and R⁴ is independently hydrogen, unsubstituted 3-pentanyl, or unsubstituted n-propyl.

In certain embodiments, each of R², R³, and R⁴ is hydrogen; and R¹ is hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, or substituted or unsubstituted heteroalkyl.

In certain embodiments, each of R², R³, and R⁴ is hydrogen; and R¹ is hydrogen, halogen, or substituted or unsubstituted alkyl. In certain embodiments, each of R², R³, and R⁴ is hydrogen; and R¹ is hydrogen, halogen, or substituted or unsubstituted C₁₋₅ alkyl. In certain embodiments, each of R², R³, and R⁴ is hydrogen; and R¹ is hydrogen, halogen, or substituted or unsubstituted C₁₋₄ alkyl. In certain embodiments, each of R², R³, and R⁴ is hydrogen; and R¹ is hydrogen, halogen, or unsubstituted alkyl. In certain embodiments, each of R², R³, and R⁴ is hydrogen; and R¹ is hydrogen, halogen, or unsubstituted C₁₋₅ alkyl. In certain embodiments, each of R², R³, and R⁴ is hydrogen; and R¹ is hydrogen, halogen, or unsubstituted C₁₋₄ alkyl. In certain embodiments, each of R², R³, and R⁴ is hydrogen; and R¹ is hydrogen, halogen, or unsubstituted pentanyl. In certain embodiments, each of R², R³, and R⁴ is hydrogen; and R¹ is hydrogen, halogen, or unsubstituted 3-pentanyl. In certain embodiments, each of R², R³, and R⁴ is hydrogen; and R¹ is hydrogen, halogen, or unsubstituted propyl. In certain embodiments, each of R², R³, and R⁴ is hydrogen; and R¹ is hydrogen, halogen, or unsubstituted n-propyl. In certain embodiments, each of R², R³, and R⁴ is hydrogen; and R¹ is hydrogen, halogen, unsubstituted 3-pentanyl, or unsubstituted n-propyl.

In certain embodiments, each of R², R³, and R⁴ is hydrogen; and R¹ is substituted or unsubstituted alkyl. In certain embodiments, each of R², R³, and R⁴ is hydrogen; and R¹ is substituted or unsubstituted C₁₋₅ alkyl. In certain embodiments, each of R², R³, and R⁴ is hydrogen; and R¹ is substituted or unsubstituted C₁₋₄ alkyl. In certain embodiments, each of R², R³, and R⁴ is hydrogen; and R¹ is unsubstituted C₁₋₅ alkyl. In certain embodiments, each of R², R³, and R⁴ is hydrogen; and R¹ is unsubstituted C₁₋₄ alkyl. In certain embodiments, each of R², R³, and R⁴ is hydrogen; and R¹ is unsubstituted pentanyl. In certain embodiments, each of R², R³, and R⁴ is hydrogen; and R¹ is unsubstituted pentanyl or unsubstituted propyl. In certain embodiments, each of R², R³, and R⁴ is hydrogen; and R¹ is unsubstituted propyl.

In certain embodiments, each of R¹, R², R³, and R⁴ is hydrogen.

R⁵ and R⁶

As described herein, each of R⁵ and R⁶ is independently hydrogen, substituted or unsubstituted alkyl, —C(═O)R^{A}, or a nitrogen protecting group.

In certain embodiments, each of R⁵ and R⁶ is independently hydrogen or substituted or unsubstituted alkyl. In certain embodiments, each of R⁵ and R⁶ is independently hydrogen or unsubstituted alkyl. In certain embodiments, each of R⁵ and R⁶ is independently hydrogen or unsubstituted C₁₋₄ alkyl. In certain embodiments, each of R⁵ and R⁶ is independently hydrogen or methyl.

In certain embodiments, each of R⁵ and R⁶ is hydrogen.

### A

As described herein, A is substituted or unsubstituted carbocyclylene, or substituted or unsubstituted heterocyclylene.

In certain embodiments, A is substituted or unsubstituted heterocyclylene.

In certain embodiments, A is substituted or unsubstituted C₄₋₆ heterocyclylene. In certain embodiments, A is substituted or unsubstituted C₄₋₅ heterocyclylene. In certain embodiments, A is substituted or unsubstituted C₅₋₆ heterocyclylene. In certain embodiments, A is substituted or unsubstituted C₄ heterocyclylene. In certain embodiments, A is substituted or unsubstituted C₅ heterocyclylene. In certain embodiments, A is substituted or unsubstituted C₆ heterocyclylene.

In certain embodiments, A is substituted or unsubstituted piperidine. In certain embodiments, A is substituted or unsubstituted morpholine. In certain embodiments, A is substituted or unsubstituted piperazine. In certain embodiments, A is substituted or unsubstituted pyrrolidine. In certain embodiments, A is substituted or unsubstituted pyrazoline. In certain embodiments, A is substituted or unsubstituted oxazolidine. In certain embodiments, A is substituted or unsubstituted thiazolidine. In certain embodiments, A is substituted or unsubstituted azetidine. In certain embodiments, A is substituted or unsubstituted oxetane.

In certain embodiments, A is substituted or unsubstituted carbocyclylene.

In certain embodiments, A is substituted or unsubstituted C₃₋₆ cycloalkylene. In certain embodiments, A is substituted or unsubstituted C₄₋₆ cycloalkylene. In certain embodiments, A is substituted or unsubstituted C₃₋₅ cycloalkylene. In certain embodiments, A is substituted or unsubstituted C₃₋₄ cycloalkylene. In certain embodiments, A is substituted or unsubstituted C₅₋₆ cycloalkylene.

In certain embodiments, A is substituted or unsubstituted cyclopropylene. In certain embodiments, A is substituted or unsubstituted cyclobutylene. In certain embodiments, A is substituted or unsubstituted cyclopentylene. In certain embodiments, A is substituted or unsubstituted cyclohexylene.

In certain embodiments, A is unsubstituted cyclopropylene. In certain embodiments, A is unsubstituted cyclobutylene. In certain embodiments, A is unsubstituted cyclopentylene. In certain embodiments, A is unsubstituted cyclohexylene.

In certain embodiments, A is

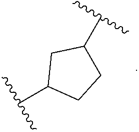 [SMILES: *C1CCC(*)C1]

In certain embodiments, A is

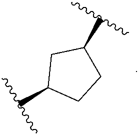 [SMILES: *[C@@H]1CC[C@H](*)C1]

In certain embodiments, A is

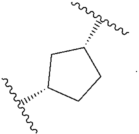 [SMILES: *[C@@H]1CC[C@H](*)C1]

In certain embodiments, A is

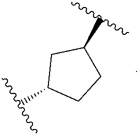 [SMILES: *[C@H]1CC[C@H](*)C1]

In certain embodiments, A is

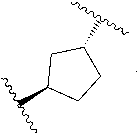 [SMILES: *[C@@H]1CC[C@@H](*)C1]

### Group L

As described herein, L is a bond, substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, —S(O)₂NR^{A}—, —NR^{A}S(O)₂—, or a combination thereof.

In certain embodiments, L is any “L” group recited in U.S. Patent Application, U.S. Ser. No. 14/792,414, filed Jul. 6, 2015, which is incorporated herein by reference. In certain embodiments, L is any “Linker” group recited in U.S. Patent Application, U.S. Ser. No. 14/707,930, filed May 8, 2015, which is incorporated herein by reference.

In certain embodiments, L is substituted or unsubstituted alkylene, substituted or unsubstituted alkenylene, substituted or unsubstituted alkynylene, substituted or unsubstituted heteroalkylene, substituted or unsubstituted heteroalkenylene, substituted or unsubstituted heteroalkynylene, substituted or unsubstituted arylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}, —NR^{A}C(═O)O—, —NR^{A}C(═O)N(R^{A})—, —OC(═O)—, —OC(═O)O—, —OC(═O)N(R^{A})—, —S(O)₂NR^{A}—, —NR^{A}S(O)₂—, or a combination thereof.

In certain embodiments, L is substituted or unsubstituted alkylene, substituted or unsubstituted alkynylene, substituted or unsubstituted heteroalkylene, substituted or unsubstituted arylene, —O—, —C(═O)—, or a combination thereof.

In certain embodiments, L is substituted or unsubstituted alkylene, substituted or unsubstituted heteroalkylene, substituted or unsubstituted arylene, —C(═O)—, or a combination thereof. In certain embodiments, L is a substituted or unsubstituted C₁₋₃₀ alkylene. In certain embodiments, L is a substituted or unsubstituted C₁₋₂₀ alkylene. In certain embodiments, L is a substituted or unsubstituted C₁₋₁₀ alkylene. In certain embodiments, L is a substituted or unsubstituted C₁₋₃₀ heteroalkylene. In certain embodiments, L is a substituted or unsubstituted C₁₋₂₀ heteroalkylene. In certain embodiments, L is a substituted or unsubstituted C₁₋₁₀ heteroalkylene. In certain embodiments, L is a substituted or unsubstituted C₁₋₃₀ alkynylene. In certain embodiments, L is a substituted or unsubstituted C₁₋₂₀ alkynylene. In certain embodiments, L is a substituted or unsubstituted C₁₋₁₀ alkynylene. In certain embodiments, L is a substituted or unsubstituted C₁₋₃₀ alkylene, wherein one or more carbons are optionally replaced with an arylene (e.g., phenylene). In certain embodiments, L is a substituted or unsubstituted C₁₋₂₀ alkylene, wherein one or more carbons are optionally replaced with an arylene (e.g., phenylene). In certain embodiments, L is a substituted or unsubstituted C₁₋₁₀ alkylene, wherein one or more carbons are optionally replaced with an arylene (e.g., phenylene). In certain embodiments, L is a substituted or unsubstituted C₂₋₅ alkylene, wherein one or more carbons are optionally replaced with an arylene (e.g., phenylene).

In certain embodiments, L is

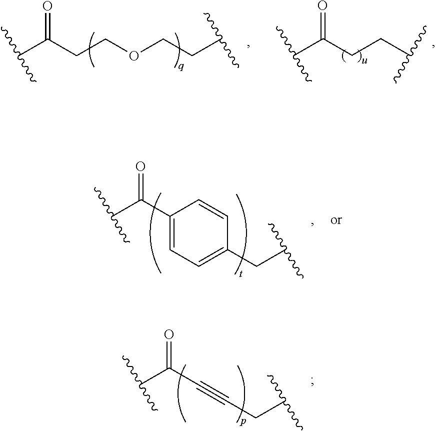

q is 1-12; u is 1-12; t is 1-3; and p is 1-3.

In certain embodiments, L is

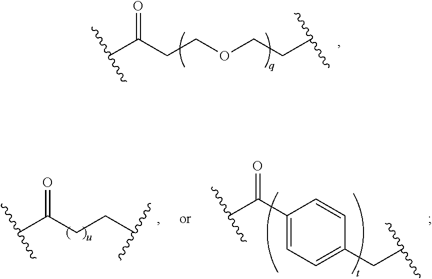

q is 1-12; u is 1-12; and t is 1-3.

In certain embodiments, L is

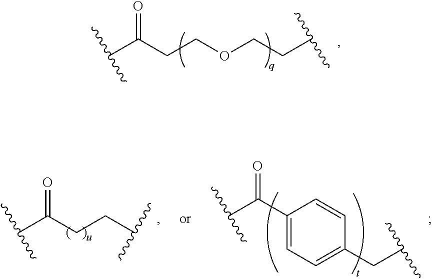

q is 1-4; u is 1-8; and t is 1-2.

In certain embodiments, L is

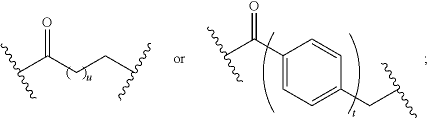

u is 1-8; and t is 1-2.

In certain embodiments, L is

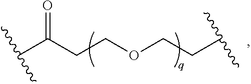

and q is 1-12. In certain embodiments, L is

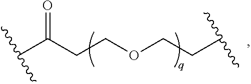

and q is 1-11. In certain embodiments, L is

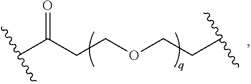

and q is 1-10. In certain embodiments, L is

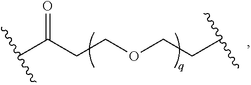

and q is 1-9. In certain embodiments, L is

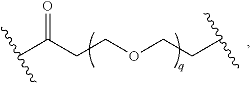

and q is 1-8. In certain embodiments, L is

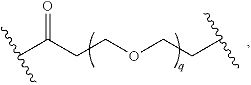

and q is 1-7. In certain embodiments, L is

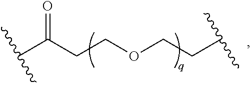

and q is 1-6. In certain embodiments, L is

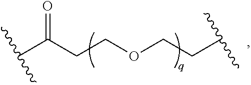

and q is 1-5. In certain embodiments, L is

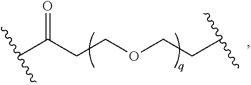

and q is 1-4. In certain embodiments, L is

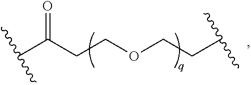

and q is 1-3. In certain embodiments, L is

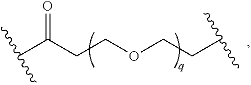

and q is 1-2.

In certain embodiments, L is

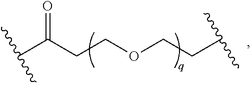

and q is 12. In certain embodiments, L is

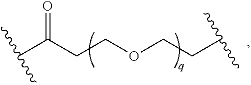

and q is 11. In certain embodiments, L is

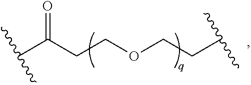

and q is 10. In certain embodiments, L is

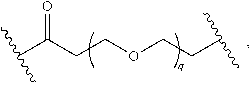

and q is 9. In certain embodiments, L is

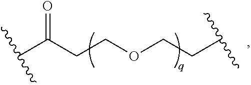

and q is 8. In certain embodiments, L is

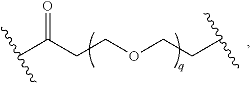

and q is 7. In certain embodiments, L is

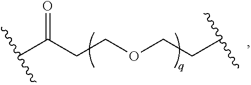

and q is 6. In certain embodiments, L is

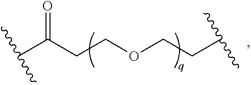

and q is 5. In certain embodiments, L is

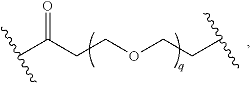

and q is 4. In certain embodiments, L is

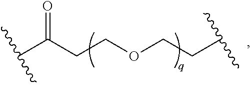

and q is 3. In certain embodiments, L is

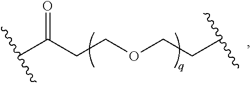

and q is 2. In certain embodiments, L is

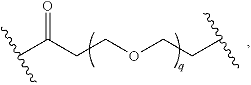

and q is 1.

In certain embodiments, L is

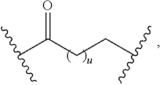

and u is 1-12. In certain embodiments, L is

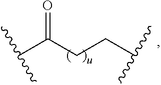

and u is 1-11. In certain embodiments, L is

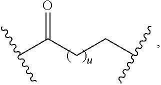

and u is 1-10. In certain embodiments, L is

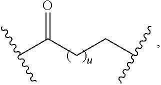

and u is 1-9. In certain embodiments, L is

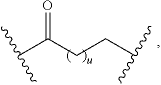

and u is 1-8. In certain embodiments, L is

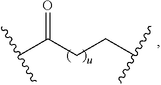

and u is 1-7. In certain embodiments, L is

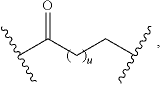

and u is 1-6. In certain embodiments, L is

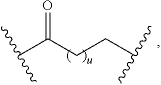

and u is 1-5. In certain embodiments, L is

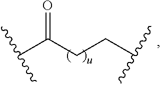

and u is 1-4. In certain embodiments, L is

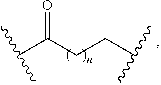

and u is 1-3. In certain embodiments, L is

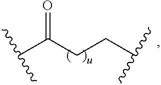

and u is 1-2.

In certain embodiments, L is

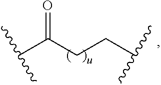

and u is 12. In certain embodiments, L is

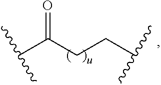

and u is 11. In certain embodiments, L is

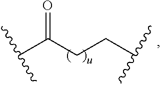

and u is 10. In certain embodiments, L is

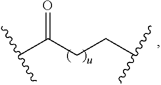

and u is 9. In certain embodiments, L is

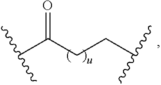

and u is 8. In certain embodiments, L is

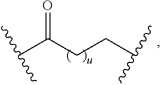

and u is 7. In certain embodiments, L is

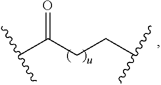

and u is 6. In certain embodiments, L is

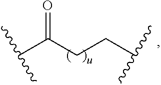

and u is 5. In certain embodiments, L is

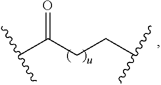

and u is 4. In certain embodiments, L is

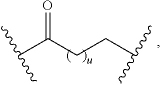

and u is 3. In certain embodiments, L is

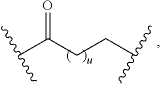

and u is 2. In certain embodiments, L is

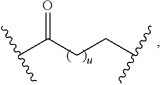

and u is 1.

In certain embodiments, L is

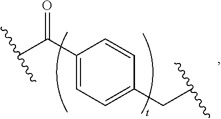

and t is 1-3. In certain embodiments, L is

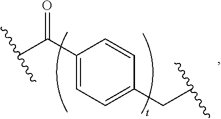

and t is 1-2. In certain embodiments, L is

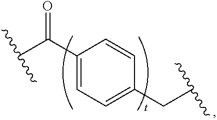

and t is 2-3.

In certain embodiments, L is

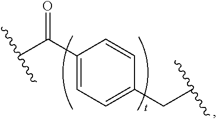

and t is 3. In certain embodiments, L is

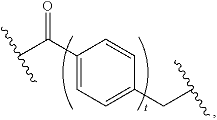

and t is 2. In certain embodiments, L is

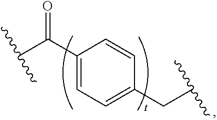

and t is 1.

In certain embodiments, L is

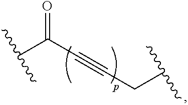

and p is 1-3. In certain embodiments, L is

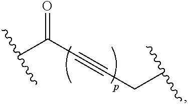

and p is 1-2. In certain embodiments, L is

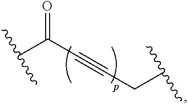

and p is 2-3.

In certain embodiments, L is

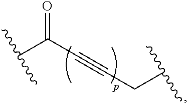

and p is 3. In certain embodiments, L is

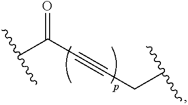

and p is 2. In certain embodiments, L is

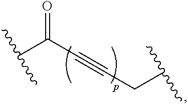

and p is 1.

In certain embodiments, L is

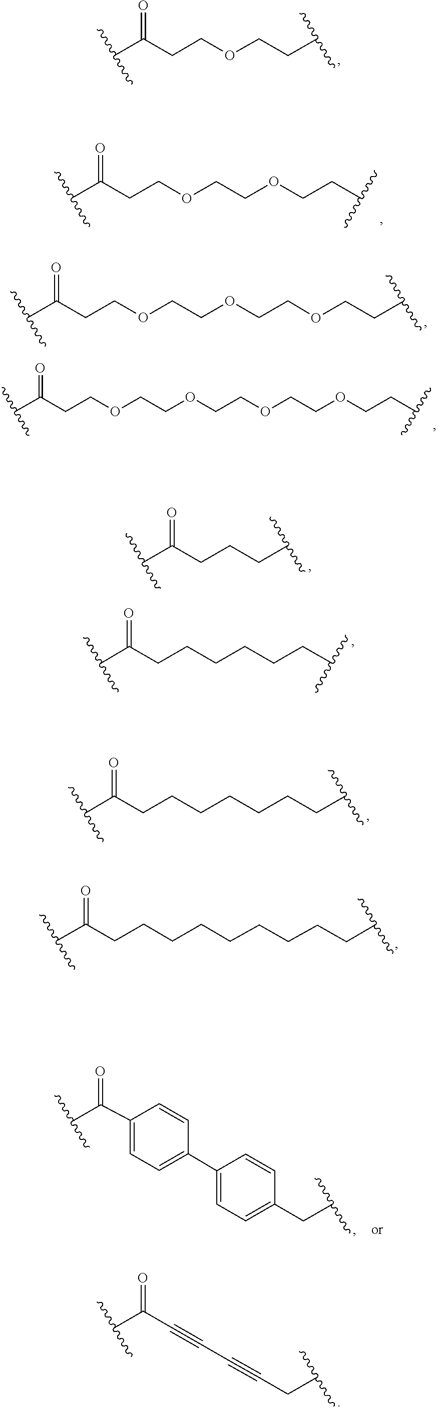

In certain embodiments, L is

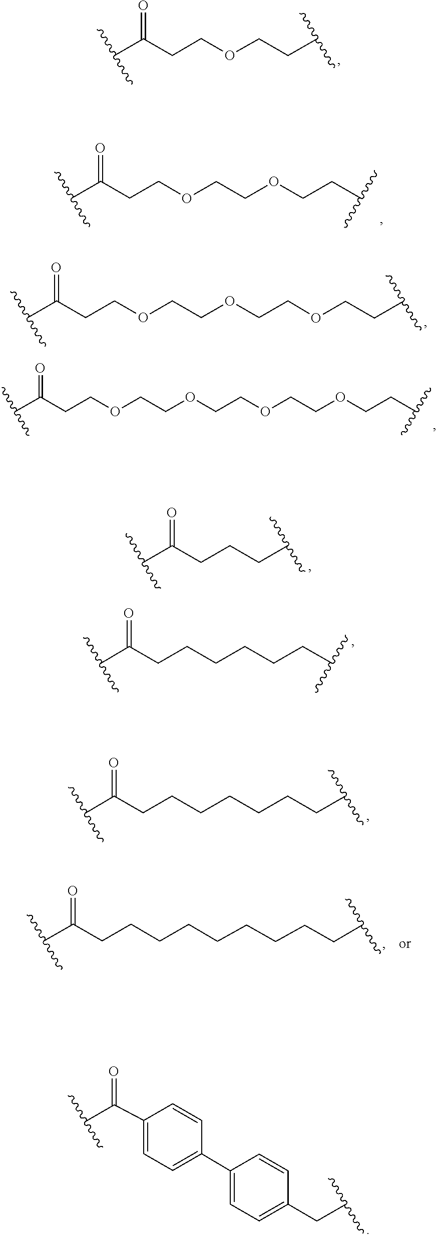

In certain embodiments, L is

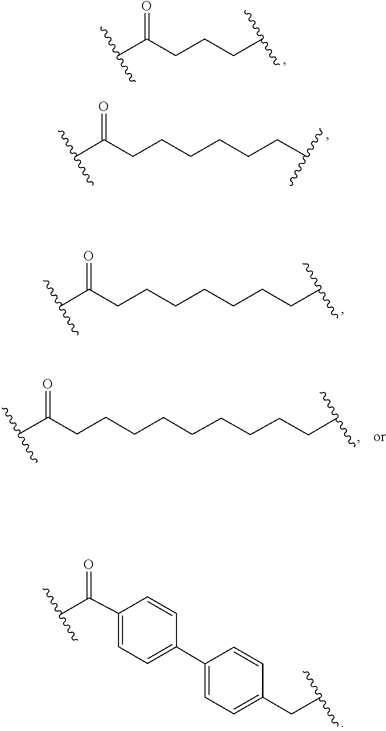 [SMILES: *CCCC(*)=O ; *CCCCCCC(*)=O ; *CCCCCCCC(*)=O ; *CCCCCCCCCC(*)=O ; *Cc1ccc(-c2ccc(C(*)=O)cc2)cc1]

In certain embodiments, L is

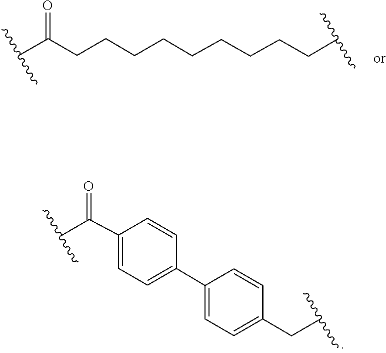 [SMILES: *CCCCCCCCCC(*)=O ; *Cc1ccc(-c2ccc(C(*)=O)cc2)cc1]

In certain embodiments, L is

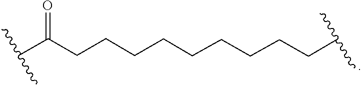 [SMILES: *CCCCCCCCCC(*)=O]

In certain embodiments, L is

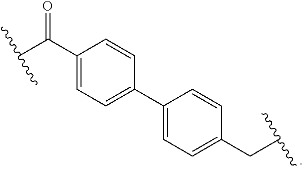 [SMILES: *Cc1ccc(-c2ccc(C(*)=O)cc2)cc1]

### Group R^{A}

As described herein, each occurrence of R^{A} is, independently, hydrogen, substituted or unsubstituted acyl, substituted or unsubstituted alkyl, substituted or unsubstituted alkenyl, substituted or unsubstituted alkynyl, substituted or unsubstituted heteroalkyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a nitrogen protecting group when attached to a nitrogen atom, or two R^{A} groups are joined to form a substituted or unsubstituted heterocyclic ring.

In certain embodiments, each occurrence of R^{A} is, independently, hydrogen, substituted or unsubstituted alkyl, substituted or unsubstituted heteroalkyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or two R^{A} groups are joined to form a substituted or unsubstituted heterocyclic ring.

In certain embodiments, each occurrence of R^{A} is, independently, hydrogen, substituted or unsubstituted alkyl, substituted or unsubstituted heteroalkyl, substituted or unsubstituted heterocyclyl, substituted or unsubstituted aryl, substituted or unsubstituted heteroaryl, or two R^{A} groups are joined to form a substituted or unsubstituted heterocyclic ring.

In certain embodiments, each occurrence of R^{A} is, independently, hydrogen, substituted or unsubstituted C₁₋₆ alkyl, substituted or unsubstituted heteroalkyl, substituted or unsubstituted 5-6 membered heterocyclyl, substituted or unsubstituted phenyl, substituted or unsubstituted 5-6 membered heteroaryl, or two R^{A} groups are joined to form a substituted or unsubstituted heterocyclic ring.

In certain embodiments, each occurrence of R^{A} is, independently, hydrogen, substituted or unsubstituted C₁₋₆ alkyl, substituted or unsubstituted C₁₋₃₀ heteroalkyl, substituted or unsubstituted 5-6 membered heterocyclyl, substituted or unsubstituted phenyl, substituted or unsubstituted 5-6 membered heteroaryl, or two R^{A} groups are joined to form a substituted or unsubstituted heterocyclic ring.

In certain embodiments, each occurrence of R^{A} is, independently, hydrogen, substituted or unsubstituted C₁₋₆ alkyl, or substituted or unsubstituted C₁₋₃₀ heteroalkyl.

In certain embodiments, each occurrence of R^{A} is, independently, hydrogen, substituted or unsubstituted C₁₋₆ alkyl, or substituted or unsubstituted C₁₋₂₀ heteroalkyl.

In certain embodiments, each occurrence of R^{A} is, independently, hydrogen, or substituted or unsubstituted C₁₋₆ alkyl.

In certain embodiments, each occurrence of R^{A} is hydrogen.

### Group E

E is an E3 ubiquitin ligase binding moiety. E is inclusive of all moieties that bind, or can bind, any E3 ubiquitin ligase. In certain embodiments, E is capable of binding an E3 ubiquitin ligase, such as Cereblon or von Hippel-Lindau tumor suppressor (VHL). In certain embodiments, E is capable of binding to multiple different E3 ubiquitin ligases. In certain embodiments, E binds to Cereblon. In certain embodiments, E binds to VHL.

Human Cereblon (CRBN) is a protein of 442 amino acids with an apparent molecular weight of ˜51 kDa (GenBank: AAH17419). (For the CRBN protein sequence see: Higgins et al., Neurology. 2004, 63, 1927-31. For additional information related to the CRBN structure see Hartmann et al., PLoS One. 2015, 10, e0128342.) Human CRBN contains the N-terminal part (237-amino acids from 81 to 317) of ATP-dependent Lon protease domain without the conserved Walker A and Walker B motifs, 11 casein kinase II phosphorylation sites, 4 protein kinase C phosphorylation sites, 1 N-linked glycosylation site, and 2 myristoylation sites. CRBN is widely expressed in testis, spleen, prostate, liver, pancreas, placenta, kidney, lung, skeletal muscle, ovary, small intestine, peripheral blood leukocyte, colon, brain, and retina. CRBN is located in the cytoplasm, nucleus, and peripheral membrane. (Chang et al., Int. J. Biochem. Mol. Biol. 2011, 2, 287-94.)

Cereblon is an E3 ubiquitin ligase, and it forms an E3 ubiquitin ligase complex with damaged DNA binding protein 1 (DDB1), Cullin-4A (CUL4A), and regulator of cullins 1 (ROC1). This complex ubiquitinates a number of other proteins. Through a mechanism which has not been completely elucidated, Cereblon ubiquitination of target proteins results in increased levels of fibroblast growth factor 8 (FGF8) and fibroblast growth factor 10 (FGF10). FGF8, in turn, regulates a number of developmental processes, such as limb and auditory vesicle formation.

In certain embodiments, E is a modulator, binder, inhibitor, or ligand of Cereblon. In certain embodiments, E is a modulator of Cereblon. In certain embodiments, E is a binder of Cereblon. In certain embodiments, E is an inhibitor of Cereblon. In certain embodiments, E is a ligand of Cereblon. In certain embodiments, E is any modulator, binder, inhibitor, or ligand of Cereblon disclosed in U.S. Patent Application, U.S. Ser. No. 16/523,219, filed Jul. 26, 2019; U.S. Patent Application, U.S. Ser. No. 16/502,529, filed Jul. 3, 2019; U.S. Patent Application, U.S. Ser. No. 16/375,643, filed Apr. 4, 2019; U.S. Patent Application, U.S. Ser. No. 16/230,792, filed Dec. 21, 2018; U.S. Patent Application, U.S. Ser. No. 15/840,950, filed Sep. 13, 2018; U.S. Patent Application, U.S. Ser. No. 15/996,151, filed Jun. 1, 2018; U.S. Patent Application, U.S. Ser. No. 15/953,108, filed Apr. 13, 2018; U.S. Patent Application, U.S. Ser. No. 15/885,671, filed Jan. 31, 2018; U.S. Patent Application, U.S. Ser. No. 15/881,318, filed Jan. 26, 2018; U.S. Patent Application, U.S. Ser. No. 15/853,166, filed Dec. 22, 2017; U.S. Patent Application, U.S. Ser. No. 15/852,854, filed Dec. 22, 2017; U.S. Patent Application, U.S. Ser. No. 15/851,053, filed Dec. 21, 2017; U.S. Patent Application, U.S. Ser. No. 15/829,541, filed Dec. 1, 2017; U.S. Patent Application, U.S. Ser. No. 15/801,243, filed Nov. 1, 2017; U.S. Patent Application, U.S. Ser. No. 15/730,728, filed Oct. 11, 2017; U.S. Patent Application, U.S. Ser. No. 15/706,064, filed Sep. 15, 2017; U.S. Patent Application, U.S. Ser. No. 15/663,273, filed Jul. 28, 2017; U.S. Patent Application, U.S. Ser. No. 15/230,354, filed Aug. 5, 2016; U.S. Patent Application, U.S. Ser. No. 15/206,497, filed Jul. 11, 2016; U.S. Patent Application, U.S. Ser. No. 15/209,648, filed Jul. 13, 2016; U.S. Patent Application, U.S. Ser. No. 14/822,309, filed Aug. 10, 2015; U.S. Patent Application, U.S. Ser. No. 14/792,414, filed Jul. 6, 2015; U.S. Patent Application, U.S. Ser. No. 14/707,930, filed May 8, 2015; International Patent Application, PCT/US2019/040545, filed Jul. 3, 2019; International Patent Application, PCT/US2019/040520, filed Jul. 3, 2019; International Patent Application, PCT/US2019/013481, filed Jan. 14, 2019; International Patent Application, PCT/US2018/052181, filed Sep. 21, 2018; and International Patent Application, PCT/US2013/054663, filed Aug. 13, 2013, each of which is incorporated herein by reference. In certain embodiments, E is a modulator, binder, inhibitor, or ligand of a Cereblon variant. In certain embodiments, E is a modulator, binder, inhibitor, or ligand of a Cereblon isoform.

In certain embodiments, E is of Formula (E-I):

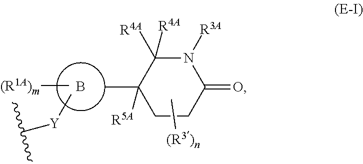

wherein:

B is a substituted or unsubstituted monocyclic, bicyclic, or tricyclic fused ring system;

Y is —(CH₂)ₖ—, —(CH₂)ₖ—O—, —O(CH₂)ₖ—, —NR^{B}(CH₂)ₖ—, —(CH₂)ₖ—NR^{B}—, —(CH₂)ₖ—(C═O)NR^{B}—, —O(CH₂)ₖ—(C═O)NR^{B}—, —O(CH₂)ₖ—NR^{B}(C═O)—, —NR^{B}(C═O)—(CH₂)ₖ—_{O—, —NR}^{B}(CH₂)ₖ—NR^{B}(C═O)—, or —(CH₂)ₖ—NR^{B}(C═O)—;

each R^{B} is, independently, hydrogen, or substituted or unsubstituted alkyl;

each R^{1A} is, independently, halogen, OH, C₁-C₆ alkyl, or C₁-C₆ alkoxy;

each R^{3A} is, independently, hydrogen or C₁-C₃ alkyl;

each R^{3′} is, independently, C₁-C₃ alkyl;

each R^{4A} is, independently, hydrogen or C₁-C₃ alkyl; or two R^{4A}, together with the carbon atom to which they are attached, form a C(O), C₃-C₆ carbocycle, or a 4-6-membered heterocycle comprising 1 or 2 heteroatoms selected from N and O;

R^{5A} is hydrogen, C₁-C₃ alkyl, F, or Cl;

k is 0, 1, 2, 3, 4, 5, or 6;

m is 0, 1, 2 or 3; and

n is 0, 1, or 2.

In certain embodiments, E is of Formula (E-I-a):

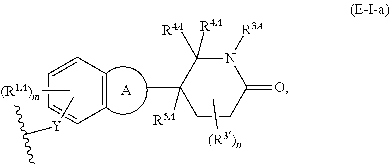

wherein:

A is a substituted or unsubstituted heterocyclyl, or substituted or unsubstituted heteroaryl ring;

Y is —(CH₂)ₖ—, —(CH₂)ₖ—O—, —O(CH₂)ₖ—, —NR^{B}(CH₂)ₖ—, —(CH₂)ₖ—NR^{B}—, —(CH₂)ₖ—(C═O)NR^{B}—, —O(CH₂)ₖ—(C═O)NR^{B}—, —O(CH₂)ₖ—NR^{B}(C═O)—, —NR^{B}(C═O)—(CH₂)ₖ—O—, —NR^{B}(CH₂)ₖ—NR^{B}(C═O)—, or —(CH₂)ₖ—NR^{B}(C═O)—;

each R^{B} is, independently, hydrogen, or substituted or unsubstituted alkyl;

each R^{1A} is, independently, halogen, OH, C₁-C₆ alkyl, or C₁-C₆ alkoxy;

each R^{3A} is, independently, hydrogen or C₁-C₃ alkyl;

each R^{3′} is, independently, C₁-C₃ alkyl;

each R^{4A} is, independently, hydrogen or C₁-C₃ alkyl; or two R^{4A}, together with the carbon atom to which they are attached, form a C(O), C₃-C₆ carbocycle, or a 4-6-membered heterocycle comprising 1 or 2 heteroatoms selected from N and O;

R^{5A} is hydrogen, C₁-C₃ alkyl, F, or Cl;

k is 0, 1, 2, 3, 4, 5, or 6;

m is 0, 1, 2 or 3; and

n is 0, 1, or 2.

In certain embodiments, E is of Formula (E-I-b):

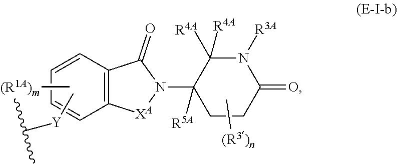

wherein:

Y is —(CH₂)ₖ—, —(CH₂)ₖ—O—, —O(CH₂)ₖ—, —NR^{B}(CH₂)ₖ—, —(CH₂)ₖ—NR^{B}—, —(CH₂)ₖ—(C═O)NR^{B}—, —O(CH₂)ₖ—(C═O)NR^{B}—, —O(CH₂)ₖ—NR^{B}(C═O)—, —NR^{B}(C═O)—(CH₂)ₖ—O—, —NR^{B}(CH₂)ₖ—NR^{B}(C═O)—, or —(CH₂)ₖ—NR^{B}(C═O)—;

X^{A} is C(O) or C(R^{3A})₂;

each R^{B} is, independently, hydrogen, or substituted or unsubstituted alkyl;

each R^{1A} is, independently, halogen, OH, C₁-C₆ alkyl, or C₁-C₆ alkoxy;

each R^{3A} is, independently, hydrogen, or C₁-C₃ alkyl;

each R^{3′} is, independently, C₁-C₃ alkyl;

each R^{4A} is, independently, hydrogen or C₁-C₃ alkyl; or two R^{4A}, together with the carbon atom to which they are attached, form a C(O), C₃-C₆ carbocycle, or a 4-, 5-, or 6-membered heterocycle comprising 1 or 2 heteroatoms selected from N and O;

R^{5A} is hydrogen, C₁-C₃ alkyl, F, or Cl;

k is 0, 1, 2, 3, 4, 5, or 6;

m is 0, 1, 2 or 3; and

n is 0, 1, or 2.

In certain embodiments of Formula (E-I-b):

Y is —(CH₂)ₖ—, —(CH₂)ₖ—O—, —O(CH₂)ₖ—, —NR^{B}(CH₂)ₖ—, —(CH₂)ₖ—NR^{B}—, —(CH₂)ₖ—(C═O)NR^{B}—, —O(CH₂)ₖ—(C═O)NR^{B}—, —O(CH₂)ₖ—NR^{B}(C═O)—, —NR^{B}(CH₂)ₖ—NR^{B}(C═O)—, or —(CH₂)ₖ—NR^{B}(C═O)—;

X^{A} is C(O) or C(R^{3A})₂;

each R^{B} is, independently, hydrogen, or substituted or unsubstituted alkyl;

each R^{1A} is, independently, halogen, OH, C₁-C₆ alkyl, or C₁-C₆ alkoxy;

each R^{3A} is, independently, hydrogen, or C₁-C₃ alkyl;

each R^{3′} is, independently, C₁-C₃ alkyl;

each R^{4A} is, independently, hydrogen, or C₁-C₃ alkyl; or two R^{4A}, together with the carbon atom to which they are attached, form a C(O), C₃-C₆ carbocycle, or a 4-, 5-, or 6-membered heterocycle comprising 1 or 2 heteroatoms selected from N and O;

R^{5A} is hydrogen, C₁-C₃ alkyl, F, or Cl;

k is 0, 1, 2, 3, 4, 5, or 6;

m is 0, 1, 2 or 3; and

n is 0, 1, or 2.

In certain embodiments of Formula (E-I-b):

Y is —(CH₂)ₖ—NR^{B}—, —O(CH₂)ₖ—(C═O)NR^{B}—, —NR^{B}(C═O)—(CH₂)ₖ—O—, or —(CH₂)ₖ—NR^{B}(C═O)—;

X^{A} is C(O) or C(R^{3A})₂;

each R^{B} is, independently, hydrogen, or substituted or unsubstituted alkyl;

each R^{1A} is, independently, halogen, OH, C₁-C₆ alkyl, or C₁-C₆ alkoxy;

each R^{3A} is, independently, hydrogen, or C₁-C₃ alkyl;

each R^{3′} is, independently, C₁-C₃ alkyl;

each R^{4A} is, independently, H or C₁-C₃ alkyl; or two R^{4A}, together with the carbon atom to which they are attached, form a C(O), C₃-C₆ carbocycle, or a 4-, 5-, or 6-membered heterocycle comprising 1 or 2 heteroatoms selected from N and O;

R^{5A} is hydrogen, C₁-C₃ alkyl, F, or Cl;

k is 0, 1, 2, 3, 4, 5, or 6;

m is 0, 1, 2 or 3; and

n is 0, 1, or 2.

In certain embodiments of Formula (E-I-b):

Y is —(CH₂)ₖ—NR^{B}—, —O(CH₂)ₖ—(C═O)NR^{B}—, or —(CH₂)ₖ—NR^{B}(C═O)—;

X^{A} is C(O) or C(R^{3A})₂;

each R^{B} is, independently, hydrogen, or substituted or unsubstituted alkyl;

each R^{1A} is, independently, halogen, OH, C₁-C₆ alkyl, or C₁-C₆ alkoxy;

each R^{3A} is, independently, H or C₁-C₃ alkyl;

each R^{3′} is, independently, C₁-C₃ alkyl;

each R^{4A} is, independently, H or C₁-C₃ alkyl; or two R^{4A}, together with the carbon atom to which they are attached, form a C(O), C₃-C₆ carbocycle, or a 4-, 5-, or 6-membered heterocycle comprising 1 or 2 heteroatoms selected from N and O;

R^{5A} is hydrogen, C₁-C₃ alkyl, F, or Cl;

k is 0, 1, 2, 3, 4, 5, or 6;

m is 0, 1, 2 or 3; and

n is 0, 1, or 2.

In certain embodiments, E is of Formula (E-I-c):

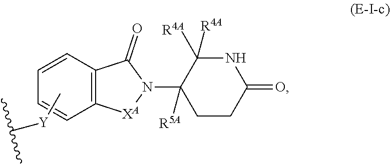

wherein Y, X^{A}, R^{5A}, and R^{4A} are as defined herein.

In certain embodiments, E is of Formula (E-I-d):

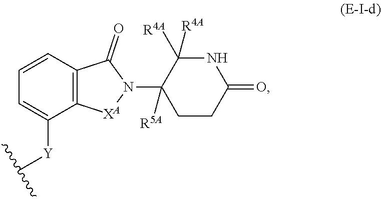

wherein:

Y is —(CH₂)ₖ—NR^{B}—, —O(CH₂)ₖ—(C═O)NR^{B}—, —NR^{B}(C═O)—(CH₂)ₖ—O—, or —(CH₂)ₖ—NR^{B}(C═O)—;

X^{A} is C(O) or C(R^{3A})₂;

each R^{B} is, independently, hydrogen, or substituted or unsubstituted alkyl;

each R^{3A} is, independently, hydrogen or C₁-C₃ alkyl;

each R^{4A} is, independently, hydrogen or C₁-C₃ alkyl; or two R^{4A}, together with the carbon atom to which they are attached, form a C(O), C₃-C₆ carbocycle, or a 4-, 5-, or 6-membered heterocycle comprising 1 or 2 heteroatoms selected from N and O;

R^{5A} is hydrogen, C₁-C₃ alkyl, F, or Cl; and

k is 0, 1, 2, 3, 4, 5, or 6.

In certain embodiments of Formula (E-I-d):

Y is —(CH₂)ₖ—NR^{B}—, —O(CH₂)ₖ—(C═O)NR^{B}—, or —(CH₂)ₖ—NR^{B}(C═O)—;

X^{A} is C(O) or C(R^{3A})₂;

each R^{B} is, independently, hydrogen, or substituted or unsubstituted alkyl;

each R^{3A} is, independently, H or C₁-C₃ alkyl;

each R^{4A} is, independently, H or C₁-C₃ alkyl; or two R^{4A}, together with the carbon atom to which they are attached, form a C(O), C₃-C₆ carbocycle, or a 4-, 5-, or 6-membered heterocycle comprising 1 or 2 heteroatoms selected from N and O;

R^{5A} is hydrogen, C₁-C₃ alkyl, F, or Cl; and

k is 0, 1, 2, 3, 4, 5, or 6.

In certain embodiments of Formula (E-I-d):

Y is —(CH₂)ₖ—NR^{B}—;

X^{A} is C(O) or C(R^{3A})₂;

each R^{B} is, independently, hydrogen, or substituted or unsubstituted alkyl;

each R^{3A} is, independently, hydrogen or C₁-C₃ alkyl;

each R^{4A} is, independently, hydrogen or C₁-C₃ alkyl; or two R^{4A}, together with the carbon atom to which they are attached, form a C(O), C₃-C₆ carbocycle, or a 4-, 5-, or 6-membered heterocycle comprising 1 or 2 heteroatoms selected from N and O;

R^{5A} is hydrogen, C₁-C₃ alkyl, F, or Cl; and

k is 0, 1, 2, 3, 4, 5, or 6.

In certain embodiments, E is of Formula (E-I-e):

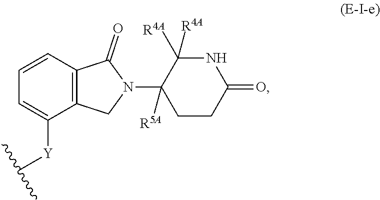

wherein:

Y is —(CH₂)ₖ—NR^{B}—, —O(CH₂)ₖ—(C═O)NR^{B}—, —NR^{B}(C═O)—(CH₂)ₖ—O—, or —(CH₂)ₖ—NR^{B}(C═O)—;

each R^{B} is, independently, hydrogen, or substituted or unsubstituted alkyl;

each R^{4A} is, independently, hydrogen or C₁-C₃ alkyl; or two R^{4A}, together with the carbon atom to which they are attached, form a C(O), C₃-C₆ carbocycle, or a 4-, 5-, or 6-membered heterocycle comprising 1 or 2 heteroatoms selected from N and O;

R^{5A} is hydrogen, C₁-C₃ alkyl, F, or Cl; and

k is 0, 1, 2, 3, 4, 5, or 6.

In certain embodiments of Formula (E-I-e):

Y is —(CH₂)ₖ—NR^{B}—;

each R^{B} is, independently, hydrogen, or substituted or unsubstituted alkyl;

each R^{4A} is, independently, hydrogen or C₁-C₃ alkyl; or two R^{4A}, together with the carbon atom to which they are attached, form a C(O), C₃-C₆ carbocycle, or a 4-, 5-, or 6-membered heterocycle comprising 1 or 2 heteroatoms selected from N and O;

R^{5A} is hydrogen, C₁-C₃ alkyl, F, or Cl; and

k is 0, 1, 2, 3, 4, 5, or 6.

In certain embodiments, E is of Formula (E-I-f):

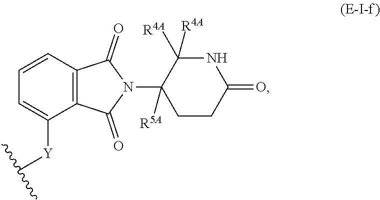

wherein:

Y is —(CH₂)ₖ—NR^{B}—, —O(CH₂)ₖ—(C═O)NR^{B}—, —NR^{B}(C═O)—(CH₂)ₖ—O—, or —(CH₂)ₖ—NR^{B}(C═O)—;

each R^{B} is, independently, hydrogen, or substituted or unsubstituted alkyl;

each R^{4A} is, independently, hydrogen, or C₁-C₃ alkyl; or two R^{4A}, together with the carbon atom to which they are attached, form a C(O), C₃-C₆ carbocycle, or a 4-, 5-, or 6-membered heterocycle comprising 1 or 2 heteroatoms selected from N and O;

R^{5A} is hydrogen, C₁-C₃ alkyl, F, or Cl; and

k is 0, 1, 2, 3, 4, 5, or 6.

In certain embodiments of Formula (E-I-f):

Y is —(CH₂)ₖ—NR^{B}—;

each R^{B} is, independently, hydrogen, or substituted or unsubstituted alkyl;

each R^{4A} is, independently, hydrogen or C₁-C₃ alkyl; or two R^{4A}, together with the carbon atom to which they are attached, form a C(O), C₃-C₆ carbocycle, or a 4-, 5-, or 6-membered heterocycle comprising 1 or 2 heteroatoms selected from N and O;

R^{5A} is hydrogen, C₁-C₃ alkyl, F, or Cl; and

k is 0, 1, 2, 3, 4, 5, or 6.

In certain embodiments, E is of formula (E-I-g):

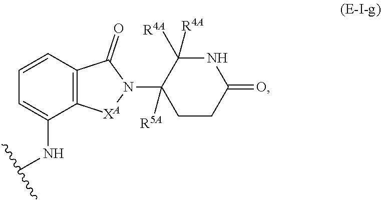

wherein:

X^{A} is C(O) or C(R^{3A})₂;

each R^{4A} is, independently, hydrogen, or C₁-C₃ alkyl; or two R^{4A}, together with the carbon atom to which they are attached, form a C(O), C₃-C₆ carbocycle, or a 4-, 5-, or 6-membered heterocycle comprising 1 or 2 heteroatoms selected from N and O;

each R^{3A} is, independently, hydrogen, or C₁-C₃ alkyl; and

R^{5A} is hydrogen, C₁-C₃ alkyl, F, or Cl.

In certain embodiments of compounds of Formula (E-I-g), X^{A} is C(O).

In certain embodiments, E is of formula (E-I-h):

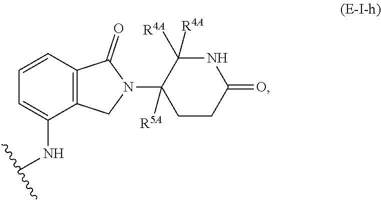

wherein:

each R^{4A} is, independently, hydrogen or C₁-C₃ alkyl; or two R^{4A}, together with the carbon atom to which they are attached, form a C(O), C₃-C₆ carbocycle, or a 4-, 5-, or 6-membered heterocycle comprising 1 or 2 heteroatoms selected from N and O; and

R^{5A} is hydrogen, C₁-C₃ alkyl, F, or Cl.

In certain embodiments, E is of formula (E-I-i):

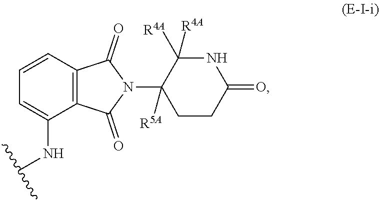

wherein:

each R^{4A} is, independently, H or C₁-C₃ alkyl; or two R^{4A}, together with the carbon atom to which they are attached, form a C(O), C₃-C₆ carbocycle, or a 4-, 5-, or 6-membered heterocycle comprising 1 or 2 heteroatoms selected from N and O; and

R^{5A} is H, C₁-C₃ alkyl, F, or Cl.

In certain embodiments, E is of Formula (E-II):

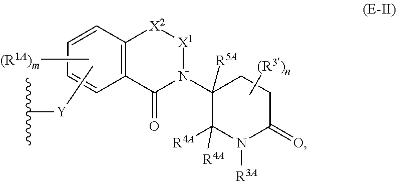

wherein:

Y is —(CH₂)ₖ—, —(CH₂)ₖ—O—, —O(CH₂)ₖ—, —NR^{B}(CH₂)ₖ—, —(CH₂)ₖ—NR^{B}—, —(CH₂)ₖ—(C═O)NR^{B}—, —O(CH₂)ₖ—(C═O)NR^{B}—, —O(CH₂)ₖ—NR^{B}(C═O)—, —NR^{B}(C═O)—(CH₂)ₖ—O—, —NR^{B}(CH₂)ₖ—NR^{B}(C═O)—, or —(CH₂)ₖ—NR^{B}(C═O)—;

X¹-X² is C(R^{3A})═N or C(R^{3A})₂—C(R^{3A})₂;

each R^{B} is, independently, hydrogen, or substituted or unsubstituted alkyl;

each R^{1A} is, independently, halogen, OH, C₁-C₆ alkyl, or C₁-C₆ alkoxy;

each R^{3A} is, independently, hydrogen, or C₁-C₃ alkyl;

each R^{3′} is, independently, C₁-C₃ alkyl;

each R^{4A} is, independently, hydrogen, or C₁-C₃ alkyl; or two R^{4A}, together with the carbon atom to which they are attached, form a C(O), C₃-C₆ carbocycle, or a 4-, 5-, or 6-membered heterocycle comprising 1 or 2 heteroatoms selected from N and O;

R^{5A} is hydrogen, C₁-C₃ alkyl, F, or Cl;

k is 0, 1, 2, 3, 4, 5, or 6;

m is 0, 1, 2 or 3; and

n is 0, 1, or 2.

In certain embodiments of Formula (E-II):

Y is —(CH₂)ₖ—, —(CH₂)ₖ—O—, —O(CH₂)ₖ—, —NR^{B}(CH₂)ₖ—, —(CH₂)ₖ—NR^{B}—, —(CH₂)ₖ—(C═O)NR^{B}—, —O(CH₂)ₖ—(C═O)NR^{B}—, —O(CH₂)ₖ—NR^{B}(C═O)—, —NR^{B}(CH₂)ₖ—NR^{B}(C═O)—, or —(CH₂)ₖ—NR^{B}(C═O)—;

X¹-X² is C(R^{3A})═N or C(R^{3A})₂—C(R^{3A})₂;

each R^{B} is, independently, hydrogen, or substituted or unsubstituted alkyl;

each R^{1A} is, independently, halogen, OH, C₁-C₆ alkyl, or C₁-C₆ alkoxy;

each R^{3A} is, independently, H or C₁-C₃ alkyl;

each R^{3′} is, independently, C₁-C₃ alkyl;

each R^{4A} is, independently, H or C₁-C₃ alkyl; or two R^{4A}, together with the carbon atom to which they are attached, form a C(O), C₃-C₆ carbocycle, or a 4-, 5-, or 6-membered heterocycle comprising 1 or 2 heteroatoms selected from N and O;

R^{5A} is hydrogen, C₁-C₃ alkyl, F, or Cl;

k is 0, 1, 2, 3, 4, 5, or 6;

m is 0, 1, 2 or 3; and

n is 0, 1,or 2.

In certain embodiments of Formula (E-II), Y is —(CH₂)ₖ—NR^{B}—, —O(CH₂)ₖ—(C═O)NR^{B}—, —(CH₂)ₖ—O—, —NR^{B}(C═O)—(CH₂)ₖ—O—, or —(CH₂)ₖ—NR^{B}(C═O)—. In certain embodiments, Y is —(CH₂)ₖ—NR^{B}—, —O(CH₂)ₖ—(C═O)NR^{B}—, or —(CH₂)ₖ—NR^{B}(C═O)—. In certain embodiments, Y is —(CH₂)ₖ—NR^{B}—, —(CH₂)ₖ—O—, or —NR^{B}(C═O)—(CH₂)ₖ—O—. In certain embodiments, Y is —(CH₂)ₖ—NH—. In certain embodiments, Y is —NH—. In certain embodiments, Y is —(CH₂)ₖ—O—. In certain embodiments, Y is —O—. In certain embodiments, Y is —NH(C═O)—(CH₂)—O—. In certain embodiments, Y is —NH—, —O—, or —NH(C═O)—(CH₂)—O—.

In certain embodiments, E is of formula (E-II-a):

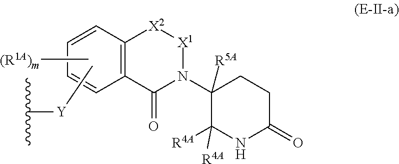

wherein:

Y is —(CH₂)ₖ—, —(CH₂)ₖ—O—, —O(CH₂)ₖ—, —NR^{B}(CH₂)ₖ—, —(CH₂)ₖ—NR^{B}—, —(CH₂)ₖ—(C═O)NR^{B}—, —O(CH₂)ₖ—(C═O)NR^{B}—, —O(CH₂)ₖ—NR^{B}(C═O)—, —NR^{B}(C═O)—(CH₂)ₖ—O—, —NR^{B}(CH₂)ₖ—NR^{B}(C═O)—, or —(CH₂)ₖ—NR^{B}(C═O)—;

X¹-X² is C(R^{3A})═N or C(R^{3A})₂—C(R^{3A})₂;

each R^{B} is, independently, hydrogen, or substituted or unsubstituted alkyl;

each R^{1A} is, independently, halogen, OH, C₁-C₆ alkyl, or C₁-C₆ alkoxy;

each R^{3A} is, independently, H or C₁-C₃ alkyl;

each R^{4A} is, independently, H or C₁-C₃ alkyl; or two R^{4A}, together with the carbon atom to which they are attached, form a C(O), C₃-C₆ carbocycle, or a 4-, 5-, or 6-membered heterocycle comprising 1 or 2 heteroatoms selected from N and O;

R^{5A} is hydrogen, C₁-C₃ alkyl, F, or Cl;

k is 0, 1, 2, 3, 4, 5, or 6; and

m is 0, 1, 2, or 3.

In certain embodiments of formula (E-II-a):

Y is —(CH₂)ₖ—, —(CH₂)ₖ—O—, —O(CH₂)ₖ—, —NR^{B}(CH₂)ₖ—, —(CH₂)ₖ—NR^{B}—, —(CH₂)ₖ—(C═O)NR^{B}—, —O(CH₂)ₖ—(C═O)NR^{B}—, —O(CH₂)ₖ—NR^{B}(C═O)—, —NR^{B}(CH₂)ₖ—NR^{B}(C═O)—, or —(CH₂)ₖ—NR^{B}(C═O)—;

X¹-X² is C(R^{3A})═N or C(R^{3A})₂—C(R^{3A})₂;

each R^{B} is, independently, hydrogen, or substituted or unsubstituted alkyl;

each R^{1A} is, independently, halogen, OH, C₁-C₆ alkyl, or C₁-C₆ alkoxy;

each R^{3A} is, independently, H or C₁-C₃ alkyl;

each R^{4A} is, independently, H or C₁-C₃ alkyl; or two R^{4A}, together with the carbon atom to which they are attached, form a C(O), C₃-C₆ carbocycle, or a 4-, 5-, or 6-membered heterocycle comprising 1 or 2 heteroatoms selected from N and O;

R^{5A} is hydrogen, C₁-C₃ alkyl, F, or Cl;

k is 0, 1, 2, 3, 4, 5, or 6; and

m is 0, 1, 2, or 3.

In certain embodiments of formula (E-II-a):

Y is —(CH₂)ₖ—NR^{B}—, —O(CH₂)ₖ—(C═O)NR^{B}—, —NR^{B}(C═O)—(CH₂)ₖ—O—, or —(CH₂)ₖ—NR^{B}(C═O)—;

X¹-X² is C(R^{3A})═N or C(R^{3A})₂—C(R^{3A})₂;

each R^{B} is, independently, hydrogen, or substituted or unsubstituted alkyl;

each R^{1A} is, independently, halogen, OH, C₁-C₆ alkyl, or C₁-C₆ alkoxy;

each R^{3A} is, independently, H or C₁-C₃ alkyl;

each R^{4A} is, independently, H or C₁-C₃ alkyl; or two R^{4A}, together with the carbon atom to which they are attached, form a C(O), C₃-C₆ carbocycle, or a 4-, 5-, or 6-membered heterocycle comprising 1 or 2 heteroatoms selected from N and O;

R^{5A} is hydrogen, C₁-C₃ alkyl, F, or Cl;

k is 0, 1, 2, 3, 4, 5, or 6; and

m is 0, 1, 2, or 3.

In certain embodiments of formula (E-II-a):

Y is —(CH₂)ₖ—NR^{B}—, —O(CH₂)ₖ—(C═O)NR^{B}—, or —(CH₂)ₖ—NR^{B}(C═O)—;

X¹-X² is C(R^{3A})═N or C(R^{3A})₂—C(R^{3A})₂;

each R^{B} is, independently, hydrogen, or substituted or unsubstituted alkyl;

each R^{1A} is, independently, halogen, OH, C₁-C₆ alkyl, or C₁-C₆ alkoxy;

each R^{3A} is, independently, H or C₁-C₃ alkyl;

each R^{4A} is, independently, H or C₁-C₃ alkyl; or two R^{4A}, together with the carbon atom to which they are attached, form a C(O), C₃-C₆ carbocycle, or a 4-, 5-, or 6-membered heterocycle comprising 1 or 2 heteroatoms selected from N and O;

R^{5A} is hydrogen, C₁-C₃ alkyl, F, or Cl;

k is 0, 1, 2, 3, 4, 5, or 6; and

m is 0, 1, 2 or 3.

In certain embodiments, E is of formula (E-II-b):

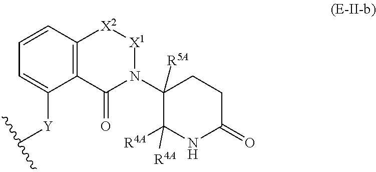

wherein:

Y is —(CH₂)ₖ—NR^{B}—, —O(CH₂)ₖ—(C═O)NR^{B}—, —NR^{B}(C═O)—(CH₂)ₖ—O—, or —(CH₂)ₖ—NR^{B}(C═O)—;

X¹-X² is C(R^{3A})═N or C(R^{3A})₂—C(R^{3A})₂;

each R^{B} is, independently, hydrogen, or substituted or unsubstituted alkyl;

each R^{3A} is, independently, H or C₁-C₃ alkyl;

each R^{4A} is, independently, H or C₁-C₃ alkyl; or two R^{4A}, together with the carbon atom to which they are attached, form a C(O), C₃-C₆ carbocycle, or a 4-, 5-, or 6-membered heterocycle comprising 1 or 2 heteroatoms selected from N and O;

R^{5A} is hydrogen, C₁-C₃ alkyl, F, or Cl; and

k is 0, 1, 2, 3, 4, 5, or 6.

In certain embodiments of formula (E-II-b):

Y is —(CH₂)ₖ—NR^{B}—, —O(CH₂)ₖ—(C═O)NR^{B}—, or —(CH₂)ₖ—NR^{B}(C═O)—;

X¹-X² is C(R^{3A})═N or C(R^{3A})₂—C(R^{3A})₂;

each R^{B} is, independently, hydrogen, or substituted or unsubstituted alkyl;

each R^{3A} is, independently, H or C₁-C₃ alkyl;

each R^{4A} is, independently, H or C₁-C₃ alkyl; or two R^{4A}, together with the carbon atom to which they are attached, form a C(O), C₃-C₆ carbocycle, or a 4-, 5-, or 6-membered heterocycle comprising 1 or 2 heteroatoms selected from N and O;

R^{5A} is hydrogen, C₁-C₃ alkyl, F, or Cl; and

k is 0, 1, 2, 3, 4, 5, or 6.

In certain embodiments, E is of formula (E-II-c):

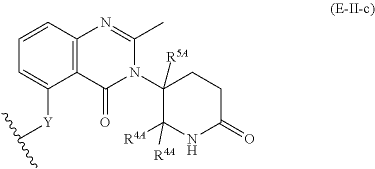

wherein:

Y is —(CH₂)ₖ—NR^{B}—, —O(CH₂)ₖ—(C═O)NR^{B}—, —NR^{B}(C═O)—(CH₂)ₖ—O—, or —(CH₂)ₖ—NR^{B}(C═O)—;

each R^{B} is, independently, hydrogen, or substituted or unsubstituted alkyl;

each R^{3A} is, independently, H or C₁-C₃ alkyl;

each R^{4A} is, independently, H or C₁-C₃ alkyl; or two R^{4A}, together with the carbon atom to which they are attached, form a C(O), C₃-C₆ carbocycle, or a 4-, 5-, or 6-membered heterocycle comprising 1 or 2 heteroatoms selected from N and O;

R^{5A} is hydrogen, C₁-C₃ alkyl, F, or Cl; and

k is 0, 1, 2, 3, 4, 5, or 6.

In certain embodiments of formula (E-II-c):

Y is —(CH₂)ₖ—NR^{B}—, —O(CH₂)ₖ—(C═O)NR^{B}—, or —(CH₂)ₖ—NR^{B}(C═O)—;

X¹-X² is C(R^{3A})═N or C(R^{3A})₂—C(R^{3A})₂;

each R^{B} is, independently, hydrogen, or substituted or unsubstituted alkyl;

each R^{3A} is, independently, H or C₁-C₃ alkyl;

each R^{4A} is, independently, H or C₁-C₃ alkyl; or two R^{4A}, together with the carbon atom to which they are attached, form a C(O), C₃-C₆ carbocycle, or a 4-, 5-, or 6-membered heterocycle comprising 1 or 2 heteroatoms selected from N and O;

R^{5A} is hydrogen, C₁-C₃ alkyl, F, or Cl; and

k is 0, 1, 2, 3, 4, 5, or 6.

In certain embodiments, E is of formula (E-II-d):

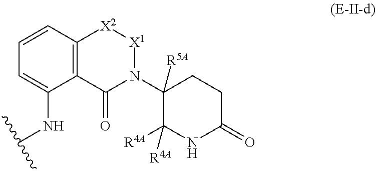

wherein:

X¹-X² is C(R^{3A})═N or C(R^{3A})₂—C(R^{3A})₂;

each R^{B} is, independently, hydrogen, or substituted or unsubstituted alkyl;

each R^{3A} is, independently, H or C₁-C₃ alkyl;

each R^{4A} is, independently, H or C₁-C₃ alkyl; or two R^{4A}, together with the carbon atom to which they are attached, form a C(O), C₃-C₆ carbocycle, or a 4-, 5-, or 6-membered heterocycle comprising 1 or 2 heteroatoms selected from N and O; and

R^{5A} is hydrogen, C₁-C₃ alkyl, F, or Cl.

In certain embodiments, E is of formula (E-II-e):

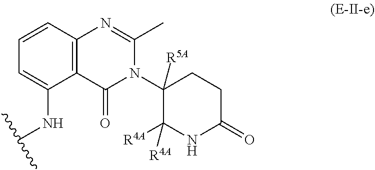

wherein:

each R^{4A} is, independently, H or C₁-C₃ alkyl; or two R^{4A}, together with the carbon atom to which they are attached, form a C(O), C₃-C₆ carbocycle, or a 4-, 5-, or 6-membered heterocycle comprising 1 or 2 heteroatoms selected from N and O; and

R^{5A} is hydrogen, C₁-C₃ alkyl, F, or Cl.

In certain embodiments, E is

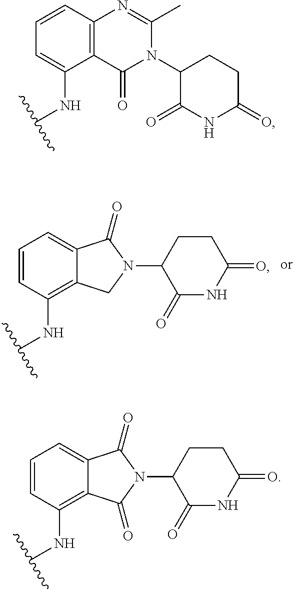 [SMILES: *Nc1cccc2nc(C)n(C3CCC(=O)NC3=O)c(=O)c12 ; *Nc1cccc2c1CN(C1CCC(=O)NC1=O)C2=O ; "or" ; *Nc1cccc2c1C(=O)N(C1CCC(=O)NC1=O)C2=O]

In certain embodiments, E is

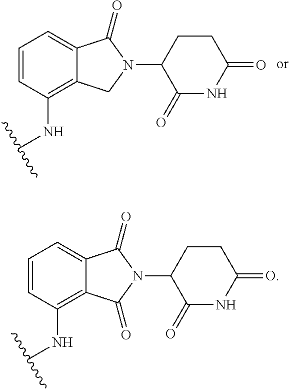 [SMILES: *Nc1cccc2c1CN(C1CCC(=O)NC1=O)C2=O ; "or" ; *Nc1cccc2c1C(=O)N(C1CCC(=O)NC1=O)C2=O]

In certain embodiments, E is

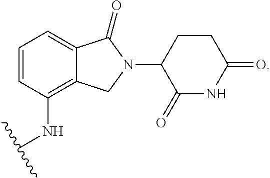 [SMILES: *Nc1cccc2c1CN(C1CCC(=O)NC1=O)C2=O]

In certain embodiments, E is

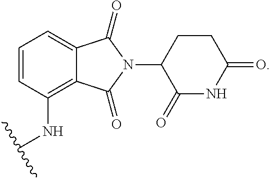 [SMILES: *Nc1cccc2c1C(=O)N(C1CCC(=O)NC1=O)C2=O]

The von Hippel-Lindau tumor suppressor (VHL) is an E3 ubiquitin ligase. VHL comprises the substrate recognition subunit/E3 ubiquitin ligase complex VCB, which includes elongins B and C, and a complex including Cullin-2 and Rbx 1. The primary substrate of VHL is Hypoxia Inducible Factor 1α (HIF-1α), a transcription factor that upregulates genes, such as the pro-angiogenic growth factor VEGF, and the red blood cell-inducing cytokine, erythropoietin, in response to low oxygen levels. VCB is a known target in cancer, chronic anemia, and ischemia.

The full-length von Hippel-Lindau tumor suppressor protein (VHL) contains 213 amino acids. (For the VHL protein sequence see: Duan et al., Proc. Natl. Acad. Sci. U.S.A. 1995, 92, 6459-63. For additional information related to the VHL structure see Stebbins et al., Science 1999, 284, 455-61 and Minervini et al., Sci. Rep. 2015, 5, 12605.) A second VHL-gene product arises by internal translation initiation from the codon 54 methionine, producing a 160 amino-acid protein (“pVHL19”). VHL has two main structural domains: an N-terminal domain composed mainly of β-sheets (β-domain) and a smaller C-terminal domain between amino acids 155-192 composed mainly of α helices (α-domain). The α-domain consists of three α helices that combines with a fourth a helix donated by elongin C. The β-domain is on the opposite side of the a domain and is free to contact other protein.

In certain embodiments, E is a modulator, binder, inhibitor, or ligand of VHL. In certain embodiments, E is a modulator of VHL. In certain embodiments, E is a binder of VHL. In certain embodiments, E is an inhibitor of VHL. In certain embodiments, E is a ligand of Cereblon. In certain embodiments, E is any ligand of VHL disclosed in U.S. Patent Application, U.S. Ser. No. 16/523,219, filed Jul. 26, 2019; U.S. Patent Application, U.S. Ser. No. 16/375,643, filed Apr. 4, 2019; U.S. Patent Application, U.S. Ser. No. 16/230,792, filed Dec. 21, 2018; U.S. Patent Application, U.S. Ser. No. 15/840,950, filed Sep. 13, 2018; U.S. Patent Application, U.S. Ser. No. 15/996,151, filed Jun. 1, 2018; U.S. Patent Application, U.S. Ser. No. 15/953,108, filed Apr. 13, 2018; U.S. Patent Application, U.S. Ser. No. 15/881,318, filed Jan. 26, 2018; U.S. Patent Application, U.S. Ser. No. 15/853,166, filed Dec. 22, 2017; U.S. Patent Application, U.S. Ser. No. 15/852,854, filed Dec. 22, 2017; U.S. Patent Application, U.S. Ser. No. 15/851,053, filed Dec. 21, 2017; U.S. Patent Application, U.S. Ser. No. 15/829,541, filed Dec. 1, 2017; U.S. Patent Application, U.S. Ser. No. 15/801,243, filed Nov. 1, 2017; U.S. Patent Application, U.S. Ser. No. 15/730,728, filed Oct. 11, 2017; U.S. Patent Application, U.S. Ser. No. 15/706,064, filed Sep. 15, 2017; U.S. Patent Application, U.S. Ser. No. 15/663,273, filed Jul. 28, 2017; U.S. Patent Application, U.S. Ser. No. 15/230,354, filed Aug. 5, 2016; U.S. Patent Application, U.S. Ser. No. 15/209,648, filed Jul. 13, 2016; U.S. Patent Application, U.S. Ser. No. 15/206,497, filed Jul. 11, 2016; U.S. Patent Application, U.S. Ser. No. 15/574,770, filed Jun. 6, 2016; U.S. Patent Application, U.S. Ser. No. 15/002,203, filed Jan. 20, 2016; U.S. Patent Application, U.S. Ser. No. 14/822,309, filed Aug. 10, 2015; U.S. Patent Application, U.S. Ser. No. 14/707,930, filed May 8, 2015; International Patent Application, PCT/US2019/040545, filed Jul. 3, 2019; International Patent Application, PCT/US2019/040520, filed Jul. 3, 2019; International Patent Application, PCT/US2019/013481, filed Jan. 14, 2019; International Patent Application, PCT/US2018/052181, filed Sep. 21, 2018; International Patent Application, PCT/US2013/054663, filed Aug. 13, 2013; and Galdeano, C. et al. J. Med. Chem. 2014, 57, 8657, each of which is incorporated herein by reference. In certain embodiments, E is a modulator, binder, inhibitor, or ligand of a VHL variant. In certain embodiments, E is a modulator, binder, inhibitor, or ligand of a VHL isoform. In certain embodiments, E is a modulator, binder, inhibitor, or ligand of a VHL gene-product (e.g., pVHL19).

In certain embodiments, E is of Formula (E-III):

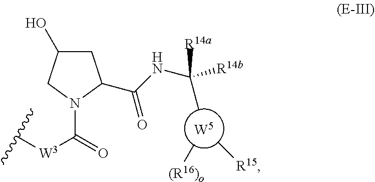

wherein:

W³ is substituted or unsubstituted arylene, substituted or unsubstituted heteroarylene, or

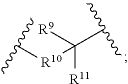

R⁹ and R¹¹ are independently hydrogen, substituted or unsubstituted alkyl, substituted or unsubstituted cycloalkyl, substituted or unsubstituted hydroxyalkyl, substituted or unsubstituted heteroaryl, or haloalkyl; or R⁹, R¹¹, and the carbon atom to which they are attached form a substituted or unsubstituted cycloalkyl;

R¹⁰ is —O—, —NH—, substituted or unsubstituted heterocyclylene, substituted or unsubstituted heteroarylene, or substituted or unsubstituted arylene;

R^{14a} and R^{14b} are each independently hydrogen, haloalkyl, or substituted or unsubstituted alkyl;

W⁵ is aryl or heteroaryl;

R₁₅ is hydrogen, halogen, CN, OH, NO₂, —NR^{14a}R^{14b}, OR^{14a}, CONR^{14a}R^{14b}, NR^{14a}COR^{14b}, SO₂NR^{14a}R^{14b}, NR^{14a}SO₂R^{14b}), substituted or unsubstituted alkyl, haloalkyl, haloalkoxy, substituted or unsubstituted aryl, substituted or unsubstituted heteroaryl, substituted or unsubstituted cycloalkyl, or substituted or unsubstituted heterocyclyl;

each R¹⁶ is independently halo, substituted or unsubstituted alkyl, haloalkyl, hydroxy, haloalkoxy, or

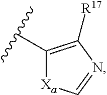

wherein R¹⁷ is hydrogen, halogen, substituted or unsubstituted C₃₋₆ cycloalkyl, substituted or unsubstituted C₁₋₆ alkyl, substituted or unsubstituted C₁₋₆ alkenyl, or C₁₋₆ haloalkyl; Xₐ is S or O; and

o is 0, 1, 2, 3, or 4.

In certain embodiments, E is of Formula (E-III-a):

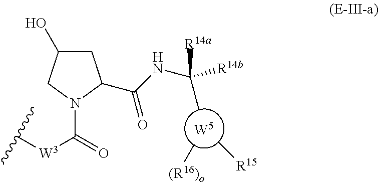

wherein W³, W⁵, R^{14a}, R^{14b}, R¹⁵, R¹⁶, and o are as defined herein.

In certain embodiments, E is of Formula (E-III-a-1):

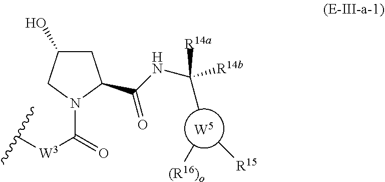

wherein W³, W⁵, R^{14a}, R^{14b}, R¹⁵, R¹⁶, and o are as defined herein.

In certain embodiments, E is of Formula (E-III-b):

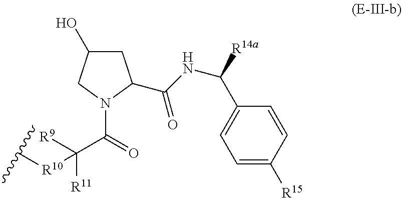

wherein W³, R⁹, R¹⁰, R¹¹, R^{14a}, and R¹⁵ are as defined herein.

In certain embodiments, E is of Formula (E-III-b-1):

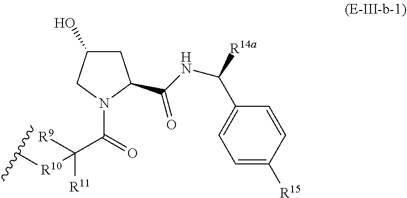

wherein W³, R⁹, R¹⁰, R¹¹, R^{14a}, and R¹⁵ are as defined herein.

In certain embodiments, E is of Formula (E-III-c):

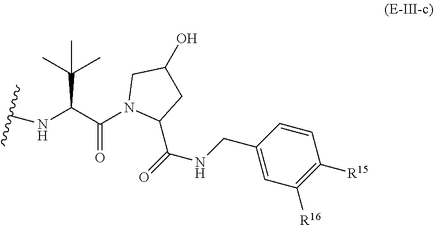

wherein R¹⁵ and R¹⁶ are as defined herein.

In certain embodiments, E is of Formula (E-III-c-1):

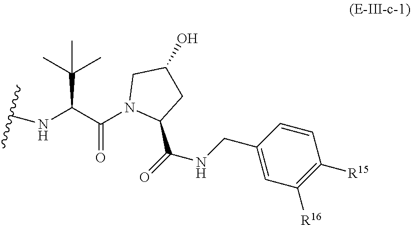

wherein R¹⁵ and R¹⁶ are as defined herein.

In certain embodiments, E is of Formula (E-III-d):

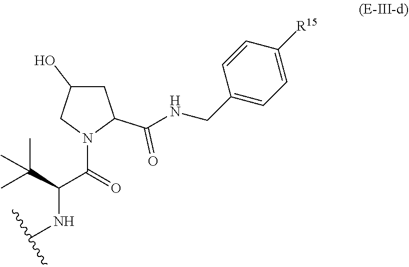

wherein R¹⁵ is as defined herein.

In certain embodiments, E is of Formula (E-III-d-1):

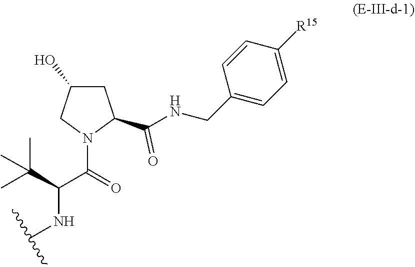

wherein R¹⁵ is as defined herein.

In certain embodiments, E is of the formula:

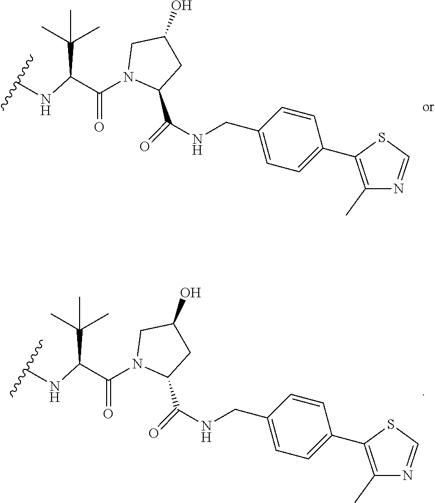 [SMILES: *N[C@H](C(=O)N1C[C@H](O)C[C@H]1C(=O)NCc1ccc(-c2scnc2C)cc1)C(C)(C)C ; *N[C@H](C(=O)N1C[C@@H](O)C[C@@H]1C(=O)NCc1ccc(-c2scnc2C)cc1)C(C)(C)C]

In certain embodiments, E is of the formula:

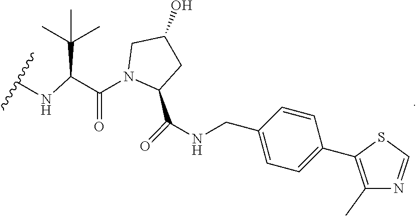 [SMILES: *N[C@H](C(=O)N1C[C@H](O)C[C@H]1C(=O)NCc1ccc(-c2scnc2C)cc1)C(C)(C)C]

In certain embodiments, E is of the formula:

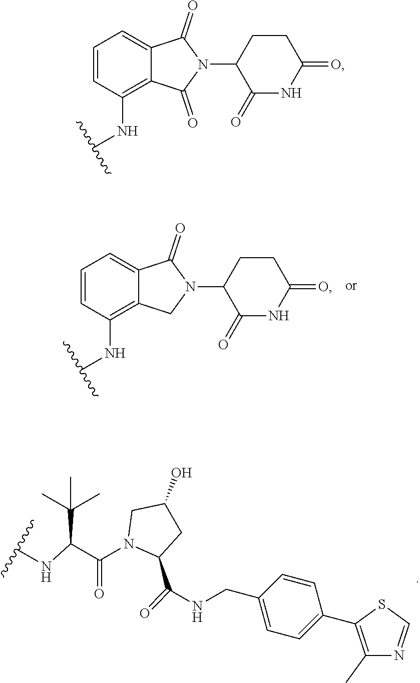 [SMILES: *Nc1cccc2c1C(=O)N(C1CCC(=O)NC1=O)C2=O ; *Nc1cccc2c1CN(C1CCC(=O)NC1=O)C2=O ; "or" ; *N[C@H](C(=O)N1C[C@H](O)C[C@H]1C(=O)NCc1ccc(-c2scnc2C)cc1)C(C)(C)C]

In certain embodiments, E is of the formula:

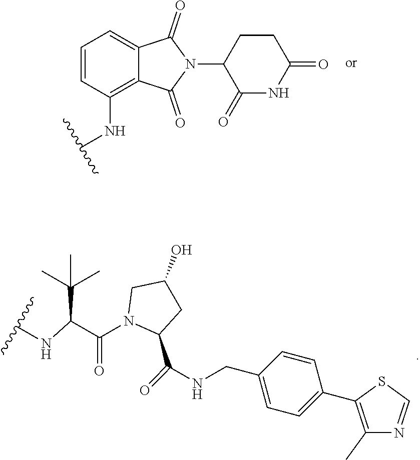 [SMILES: *Nc1cccc2c1C(=O)N(C1CCC(=O)NC1=O)C2=O ; "or" ; *N[C@H](C(=O)N1C[C@H](O)C[C@H]1C(=O)NCc1ccc(-c2scnc2C)cc1)C(C)(C)C]

In certain embodiments, the E3 ligase binding moiety binds an E3 ubiquitin ligase with a K_{d} of less than 100,000 nM, less than 50,000 nM, less than 20,000 nM, less than 10,000 nM, less than 5,000 nM, less than 2,500 nM, less than 1,000 nM, less than 900 nM, less than 800 nM, less than 700 nM, less than 600 nM, less than 500 nM, less than 400 nM, less than 300 nM, less than 200 nM, less than 100 nM, less than 90 nM, less than 80 nM, less than 70 nM, less than 60 nM, less than 50 nM, less than 40 nM, less than 30 nM, less than 20 nM, less than 10 nM, less than 5 nM, less than 4 nM, less than 3 nM, less than 2 nM, or less than 1 nM.

In certain embodiments, the E3 ligase binding moiety binds Cereblon with a K_{d} of less than 100,000 nM, less than 50,000 nM, less than 20,000 nM, less than 10,000 nM, less than 5,000 nM, less than 2,500 nM, less than 1,000 nM, less than 900 nM, less than 800 nM, less than 700 nM, less than 600 nM, less than 500 nM, less than 400 nM, less than 300 nM, less than 200 nM, less than 100 nM, less than 90 nM, less than 80 nM, less than 70 nM, less than 60 nM, less than 50 nM, less than 40 nM, less than 30 nM, less than 20 nM, less than 10 nM, less than 5 nM, less than 4 nM, less than 3 nM, less than 2 nM, or less than 1 nM.

In certain embodiments, the E3 ligase binding moiety binds VHL with a K_{d} of less than 100,000 nM, less than 50,000 nM, less than 20,000 nM, less than 10,000 nM, less than 5,000 nM, less than 2,500 nM, less than 1,000 nM, less than 900 nM, less than 800 nM, less than 700 nM, less than 600 nM, less than 500 nM, less than 400 nM, less than 300 nM, less than 200 nM, less than 100 nM, less than 90 nM, less than 80 nM, less than 70 nM, less than 60 nM, less than 50 nM, less than 40 nM, less than 30 nM, less than 20 nM, less than 10 nM, less than 5 nM, less than 4 nM, less than 3 nM, less than 2 nM, or less than 1 nM.

In certain embodiments, the E3 ligase binding moiety selectively binds an E3 ubiquitin ligase as compared to another protein. In some embodiments, the E3 ligase binding moiety selectively binds Cereblon over another protein. In some embodiments, the E3 ligase binding moiety selectively binds Cereblon over another E3 ubiquitin ligase. In some embodiments, the E3 ligase binding moiety selectively binds VHL over another protein. In some embodiments, the E3 ligase binding moiety selectively binds VHL over another E3 ubiquitin ligase. In certain embodiments, the selectivity is between about 2-fold and about 5-fold. In certain embodiments, the selectivity is between about 5-fold and about 10-fold. In certain embodiments, the selectivity is between about 10-fold and about 20-fold. In certain embodiments, the selectivity is between about 20-fold and about 50-fold. In certain embodiments, the selectivity is between about 50-fold and about 100-fold. In certain embodiments, the selectivity is between about 100-fold and about 200-fold. In certain embodiments, the selectivity is between about 200-fold and about 500-fold. In certain embodiments, the selectivity is between about 500-fold and about 1000-fold. In certain embodiments, the selectivity is at least about 1000-fold.

### Further Embodiments of Formula (I)

In certain embodiments, the compound of Formula (I) is a compound of Formula (I-a):

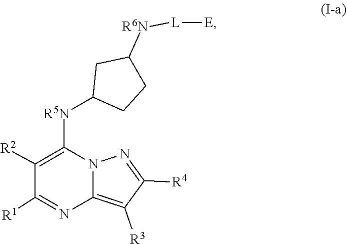

or a pharmaceutically acceptable salt, co-crystal, tautomer, stereoisomer, solvate, hydrate, polymorph, isotopically enriched derivative, or prodrug thereof, wherein E, L, R¹, R², R³, R⁴, R⁵, and R⁶ are as defined herein.

In certain embodiments, the compound of Formula (I) is a compound of Formula (I-b):

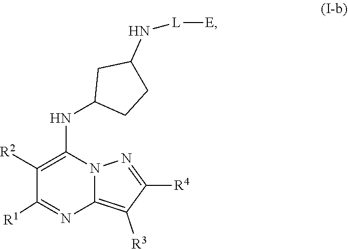

or a pharmaceutically acceptable salt, co-crystal, tautomer, stereoisomer, solvate, hydrate, polymorph, isotopically enriched derivative, or prodrug thereof, wherein E, L, R¹, R², R³, and R⁴ are as defined herein.

In certain embodiments, the compound of Formula (I) is a compound of Formula (I-c):

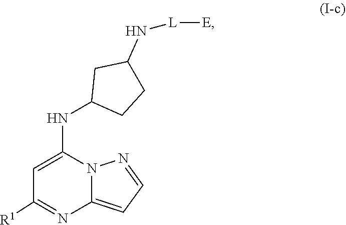

or a pharmaceutically acceptable salt, co-crystal, tautomer, stereoisomer, solvate, hydrate, polymorph, isotopically enriched derivative, or prodrug thereof, wherein E, L, and R¹ are as defined herein.

In certain embodiments, the compound of Formula (I) is a compound of Formula (I-d):

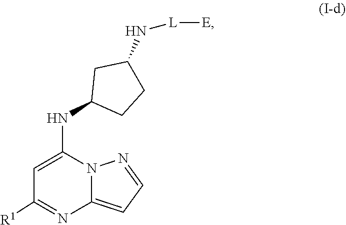

or a pharmaceutically acceptable salt, co-crystal, tautomer, stereoisomer, solvate, hydrate, polymorph, isotopically enriched derivative, or prodrug thereof, wherein E, L, and R¹ are as defined herein.

In certain embodiments, the compound of Formula (I) is a compound of Formula (I-d-1):

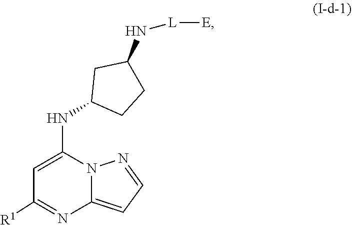

or a pharmaceutically acceptable salt, co-crystal, tautomer, stereoisomer, solvate, hydrate, polymorph, isotopically enriched derivative, or prodrug thereof, wherein E, L, and R¹ are as defined herein.

In certain embodiments, the compound of Formula (I) is a compound of Formula (I-e):

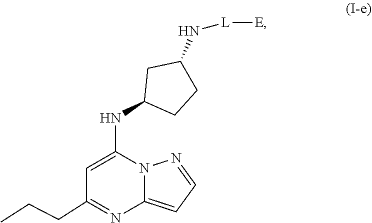

or a pharmaceutically acceptable salt, co-crystal, tautomer, stereoisomer, solvate, hydrate, polymorph, isotopically enriched derivative, or prodrug thereof, wherein E and L are as defined herein.

In certain embodiments, the compound of Formula (I) is a compound of Formula (I-e-1):

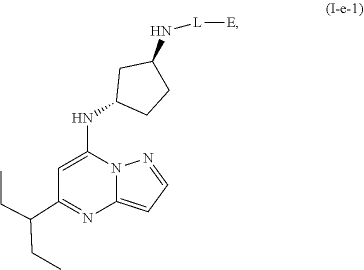

or a pharmaceutically acceptable salt, co-crystal, tautomer, stereoisomer, solvate, hydrate, polymorph, isotopically enriched derivative, or prodrug thereof, wherein E, L, and R¹ are as defined herein.

In certain embodiments, the compound of Formula (I) is a compound of Formula (I-f):

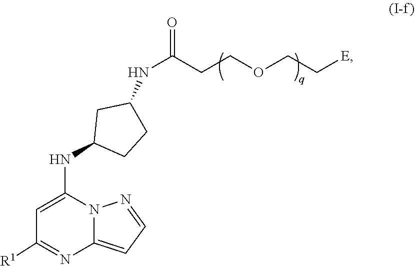

or a pharmaceutically acceptable salt, co-crystal, tautomer, stereoisomer, solvate, hydrate, polymorph, isotopically enriched derivative, or prodrug thereof, wherein q is 1-12, and E and R¹ are as defined herein.

In certain embodiments, the compound of Formula (I) is a compound of Formula (I-f-1):

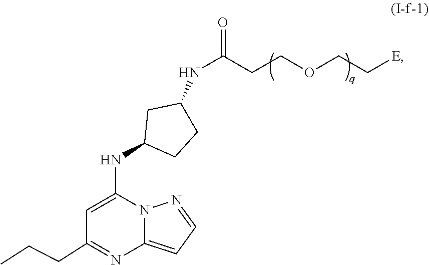

or a pharmaceutically acceptable salt, co-crystal, tautomer, stereoisomer, solvate, hydrate, polymorph, isotopically enriched derivative, or prodrug thereof, wherein q is 1-12, and E is as defined herein.

In certain embodiments, the compound of Formula (I) is a compound of Formula (I-f-2):

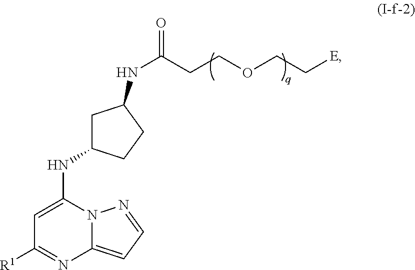

or a pharmaceutically acceptable salt, co-crystal, tautomer, stereoisomer, solvate, hydrate, polymorph, isotopically enriched derivative, or prodrug thereof, wherein q is 1-12, and E and R¹ are as defined herein.

In certain embodiments, the compound of Formula (I) is a compound of Formula (I-f-3):

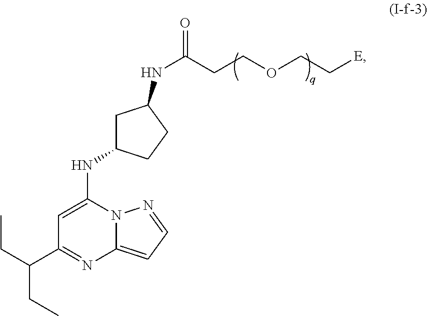

or a pharmaceutically acceptable salt, co-crystal, tautomer, stereoisomer, solvate, hydrate, polymorph, isotopically enriched derivative, or prodrug thereof, wherein q is 1-12, and E and R¹ are as defined herein.

In certain embodiments, the compound of Formula (I) is a compound of Formula (I-g):

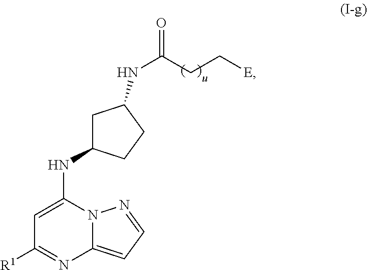

or a pharmaceutically acceptable salt, co-crystal, tautomer, stereoisomer, solvate, hydrate, polymorph, isotopically enriched derivative, or prodrug thereof, wherein u is 1-8, and E and R¹ are as defined herein.

In certain embodiments, the compound of Formula (I) is a compound of Formula (I-g-1):

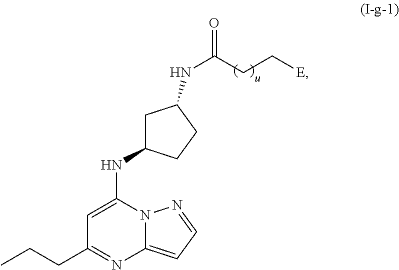

or a pharmaceutically acceptable salt, co-crystal, tautomer, stereoisomer, solvate, hydrate, polymorph, isotopically enriched derivative, or prodrug thereof, wherein u is 1-8, and E is as defined herein.

In certain embodiments, the compound of Formula (I) is a compound of Formula (I-g-2):

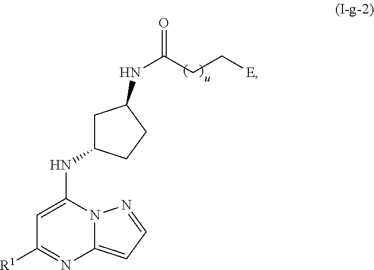

or a pharmaceutically acceptable salt, co-crystal, tautomer, stereoisomer, solvate, hydrate, polymorph, isotopically enriched derivative, or prodrug thereof, wherein u is 1-8, and E and R¹ are as defined herein.

In certain embodiments, the compound of Formula (I) is a compound of Formula (I-g-3):

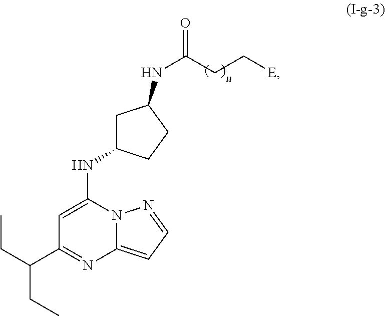

or a pharmaceutically acceptable salt, co-crystal, tautomer, stereoisomer, solvate, hydrate, polymorph, isotopically enriched derivative, or prodrug thereof, wherein u is 1-8, and E is as defined herein.

In certain embodiments, the compound of Formula (I) is a compound of Formula (I-h):

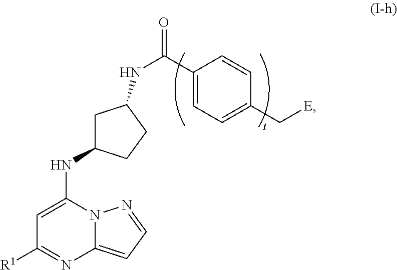

or a pharmaceutically acceptable salt, co-crystal, tautomer, stereoisomer, solvate, hydrate, polymorph, isotopically enriched derivative, or prodrug thereof, wherein t is 1 or 2, and E and R¹ are as defined herein.

In certain embodiments, the compound of Formula (I) is a compound of Formula (I-h-1):

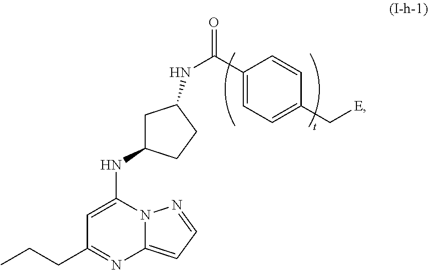

or a pharmaceutically acceptable salt, co-crystal, tautomer, stereoisomer, solvate, hydrate, polymorph, isotopically enriched derivative, or prodrug thereof, wherein t is 1 or 2, and E is as defined herein.

In certain embodiments, the compound of Formula (I) is a compound of Formula (I-h-2):

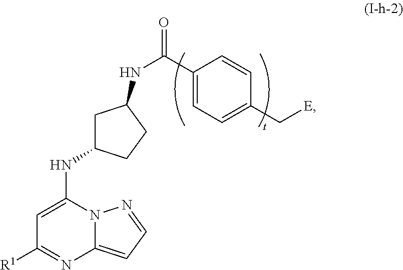

or a pharmaceutically acceptable salt, co-crystal, tautomer, stereoisomer, solvate, hydrate, polymorph, isotopically enriched derivative, or prodrug thereof, wherein t is 1 or 2, and E and R¹ are as defined herein.

In certain embodiments, the compound of Formula (I) is a compound of Formula (I-h-3):

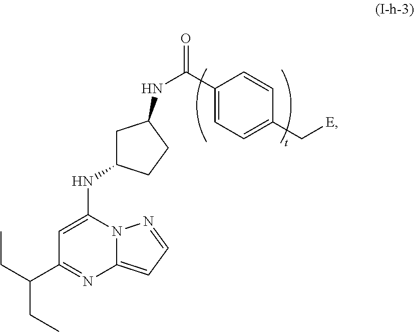

or a pharmaceutically acceptable salt, co-crystal, tautomer, stereoisomer, solvate, hydrate, polymorph, isotopically enriched derivative, or prodrug thereof, wherein t is 1 or 2, and E is as defined herein.

In certain embodiments, the compound of Formula (I) is a compound of Formula (I-i):

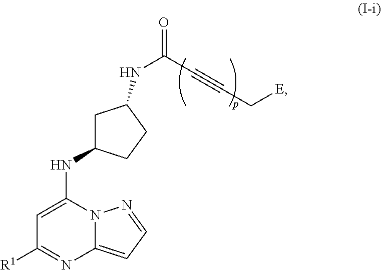

or a pharmaceutically acceptable salt, co-crystal, tautomer, stereoisomer, solvate, hydrate, polymorph, isotopically enriched derivative, or prodrug thereof, wherein p is 1 or 2, and E and R¹ are as defined herein.

In certain embodiments, the compound of Formula (I) is a compound of Formula (I-i-1):

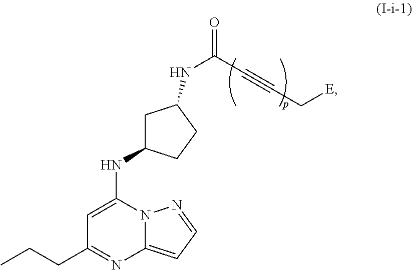

or a pharmaceutically acceptable salt, co-crystal, tautomer, stereoisomer, solvate, hydrate, polymorph, isotopically enriched derivative, or prodrug thereof, wherein p is 1 or 2, and E is as defined herein.

In certain embodiments, the compound of Formula (I) is a compound of Formula (I-i-2):

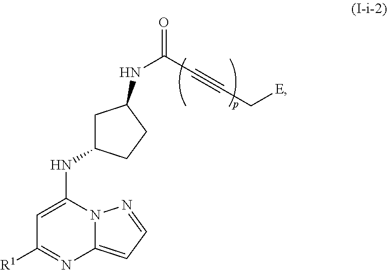

or a pharmaceutically acceptable salt, co-crystal, tautomer, stereoisomer, solvate, hydrate, polymorph, isotopically enriched derivative, or prodrug thereof, wherein p is 1 or 2, and E and R¹ are as defined herein.

In certain embodiments, the compound of Formula (I) is a compound of Formula (I-i-3):

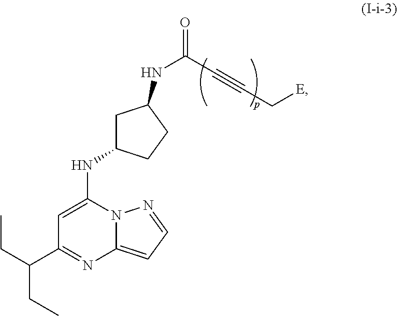

or a pharmaceutically acceptable salt, co-crystal, tautomer, stereoisomer, solvate, hydrate, polymorph, isotopically enriched derivative, or prodrug thereof, wherein p is 1 or 2, and E is as defined herein.

In certain embodiments, the compound of Formula (I) is a compound of Formula (I-j):

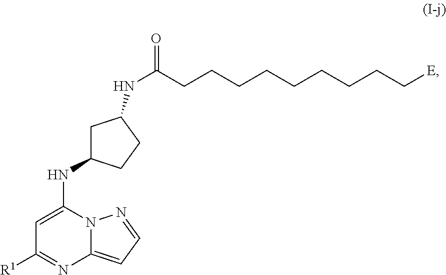

or a pharmaceutically acceptable salt, co-crystal, tautomer, stereoisomer, solvate, hydrate, polymorph, isotopically enriched derivative, or prodrug thereof, wherein E and R¹ are as defined herein.

In certain embodiments, the compound of Formula (I) is a compound of Formula (I-j-1):

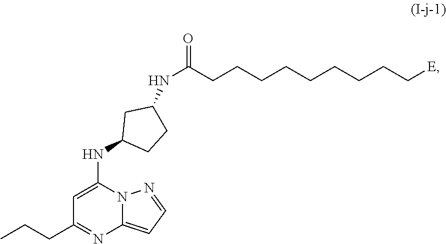

or a pharmaceutically acceptable salt, co-crystal, tautomer, stereoisomer, solvate, hydrate, polymorph, isotopically enriched derivative, or prodrug thereof, wherein E is as defined herein.

In certain embodiments, the compound of Formula (I) is a compound of Formula (I-j-2):

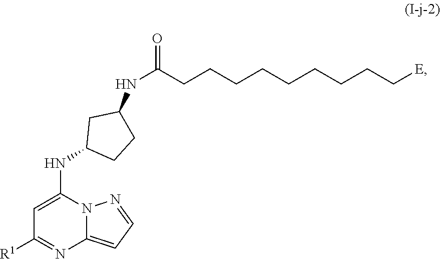

or a pharmaceutically acceptable salt, co-crystal, tautomer, stereoisomer, solvate, hydrate, polymorph, isotopically enriched derivative, or prodrug thereof, wherein E and R¹ are as defined herein.

In certain embodiments, the compound of Formula (I) is a compound of Formula (I-j-3):

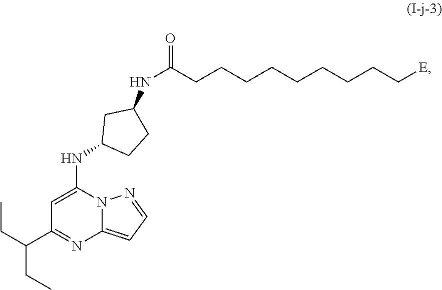

or a pharmaceutically acceptable salt, co-crystal, tautomer, stereoisomer, solvate, hydrate, polymorph, isotopically enriched derivative, or prodrug thereof, wherein E is as defined herein.

In certain embodiments, the compound of Formula (I) is a compound of Formula (I-k):

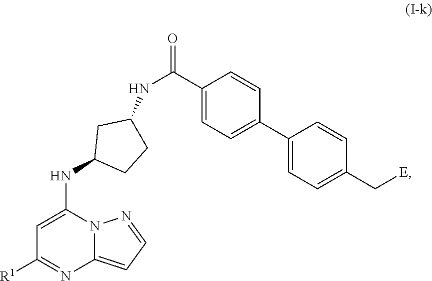

or a pharmaceutically acceptable salt, co-crystal, tautomer, stereoisomer, solvate, hydrate, polymorph, isotopically enriched derivative, or prodrug thereof, wherein E and R¹ are as defined herein.

In certain embodiments, the compound of Formula (I) is a compound of Formula (I-k-1):

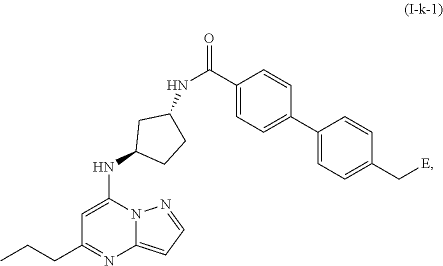

or a pharmaceutically acceptable salt, co-crystal, tautomer, stereoisomer, solvate, hydrate, polymorph, isotopically enriched derivative, or prodrug thereof, wherein E is as defined herein.

In certain embodiments, the compound of Formula (I) is a compound of Formula (I-k-2):

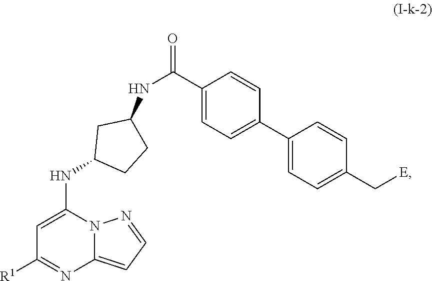

or a pharmaceutically acceptable salt, co-crystal, tautomer, stereoisomer, solvate, hydrate, polymorph, isotopically enriched derivative, or prodrug thereof, wherein E and R¹ are as defined herein.

In certain embodiments, the compound of Formula (I) is a compound of Formula (I-k-3):

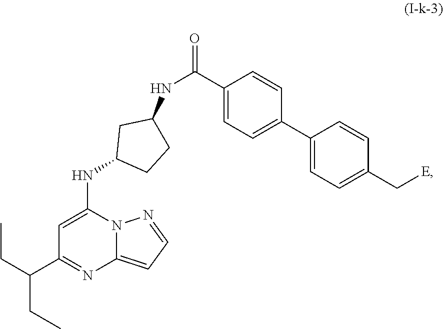

or a pharmaceutically acceptable salt, co-crystal, tautomer, stereoisomer, solvate, hydrate, polymorph, isotopically enriched derivative, or prodrug thereof, wherein E is as defined herein.

In certain embodiments, the compound of Formula (I) is a compound of the formula:

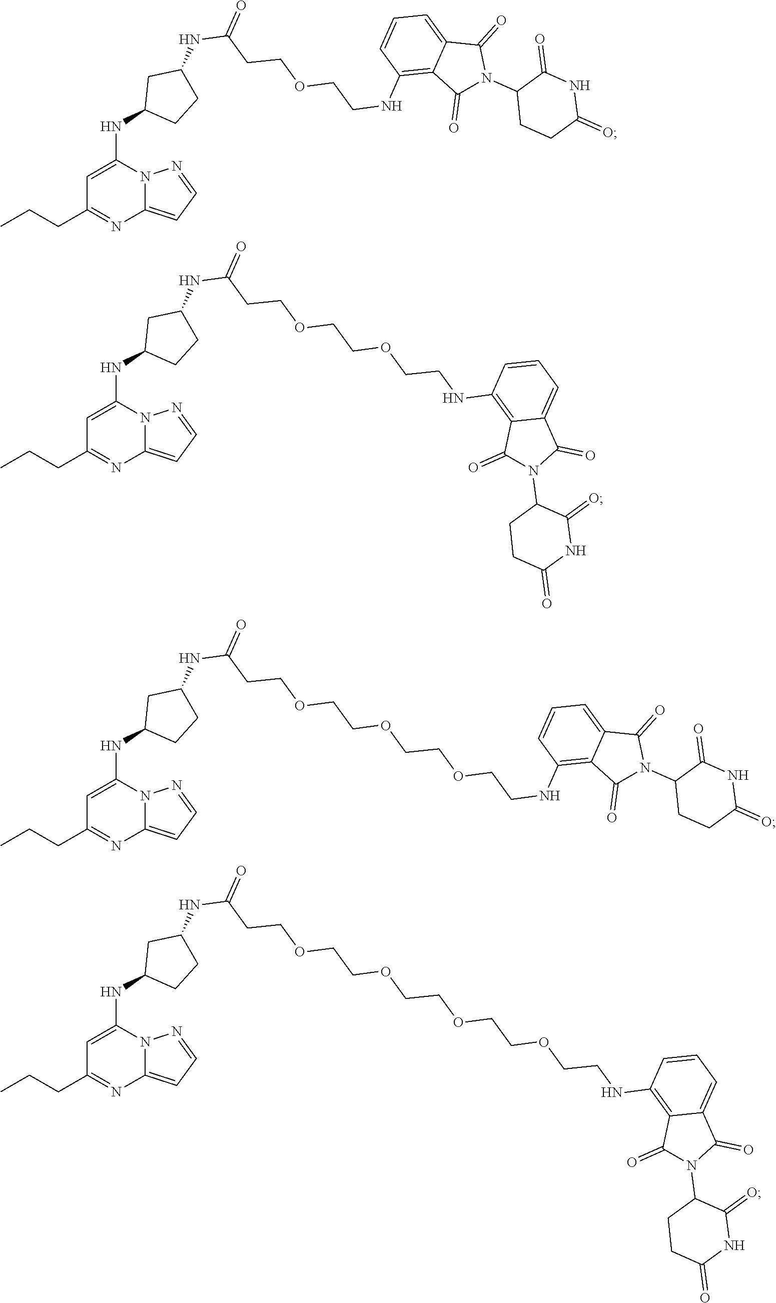 [SMILES: CCCc1cc(N[C@@H]2CC[C@@H](NC(=O)CCOCCNc3cccc4c3C(=O)N(C3CCC(=O)NC3=O)C4=O)C2)n2nccc2n1 ; CCCc1cc(N[C@@H]2CC[C@@H](NC(=O)CCOCCOCCNc3cccc4c3C(=O)N(C3CCC(=O)NC3=O)C4=O)C2)n2nccc2n1 ; CCCc1cc(N[C@@H]2CC[C@@H](NC(=O)CCOCCOCCOCCNc3cccc4c3C(=O)N(C3CCC(=O)NC3=O)C4=O)C2)n2nccc2n1 ; CCCc1cc(N[C@@H]2CC[C@@H](NC(=O)CCOCCOCCOCCOCCNc3cccc4c3C(=O)N(C3CCC(=O)NC3=O)C4=O)C2)n2nccc2n1]

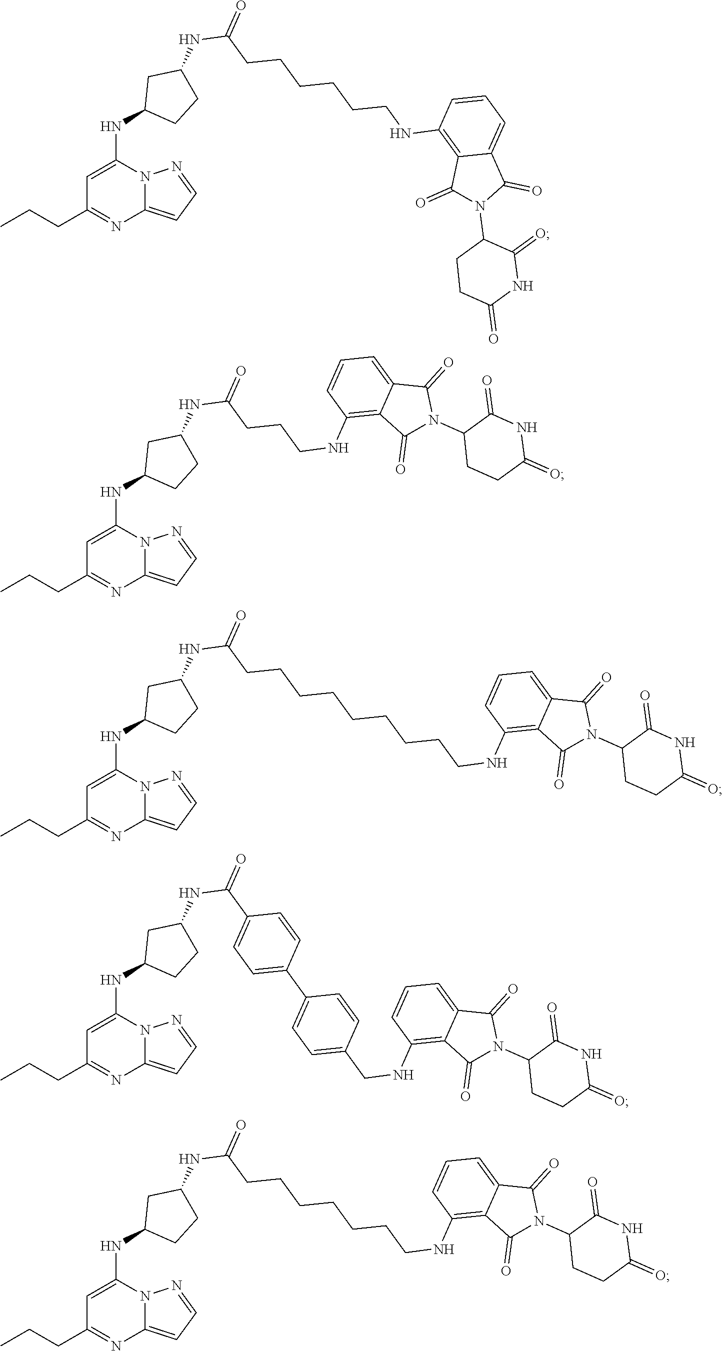

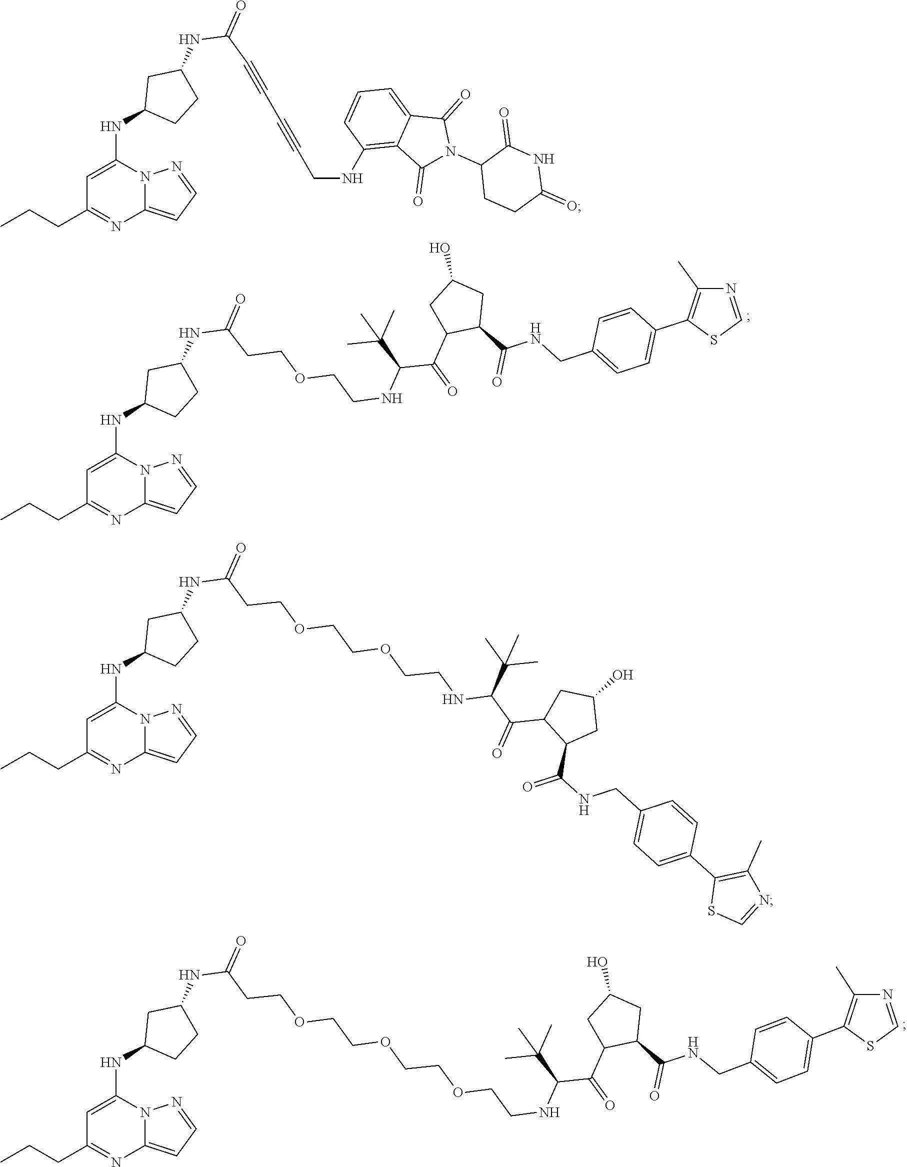 [SMILES: CCCc1cc(N[C@@H]2CC[C@@H](NC(=O)C#CC#CCNc3cccc4c3C(=O)N(C3CCC(=O)NC3=O)C4=O)C2)n2nccc2n1 ; CCCc1cc(N[C@@H]2CC[C@@H](NC(=O)CCOCCN[C@H](C(=O)C3C[C@H](O)C[C@H]3C(=O)NCc3ccc(-c4scnc4C)cc3)C(C)(C)C)C2)n2nccc2n1 ; CCCc1cc(N[C@@H]2CC[C@@H](NC(=O)CCOCCOCCN[C@H](C(=O)C3C[C@H](O)C[C@H]3C(=O)NCc3ccc(-c4scnc4C)cc3)C(C)(C)C)C2)n2nccc2n1 ; CCCc1cc(N[C@@H]2CC[C@@H](NC(=O)CCOCCOCCOCCN[C@H](C(=O)C3C[C@H](O)C[C@H]3C(=O)NCc3ccc(-c4scnc4C)cc3)C(C)(C)C)C2)n2nccc2n1]

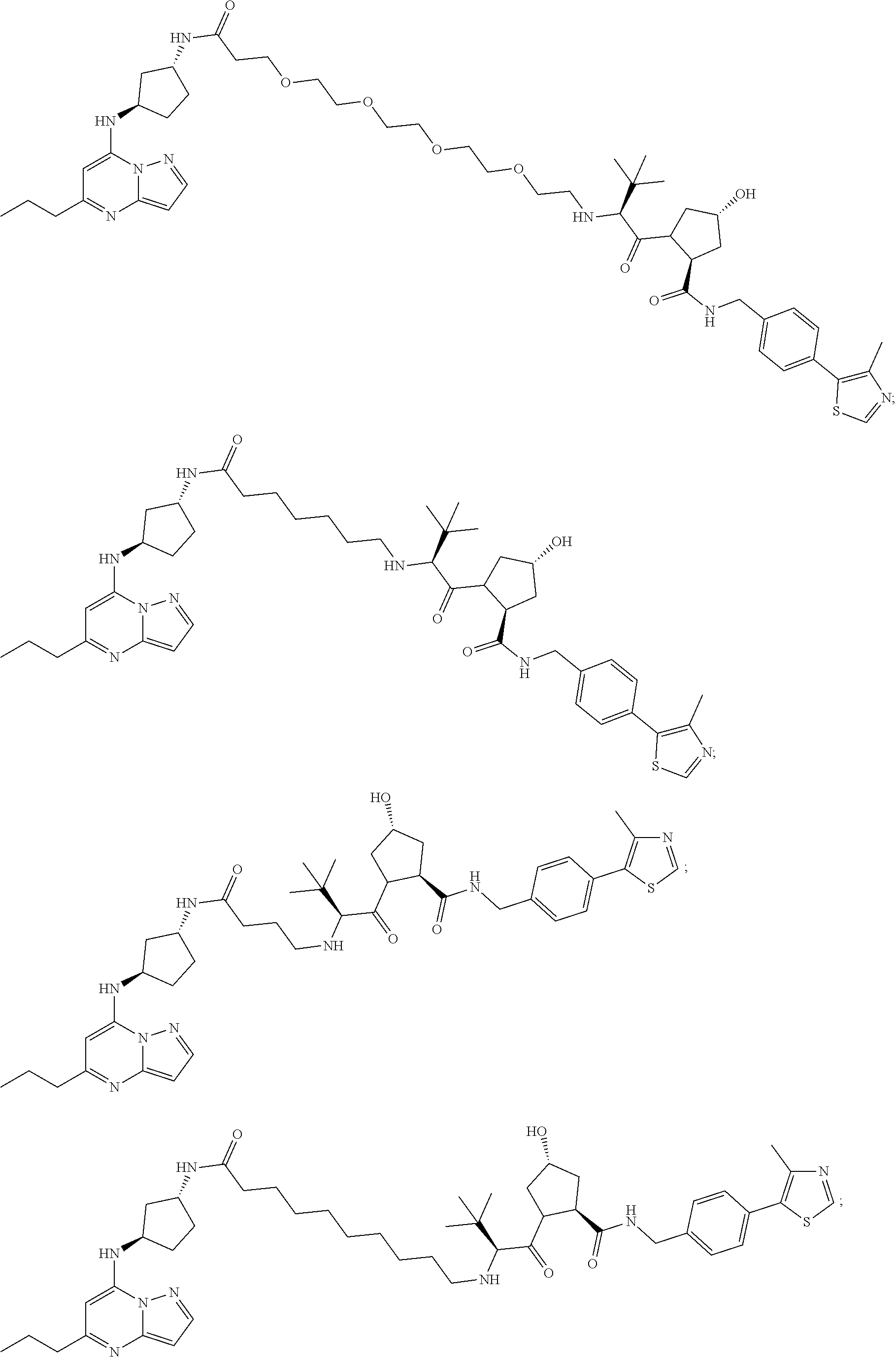 [SMILES: CCCc1cc(N[C@@H]2CC[C@@H](NC(=O)CCOCCOCCOCCOCCN[C@H](C(=O)C3C[C@H](O)C[C@H]3C(=O)NCc3ccc(-c4scnc4C)cc3)C(C)(C)C)C2)n2nccc2n1 ; CCCc1cc(N[C@@H]2CC[C@@H](NC(=O)CCCCCCN[C@H](C(=O)C3C[C@H](O)C[C@H]3C(=O)NCc3ccc(-c4scnc4C)cc3)C(C)(C)C)C2)n2nccc2n1 ; CCCc1cc(N[C@@H]2CC[C@@H](NC(=O)CCCN[C@H](C(=O)C3C[C@H](O)C[C@H]3C(=O)NCc3ccc(-c4scnc4C)cc3)C(C)(C)C)C2)n2nccc2n1 ; CCCc1cc(N[C@@H]2CC[C@@H](NC(=O)CCCCCCCCCN[C@H](C(=O)C3C[C@H](O)C[C@H]3C(=O)NCc3ccc(-c4scnc4C)cc3)C(C)(C)C)C2)n2nccc2n1]

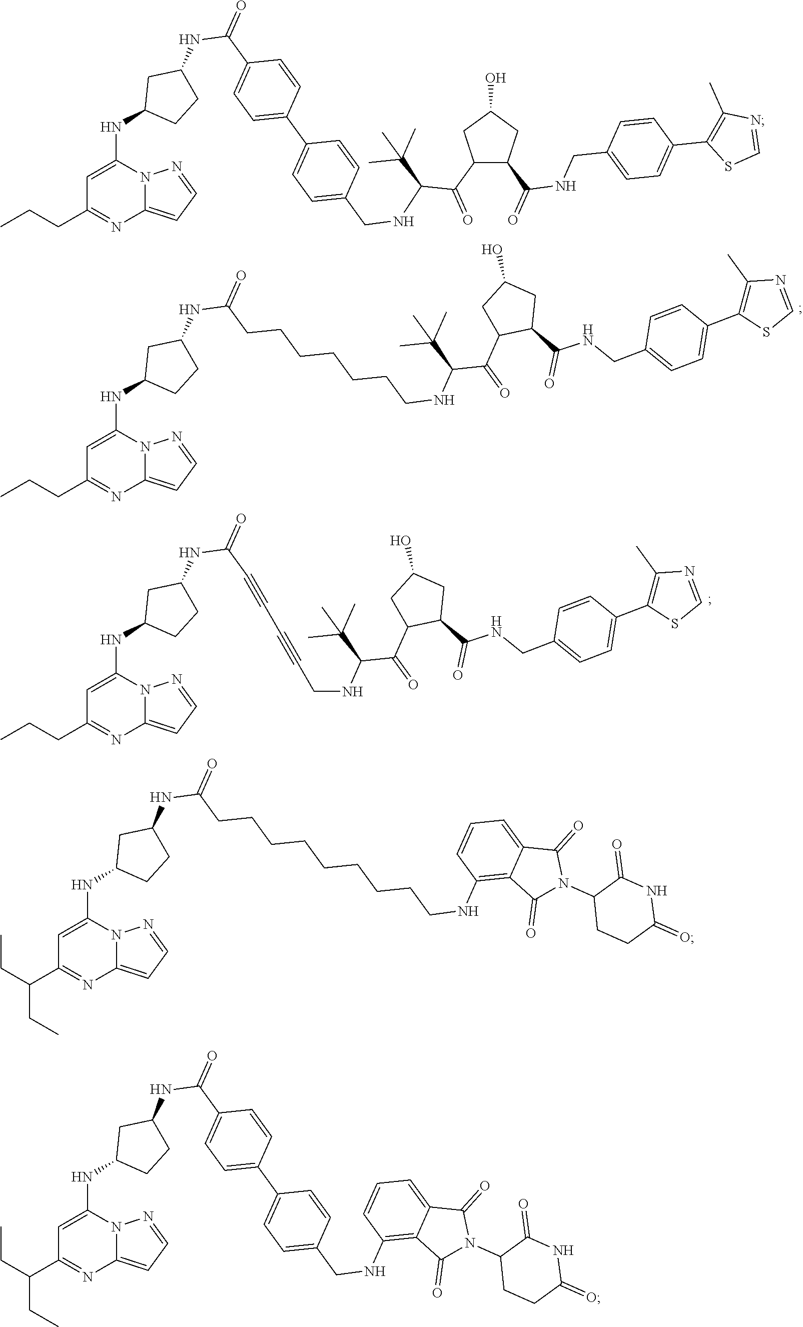

or a pharmaceutically acceptable salt, co-crystal, tautomer, stereoisomer, solvate, hydrate, polymorph, isotopically enriched derivative, or prodrug thereof.

In certain embodiments, the compound of Formula (I) is a compound of the formula:

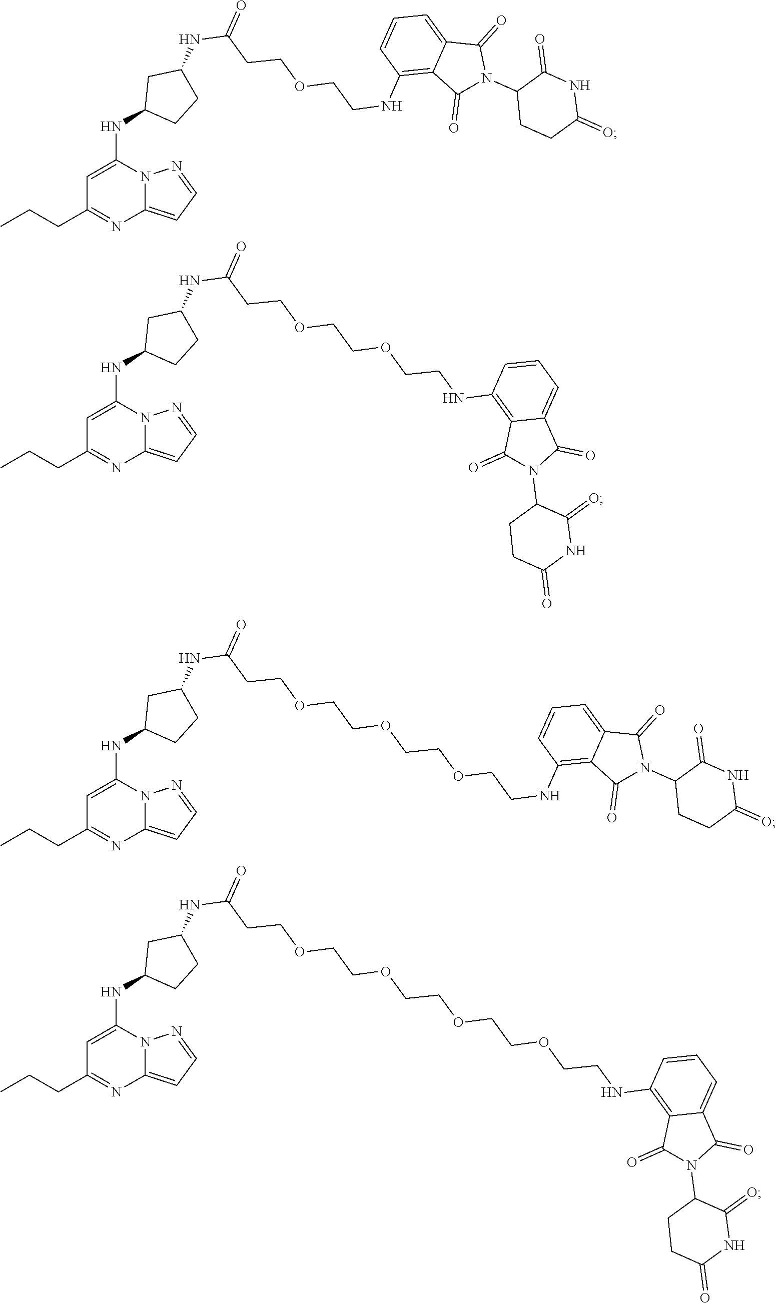 [SMILES: CCCc1cc(N[C@@H]2CC[C@@H](NC(=O)CCOCCNc3cccc4c3C(=O)N(C3CCC(=O)NC3=O)C4=O)C2)n2nccc2n1 ; CCCc1cc(N[C@@H]2CC[C@@H](NC(=O)CCOCCOCCNc3cccc4c3C(=O)N(C3CCC(=O)NC3=O)C4=O)C2)n2nccc2n1 ; CCCc1cc(N[C@@H]2CC[C@@H](NC(=O)CCOCCOCCOCCNc3cccc4c3C(=O)N(C3CCC(=O)NC3=O)C4=O)C2)n2nccc2n1 ; CCCc1cc(N[C@@H]2CC[C@@H](NC(=O)CCOCCOCCOCCOCCNc3cccc4c3C(=O)N(C3CCC(=O)NC3=O)C4=O)C2)n2nccc2n1]

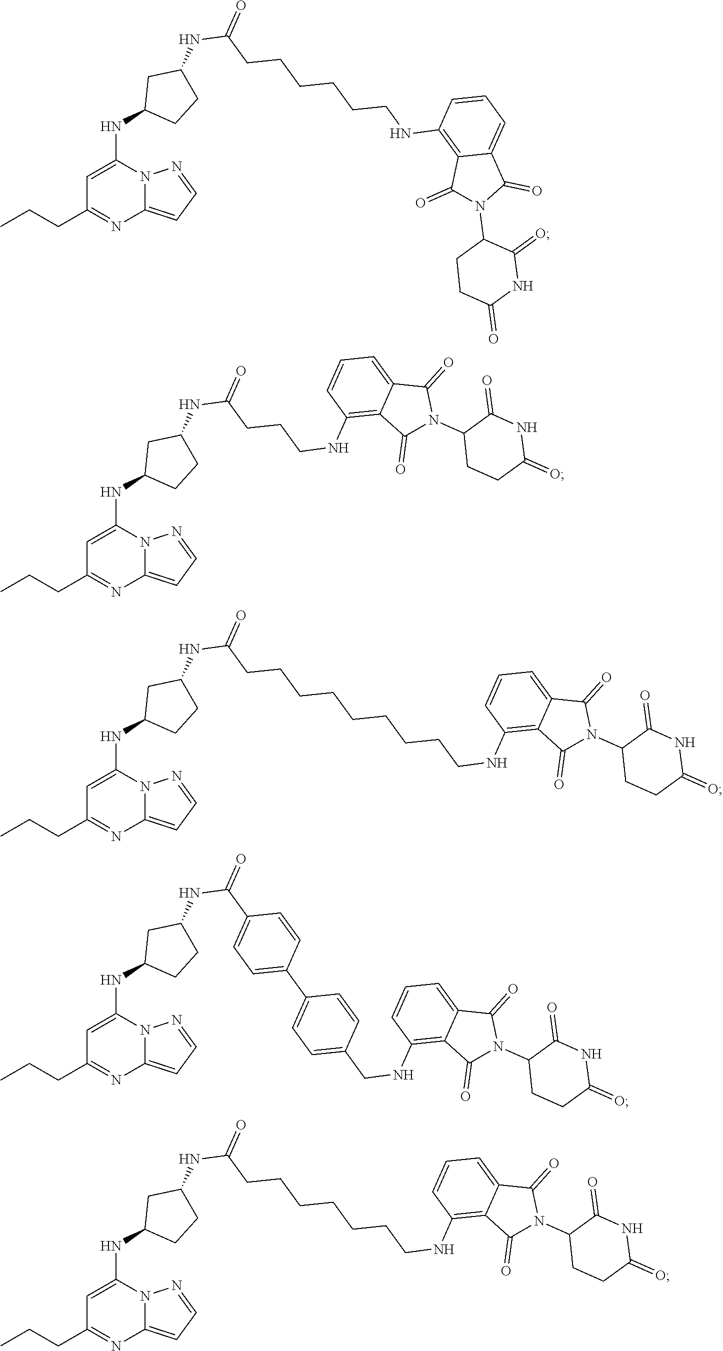

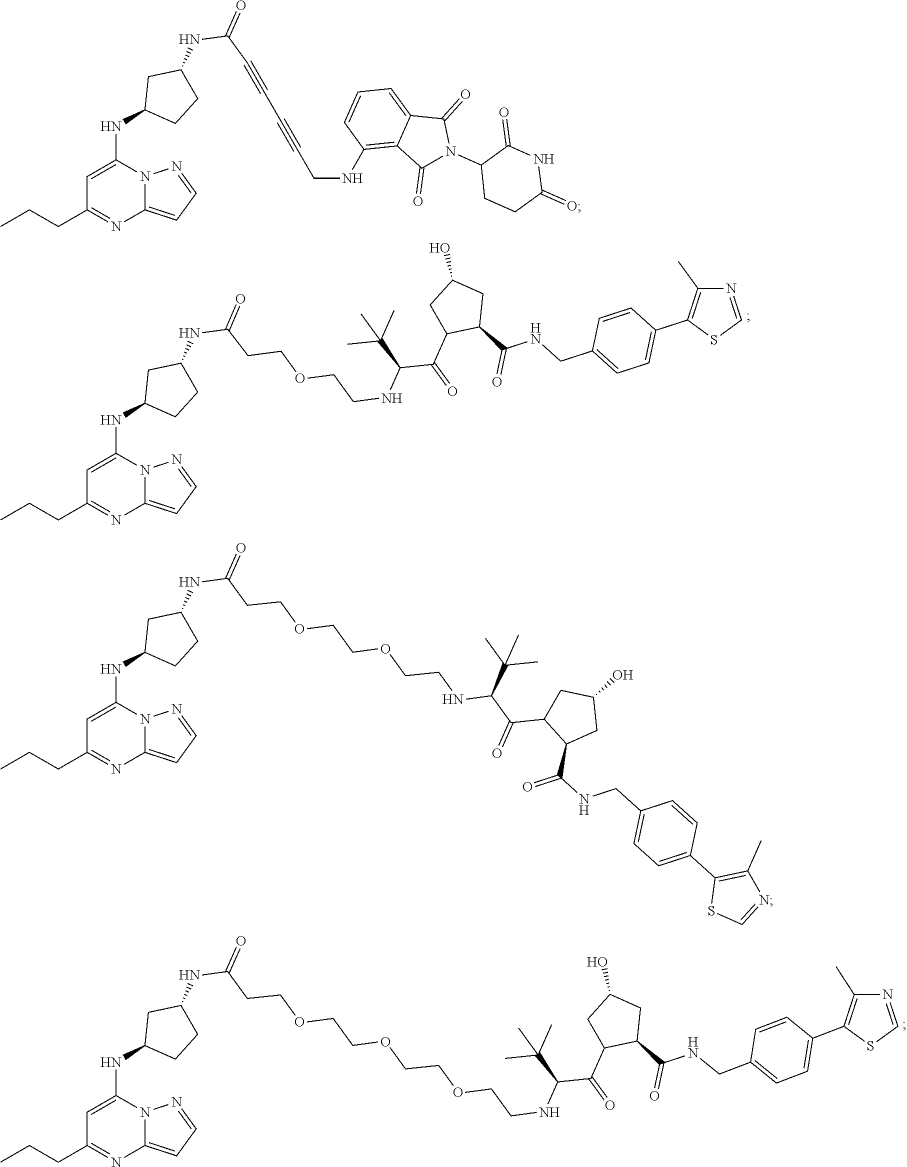 [SMILES: CCCc1cc(N[C@@H]2CC[C@@H](NC(=O)C#CC#CCNc3cccc4c3C(=O)N(C3CCC(=O)NC3=O)C4=O)C2)n2nccc2n1 ; CCCc1cc(N[C@@H]2CC[C@@H](NC(=O)CCOCCN[C@H](C(=O)C3C[C@H](O)C[C@H]3C(=O)NCc3ccc(-c4scnc4C)cc3)C(C)(C)C)C2)n2nccc2n1 ; CCCc1cc(N[C@@H]2CC[C@@H](NC(=O)CCOCCOCCN[C@H](C(=O)C3C[C@H](O)C[C@H]3C(=O)NCc3ccc(-c4scnc4C)cc3)C(C)(C)C)C2)n2nccc2n1 ; CCCc1cc(N[C@@H]2CC[C@@H](NC(=O)CCOCCOCCOCCN[C@H](C(=O)C3C[C@H](O)C[C@H]3C(=O)NCc3ccc(-c4scnc4C)cc3)C(C)(C)C)C2)n2nccc2n1]

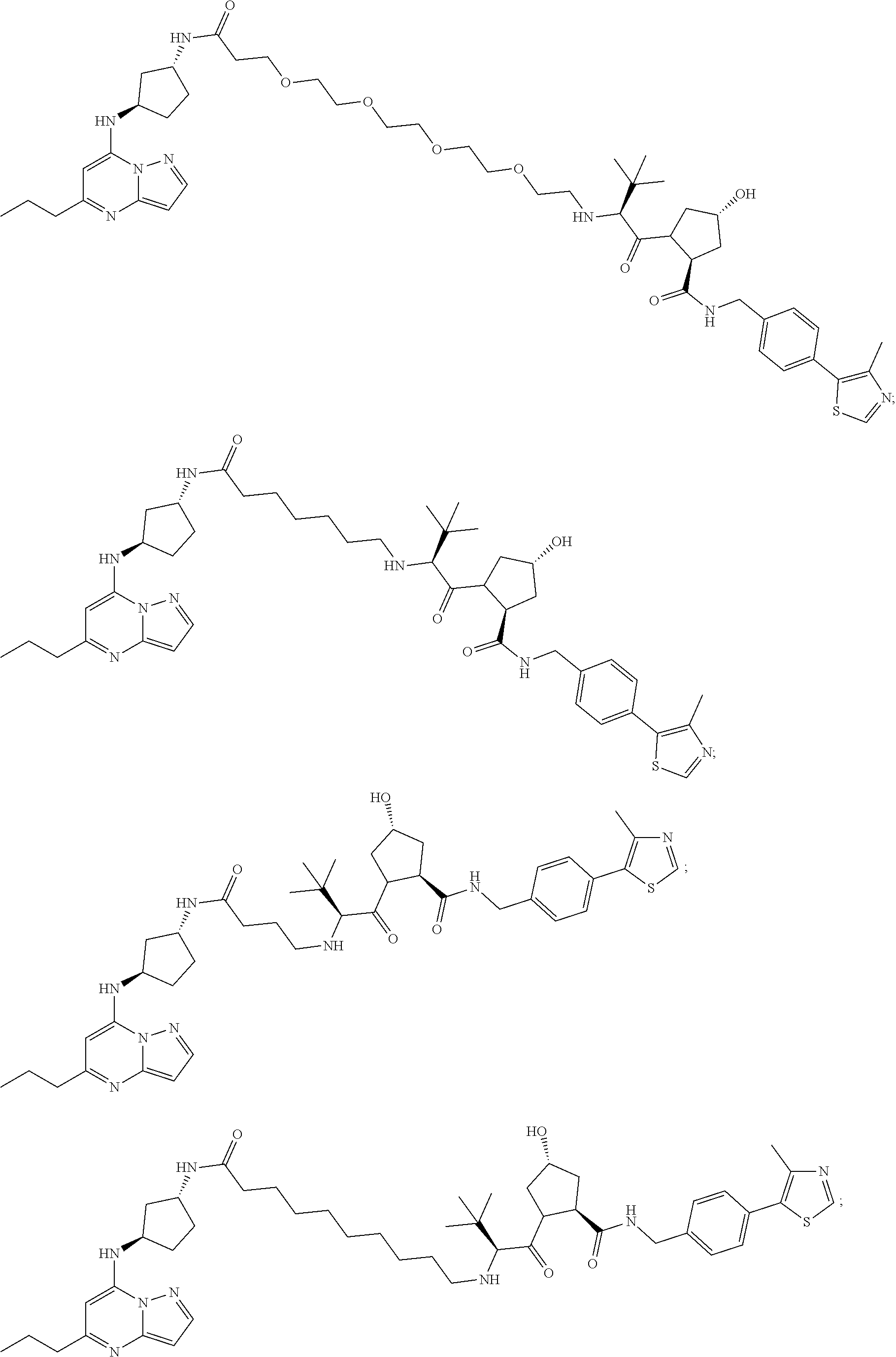 [SMILES: CCCc1cc(N[C@@H]2CC[C@@H](NC(=O)CCOCCOCCOCCOCCN[C@H](C(=O)C3C[C@H](O)C[C@H]3C(=O)NCc3ccc(-c4scnc4C)cc3)C(C)(C)C)C2)n2nccc2n1 ; CCCc1cc(N[C@@H]2CC[C@@H](NC(=O)CCCCCCN[C@H](C(=O)C3C[C@H](O)C[C@H]3C(=O)NCc3ccc(-c4scnc4C)cc3)C(C)(C)C)C2)n2nccc2n1 ; CCCc1cc(N[C@@H]2CC[C@@H](NC(=O)CCCN[C@H](C(=O)C3C[C@H](O)C[C@H]3C(=O)NCc3ccc(-c4scnc4C)cc3)C(C)(C)C)C2)n2nccc2n1 ; CCCc1cc(N[C@@H]2CC[C@@H](NC(=O)CCCCCCCCCN[C@H](C(=O)C3C[C@H](O)C[C@H]3C(=O)NCc3ccc(-c4scnc4C)cc3)C(C)(C)C)C2)n2nccc2n1]

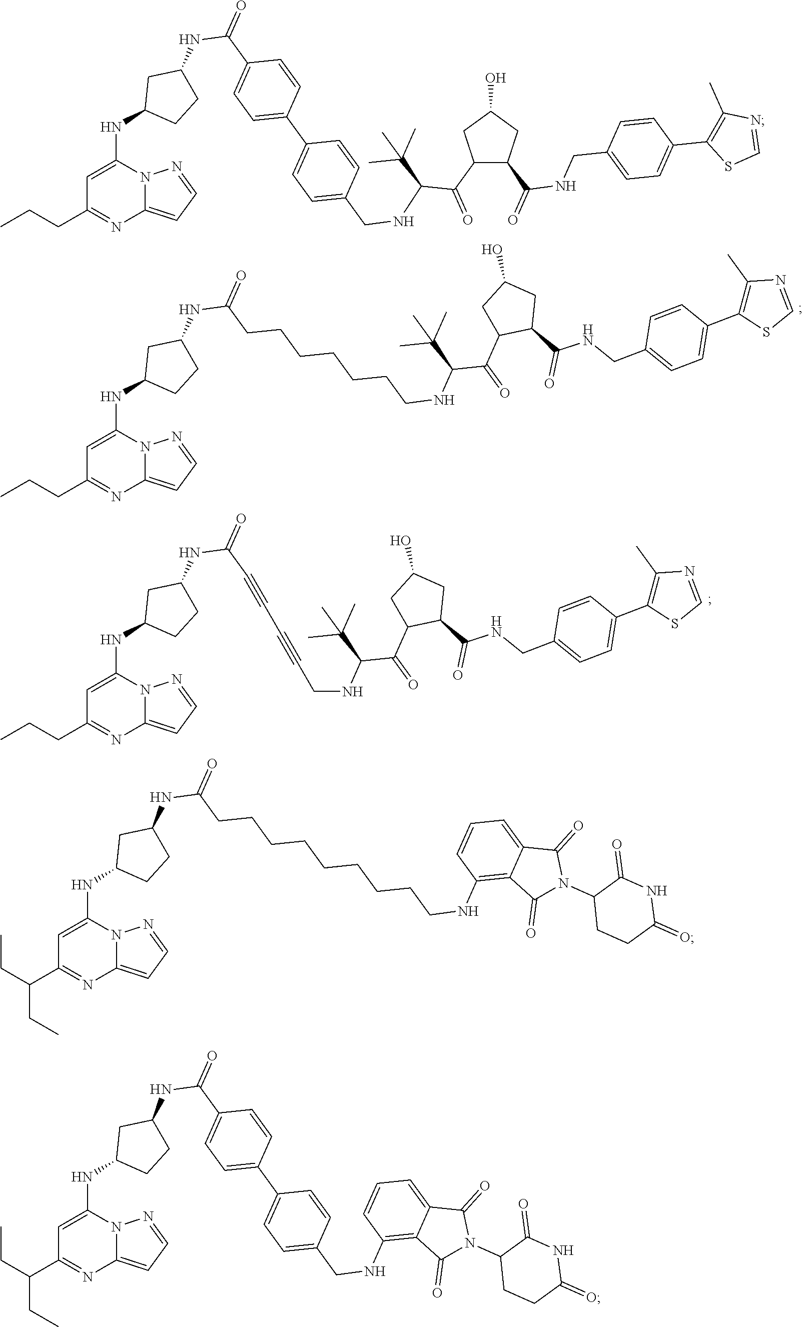

or a pharmaceutically acceptable salt, co-crystal, tautomer, stereoisomer, solvate, hydrate, polymorph, isotopically enriched derivative, or prodrug thereof.

In certain embodiments, the compound of Formula (I) is a compound of the formula:

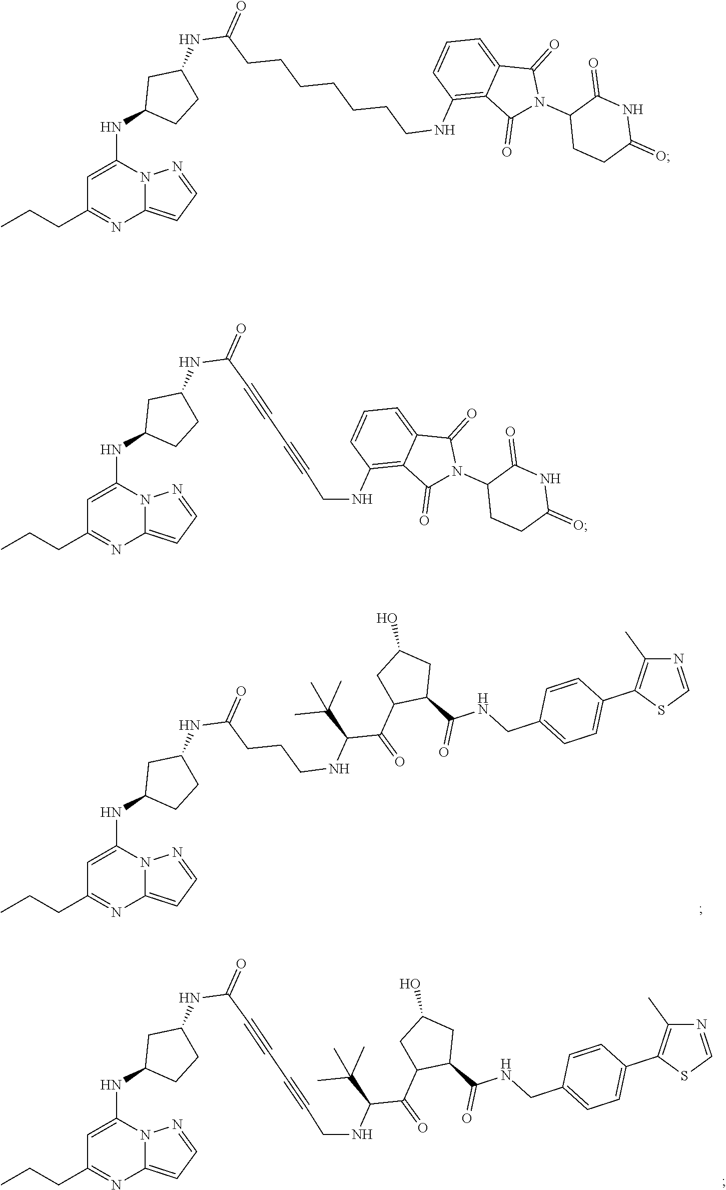 [SMILES: CCCc1cc(N[C@@H]2CC[C@@H](NC(=O)CCCCCCCNc3cccc4c3C(=O)N(C3CCC(=O)NC3=O)C4=O)C2)n2nccc2n1 ; CCCc1cc(N[C@@H]2CC[C@@H](NC(=O)C#CC#CCNc3cccc4c3C(=O)N(C3CCC(=O)NC3=O)C4=O)C2)n2nccc2n1 ; CCCc1cc(N[C@@H]2CC[C@@H](NC(=O)CCCN[C@H](C(=O)C3C[C@H](O)C[C@H]3C(=O)NCc3ccc(-c4scnc4C)cc3)C(C)(C)C)C2)n2nccc2n1 ; CCCc1cc(N[C@@H]2CC[C@@H](NC(=O)C#CC#CCN[C@H](C(=O)C3C[C@H](O)C[C@H]3C(=O)NCc3ccc(-c4scnc4C)cc3)C(C)(C)C)C2)n2nccc2n1]

or a pharmaceutically acceptable salt, co-crystal, tautomer, stereoisomer, solvate, hydrate, polymorph, isotopically enriched derivative, or prodrug thereof.

In another aspect, disclosed are compounds of the formula:

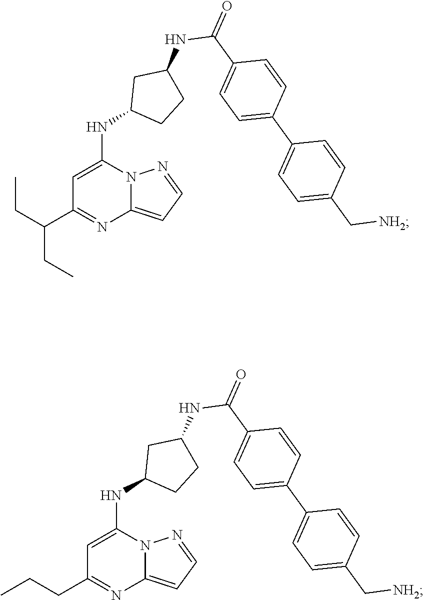 [SMILES: CCC(CC)c1cc(N[C@H]2CC[C@H](NC(=O)c3ccc(-c4ccc(CN)cc4)cc3)C2)n2nccc2n1 ; CCCc1cc(N[C@@H]2CC[C@@H](NC(=O)c3ccc(-c4ccc(CN)cc4)cc3)C2)n2nccc2n1]

and pharmaceutically acceptable salts, co-crystals, tautomers, stereoisomers, solvates, hydrates, polymorphs, isotopically enriched derivatives, or prodrugs thereof

In certain embodiments, the compounds of the disclosure (e.g., a compound of Formula (I)) bind CDK9 with a K_{d} of less than 100,000 nM, less than 50,000 nM, less than 20,000 nM, less than 10,000 nM, less than 5,000 nM, less than 2,500 nM, less than 1,000 nM, less than 900 nM, less than 800 nM, less than 700 nM, less than 600 nM, less than 500 nM, less than 400 nM, less than 300 nM, less than 200 nM, less than 100 nM, less than 90 nM, less than 80 nM, less than 70 nM, less than 60 nM, less than 50 nM, less than 40 nM, less than 30 nM, less than 20 nM, less than 10 nM, less than 5 nM, less than 4 nM, less than 3 nM, less than 2 nM, or less than 1 nM.

In certain embodiments, the compounds of the disclosure (e.g., a compound of Formula (I)) inhibit CDK9 with an IC₅₀ of less than 100,000 nM, less than 50,000 nM, less than 20,000 nM, less than 10,000 nM, less than 5,000 nM, less than 2,500 nM, less than 1,000 nM, less than 900 nM, less than 800 nM, less than 700 nM, less than 600 nM, less than 500 nM, less than 400 nM, less than 300 nM, less than 200 nM, less than 100 nM, less than 90 nM, less than 80 nM, less than 70 nM, less than 60 nM, less than 50 nM, less than 40 nM, less than 30 nM, less than 20 nM, less than 10 nM, less than 5 nM, less than 4 nM, less than 3 nM, less than 2 nM, or less than 1 nM.

In certain embodiments, the compounds of the disclosure (e.g., a compound of Formula (I)) selectively bind and/or inhibit CDK9 over another protein. In some embodiments, the compounds of the disclosure (e.g., a compound of Formula (I)) selectively bind and/or inhibit CDK9 over a different cyclin-dependent kinase (e.g., CDK1, CDK2, CDK3, CDK4, CDK5, CDK6, CDK7, CDK8, CDK10, CDK11, CDK12, CDK13). In some embodiments, the compounds of the disclosure (e.g., a compound of Formula (I)) selectively bind and/or inhibit CDK9 over one or more of CDK1, CDK2, CDK3, CDK4, CDK5, CDK6, CDK7, CDK8, CDK10, CDK11, CDK12, and CDK13. In certain embodiments, the selectivity is between about 2-fold and about 5-fold. In certain embodiments, the selectivity is between about 5-fold and about 10-fold. In certain embodiments, the selectivity is between about 10-fold and about 20-fold. In certain embodiments, the selectivity is between about 20-fold and about 50-fold. In certain embodiments, the selectivity is between about 50-fold and about 100-fold. In certain embodiments, the selectivity is between about 100-fold and about 200-fold. In certain embodiments, the selectivity is between about 200-fold and about 500-fold. In certain embodiments, the selectivity is between about 500-fold and about 1000-fold. In certain embodiments, the selectivity is at least about 1000-fold.

In certain embodiments, the compounds of the disclosure (e.g., a compound of Formula (I)) bind an E3 ubiquitin ligase with a K_{d} of less than 100,000 nM, less than 50,000 nM, less than 20,000 nM, less than 10,000 nM, less than 5,000 nM, less than 2,500 nM, less than 1,000 nM, less than 900 nM, less than 800 nM, less than 700 nM, less than 600 nM, less than 500 nM, less than 400 nM, less than 300 nM, less than 200 nM, less than 100 nM, less than 90 nM, less than 80 nM, less than 70 nM, less than 60 nM, less than 50 nM, less than 40 nM, less than 30 nM, less than 20 nM, less than 10 nM, less than 5 nM, less than 4 nM, less than 3 nM, less than 2 nM, or less than 1 nM.

In certain embodiments, the compounds of the disclosure (e.g., a compound of Formula (I)) bind Cereblon with a K_{d} of less than 100,000 nM, less than 50,000 nM, less than 20,000 nM, less than 10,000 nM, less than 5,000 nM, less than 2,500 nM, less than 1,000 nM, less than 900 nM, less than 800 nM, less than 700 nM, less than 600 nM, less than 500 nM, less than 400 nM, less than 300 nM, less than 200 nM, less than 100 nM, less than 90 nM, less than 80 nM, less than 70 nM, less than 60 nM, less than 50 nM, less than 40 nM, less than 30 nM, less than 20 nM, less than 10 nM, less than 5 nM, less than 4 nM, less than 3 nM, less than 2 nM, or less than 1 nM.

In certain embodiments, the compounds of the disclosure (e.g., a compound of Formula (I)) selectively bind an E3 ubiquitin ligase as compared to another protein. In some embodiments, the compounds of the disclosure (e.g., a compound of Formula (I)) selectively bind Cereblon over another protein. In some embodiments, the compounds of the disclosure (e.g., a compound of Formula (I)) selectively bind Cereblon over another E3 ubiquitin ligase. In certain embodiments, the selectivity is between about 2-fold and about 5-fold. In certain embodiments, the selectivity is between about 5-fold and about 10-fold. In certain embodiments, the selectivity is between about 10-fold and about 20-fold. In certain embodiments, the selectivity is between about 20-fold and about 50-fold. In certain embodiments, the selectivity is between about 50-fold and about 100-fold. In certain embodiments, the selectivity is between about 100-fold and about 200-fold. In certain embodiments, the selectivity is between about 200-fold and about 500-fold. In certain embodiments, the selectivity is between about 500-fold and about 1000-fold. In certain embodiments, the selectivity is at least about 1000-fold.

In certain embodiments, the compounds of the disclosure (e.g., a compound of Formula (I)) bind VHL with a K_{d} of less than 100,000 nM, less than 50,000 nM, less than 20,000 nM, less than 10,000 nM, less than 5,000 nM, less than 2,500 nM, less than 1,000 nM, less than 900 nM, less than 800 nM, less than 700 nM, less than 600 nM, less than 500 nM, less than 400 nM, less than 300 nM, less than 200 nM, less than 100 nM, less than 90 nM, less than 80 nM, less than 70 nM, less than 60 nM, less than 50 nM, less than 40 nM, less than 30 nM, less than 20 nM, less than 10 nM, less than 5 nM, less than 4 nM, less than 3 nM, less than 2 nM, or less than 1 nM.

In some embodiments, the compounds of the disclosure (e.g., a compound of Formula (I)) selectively bind VHL over another protein. In some embodiments, the compounds of the disclosure (e.g., a compound of Formula (I)) selectively bind VHL over another E3 ubiquitin ligase. In certain embodiments, the selectivity is between about 2-fold and about 5-fold. In certain embodiments, the selectivity is between about 5-fold and about 10-fold. In certain embodiments, the selectivity is between about 10-fold and about 20-fold. In certain embodiments, the selectivity is between about 20-fold and about 50-fold. In certain embodiments, the selectivity is between about 50-fold and about 100-fold. In certain embodiments, the selectivity is between about 100-fold and about 200-fold. In certain embodiments, the selectivity is between about 200-fold and about 500-fold. In certain embodiments, the selectivity is between about 500-fold and about 1000-fold. In certain embodiments, the selectivity is at least about 1000-fold.

In certain embodiments, the compounds of the disclosure (e.g., a compound of Formula (I)) promote the degradation of up to 10%, up to 15%, up to 20%, up to 25%, up to 30%, up to 35%, up to 40%, up to 45%, up to 50%, up to 55%, up to 60%, up to 65%, up to 70%, up to 75%, up to 80%, up to 85%, up to 90%, up to 95%, up to 99%, or up to 100% of CDK9 at a concentration of 100,000 nM or less, 50,000 nM or less, 20,000 nM or less, 10,000 nM or less, 5,000 nM or less, 3,500 nM or less, 2,500 nM or less, 1,000 nM or less, 900 nM or less, 800 nM or less, 700 nM or less, 600 nM or less, 500 nM or less, 400 nM or less, 300 nM or less, 200 nM or less, 100 nM or less, 90 nM or less, 80 nM or less, 70 nM or less, 60 nM or less, 50 nM or less, 40 nM or less, 30 nM or less, 20 nM or less, 10 nM or less, 5 nM or less, 4 nM or less, 3 nM or less, 2 nM or less, or 1 nM or less.

In certain embodiments, the compounds of the disclosure (e.g., a compound of Formula (I)) increase the rate of CDK9 degradation of up to 10%, up to 15%, up to 20%, up to 25%, up to 30%, up to 35%, up to 40%, up to 45%, up to 50%, up to 55%, up to 60%, up to 65%, up to 70%, up to 75%, up to 80%, up to 85%, up to 90%, up to 95%, up to 99%, or up to 100% at a concentration of 100,000 nM or less, 50,000 nM or less, 20,000 nM or less, 10,000 nM or less, 5,000 nM or less, 3,500 nM or less, 2,500 nM or less, 1,000 nM or less, 900 nM or less, 800 nM or less, 700 nM or less, 600 nM or less, 500 nM or less, 400 nM or less, 300 nM or less, 200 nM or less, 100 nM or less, 90 nM or less, 80 nM or less, 70 nM or less, 60 nM or less, 50 nM or less, 40 nM or less, 30 nM or less, 20 nM or less, 10 nM or less, 5 nM or less, 4 nM or less, 3 nM or less, 2 nM or less, or 1 nM or less.

### Pharmaceutical Compositions, Kits, and Administration

The present disclosure provides pharmaceutical compositions comprising a compound of the disclosure (e.g., a compound of Formula (I)), or a pharmaceutically acceptable salt, co-crystal, tautomer, stereoisomer, solvate, hydrate, polymorph, isotopically enriched derivative, or prodrug thereof, and optionally a pharmaceutically acceptable excipient. In certain embodiments, the pharmaceutical composition described herein comprises a compound of the disclosure (e.g., a compound of Formula (I)), or a pharmaceutically acceptable salt thereof, and a pharmaceutically acceptable excipient.

In certain embodiments, a compound of the disclosure (e.g., a compound of Formula (I)) is provided in an effective amount in the pharmaceutical composition. In certain embodiments, the effective amount is a therapeutically effective amount. In certain embodiments, the effective amount is a prophylactically effective amount. In certain embodiments, the effective amount is an amount effective for treating cancer in a subject in need thereof. In certain embodiments, the effective amount is an amount effective for preventing cancer in a subject in need thereof. In certain embodiments, the effective amount is an amount effective for treating a solid tumor or a hematological cancer in a subject in need thereof. In certain embodiments, the effective amount is an amount effective for treating a leukemia or a lymphoma in a subject in need thereof. In certain embodiments, the effective amount is an amount effective for acute myeloid leukemia (AML) or acute lymphoblastic leukemia (ALL) in a subject in need thereof. In certain embodiments, the effective amount is an amount effective for treating hepatocellular carcinoma, prostate cancer, or neuroblastoma in a subject in need thereof.

In certain embodiments, the subject is an animal. The animal may be of either sex and may be at any stage of development. In certain embodiments, the subject described herein is a human. In certain embodiments, the subject is a non-human animal. In certain embodiments, the subject is a mammal. In certain embodiments, the subject is a non-human mammal. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal, such as a rodent (e.g., mouse, rat), dog, pig, or non-human primate. In certain embodiments, the animal is a genetically engineered animal. In certain embodiments, the animal is a transgenic animal (e.g., transgenic mice and transgenic pigs). In certain embodiments, the subject is a fish or reptile.

In certain embodiments, the effective amount is an amount effective for promoting the degradation of at least about 10%, at least about 20%, at least about 30%, at least about 40%, at least about 50%, at least about 60%, at least about 70%, at least about 80%, at least about 90%, at least about 95%, at least about 98%, or at least about 99% of CDK9. In certain embodiments, the effective amount is an amount effective for promoting the degradation of CDK9 by a range between a percentage described in this paragraph and another percentage described in this paragraph, inclusive.

The present disclosure provides pharmaceutical compositions comprising a compound that interacts with CDK9 and/or an E3 ubiquitin ligase (e.g., Cereblon) for use in treating cancer in a subject in need thereof. In certain embodiments, the composition is for use in treating a solid tumor or a hematological cancer. In certain embodiments, the composition is for use in treating a leukemia or a lymphoma. In certain embodiments, the composition is for use in treating acute myeloid leukemia (AML) or acute lymphoblastic leukemia (ALL). In certain embodiments, the composition is for use in treating hepatocellular carcinoma, prostate cancer, or neuroblastoma.

A compound or composition, as described herein, can be administered in combination with one or more additional pharmaceutical agents (e.g., therapeutically and/or prophylactically active agents). The compounds or compositions can be administered in combination with additional pharmaceutical agents that improve their activity (e.g., activity (e.g., potency and/or efficacy) in treating a disease in a subject in need thereof, in preventing a disease in a subject in need thereof, and/or in reducing the risk to develop a disease in a subject in need thereof), improve bioavailability, improve their ability to cross the blood-brain barrier, improve safety, reduce drug resistance, reduce and/or modify metabolism, inhibit excretion, and/or modify distribution in a subject or cell. It will also be appreciated that the therapy employed may achieve a desired effect for the same disorder, and/or it may achieve different effects. In certain embodiments, a pharmaceutical composition described herein including a compound described herein and an additional pharmaceutical agent exhibit a synergistic effect that is absent in a pharmaceutical composition including one of the compound and the additional pharmaceutical agent, but not both.

The compound or composition can be administered concurrently with, prior to, or subsequent to one or more additional pharmaceutical agents, which may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease (e.g., neurological disorder, neurodegenerative disease, and/or tauopathy). Each additional pharmaceutical agent may be administered at a dose and/or on a time schedule determined for that pharmaceutical agent. The additional pharmaceutical agents may also be administered together with each other and/or with the compound or composition described herein in a single dose or administered separately in different doses. The particular combination to employ in a regimen will take into account compatibility of the compound described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

In certain embodiments, the compound or pharmaceutical composition is a solid. In certain embodiments, the compound or pharmaceutical composition is a powder. In certain embodiments, the compound or pharmaceutical composition can be dissolved in a liquid to make a solution. In certain embodiments, the compound or pharmaceutical composition is dissolved in water to make an aqueous solution. In certain embodiments, the pharmaceutical composition is a liquid for parental injection. In certain embodiments, the pharmaceutical composition is a liquid for oral administration (e.g., ingestion). In certain embodiments, the pharmaceutical composition is a liquid (e.g., aqueous solution) for intravenous injection. In certain embodiments, the pharmaceutical composition is a liquid (e.g., aqueous solution) for subcutaneous injection.

After formulation with an appropriate pharmaceutically acceptable excipient in a desired dosage, the pharmaceutical compositions of this invention can be administered to humans and other animals orally, parenterally, intracisternally, intraperitoneally, topically, bucally, or the like, depending on the disease or condition being treated.

In certain embodiments, a pharmaceutical composition comprising a compound the disclosure (e.g., a compound of Formula (I)) is administered, orally or parenterally, at dosage levels of each pharmaceutical composition sufficient to deliver from about 0.001 mg/kg to about 200 mg/kg in one or more dose administrations for one or several days (depending on the mode of administration). In certain embodiments, the effective amount per dose varies from about 0.001 mg/kg to about 200 mg/kg, about 0.001 mg/kg to about 100 mg/kg, about 0.01 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, of subject body weight per day, one or more times a day, to obtain the desired therapeutic and/or prophylactic effect. In certain embodiments, the compounds described herein may be at dosage levels sufficient to deliver from about 0.001 mg/kg to about 200 mg/kg, from about 0.001 mg/kg to about 100 mg/kg, from about 0.01 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic and/or prophylactic effect. The desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations). In certain embodiments, the composition described herein is administered at a dose that is below the dose at which the agent causes non-specific effects.

In certain embodiments, the pharmaceutical composition is administered at a dose of about 0.001 mg to about 1000 mg per unit dose. In certain embodiments, the pharmaceutical composition is administered at a dose of about 0.01 mg to about 200 mg per unit dose. In certain embodiments, the pharmaceutical composition is administered at a dose of about 0.01 mg to about 100 mg per unit dose. In certain embodiments, pharmaceutical composition is administered at a dose of about 0.01 mg to about 50 mg per unit dose. In certain embodiments, the pharmaceutical composition is administered at a dose of about 0.01 mg to about 10 mg per unit dose. In certain embodiments, the pharmaceutical composition is administered at a dose of about 0.1 mg to about 10 mg per unit dose.

Pharmaceutical compositions described herein can be prepared by any method known in the art of pharmacology. In general, such preparatory methods include the steps of bringing the composition comprising a compound of the disclosure (e.g., a compound of Formula (I)) into association with a carrier and/or one or more other accessory ingredients, and then, if necessary and/or desirable, shaping and/or packaging the product into a desired single- or multi-dose unit.

Pharmaceutical compositions can be prepared, packaged, and/or sold in bulk, as a single unit dose, and/or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as, for example, one-half or one-third of such a dosage.

Relative amounts of the active ingredient, the pharmaceutically acceptable excipient, and/or any additional ingredients in a pharmaceutical composition of the invention will vary, depending upon the identity, size, and/or condition of the subject treated and further depending upon the route by which the composition is to be administered. By way of example, the composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite (aluminum silicate) and Veegum (magnesium aluminum silicate)), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate (Tween 20), polyoxyethylene sorbitan (Tween 60), polyoxyethylene sorbitan monooleate (Tween 80), sorbitan monopalmitate (Span 40), sorbitan monostearate (Span 60), sorbitan tristearate (Span 65), glyceryl monooleate, sorbitan monooleate (Span 80)), polyoxyethylene esters (e.g. polyoxyethylene monostearate (Myrj 45), polyoxyethylene hydrogenated castor oil, polyethoxylated castor oil, polyoxymethylene stearate, and Solutol), sucrose fatty acid esters, polyethylene glycol fatty acid esters (e.g. Cremophor™), polyoxyethylene ethers, (e.g. polyoxyethylene lauryl ether (Brij 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F-68, Poloxamer-188, cetrimonium bromide, cetylpyridinium chloride, benzalkonium chloride, docusate sodium, and/or mixtures thereof.

Exemplary binding agents include starch (e.g. cornstarch and starch paste), gelatin, sugars (e.g. sucrose, glucose, dextrose, dextrin, molasses, lactose, lactitol, mannitol, etc.), natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazelnut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include, but are not limited to, pharmaceutically acceptable emulsions, microemulsions, solutions, suspensions, syrups, and elixirs. In addition to the active agents, the liquid dosage forms may contain inert diluents commonly used in the art such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can also include adjuvants such as wetting agents, emulsifying and suspending agents, sweetening, flavoring, and perfuming agents. In certain embodiments for parenteral administration, agents of the invention are mixed with solubilizing agents such CREMOPHOR EL® (polyethoxylated castor oil), alcohols, oils, modified oils, glycols, polysorbates, cyclodextrins, polymers, and combinations thereof.

Injectable preparations, for example, sterile injectable aqueous or oleaginous suspensions, may be formulated according to the known art using suitable dispersing or wetting agents and suspending agents. Sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

Solid dosage forms for oral administration include capsules, tablets, pills, powders, and granules. In such solid dosage forms, the active agent is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active agents can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active agent may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets, and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Formulations suitable for topical administration include liquid or semi-liquid preparations such as liniments, lotions, gels, applicants, oil-in-water or water-in-oil emulsions such as creams, ointments, or pastes; or solutions or suspensions such as drops. Formulations for topical administration to the skin surface can be prepared by dispersing the drug with a dermatologically acceptable carrier such as a lotion, cream, ointment, or soap. Useful carriers are capable of forming a film or layer over the skin to localize application and inhibit removal. For topical administration to internal tissue surfaces, the agent can be dispersed in a liquid tissue adhesive or other substance known to enhance adsorption to a tissue surface. For example, hydroxypropylcellulose or fibrinogen/thrombin solutions can be used to advantage. Alternatively, tissue-coating solutions, such as pectin-containing formulations can be used. Ophthalmic formulation, ear drops, and eye drops are also contemplated as being within the scope of this invention. Additionally, the present disclosure contemplates the use of transdermal patches, which have the added advantage of providing controlled delivery of an agent to the body. Such dosage forms can be made by dissolving or dispensing the agent in the proper medium. Absorption enhancers can also be used to increase the flux of the agent across the skin. The rate can be controlled by either providing a rate controlling membrane or by dispersing the agent in a polymer matrix or gel.

Additionally, the carrier for a topical formulation can be in the form of a hydroalcoholic system (e.g., quids and gels), an anhydrous oil or silicone based system, or an emulsion system, including, but not limited to, oil-in-water, water-in-oil, water-in-oil-in-water, and oil-in-water-in-silicone emulsions. The emulsions can cover a broad range of consistencies including thin lotions (which can also be suitable for spray or aerosol delivery), creamy lotions, light creams, heavy creams, and the like. The emulsions can also include microemulsion systems. Other suitable topical carriers include anhydrous solids and semisolids (such as gels and sticks); and aqueous based mousse systems.

Also encompassed by the disclosure are kits (e.g., pharmaceutical packs). The kits provided may comprise a pharmaceutical composition or compound described herein and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or compound described herein. In some embodiments, the pharmaceutical composition or compound described herein provided in the first container and the second container are combined to form one unit dosage form.

Thus, in one aspect, provided are kits including a first container comprising a compound or pharmaceutical composition described herein. In certain embodiments, the kits are useful for treating cancer (e.g., a solid tumor or a hematological cancer) in a subject in need thereof. In certain embodiments, the kits are useful for preventing cancer (e.g., a solid tumor or a hematological cancer) in a subject in need thereof. In certain embodiments, the kits are useful for reducing the risk of developing cancer (e.g., a solid tumor or a hematological cancer) in a subject in need thereof. In certain embodiments, the kits are useful for promoting the degradation of CDK9 in a subject or cell.

In certain embodiments, a kit described herein further includes instructions for using the kit. A kit described herein may also include information as required by a regulatory agency such as the U.S. Food and Drug Administration (FDA). In certain embodiments, the information included in the kits is prescribing information. In certain embodiments, a kit described herein may include one or more additional pharmaceutical agents described herein as a separate composition.

### Methods of Treatment

CDK9 has a central role in transcriptional regulation, which is frequently dysregulated in cancer. CDK9 is dysregulated in a number of solid tumors, including prostate cancer, neuroblastoma, hepatocellular carcinoma, and lymphoma. CDK9 pathway dysregulation has likewise been observed in liquid tumors, such as acute myeloid leukemia (AML) and acute lymphoblastic leukemia (ALL). Thus, inhibitions and/or degradation is an attractive target for the treatment of cancer.

Immunomodulatory agents, including thalidomide, lenalidomide, and pomalidomide bind Cereblon. In addition, compounds such as (2S,4R)-1-((S)-2-acetamido-3,3-dimethylbutanoyl)-4-hydroxy-N-(4-(4-methylthiazol-5-yl)benzyl)pyrrolidine-2-carboxamide, effectively bind VHL.

Accordingly, use of a bifunctional compound that binds and/or inhibits CDK9 and binds an E3 ubiquitin ligase (e.g., Cereblon, VHL) provides a method of treating diseases that rely on CDK9 activity.

Thus, the present disclosure provides methods for treating cancer. In certain embodiments, the application provides a method of treating a solid tumor or a hematological cancer. In certain embodiments, the application provides a method of treating a hematological cancer. In certain embodiments, the application provides a method of treating a leukemia or a lymphoma. In certain embodiments, the application provides a method of treating acute myeloid leukemia (AML) or acute lymphoblastic leukemia (ALL). In certain embodiments, the application provides a method of treating acute myeloid leukemia (AML). In certain embodiments, the application provides a method of treating acute lymphoblastic leukemia (ALL). In certain embodiments, the application provides a method of treating a solid tumor. In certain embodiments, the application provides a method of treating ovarian cancer, osteosarcoma, hepatocellular carcinoma, prostate cancer, or neuroblastoma. In certain embodiments, the application provides a method of treating osteosarcoma, hepatocellular carcinoma, prostate cancer, or neuroblastoma. In certain embodiments, the application provides a method of treating hepatocellular carcinoma, prostate cancer, or neuroblastoma. In certain embodiments, the application provides a method of treating osteosarcoma. In certain embodiments, the application provides a method of treating hepatocellular carcinoma. In certain embodiments, the application provides a method of treating prostate cancer. In certain embodiments, the application provides a method of treating neuroblastoma. In certain embodiments, the application provides a method of promoting the degradation of CDK9.

In certain embodiments, the methods comprise administering to a subject in need thereof (e.g., a subject with a cancer) a compound that interacts with CDK9, for example, a compound that is an inhibitor of CDK9, a modulator of CDK9, a binder of CDK9, a compound that modifies CDK9, or a compound that promotes the degradation of CDK9. The compound may also be an inhibitor of an E3 ubiquitin ligase, a modulator of an E3 ubiquitin ligase, a binder of an E3 ubiquitin ligase, a compound that modifies an E3 ubiquitin ligase, or a compound that disrupts the interaction of the E3 ubiquitin ligase with another protein. In certain embodiments, the methods comprise administering a compound the disclosure (e.g., a compound of Formula (I)), or a pharmaceutically acceptable salt, co-crystal, tautomer, stereoisomer, solvate, hydrate, polymorph, isotopically enriched derivative, or prodrug, or composition thereof, to a subject in need thereof. In some embodiments, the method comprises administering a pharmaceutical composition comprising a compound of the disclosure (e.g., a compound of Formula (I)), or a pharmaceutically acceptable salt, co-crystal, tautomer, stereoisomer, solvate, hydrate, polymorph, isotopically enriched derivative, or prodrug, or composition thereof, to a subject in need thereof.

In certain embodiments, the methods of the disclosure comprise administering to the subject an effective amount of a compound of the disclosure (e.g., a compound of Formula (I)), or a pharmaceutically acceptable salt, co-crystal, tautomer, stereoisomer, solvate, hydrate, polymorph, isotopically enriched derivative, or prodrug, or composition thereof. In some embodiments, the effective amount is a therapeutically effective amount. In some embodiments, the effective amount is a prophylactically effective amount.

In certain embodiments, the application provides a compound for use in treating a solid tumor or a hematological cancer. In certain embodiments, the application provides a compound for use in treating a hematological cancer. In certain embodiments, the application provides a compound for use in treating a leukemia or a lymphoma. In certain embodiments, the application provides compound for use in treating acute myeloid leukemia (AML) or acute lymphoblastic leukemia (ALL). In certain embodiments, the application provides a compound for use in treating acute myeloid leukemia (AML). In certain embodiments, the application provides a compound for use in treating acute lymphoblastic leukemia (ALL). In certain embodiments, the application provides a compound for use in treating a solid tumor. In certain embodiments, the application provides a compound for use in treating ovarian cancer, osteosarcoma, hepatocellular carcinoma, prostate cancer, or neuroblastoma. In certain embodiments, the application provides a compound for use in treating osteosarcoma, hepatocellular carcinoma, prostate cancer, or neuroblastoma. In certain embodiments, the application provides a compound for use in treating hepatocellular carcinoma, prostate cancer, or neuroblastoma. In certain embodiments, the application provides a compound for use in treating osteosarcoma. In certain embodiments, the application provides a compound for use in treating hepatocellular carcinoma. In certain embodiments, the application provides a compound for use in treating prostate cancer. In certain embodiments, the application provides a compound for use in treating neuroblastoma. In certain embodiments, the application provides a compound for use in promoting the degradation of CDK9.

In certain embodiments, the application provides a compound for use in the manufacture of a medicament for treating a solid tumor or a hematological cancer. In certain embodiments, the application provides a compound for use in the manufacture of a medicament for treating a hematological cancer. In certain embodiments, the application provides a compound for use in the manufacture of a medicament for treating a leukemia or a lymphoma. In certain embodiments, the application provides a compound for use in the manufacture of a medicament for treating acute myeloid leukemia (AML) or acute lymphoblastic leukemia (ALL). In certain embodiments, the application provides a compound for use in the manufacture of a medicament for treating acute myeloid leukemia (AML). In certain embodiments, the application provides a compound for use in the manufacture of a medicament for treating acute lymphoblastic leukemia (ALL). In certain embodiments, the application provides a compound for use in the manufacture of a medicament for treating a solid tumor. In certain embodiments, the application provides a compound for use in the manufacture of a medicament for treating ovarian cancer, osteosarcoma, hepatocellular carcinoma, prostate cancer, or neuroblastoma. In certain embodiments, the application provides a compound for use in the manufacture of a medicament for treating osteosarcoma, hepatocellular carcinoma, prostate cancer, or neuroblastoma. In certain embodiments, the application provides a compound for use in the manufacture of a medicament for treating hepatocellular carcinoma, prostate cancer, or neuroblastoma. In certain embodiments, the application provides a compound for use in the manufacture of a medicament for treating osteosarcoma. In certain embodiments, the application provides a compound for use in the manufacture of a medicament for treating hepatocellular carcinoma. In certain embodiments, the application provides a compound for use in the manufacture of a medicament for treating prostate cancer. In certain embodiments, the application provides a compound for use in the manufacture of a medicament for treating neuroblastoma. In certain embodiments, the application provides a compound for use in the manufacture of a medicament for promoting the degradation of CDK9.

In certain embodiments, the subject being treated is an animal. The animal may be of either sex and may be at any stage of development. In certain embodiments, the subject is a mammal. In certain embodiments, the subject being treated is a human. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal such as a rodent (e.g., mouse, rat), dog, pig, or non-human primate. In certain embodiments, the animal is a genetically engineered animal. In certain embodiments, the animal is a transgenic animal.

Certain methods described herein may comprise administering one or more additional pharmaceutical agent(s) in combination with the compounds described herein. The additional pharmaceutical agent(s) may be administered at the same time as a compound of the disclosure (e.g., a compound of Formula (I)), or at different times than a compound of the disclosure (e.g., a compound of Formula (I)). For example, a compound of the disclosure (e.g., a compound of Formula (I)) and any additional pharmaceutical agent(s) may be on the same dosing schedule or different dosing schedules. All or some doses of a compound of the disclosure (e.g., a compound of Formula (I)) may be administered before all or some doses of an additional pharmaceutical agent, after all or some does an additional pharmaceutical agent, within a dosing schedule of an additional pharmaceutical agent, or a combination thereof. The timing of administration of a compound of the disclosure (e.g., a compound of Formula (I)) and additional pharmaceutical agents may be different for different additional pharmaceutical agents.

In certain embodiments, the additional pharmaceutical agent comprises an agent useful in the treatment of cancer. In certain embodiments, the additional pharmaceutical agent is useful in the treatment of a solid tumor or a hematological cancer. In certain embodiments, the additional pharmaceutical agent is useful in the treatment of a hematological cancer. In certain embodiments, the additional pharmaceutical agent cancer is useful in the treatment of a leukemia or a lymphoma. In certain embodiments, the additional pharmaceutical agent is useful in the treatment of acute myeloid leukemia (AML) or acute lymphoblastic leukemia (ALL). In certain embodiments, the additional pharmaceutical agent is useful in the treatment of a solid tumor. In certain embodiments, the additional pharmaceutical agent is useful in the treatment of hepatocellular carcinoma, prostate cancer, or neuroblastoma. In certain embodiments, the additional pharmaceutical agent is an anti-cancer agent. In certain embodiments, the additional pharmaceutical agent is any anti-cancer agent recited herein. In certain embodiments, the additional pharmaceutical agent is an immunotherapy.

In another aspect, the present disclosure provides methods for promoting the degradation of CDK9, the method comprising administering to the subject a compound of the disclosure (e.g., a compound of Formula (I)), or a pharmaceutically acceptable salt, co-crystal, tautomer, stereoisomer, solvate, hydrate, polymorph, isotopically enriched derivative, or prodrug, or composition thereof. In certain embodiments, the E3 ubiquitin ligase is Cereblon. In certain embodiments, the E3 ubiquitin ligase is VHL.

In another aspect, the present disclosure provides methods for promoting the degradation of CDK9 and binding an E3 ubiquitin ligase, the method comprising administering to the subject a compound of the disclosure (e.g., a compound of Formula (I)), or a pharmaceutically acceptable salt, co-crystal, tautomer, stereoisomer, solvate, hydrate, polymorph, isotopically enriched derivative, or prodrug, or composition thereof. In certain embodiments, the E3 ubiquitin ligase is Cereblon. In certain embodiments, the E3 ubiquitin ligase is VHL.

In certain embodiments, the application provides a method of promoting the ubiquitination of CDK9 by an E3 ubiquitin ligase, the method comprising administering to the subject a compound of the disclosure (e.g., a compound of Formula (I)), or a pharmaceutically acceptable salt, co-crystal, tautomer, stereoisomer, solvate, hydrate, polymorph, isotopically enriched derivative, or prodrug, or composition thereof. In certain embodiments, the E3 ubiquitin ligase is Cereblon. In certain embodiments, the E3 ubiquitin ligase is VHL.

### EXAMPLES

In order that the invention described herein may be more fully understood, the following examples are set forth. The examples described in this application are offered to illustrate the compounds, pharmaceutical compositions, and methods provided herein and are not to be construed in any way as limiting their scope.

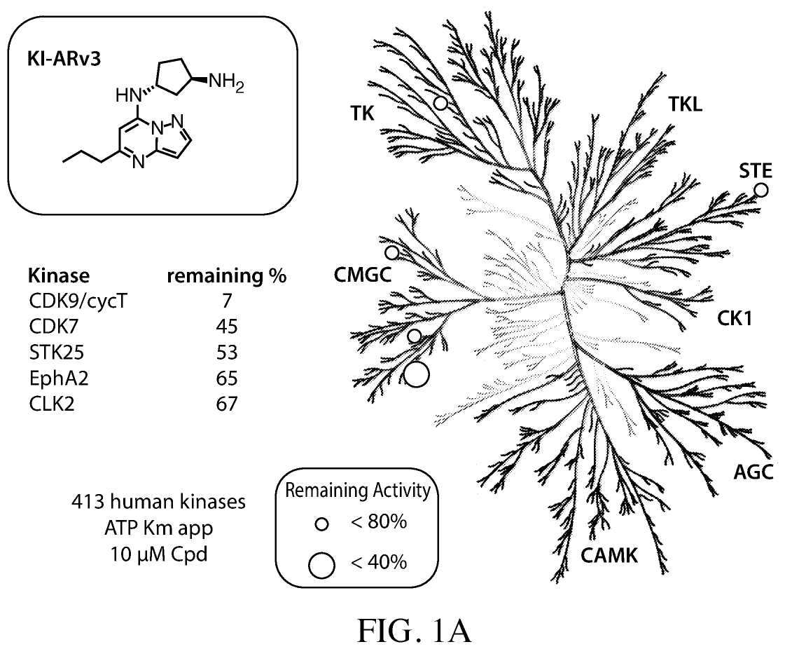

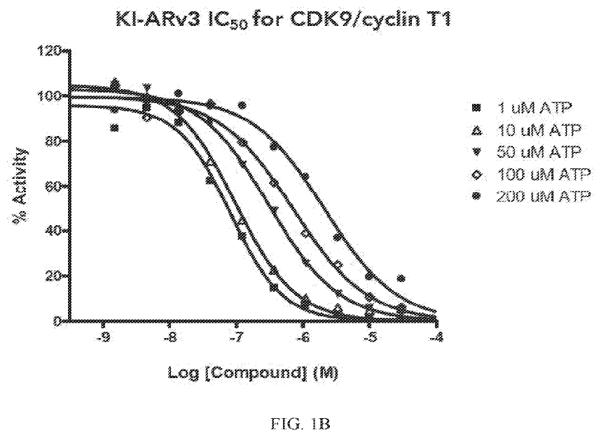

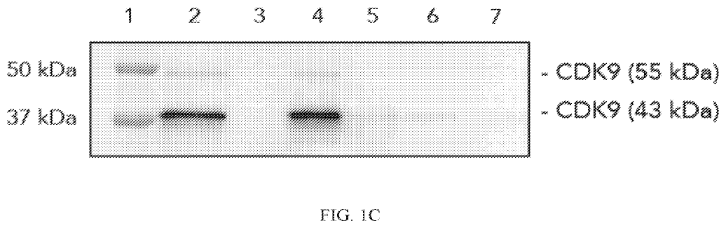

Initial studies showed that KI-ARv3 is a highly selective and potent ATP-competitive inhibitor of CDK9. These studies involved kinase selectivity screening, in vitro IC₅₀ determination and target engagement studies (FIGS. 1A-C). KI-ARv3 was originally identified in a screening targeted at the splice variant 7 of the Androgen Receptor (Arv7) and therefore primarily evaluated in ARv7 positive and negative prostate cancer cell lines (22Rv1, LNCaP). CDK9 represents a major target in AML and ALL and therefore subsequent studies were focused on both prostate cancer (22Rv1) as well as leukemia (MOLT4) cell lines.

Next, compounds incorporating KI-ARv3 as a CDK9 binding moiety were prepared and evaluated. Compounds of Formula (I) may be prepared using the synthetic schemes and procedures described in detail below.

### Preparation of KI-ARv-3

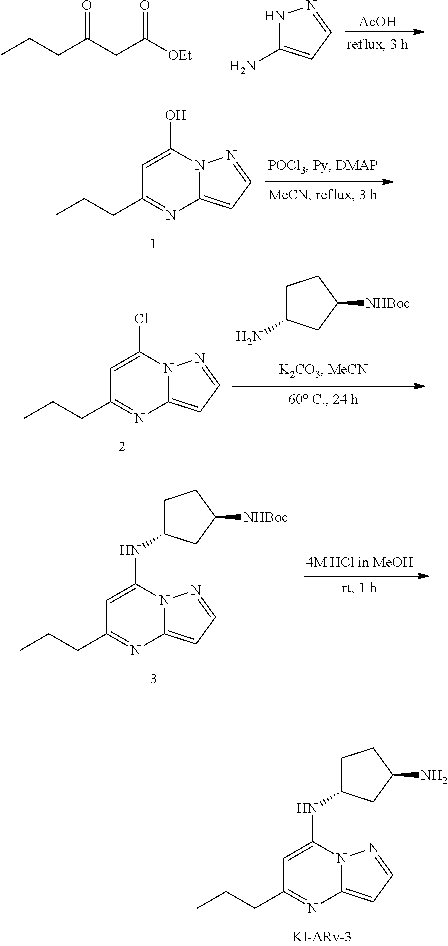

5-propylpyrazolo[1,5-a]pyrimidin-7-ol (1): A solution of 3-aminopyrazole (14.5 g, 175 mmol) and ethyl 3-oxoethanoate (29.4 mL, 184 mmol) in glacial acetic acid (100 mL) was refluxed for 3 h. After cooling to room temperature, the solvent was removed under reduced pressure and residuals were suspended in EtOAc. The resulting mixture was filtered and the remaining solid was washed with EtOAc (3×100 mL) to yield 1 as an off-white solid (25.1 g, 142 mmol, 81%). ¹H NMR (400 MHz, DMSO-d₆): δ 12.22 (s, 1H), 7.82 (d, J=2.0 Hz, 1H), 6.10 (d, J=2.0 Hz, 1H), 5.58 (s, 1H), 2.55-2.49 (m, 2H), 1.66 (h, J=7.4 Hz, 2H), 0.91 (t, J=7.4 Hz, 3H). ¹³C NMR (101 MHz, DMSO-d₆): δ 156.53, 153.79, 142.75, 141.71, 94.28, 88.46, 34.21, 21.27, 13.31. LC-MS (ES⁺): m/z 178.1.

7-chloro-5-propylpyrazolo[1,5-a]pyrimidine (2): To a suspension of 5-propylpyrazolo[1,5-a]pyrimidin-7-ol (1, 801 mg, 4.52 mmol) in dry MeCN were added phosphorous oxychloride (1.68 mL, 18.1 mmol, dropwise), pyridine (438 μL, 5.42 mmol), and dimethylaminopyridine (28 mg, 0.23 mmol). The resulting suspension was refluxed for 3 h. After cooling to room temperature, the solvent was removed in vacuo and the remaining residue was treated with ice water and immediately extracted with EtOAc (3×100 mL). The combined organic layers were dried with Na₂SO₄ and the crude was purified by silica gel flash column chromatography (0-30% EtOAc/hexane) to yield 2 as yellow/green liquid (611 mg, 3.12 mmol, 69%). The product was used immediately for subsequent reactions. ¹H NMR (500 MHz, DMSO-d₆): δ 8.27 (d, J=2.3 Hz, 1H), 7.36 (s, 1H), 6.75 (d, J=2.3 Hz, 1H), 2.77 (t, J=7.5 Hz, 2H), 1.74 (h, J=7.4 Hz, 2H), 0.92 (t, J=7.4 Hz, 3H). ¹³C NMR (DMSO-d₆): δ 162.27, 149.05, 145.14, 137.33, 108.79, 97.04, 39.08, 21.23, 13.58. LC-MS (ES⁺): m/z 197.1 and 198.0 [M+H]⁺.

tert-butyl ((1R,3R)-3-((5-propylpyrazolo[1,5-a]pyrimidin-7-yl)amino)cyclopentyl)carbamate (3): To a solution of 7-chloro-5-propylpyrazolo[1,5-a]pyrimidine (2, 400 mg, 2.04 mmol) in MeCN were added tert-butyl ((1R,3R)-3-aminocyclopentyl)carbamate (429 mg, 2.14 mmol) and K₂CO₃ (563 mg, 4.08 mmol). The resulting suspension was stirred at 60° C. for 16 h. After cooling to room temperature, the reaction mixture was diluted with water and extracted with DCM (3×100 mL). The combined organic layers were dried over Na₂SO₄ and the crude was purified by flash column chromatography (0-70% EtOAc/hexane) to yield 3 as a light-brown resin (481 mg, 1.34 mmol, 66%). ¹H NMR (500 MHz, DMSO-d₆): δ 7.99 (d, J=2.2 Hz, 1H), 7.61 (d, J=7.9 Hz, 1H), 6.97 (d, J=7.5 Hz, 1H), 6.28 (d, J=2.3 Hz, 1H), 6.05 (s, 1H), 4.17 (h, J=7.3 Hz, 1H), 3.97 (h, J=6.6 Hz, 1H), 2.61 (dd, J=8.4, 6.7 Hz, 2H), 2.13 (dtd, J=12.6, 7.9, 4.6 Hz, 1H), 2.08-1.94 (m, 1H), 1.96-1.84 (m, 2H), 1.76-1.64 (m, 3H), 1.52-1.41 (m, 1H), 1.38 (s, 9H), 0.92 (t, J=7.3 Hz, 3H). LC-MS (ES⁺): m/z 360.5 [M+H]⁺.

(1R,3R)—N1-(5-propylpyrazolo[1,5-a]pyrimidin-7-yl)cyclopentane-1,3-diamine (KI-ARv-3): tert-butyl ((1R,3R)-3-(5-propylpyrazolo[1,5-a]pyrimidin-7-yl)amino)cyclopentyl)carbamate (3, 481 mg, 1.34 mmol) was treated with a solution of 4 M HCl in MeOH for 1.5 h at room temperature. The solution was basified with saturated Na₂HCO₃ solution and extracted with DCM. The combined organic layers were dried over Na₂SO₄ and the solvent was removed in vacuo to yield KI-ARv-3 as brown syrup (343 mg, 1.33 mmol, 99%). ¹H NMR (DMSO-d₆): δ 7.99 (d, J=2.3 Hz, 1H), 7.44 (s, 1H), 6.28 (d, J=2.2 Hz, 1H), 6.03 (s, 1H), 4.21 (t, J=7.3 Hz, 1H), 3.43 (p, J=5.8 Hz, 1H), 2.61 (dd, J=8.4, 6.7 Hz, 2H), 2.20 (dtd, J=12.9, 7.9, 4.9 Hz, 1H), 2.00-1.83 (m, 3H), 1.83-1.58 (m, 5H), 1.30 (dtd, J=13.2, 8.0, 5.4 Hz, 1H), 0.92 (t, J=7.4 Hz, 3H). ¹³C NMR δ (101 MHz, DMSO-d₆) δ 162.26, 148.77, 146.00, 142.99, 93.59, 84.91, 51.59, 51.07, 41.97, 39.84 34.19, 30.66, 21.93, 13.76. LC-MS (ES⁺): m/z 260.4 [M+H]⁺.

### Preparation of Exemplary Compounds

KI-ARv3-D01 through -D07 were synthesized according to General Procedure A.

General Procedure A: To a solution of pomalidomide-linker-COOH (10 mg, 1 eq.) in DMF (0.6 mL), DIPEA (≥2 eq.), and HATU (1 eq.) were added. Then a solution of KI-ARv3 (˜1.3 eq.) in DMF (0.6 mL) was added and the mixture was allowed to stir at room temperature for 3 h. The crude mixture was then purified by HPLC (0.1% TFA H₂O/0.1% TFA MeCN) to yield degraders KI-ARv3-D01 through -D07.

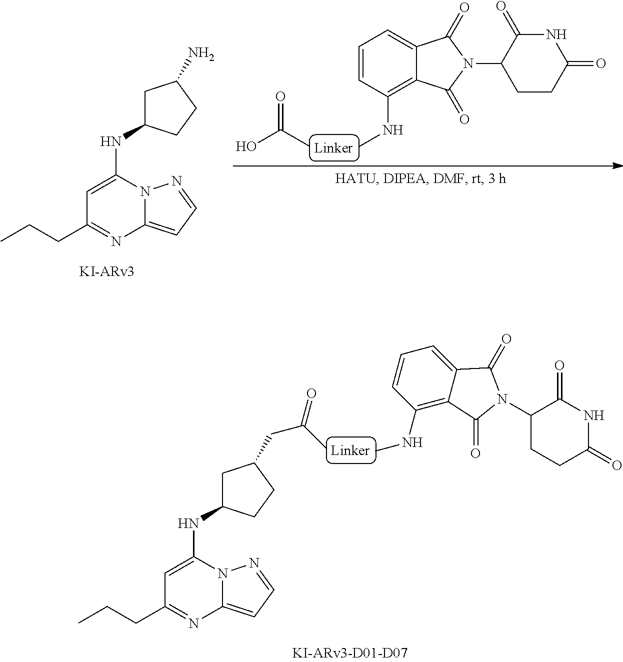

### 3-(2-((2-(2,6-Dioxopiperidin-3-yl)-1,3-dioxoisoindolin-4-yl)amino)ethoxy)-N-((1R,3R)-3-((5-propylpyrazolo[1,5-a]pyrimidin-7-yl)amino)cyclopentyl)propenamide (KI-ARv3-D01)

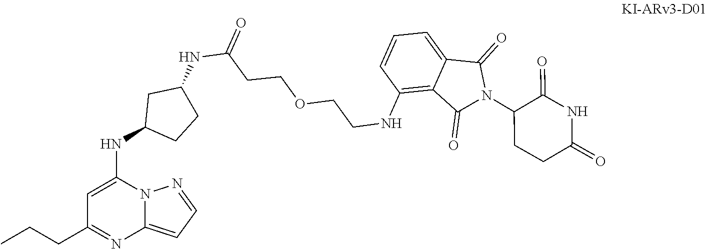 [SMILES: CCCc1cc(N[C@@H]2CC[C@@H](NC(=O)CCOCCNc3cccc4c3C(=O)N(C3CCC(=O)NC3=O)C4=O)C2)n2nccc2n1]

KI-ARv3-D01 was synthesized according to General Procedure A using pomalidomide-PEG₁-COOH (10 mg, 0.026 mmol), DIPEA (9 μL, 0.052 mmol), HATU (10 mg, 0.026 mmol), and KI-ARv-3 (8.6 mg, 0.033 mmol). Yield: 17% (2.8 mg, 4.42 mol). ¹H NMR (Methanol-d₄): δ 7.97 (dd, J=7.0, 2.3 Hz, 1H), 7.49 (ddd, J=8.7, 7.0, 5.8 Hz, 1H), 7.05 (dd, J=8.6, 4.7 Hz, 1H), 6.98 (dd, J=9.6, 7.1 Hz, 1H), 6.29 (t, J=2.2 Hz, 1H), 5.97 (d, J=5.6 Hz, 1H), 5.07-4.98 (m, 1H), 4.33 (tt, J=11.6, 6.1 Hz, 1H), 4.26-4.12 (m, 1H), 3.84-3.73 (m, 2H), 3.70 (t, J=5.1 Hz, 2H), 3.49 (t, J=5.1 Hz, 2H), 2.90-2.75 (m, 1H), 2.74-2.62 (m, 4H), 2.53-2.38 (m, 2H), 2.37-2.11 (m, 2H), 2.05 (td, J=9.8, 8.8, 4.5 Hz, 3H), 1.74 (dtd, J=15.5, 7.7, 2.1 Hz, 3H), 1.58 (ddq, J=22.1, 11.3, 8.0, 5.7 Hz, 1H), 0.98 (td, J=7.4, 2.4 Hz, 3H). LC-MS (ES⁺): m/z 631.7 [M+H]⁺

### 3-(2-(2-((2-(2,6-Dioxopiperidin-3-yl)-1,3-dioxoisoindolin-4-yl)amino)ethoxy)ethoxy)-N-((1R,3R)-3-((5-propylpyrazolo[1,5-a]pyrimidin-7-yl)amino)cyclopentyl)propenamide (KI-ARv3-D02)

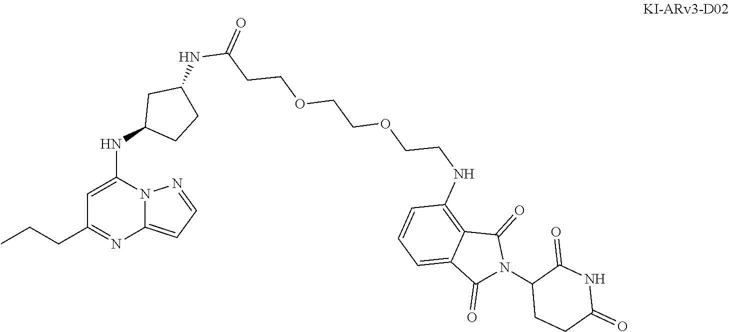 [SMILES: CCCc1cc(N[C@@H]2CC[C@@H](NC(=O)CCOCCOCCNc3cccc4c3C(=O)N(C3CCC(=O)NC3=O)C4=O)C2)n2nccc2n1]

KI-ARv3-D02 was synthesized according to General Procedure A using pomalidomide-PEG₂-COOH (10 mg, 0.023 mmol), DIPEA (8 μL, 0.046 mmol), HATU (8.7 mg, 0.023 mmol), and KI-ARv-3 (7.8 mg, 0.03 mmol). Yield: 90% (14.2 mg, 0.021 mmol). ¹H NMR (400 MHz, Methanol-d₄) δ 7.96 (d, J=2.3 Hz, 1H), 7.45 (ddd, J=8.7, 7.1, 5.1 Hz, 1H), 6.98 (ddd, J=14.4, 7.8, 3.8 Hz, 2H), 6.29 (d, J=2.3 Hz, 1H), 5.98 (s, 1H), 5.05 (dd, J=12.6, 5.4 Hz, 1H), 4.33 (p, J=6.8 Hz, 1H), 4.20 (p, J=6.8 Hz, 1H), 3.73 (dt, J=10.2, 5.6 Hz, 4H), 3.69-3.59 (m, 4H), 3.46 (t, J=5.3 Hz, 2H), 2.85 (ddd, J=17.7, 14.5, 5.1 Hz, 1H), 2.79-2.60 (m, 4H), 2.42 (dt, J=6.0, 4.0 Hz, 2H), 2.30 (dtt, J=12.1, 7.8, 4.5 Hz, 1H), 2.19 (ddt, J=10.4, 5.3, 2.5 Hz, 1H), 2.07 (tq, J=12.6, 5.7 Hz, 3H), 1.74 (dp, J=16.4, 8.8, 8.1 Hz, 3H), 1.60 (dddd, J=13.0, 8.5, 6.6, 4.4 Hz, 1H), 0.98 (t, J=7.4 Hz, 3H). LC-MS (ES⁺): m/z 675.8 [M+H]⁺

### 3-(2-(2-(2-((2-(2,6-Dioxopiperidin-3-yl)-1,3-dioxoisoindolin-4-yl)amino)ethoxy)ethoxy)ethoxy)-N-((1R,3R)-3-((5-propylpyrazolo[1,5-a]pyrimidin-7-yl)amino)cyclopentyl)propanamide (KI-ARv3-D03)

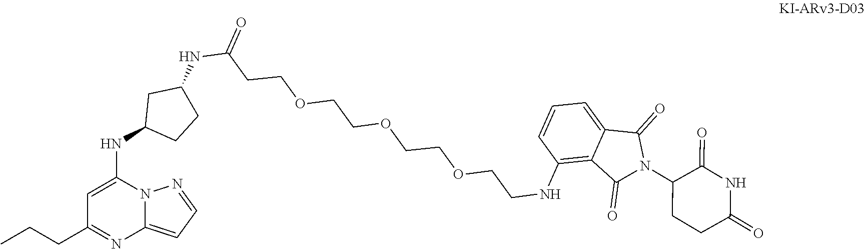 [SMILES: CCCc1cc(N[C@@H]2CC[C@@H](NC(=O)CCOCCOCCOCCNc3cccc4c3C(=O)N(C3CCC(=O)NC3=O)C4=O)C2)n2nccc2n1]

KI-ARv3-D03 was synthesized according to General Procedure A using pomalidomide-PEG₃-COOH (10 mg, 0.021 mmol), DIPEA (7.3 μL, 0.042 mmol), HATU (8 mg, 0.021 mmol), and KI-ARv-3 (7 mg, 0.027 mmol). Yield: 93% (14.4 mg, 0.02 mmol). ¹H NMR (400 MHz, Methanol-d₄): δ 7.95 (d, J=2.3 Hz, 1H), 7.47 (ddd, J=8.7, 7.0, 2.7 Hz, 1H), 7.06-6.94 (m, 2H), 6.29 (d, J=2.3 Hz, 1H), 6.00 (s, 1H), 5.04 (dd, J=12.5, 5.5 Hz, 1H), 4.34 (p, J=6.8 Hz, 1H), 4.23 (p, J=6.8 Hz, 1H), 3.76-3.56 (m, 12H), 3.44 (td, J=5.3, 2.3 Hz, 2H), 2.85 (ddd, J=17.7, 14.2, 5.2 Hz, 1H), 2.79-2.63 (m, 4H), 2.41 (td, J=6.0, 1.6 Hz, 2H), 2.32 (dtd, J=12.6, 7.6, 5.0 Hz, 1H), 2.19 (ddt, J=14.5, 7.5, 4.4 Hz, 1H), 2.14-1.99 (m, 3H), 1.84-1.68 (m, 3H), 1.67-1.55 (m, 1H), 0.98 (t, J=7.4 Hz, 3H). LC-MS (ES⁺): m/z 719.8 [M+H]⁺

### 1-((2-(2,6-Dioxopiperidin-3-yl)-1,3-dioxoisoindolin-4-yl)amino)-N-((1R,3R)-3-((5-propylpyrazolo[1,5-a]pyrimidin-7-yl)amino)cyclopentyl)-3,6,9,12-tetraoxapentadecan-15-amide (KI-ARv3-D04)

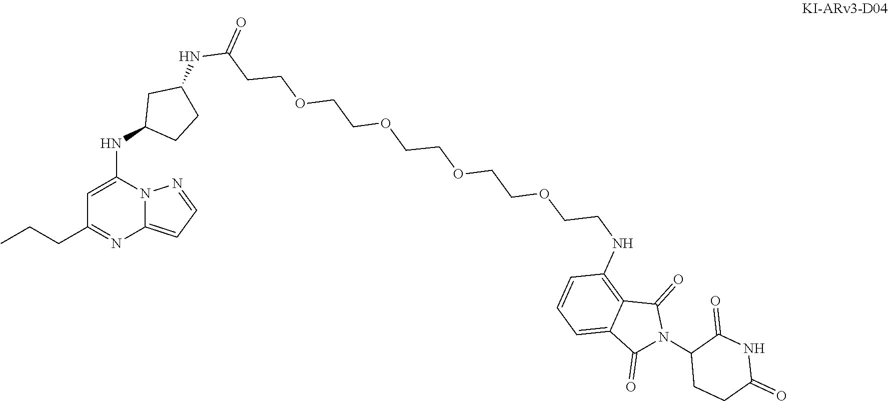 [SMILES: CCCc1cc(N[C@@H]2CC[C@@H](NC(=O)CCOCCOCCOCCOCCNc3cccc4c3C(=O)N(C3CCC(=O)NC3=O)C4=O)C2)n2nccc2n1]

KI-ARv3-D04 was synthesized according to General Procedure A using pomalidomide-PEG₄-COOH (11 mg, 0.019 mmol), DIPEA (7.3 μL, 0.042 mmol), HATU (8 mg, 0.021 mmol), and KI-ARv-3 (6 mg, 0.023 mmol). Yield: 88% (13 mg, 0.17 mmol).

¹H NMR (400 MHz, Methanol-d₄) δ 7.96 (d, J=2.3 Hz, 1H), 7.48 (dd, J=8.6, 7.1 Hz, 1H), 7.01 (dd, J=7.8, 5.2 Hz, 2H), 6.29 (d, J=2.3 Hz, 1H), 6.01 (s, 1H), 5.04 (dd, J=12.5, 5.5 Hz, 1H), 4.34 (p, J=8.1, 7.4 Hz, 1H), 4.25 (p, J=6.8 Hz, 1H), 3.70 (dt, J=13.0, 5.6 Hz, 4H), 3.60 (dd, J=9.2, 2.9 Hz, 12H), 3.45 (t, J=5.2 Hz, 2H), 2.85 (ddd, J=17.7, 14.2, 5.1 Hz, 1H), 2.79-2.62 (m, 4H), 2.42 (t, J=6.1 Hz, 2H), 2.33 (dtd, J=12.7, 7.9, 4.9 Hz, 1H), 2.27-2.12 (m, 1H), 2.13-2.02 (m, 3H), 1.74 (dq, J=14.2, 7.3 Hz, 3H), 1.68-1.56 (m, 1H), 0.99 (t, J=7.4 Hz, 3H). LC-MS (ES⁺): m/z 763.8 [M+H]⁺

### 7-((2-(2,6-Dioxopiperidin-3-yl)-1,3-dioxoisoindolin-4-yl)amino)-N-((1R,3R)-3-((5-propylpyrazolo[1,5-a]pyrimidin-7-yl)amino)cyclopentypheptanamide (KI-ARv3-D05)

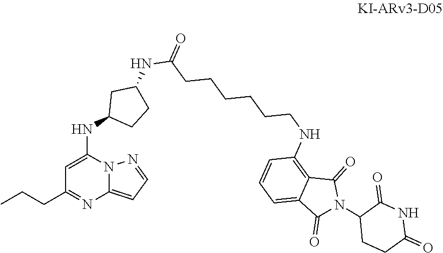 [SMILES: CCCc1cc(N[C@@H]2CC[C@@H](NC(=O)CCCCCCNc3cccc4c3C(=O)N(C3CCC(=O)NC3=O)C4=O)C2)n2nccc2n1]

KI-ARv3-D05 was synthesized according to General Procedure A using pomalidomide-C₆-COOH (11 mg, 0.025 mmol), DIPEA (9.6 μL, 0.055 mmol), HATU (10.6 mg, 0.028 mmol), and KI-ARv-3 (7.8 mg, 0.03 mmol). Yield: 87% (14 mg, 0.22 mmol). ¹H NMR (400 MHz, Methanol-d₄) δ 8.00 (d, J=2.3 Hz, 1H), 7.52 (dd, J=8.6, 7.1 Hz, 1H), 7.01 (dd, J=7.8, 4.0 Hz, 2H), 6.33 (d, J=2.3 Hz, 1H), 6.06 (s, 1H), 5.06 (dd, J=12.6, 5.4 Hz, 1H), 4.32 (dp, J=30.4, 6.9 Hz, 2H), 3.32-3.29 (m, 2H), 2.86 (ddd, J=17.7, 14.5, 5.1 Hz, 1H), 2.80-2.65 (m, 4H), 2.35 (dtd, J=12.8, 7.8, 4.8 Hz, 1H), 2.27-1.98 (m, 6H), 1.86-1.56 (m, 8H), 1.44 (dq, J=19.3, 7.0, 5.9 Hz, 4H), 1.01 (t, J=7.4 Hz, 3H). LC-MS (ES⁺): m/z 643.7 [M+H]⁺

### 4-((2-(2,6-Dioxopiperidin-3-yl)-1,3-dioxoisoindolin-4-yl)amino)-N-((1R,3R)-3-((5-propylpyrazolo[1,5-a]pyrimidin-7-yl)amino)cyclopentyl)butanamide (KI-ARv3-D06)

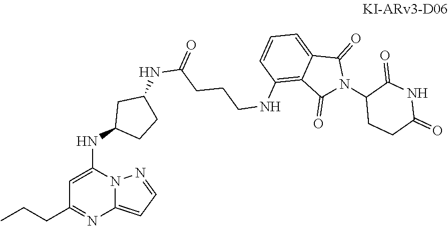 [SMILES: CCCc1cc(N[C@@H]2CC[C@@H](NC(=O)CCCNc3cccc4c3C(=O)N(C3CCC(=O)NC3=O)C4=O)C2)n2nccc2n1]

KI-ARv3-D06 was synthesized according to General Procedure A using pomalidomide-C₃-COOH (10 mg, 0.025 mmol), DIPEA (10 μL, 0.056 mmol), HATU (11 mg, 0.028 mmol), and KI-ARv-3 (10 mg, 0.036 mmol). Yield: 63% (9.6 mg, 0.16 mmol). ¹H NMR (400 MHz, Methanol-d₄) δ 8.02 (dd, J=17.2, 2.3 Hz, 1H), 7.55 (t, J=7.8 Hz, 1H), 7.10-7.00 (m, 2H), 6.32 (t, J=2.7 Hz, 1H), 6.01 (d, J=4.0 Hz, 1H), 5.07 (dt, J=12.4, 6.0 Hz, 1H), 4.29 (dp, J=44.7, 6.7 Hz, 2H), 3.40 (t, J=6.7 Hz, 2H), 2.86 (ddt, J=18.7, 14.5, 4.5 Hz, 1H), 2.80-2.64 (m, 4H), 2.33 (td, J=7.1, 3.7 Hz, 3H), 2.25-1.91 (m, 6H), 1.86-1.71 (m, 3H), 1.57 (tt, J=13.6, 7.6 Hz, 1H), 1.01 (t, J=7.3 Hz, 3H). LC-MS (ES⁺): m/z 601.4 [M+H]⁺

### 10-((2-(2,6-Dioxopiperidin-3-yl)-1,3-dioxoisoindolin-4-yl)amino)-N-((1R,3R)-3-((5-propylpyrazolo[1,5-a]pyrimidin-7-yl)amino)cyclopentyl)decanamide (KI-ARv3-D07)

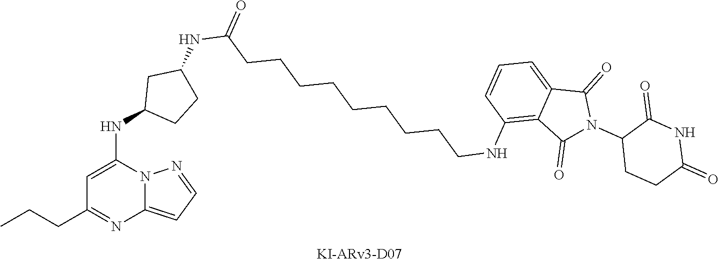 [SMILES: CCCc1cc(N[C@@H]2CC[C@@H](NC(=O)CCCCCCCCCNc3cccc4c3C(=O)N(C3CCC(=O)NC3=O)C4=O)C2)n2nccc2n1]

KI-ARv3-D07 was synthesized according to General Procedure A using pomalidomide-C₉-COOH (10 mg, 0.023 mmol), DIPEA (8 μL, 0.045 mmol), HATU (8.6 mg, 0.023 mmol), and KI-ARv-3 (8 mg, 0.029 mmol). Yield: 35% (5.5 mg, 0.008 mmol). ¹H NMR (Methanol-d₄): δ 7.99 (d, J=2.3 Hz, 1H), 7.54 (t, J=7.8 Hz, 1H), 7.08-6.97 (m, 2H), 6.32 (d, J=2.3 Hz, 1H), 6.04 (s, 1H), 5.06 (dd, J=12.5, 5.5 Hz, 1H), 4.32 (dp, J=36.7, 6.8 Hz, 2H), 3.31 (s, 2H), 2.93-2.63 (m, 5H), 2.35 (ddd, J=12.6, 8.9, 5.2 Hz, 1H), 2.30-2.00 (m, 6H), 1.88-1.73 (m, 3H), 1.73-1.55 (m, 5H), 1.49-1.33 (m, 10H), 1.01 (t, J=7.4 Hz, 3H). LC-MS (ES⁺): m/z 685.8 [M+H]⁺

### 4′-(((2-(2,6-dioxopiperidin-3-yl)-1,3-dioxoisoindolin-4-yl)amino)methyl)-N-((1R,3R)-3-((5-propylpyrazolo[1,5-a]pyrimidin-7-yl)amino)cyclopentyl)-[1,1′-biphenyl]-4-carboxamide (KI-ARv3-D08)

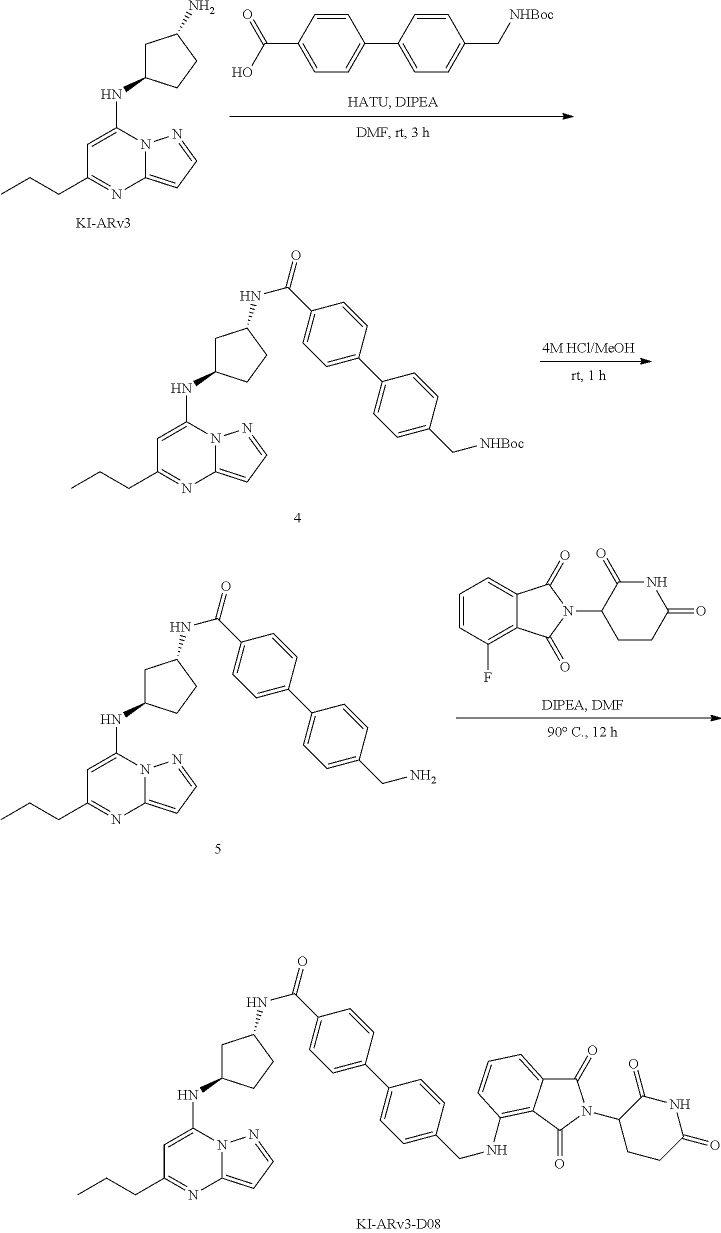 [SMILES: CCCc1cc(N[C@@H]2CC[C@@H](N)C2)n2nccc2n1 ; CC(C)(C)OC(=O)NCc1ccc(-c2ccc(C(=O)O)cc2)cc1 ; CCCc1cc(N[C@@H]2CC[C@@H](NC(=O)c3ccc(-c4ccc(CNC(=O)OC(C)(C)C)cc4)cc3)C2)n2nccc2n1 ; CCCc1cc(N[C@@H]2CC[C@@H](NC(=O)c3ccc(-c4ccc(CN)cc4)cc3)C2)n2nccc2n1 ; O=C1CCC(N2C(=O)c3cccc(F)c3C2=O)C(=O)N1 ; CCCc1cc(N[C@@H]2CC[C@@H](NC(=O)c3ccc(-c4ccc(CNc5cccc6c5C(=O)N(C5CCC(=O)NC5=O)C6=O)cc4)cc3)C2)n2nccc2n1]

To a solution of 4′-(((tert-butoxycarbonyl)amino)methyl)-[1,1′-biphenyl]-4-carboxylic acid (86 mg, 0.263 mmol.) in DMF (3 mL) was added DIPEA (≥2 eq.), and HATU (100 mg., 0.263 mmol). Then a solution of KI-ARv-3 (88.7 mg., 0.342 mmol) in DMF (1 mL) was added and the mixture was allowed to stir at room temperature for 3 h. The crude mixture was then purified by HPLC (0.1% TFA H₂O/0.1% TFA MeCN). Product containing fractions were basified with NaHCO₃ solution and extracted with DCM (3×20 mL). Combined organic fractions were dried with Na₂SO₄ and removal of the solvent under reduced pressure yielded intermediate 4 (KI-ARv3-D09). ¹H NMR (400 MHz, DMSO-d₆) δ 9.85 (s, 1H), 8.45 (d, J=7.2 Hz, 1H), 8.31 (d, J=2.3 Hz, 1H), 7.97-7.91 (m, 2H), 7.76 (d, J=8.3 Hz, 2H), 7.69 (d, J=7.9 Hz, 2H), 7.45 (t, J=6.2 Hz, 1H), 7.35 (d, J=8.0 Hz, 2H), 6.59-6.51 (m, 2H), 4.56 (p, J=7.1 Hz, 2H), 4.17 (d, J=5.9 Hz, 2H), 2.78 (dd, J=8.5, 6.7 Hz, 2H), 2.35-2.06 (m, 4H), 1.89 (dq, J=14.8, 8.0 Hz, 1H), 1.73 (ddd, J=32.2, 14.0, 7.9 Hz, 3H), 1.40 (s, 9H), 0.96 (t, J=7.3 Hz, 3H). LC-MS (ES⁺): m/z 569.6 [M+H]⁺

Intermediate 4 (KI-ARv3-D09) was treated with 4 M HCl/MeOH (2 mL) at RT for 1 h. The reaction mixture was basified with a saturated solution of NaHCO₃ and extracted with DCM. Organic fractions were dried with Na₂SO₄ and removal of the solvent under reduced pressure yielded intermediate 5 (KI-ARv3-D10) as white solid. ¹H NMR (400 MHz, Methanol-d₄): δ 7.99 (d, J=2.3 Hz, 1H), 7.94-7.88 (m, 2H), 7.75-7.69 (m, 2H), 7.68-7.62 (m, 2H), 7.45 (d, J=8.0 Hz, 2H), 6.32 (d, J=2.2 Hz, 1H), 6.08 (s, 1H), 4.63 (p, J=7.1 Hz, 1H), 4.37 (p, J=6.6 Hz, 1H), 3.85 (s, 2H), 2.69 (dd, J=8.6, 6.8 Hz, 2H), 2.49-2.30 (m, 2H), 2.24 (td, J=7.2, 6.8, 1.9 Hz, 2H), 1.91-1.72 (m, 4H), 1.00 (t, J=7.3 Hz, 3H).

A mixture of 2-(2,6-dioxopiperidin-3-yl)-4-fluoroisoindoline-1,3-dione (17 mg, 0.062 mmol) and 4′-(aminomethyl)-N-(3-((5-propylpyrazolo[1,5-a]pyrimidin-7-yl)amino)cyclopentyl)-[1,1′-biphenyl]-4-carboxamide 5 (KI-ARv3-D10) (29 mg, 0.062 mmol) and DIPEA (129 mg, 0.123 mmol) in dry DMF (2 mL) was stirred at 90° C. for 12 h. The reaction mixture was concentrated under reduced pressure and purified by preparative HPLC (0.1% TFA H₂O, 0.1% TFA MeCN) to yield KI-ARv3-D08 (7.2 mg, 0.01 mmol, 16%). ¹H NMR (400 MHz, Methanol-d₄): δ 7.99 (d, J=2.3 Hz, 1H), 7.91 (d, J=8.2 Hz, 2H), 7.72 (d, J=8.1 Hz, 2H), 7.66 (d, J=8.0 Hz, 2H), 7.48 (dd, J=7.9, 5.5 Hz, 3H), 7.06 (d, J=7.1 Hz, 1H), 6.97 (d, J=8.5 Hz, 1H), 6.32 (d, J=2.3 Hz, 1H), 6.07 (s, 1H), 5.09 (dd, J=12.4, 5.4 Hz, 1H), 4.63 (d, J=3.1 Hz, 3H), 4.36 (p, J=6.4 Hz, 1H), 2.93-2.64 (m, 5H), 2.49-2.29 (m, 2H), 2.23 (t, J=7.6 Hz, 2H), 2.19-2.10 (m, 1H), 1.81 (dh, J=22.6, 7.4 Hz, 4H), 1.00 (t, J=7.4 Hz, 3H). LC-MS (ES⁺): m/z 725.5 [M+H]⁺

### Synthesis of VHL-Based Degraders

An orthogonal PROTAC library (KI-ARv3-D11 through -D18) based on CDK inhibitor KI-ARv-03 and the VHL-ligand as E3 ubiquitin ligase binder was also synthesized and evaluated. Various PEG (KI-ARv3-D11 through -D14) and carbon-based (KI-ARv3-D15 through D17) connections as well as a rigid biphenyl linker (KI-ARv3-D018) were utilized. With such compounds, ubiquitination of the target protein is performed through VHL.

The VHL ligand was synthesized as described by Crew et al. (J. Med. Chem. 2018, 61, 583-598) and equipped with various PEG and carbon linkers (VHL-linker-COOHs). Degraders KI-ARv3-D11 through -D18 were synthesized according to General Procedure B.

General Procedure B: To a solution of VHL-linker-COOH (crude from previous reaction step or purified, 1 eq.) in DMF (3-5 mL), DIPEA (3-5 eq.) and HATU (1.5 eq.) were added. Then KI-ARv3 (1.5 eq.) was added and the mixture was allowed to stir at room temperature for 3 h. The crude mixture was then purified by HPLC (0.1% TFA H₂O/0.1% TFA MeCN) to yield degraders KI-ARv3-D11 through -D18.

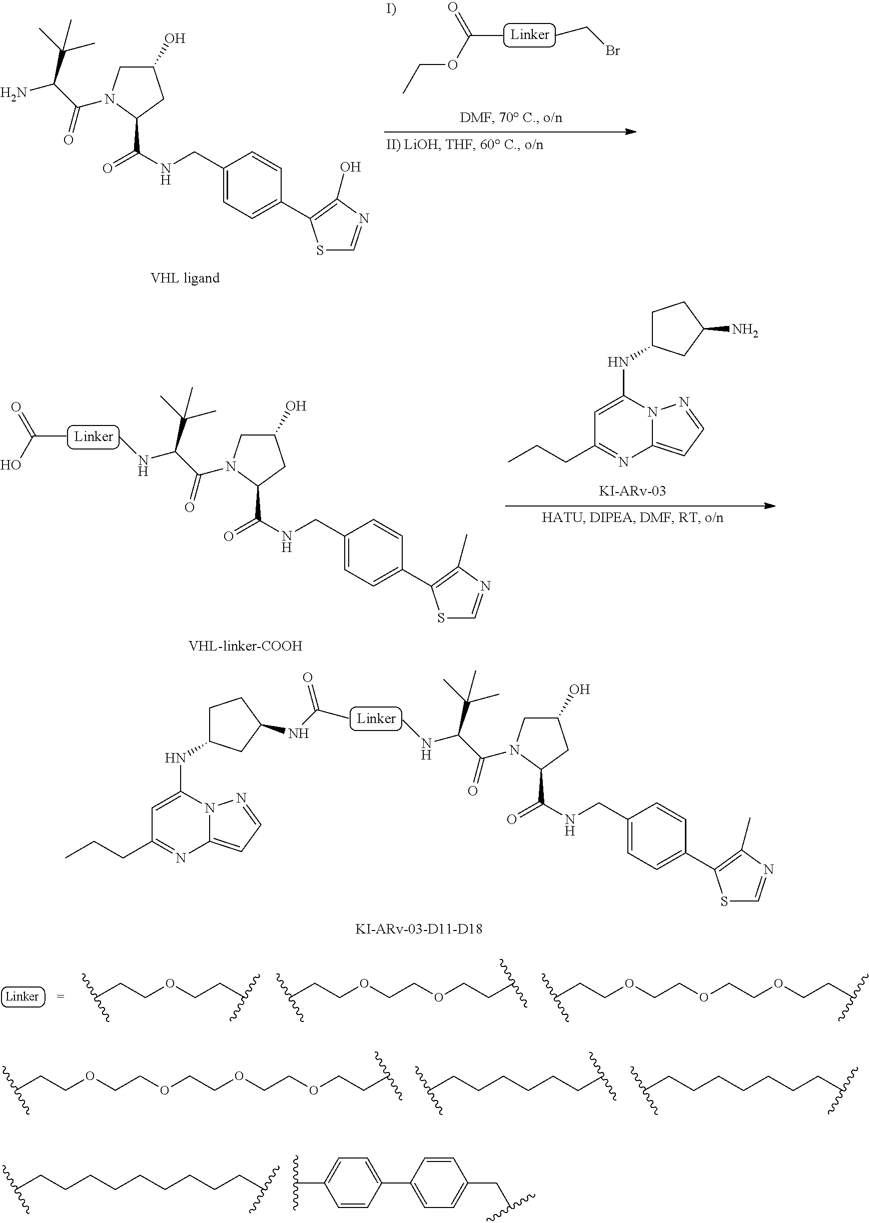

### (2S,4R)-1-((S)-3,3-dimethyl-2-((2-(3-oxo-3-(((1R,3R)-3-((5-propylpyrazolo[1,5-a]pyrimidin-7-yl)amino)cyclopentyl)amino)propoxy)ethyl)amino)butanoyl)-4-hydroxy-N-(4-(4-methylthiazol-5-yl)benzyl)pyrrolidine-2-carboxamide TFA-salt (KI-ARv3-D11)

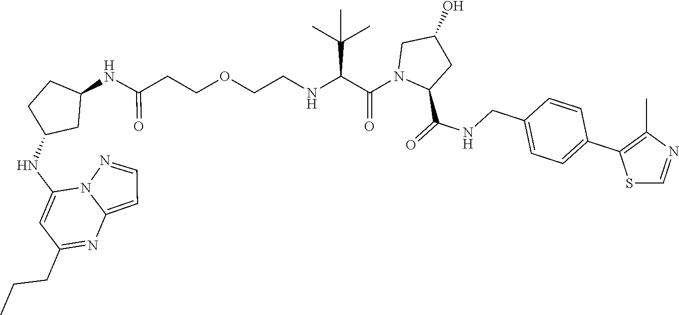 [SMILES: CCCc1cc(N[C@@H]2CC[C@@H](NC(=O)CCOCCN[C@H](C(=O)N3C[C@H](O)C[C@H]3C(=O)NCc3ccc(-c4scnc4C)cc3)C(C)(C)C)C2)n2nccc2n1]

KI-ARv3-D11 was synthesized according to General Procedure B using VHL-PEG1-COOH (crude, 0.061 mmol), DIPEA (54.3 μL, 0.305 mmol), HATU (34.2 mg, 0.092 mmol), and KI-ARv-3 (23.9 mg, 0.092 mmol). Yield: 46% (24.4 mg, 0.028 mmol, two steps). ¹H NMR (400 MHz, MeOD) δ 9.11 (s, 1H), 8.18 (d, J=2.2 Hz, 1H), 7.53-7.39 (m, 4H), 6.49 (q, J=2.9, 2.2 Hz, 2H), 4.70 (dd, J=9.5, 7.6 Hz, 1H), 4.56 (dq, J=13.3, 2.8 Hz, 3H), 4.47-4.39 (m, 1H), 4.36 (d, J=15.6 Hz, 1H), 4.22 (d, J=2.2 Hz, 1H), 3.92 (d, J=11.4 Hz, 1H), 3.84-3.64 (m, 5H), 3.27 (dt, J=6.9, 3.3 Hz, 1H), 3.14 (ddd, J=13.8, 6.3, 3.8 Hz, 1H), 2.85 (t, J=7.7 Hz, 2H), 2.57-2.47 (m, 5H), 2.43-2.16 (m, 4H), 2.16-2.05 (m, 2H), 1.91-1.79 (m, 3H), 1.72-1.60 (m, 1H), 1.16 (s, 9H), 1.06 (t, J=7.3 Hz, 3H). LC-MS (ES⁺): m/z 789.07 [M+H]⁺

### (2S,4R)-1-((S)-3,3-dimethyl-2-((2-(2-(3-oxo-3-(((1R,3R)-3-((5-propylpyrazolo[1,5-a]pyrimidin-7-yl)amino)cyclopentyl)amino)propoxy)ethoxy)ethyl)amino)butanoyl)-4-hydroxy-N-(4-(4-methylthiazol-5-yl)benzyl)pyrrolidine-2-carboxamide TFA-salt (KI-ARv3-D12)

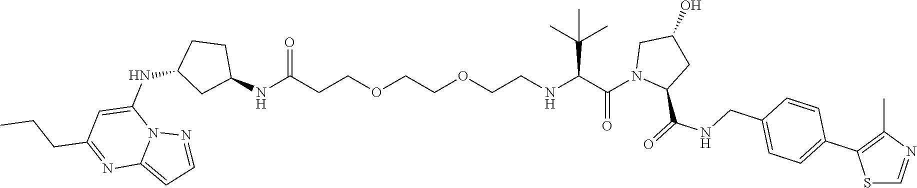 [SMILES: CCCc1cc(N[C@@H]2CC[C@@H](NC(=O)CCOCCOCCN[C@H](C(=O)N3C[C@H](O)C[C@H]3C(=O)NCc3ccc(-c4scnc4C)cc3)C(C)(C)C)C2)n2nccc2n1]

KI-ARv3-D12 was synthesized according to General Procedure B using VHL-PEG2-COOH (crude, 0.065 mmol), DIPEA (57.9 μL, 0.325 mmol), HATU (37.3 mg, 0.092 mmol), and KI-ARv-3 (25.4 mg, 0.098 mmol). Yield: 38% (23.5 mg, 0.025 mmol, two steps). ¹H NMR (400 MHz, MeOD) δ 9.13 (s, 1H), 8.18 (d, J=2.2 Hz, 1H), 7.52-7.41 (m, 4H), 6.48 (d, J=2.7 Hz, 2H), 4.70 (dd, J=9.5, 7.6 Hz, 1H), 4.57 (d, J=15.3 Hz, 3H), 4.46-4.26 (m, 3H), 3.91 (d, J=11.3 Hz, 1H), 3.83-3.56 (m, 9H), 3.26 (ddd, J=10.2, 6.3, 3.2 Hz, 1H), 3.16 (ddd, J=13.7, 6.2, 3.7 Hz, 1H), 2.87-2.82 (m, 2H), 2.53-2.05 (m, 11H), 1.85 (tdd, J=15.0, 8.0, 3.6 Hz, 3H), 1.73-1.60 (m, 1H), 1.17 (s, 9H), 1.06 (t, J=7.3 Hz, 3H). LC-MS (ES⁺): m/z 832.85 [M+H]⁺

### (2S,4R)-1-((S)-2-(tert-butyl)-15-oxo-15-(((1R,3R)-3-((5-propylpyrazolo[1,5-a]pyrimidin-7-yl)amino)cyclopentyl)amino)-6,9,12-trioxa-3-azapentadecanoyl)-4-hydroxy-N-(4-(4-methylthiazol-5-yl)benzyl)pyrrolidine-2-carboxamide TFA-salt (KI-ARv3-D13)

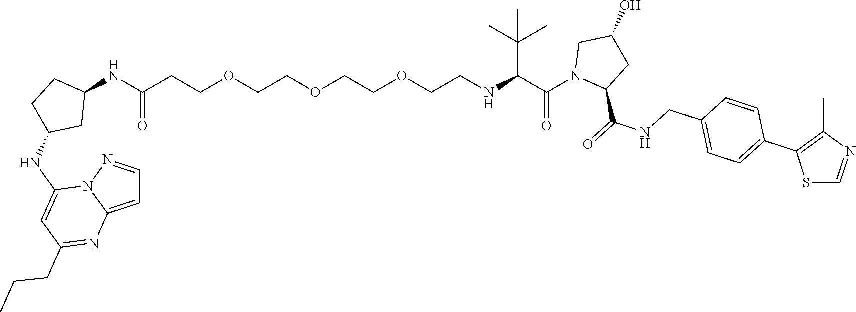 [SMILES: CCCc1cc(N[C@@H]2CC[C@@H](NC(=O)CCOCCOCCOCCN[C@H](C(=O)N3C[C@H](O)C[C@H]3C(=O)NCc3ccc(-c4scnc4C)cc3)C(C)(C)C)C2)n2nccc2n1]

KI-ARv3-D13 was synthesized according to General Procedure B using VHL-PEG3-COOH (crude, 0.060 mmol), DIPEA (53.4 μL, 0.325 mmol), HATU (34.2 mg, 0.090 mmol), and KI-ARv-3 (23.3 mg, 0.090 mmol). Yield: 57% (33.0 mg, 0.034 mmol, two steps). ¹H NMR (400 MHz, MeOD) δ 9.11 (d, J=1.4 Hz, 1H), 8.18 (d, J=2.2 Hz, 1H), 7.54-7.39 (m, 4H), 6.48 (d, J=1.9 Hz, 2H), 4.70 (td, J=8.5, 7.5, 1.8 Hz, 1H), 4.62-4.47 (m, 3H), 4.45-4.29 (m, 3H), 3.93 (d, J=11.3 Hz, 1H), 3.86-3.58 (m, 13H), 3.31-3.10 (m, 4H), 2.84 (t, J=7.4 Hz, 2H), 2.48 (d, J=9.2 Hz, 5H), 2.43-2.02 (m, 6H), 1.94-1.78 (m, 3H), 1.65 (dq, J=12.6, 8.1 Hz, 1H), 1.17 (s, 9H), 1.06 (t, J=7.4 Hz, 3H). ¹³C NMR (101 MHz, MeOD) δ 173.89, 173.46, 167.34, 159.72, 153.67, 150.80, 147.54, 147.13, 140.75, 140.70, 130.74, 130.36, 129.01, 91.93, 87.52, 71.48, 71.45, 71.34, 71.31, 71.10, 68.26, 67.55, 66.59, 61.08, 58.10, 54.48, 50.66, 47.87, 43.67, 39.39, 39.07, 37.49, 36.73, 36.36, 32.03, 31.72, 28.28, 26.71, 23.46, 15.18, 13.80. LC-MS (ES⁺): m/z 876.98 [M+H]⁺

### (2S,4R)-1-((S)-2-(tert-butyl)-18-oxo-18-(((1R,3R)-3-((5-propylpyrazolo[1,5-a]pyrimidin-7-yl)amino)cyclopentyl)amino)-6,9,12,15-tetraoxa-3-azaoctadecanoyl)-4-hydroxy-N-(4-(4-methylthiazol-5-yl)benzyl)pyrrolidine-2-carboxamide TFA salt (KI-ARv3-D14)

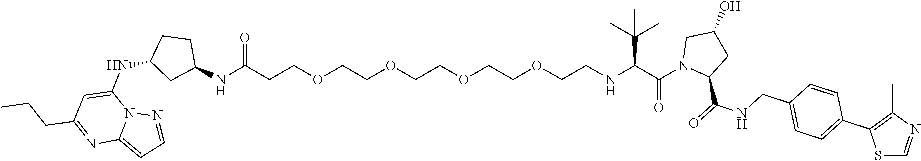 [SMILES: CCCc1cc(N[C@@H]2CC[C@@H](NC(=O)CCOCCOCCOCCOCCN[C@H](C(=O)N3C[C@H](O)C[C@H]3C(=O)NCc3ccc(-c4scnc4C)cc3)C(C)(C)C)C2)n2nccc2n1]

KI-ARv3-D14 was synthesized according to General Procedure B using VHL-PEG4-COOH (crude, 0.073 mmol), DIPEA (64.7 μL, 0.365 mmol), HATU (41.8 mg, 0.110 mmol), and KI-ARv-3 (28.5 mg, 0.090 mmol). Yield: 49% (37.0 mg, 0.036 mmol, two steps). ¹H NMR (400 MHz, MeOD) δ 9.13 (s, 1H), 8.18 (d, J=2.2 Hz, 1H), 7.52-7.41 (m, 4H), 6.48 (d, J=2.6 Hz, 2H), 4.70 (t, J=8.6 Hz, 1H), 4.64-4.49 (m, 3H), 4.35 (dd, J=24.8, 8.9 Hz, 3H), 3.93 (d, J=11.3 Hz, 1H), 3.84-3.58 (m, 17H), 3.27 (dt, J=6.4, 3.7 Hz, 1H), 3.15 (ddd, J=13.8, 6.4, 3.7 Hz, 1H), 2.85 (t, J=7.7 Hz, 2H), 2.52-2.43 (m, 5H), 2.43-2.27 (m, 2H), 2.28-2.04 (m, 4H), 1.84 (dtd, J=14.7, 8.5, 7.3, 2.4 Hz, 3H), 1.66 (dq, J=13.0, 8.1 Hz, 1H), 1.17 (s, 9H), 1.06 (t, J=7.4 Hz, 3H). LC-MS (ES⁺): m/z 921.08 [M+H]⁺

### (2S,4R)-1-((S)-3,3-dimethyl-2-47-oxo-7-(((1R,3R)-3-((5-propylpyrazolo[1,5-a]pyrimidin-7-yl)amino)cyclopentyl)amino)heptyl)amino)butanoyl)-4-hydroxy-N-(4-(4-methylthiazol-5-yl)benzyl)pyrrolidine-2-carboxamide TFA salt (KI-ARv3-D15)

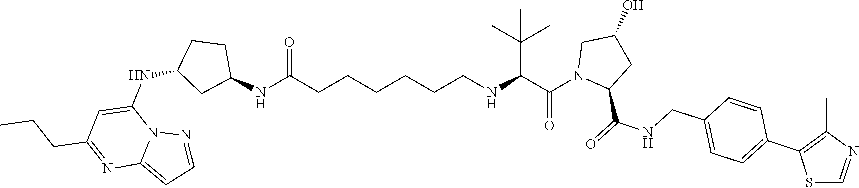 [SMILES: CCCc1cc(N[C@@H]2CC[C@@H](NC(=O)CCCCCCN[C@H](C(=O)N3C[C@H](O)C[C@H]3C(=O)NCc3ccc(-c4scnc4C)cc3)C(C)(C)C)C2)n2nccc2n1]

KI-ARv3-D15 was synthesized according to General Procedure B using VHL-C6-COOH (22 mg, 0.039 mmol), DIPEA (20.6 μL, 0.117 mmol), HATU (22.4 mg, 0.059 mmol), and KI-ARv-3 (15.3 mg, 0.059 mmol). Yield: 69% (23.9 mg, 0.027 mmol). ¹H NMR (400 MHz, MeOD) δ 9.05 (s, 1H), 8.18 (d, J=2.2 Hz, 1H), 7.53-7.38 (m, 4H), 6.49 (d, J=1.9 Hz, 2H), 4.70 (dd, J=9.6, 7.6 Hz, 1H), 4.61-4.47 (m, 3H), 4.46-4.31 (m, 2H), 4.10 (s, 1H), 3.89 (d, J=11.4 Hz, 1H), 3.68 (dd, J=11.4, 3.2 Hz, 1H), 3.05-2.90 (m, 2H), 2.85 (t, J=7.7 Hz, 2H), 2.49 (s, 3H), 2.44-2.28 (m, 2H), 2.21 (q, J=7.4 Hz, 4H), 2.08 (dtt, J=14.3, 6.0, 3.1 Hz, 2H), 1.92-1.80 (m, 3H), 1.78-1.58 (m, 5H), 1.43-1.34 (m, 4H), 1.16 (s, 9H), 1.06 (t, J=7.3 Hz, 3H). LC-MS (ES⁺): m/z 800.93 [M+H]⁺

### (2S,4R)-1-((S)-3,3-dimethyl-2-((8-oxo-8-(((1R,3R)-3-((5-propylpyrazolo[1,5-a]pyrimidin-7-yl)amino)cyclopentyl)amino)octyl)amino)butanoyl)-4-hydroxy-N-(4-(4-methylthiazol-5-yl)benzyl)pyrrolidine-2-carboxamide TFA salt (KI-ARv3-D16)

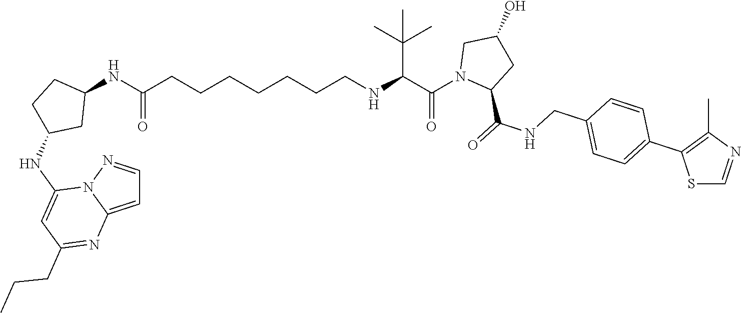 [SMILES: CCCc1cc(N[C@@H]2CC[C@@H](NC(=O)CCCCCCCN[C@H](C(=O)N3C[C@H](O)C[C@H]3C(=O)NCc3ccc(-c4scnc4C)cc3)C(C)(C)C)C2)n2nccc2n1]

KI-ARv3-D16 was synthesized according to General Procedure B using VHL-C7-COOH (21 mg, 0.037 mmol), DIPEA (20.6 μL, 0.148 mmol), HATU (21.3 mg, 0.056 mmol), and KI-ARv-3 (18.6 mg, 0.056 mmol). Yield: 54% (17.9 mg, 0.020 mmol). ¹H NMR (400 MHz, MeOD) δ 9.20 (d, J=0.9 Hz, 1H), 8.19 (d, J=2.3 Hz, 1H), 7.52-7.39 (m, 4H), 6.48 (d, J=2.4 Hz, 2H), 4.70 (dd, J=9.6, 7.6 Hz, 1H), 4.61-4.49 (m, 3H), 4.41-4.31 (m, 2H), 4.09 (s, 1H), 3.88 (d, J=11.4 Hz, 1H), 3.68 (dd, J=11.3, 3.3 Hz, 1H), 3.04-2.90 (m, 2H), 2.85 (dd, J=8.6, 6.8 Hz, 2H), 2.51 (d, J=2.7 Hz, 3H), 2.36 (tdd, J=21.4, 10.7, 6.0 Hz, 2H), 2.20 (t, J=7.6 Hz, 4H), 2.15-2.02 (m, 2H), 1.85 (h, J=7.4 Hz, 3H), 1.76-1.56 (m, 5H), 1.41-1.32 (m, 6H), 1.16 (s, 9H), 1.06 (t, J=7.3 Hz, 3H). LC-MS (ES⁺): m/z 814.98 [M+H]⁺

### (2S,4R)-1-((S)-3,3-dimethyl-2-((10-oxo-10-(((1R,3R)-3-((5-propylpyrazolo[1,5-a]pyrimidin-7-yl)amino)cyclopentyl)amino)decyl)amino)butanoyl)-4-hydroxy-N-(4-(4-methylthiazol-5-yl)benzyl)pyrrolidine-2-carboxamide TFA salt (KI-ARv3-D17)

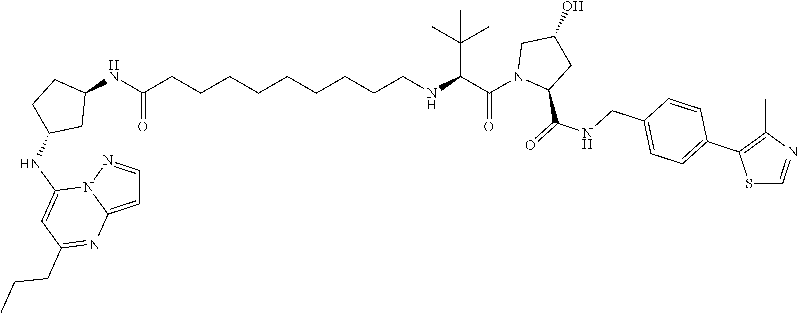 [SMILES: CCCc1cc(N[C@@H]2CC[C@@H](NC(=O)CCCCCCCCCN[C@H](C(=O)N3C[C@H](O)C[C@H]3C(=O)NCc3ccc(-c4scnc4C)cc3)C(C)(C)C)C2)n2nccc2n1]

KI-ARv3-D17 was synthesized according to General Procedure A using VHL-C9-COOH (18.6 mg, 0.031 mmol), DIPEA (22.0 μL, 0.124 mmol), HATU (17.8 mg, 0.056 mmol), and KI-ARv-3 (15.6 mg, 0.047 mmol). Yield: 52% (15.3 mg, 0.016 mmol). ¹H NMR (400 MHz, MeOD) δ 9.10 (s, 1H), 8.19 (d, J=2.2 Hz, 1H), 7.52-7.39 (m, 4H), 6.51-6.45 (m, 2H), 4.69 (dd, J=9.6, 7.7 Hz, 1H), 4.60-4.49 (m, 3H), 4.40-4.31 (m, 2H), 4.09 (s, 1H), 3.88 (d, J=11.4 Hz, 1H), 3.68 (dd, J=11.4, 3.3 Hz, 1H), 3.03-2.88 (m, 2H), 2.87-2.81 (m, 2H), 2.49 (s, 3H), 2.43-2.28 (m, 2H), 2.28-2.15 (m, 4H), 2.14-2.02 (m, 2H), 1.92-1.79 (m, 3H), 1.81-1.55 (m, 5H), 1.33 (s, 10H), 1.16 (s, 9H), 1.07 (t, J=7.4 Hz, 3H). LC-MS (ES⁺): m/z 842.91 [M+H]⁺

### (2S,4R)-1-((S)-3,3-dimethyl-2-(((4′-(((1R,3R)-3-((5-propylpyrazolo[1,5-a]pyrimidin-7-yl)amino)cyclopentyl)carbamoyl)-[1,1′-biphenyl]-4-yl)methyl)amino)butanoyl)-4-hydroxy-N-(4-(4-methylthiazol-5-yl)benzyl)pyrrolidine-2-carboxamide TFA salt (KI-ARv3-D18).

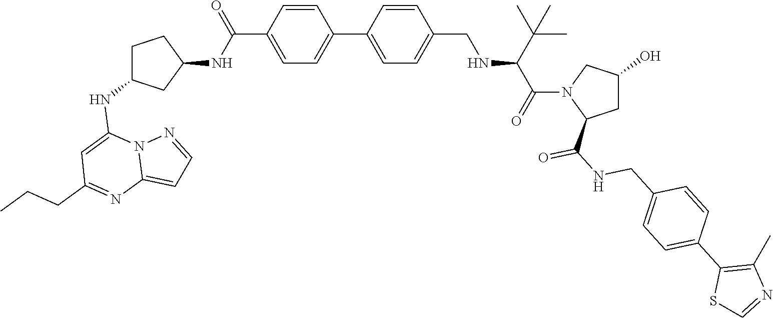 [SMILES: CCCc1cc(N[C@@H]2CC[C@@H](NC(=O)c3ccc(-c4ccc(CN[C@H](C(=O)N5C[C@H](O)C[C@H]5C(=O)NCc5ccc(-c6scnc6C)cc5)C(C)(C)C)cc4)cc3)C2)n2nccc2n1]

KI-ARv3-D18 was synthesized according to General Procedure A using VHL-biphenyl-COOH (57.3 mg, 0.089 mmol), DIPEA (48.2 μL, 0.268 mmol), HATU (51.0 mg, 0.134 mmol), and KI-ARv-3 (15.6 mg, 0.134 mmol). Yield: 71% (61.4 mg, 0.063 mmol). ¹H NMR (400 MHz, MeOD) δ 9.08 (s, 1H), 8.20 (d, J=2.2 Hz, 1H), 8.00-7.91 (m, 2H), 7.84-7.74 (m, 4H), 7.65 (d, J=8.0 Hz, 2H), 7.52-7.39 (m, 4H), 6.54-6.47 (m, 2H), 4.70-4.47 (m, 5H), 4.39-4.31 (m, 2H), 4.15 (d, J=13.6 Hz, 1H), 3.83 (s, 1H), 3.64 (dd, J=11.1, 3.9 Hz, 1H), 3.49 (d, J=11.3 Hz, 1H), 2.86 (dd, J=8.7, 6.7 Hz, 2H), 2.52-2.41 (m, 4H), 2.40-2.16 (m, 4H), 2.08 (ddd, J=13.2, 8.7, 4.5 Hz, 1H), 2.00-1.78 (m, 4H), 1.14 (s, 9H), 1.07 (t, J=7.3 Hz, 3H). ¹³C NMR (101 MHz, MeOD) δ 173.86, 169.69, 166.98, 159.70, 153.57, 150.85, 147.73, 147.14, 144.36, 142.82, 140.77, 140.62, 134.90, 134.15, 132.34, 130.85, 130.78, 130.36, 129.15, 129.14, 129.01, 128.15, 91.95, 87.53, 70.97, 66.77, 61.28, 57.58, 54.64, 51.72, 51.33, 43.67, 39.33, 39.00, 36.75, 36.45, 31.98, 31.91, 26.75, 23.46, 15.25, 13.80. LC-MS (ES⁺): m/z 882.84 [M+H]⁺

### Synthesis of KB-0472 Based Degraders

The CDK9 inhibitor KB-0472 was also employed for degrader design and synthesis.

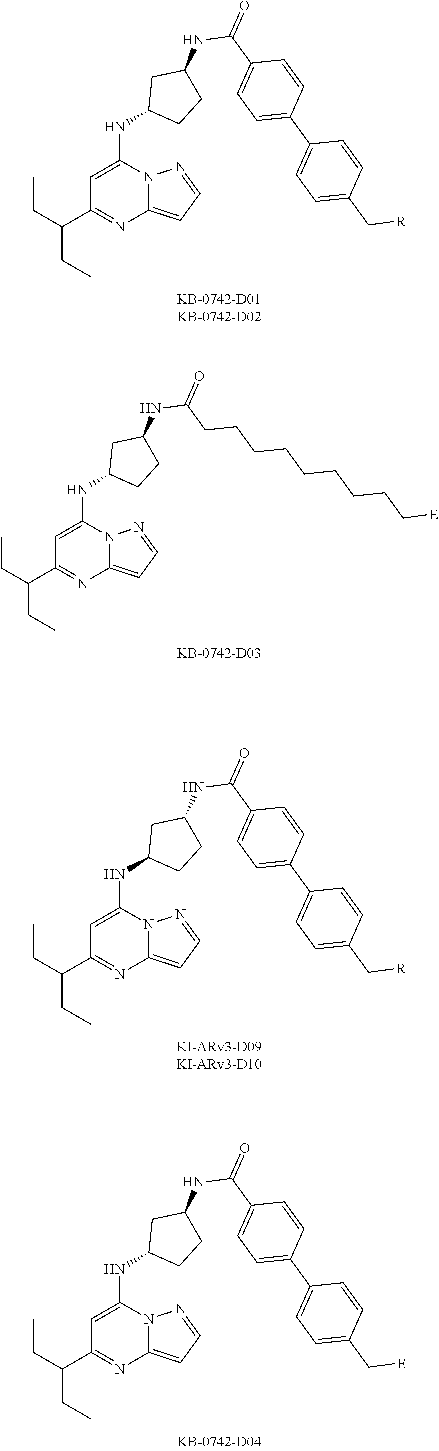

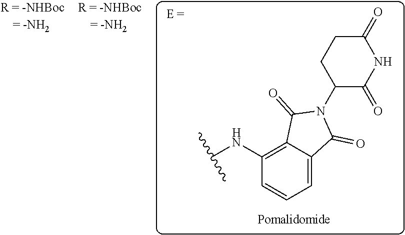 [SMILES: *Nc1cccc2c1C(=O)N(C1CCC(=O)NC1=O)C2=O]

### (1S,3S)-N¹-(5-(pentan-3-yl)pyrazolo[1,5-a]pyrimidin-7-yl)cyclopentane-1,3-diamine (KB-0742)

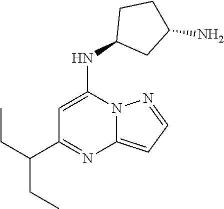 [SMILES: CCC(CC)c1cc(N[C@H]2CC[C@H](N)C2)n2nccc2n1]

KB-0742 was synthesized as described by Richters, et al. (Cell. Chem. Biol. 2021, 28, 134-147 e114)

### tert-butyl ((4′-(((1S,3S)-3-((5-(pentan-3-yl)pyrazolo[1,5-a]pyrimidin-7-yl)amino)cyclopentyl)carbamoyl)-[1,1′-biphenyl]-4-yl)methyl)carbamate TFA salt (KB-0742-D01)

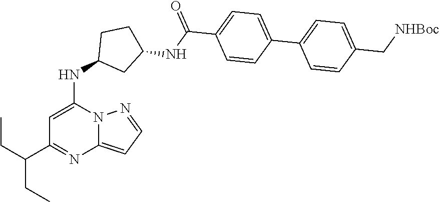 [SMILES: CCC(CC)c1cc(N[C@H]2CC[C@H](NC(=O)c3ccc(-c4ccc(CNC(=O)OC(C)(C)C)cc4)cc3)C2)n2nccc2n1]

To a solution of 4′-(((tert-butoxycarbonyl)amino)methyl)-[1,1′-biphenyl]-4-carboxylic acid (262 mg, 0.8 mmol.) in DMF (10 mL) was added DIPEA (418 μL, 2.4 mmol), and HATU (304 mg., 0.8 mmol). Then a solution of KB-0742 (150 mg., 0.52 mmol) was added and the mixture was allowed to stir at room temperature overnight. The reaction mixture was basified with saturated NaHCO₃ solution, extracted with DCM and dried over Na₂SO₄. The crude was purified by HPLC (0.1% TFA H₂O/0.1% TFA MeCN) to yield KB-0742-D01 (276.7 mg, 0.397 mmol, 76%). ¹H NMR (400 MHz, MeOD) δ 8.21 (d, J=2.2 Hz, 1H), 7.92 (d, J=8.3 Hz, 2H), 7.70 (d, J=8.1 Hz, 2H), 7.61 (d, J=7.8 Hz, 2H), 7.37 (d, J=8.1 Hz, 2H), 6.52 (d, J=2.2 Hz, 1H), 6.48 (s, 1H), 4.65 (tt, J=14.4, 6.2 Hz, 2H), 4.27 (s, 2H), 2.74-2.61 (m, 1H), 2.46 (dtd, J=11.1, 7.3, 6.9, 3.8 Hz, 1H), 2.31 (dddd, J=31.6, 14.0, 7.6, 5.6 Hz, 3H), 1.99-1.70 (m, 6H), 1.46 (s, 9H), 0.93 (t, J=7.4 Hz, 6H). LC-MS (ES⁺): m/z 597.71 [M+H]⁺

### 4′-(aminomethyl)-N-((1S,3S)-3-((5-(pentan-3-yl)pyrazolo[1,5-a]pyrimidin-7-yl)amino)cyclopentyl)-[1,1′-biphenyl]-4-carboxamide (KB-0742-D02)

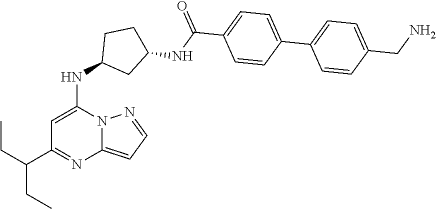 [SMILES: CCC(CC)c1cc(N[C@H]2CC[C@H](NC(=O)c3ccc(-c4ccc(CN)cc4)cc3)C2)n2nccc2n1]

KB-0742-D01(115.3 mg, 0.166 mmol (impure fractions used)) was treated with 4 M HCl/MeOH (6 mL) at RT for 1 h. The reaction mixture was purified by HPLC (0.1% TFA H₂O/0.1% TFA MeCN) to yield KB-0742-D01 (71.8 mg, 0.124 mmol, 75%). ¹H NMR (400 MHz, MeOD) δ 8.21 (d, J=2.2 Hz, 1H), 8.01-7.92 (m, 2H), 7.74 (dd, J=8.3, 2.7 Hz, 4H), 7.57 (d, J=8.2 Hz, 2H), 6.53 (d, J=2.2 Hz, 1H), 6.50 (s, 1H), 4.75-4.58 (m, 2H), 4.18 (s, 2H), 2.71 (tt, J=9.2, 5.7 Hz, 1H), 2.47 (dq, J=13.1, 4.0, 3.4 Hz, 1H), 2.32 (dddd, J=22.3, 13.9, 7.7, 5.7 Hz, 3H), 2.00-1.72 (m, 6H), 0.93 (t, J=7.4 Hz, 6H). LC-MS (ES⁺): m/z 497.37 [M+H]⁺

### 10-((2-(2,6-dioxopiperidin-3-yl)-1,3-dioxoisoindolin-4-yl)amino)-N-((1R,3R)-3-((5-(pentan-3-yl)pyrazolo[1,5-a]pyrimidin-7-yl)amino)cyclopentyl)decanamide TFA salt (KB-0742-D03)

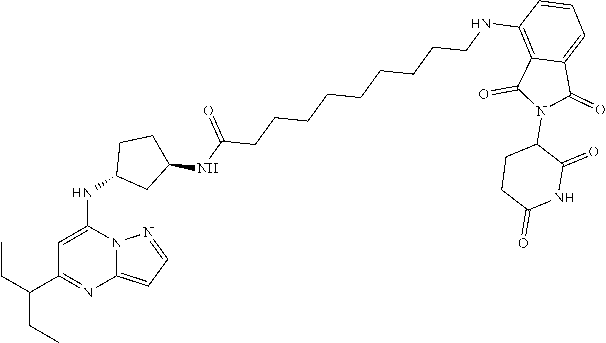 [SMILES: CCC(CC)c1cc(N[C@@H]2CC[C@@H](NC(=O)CCCCCCCCCNc3cccc4c3C(=O)N(C3CCC(=O)NC3=O)C4=O)C2)n2nccc2n1]

KB-0742-D03 was synthesized according to General Procedure A using pomalidomide-C₉-COOH (25 mg, 0.056 mmol), DIPEA (29.5 μL, 0.169 mmol), HATU (21.4 mg, 0.056 mmol), and KB-0742 (21.1 mg, 0.073 mmol). Yield: 73% (33.2 mg, 0.041 mmol). ¹H NMR (500 MHz, MeOD) δ 8.18 (d, J=2.2 Hz, 1H), 7.50 (dd, J=8.6, 7.1 Hz, 1H), 6.99 (dd, J=7.8, 3.8 Hz, 2H), 6.50 (d, J=2.2 Hz, 1H), 6.42 (s, 1H), 5.03 (dd, J=12.5, 5.4 Hz, 1H), 4.55 (p, J=7.1 Hz, 1H), 4.34 (p, J=7.2 Hz, 1H), 3.29-3.26 (m, 2H), 2.91-2.79 (m, 1H), 2.77-2.62 (m, 3H), 2.36 (dtd, J=12.4, 7.9, 4.2 Hz, 1H), 2.25-2.15 (m, 4H), 2.13-2.01 (m, 2H), 1.93-1.71 (m, 5H), 1.70-1.53 (m, 5H), 1.43-1.28 (m, 10H), 0.91 (t, J=7.4 Hz, 6H). ¹³C NMR (126 MHz, MeOD) δ 176.11, 174.63, 171.66, 170.78, 169.29, 162.95, 150.82, 148.27, 147.19, 140.70, 137.21, 133.86, 117.96, 111.69, 110.91, 92.22, 86.73, 54.52, 50.66, 50.17, 50.11, 43.38, 39.42, 37.05, 32.21, 32.01, 31.71, 30.43, 30.30, 30.25, 30.20, 28.41, 28.38, 27.87, 26.98, 23.79, 12.18. LC-MS (ES⁺): m/z 713.51 [M+H]⁺

### 4′-(((2-(2,6-dioxopiperidin-3-yl)-1,3-dioxoisoindolin-4-yl)amino)methyl)-N-((1S,3S)-3-((5-(pentan-3-yl)pyrazolo[1,5-a]pyrimidin-7-yl)amino)cyclopentyl)-[1,1′-biphenyl]-4-carboxamide TFA salt (KB-0742-D04)

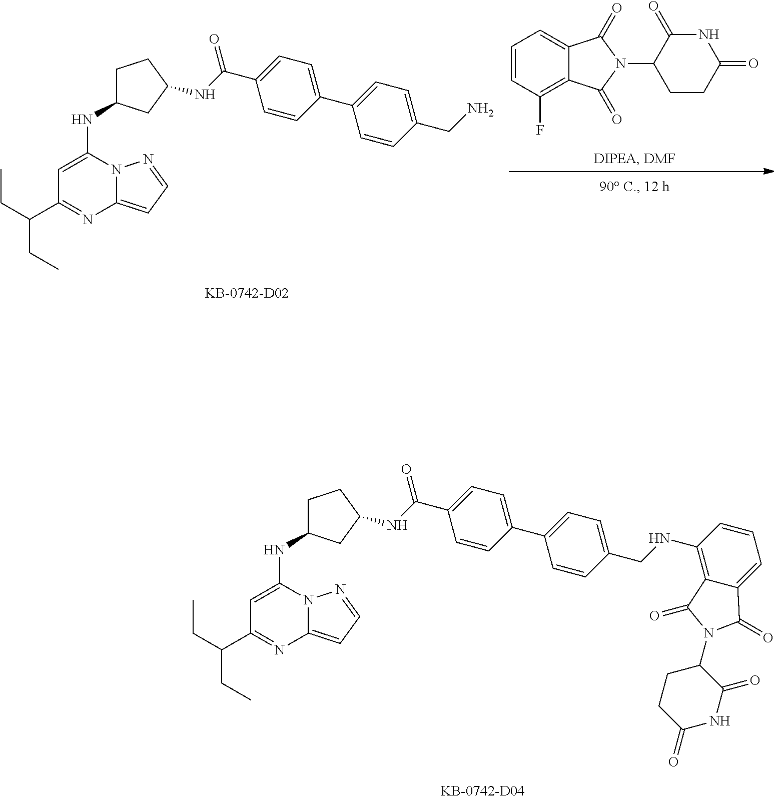 [SMILES: CCC(CC)c1cc(N[C@H]2CC[C@H](NC(=O)c3ccc(-c4ccc(CN)cc4)cc3)C2)n2nccc2n1 ; O=C1CCC(N2C(=O)c3cccc(F)c3C2=O)C(=O)N1 ; CCC(CC)c1cc(N[C@H]2CC[C@H](NC(=O)c3ccc(-c4ccc(CNc5cccc6c5C(=O)N(C5CCC(=O)NC5=O)C6=O)cc4)cc3)C2)n2nccc2n1]

A mixture of 2-(2,6-dioxopiperidin-3-yl)-4-fluoroisoindoline-1,3-dione (70.7 mg, 0.256 mmol), KB-0742-D02 (135.6 mg, 0.256 mmol) and DIPEA (133 μL, 0.768 mmol) in dry DMF (5 mL) was stirred at 90° C. for 12 h. The reaction mixture was concentrated under reduced pressure and purified by preparative HPLC (0.1% TFA H₂O, 0.1% TFA MeCN) to yield KB-0742-D04 (59 mg, 0.069 mmol, 27%). ¹H NMR (500 MHz, MeOD) δ 8.22 (d, J=2.2 Hz, 1H), 7.90 (d, J=8.4 Hz, 2H), 7.70 (d, J=8.6 Hz, 2H), 7.63 (d, J=8.2 Hz, 2H), 7.48-7.43 (m, 3H), 7.03 (dd, J=7.2, 2.7 Hz, 1H), 6.94 (dd, J=8.6, 2.8 Hz, 1H), 6.52 (d, J=2.2 Hz, 1H), 6.48 (s, 1H), 5.11-5.03 (m, 1H), 4.65 (dq, J=17.8, 7.2 Hz, 2H), 4.59 (s, 2H), 2.89-2.64 (m, 5H), 2.46 (dtd, J=11.6, 7.7, 3.9 Hz, 1H), 2.35 (ddt, J=13.4, 7.9, 4.0 Hz, 2H), 2.26 (ddd, J=14.1, 8.1, 6.4 Hz, 1H), 2.17-2.07 (m, 1H), 2.00-1.72 (m, 5H), 0.93 (t, J=7.4 Hz, 6H). LC-MS (ES⁺): m/z 753.40 [M+H]⁺.

### CDK9 Degradation Assays

Assays were performed to demonstrate the ability of the exemplary compounds to degrade CDK9 in cells.

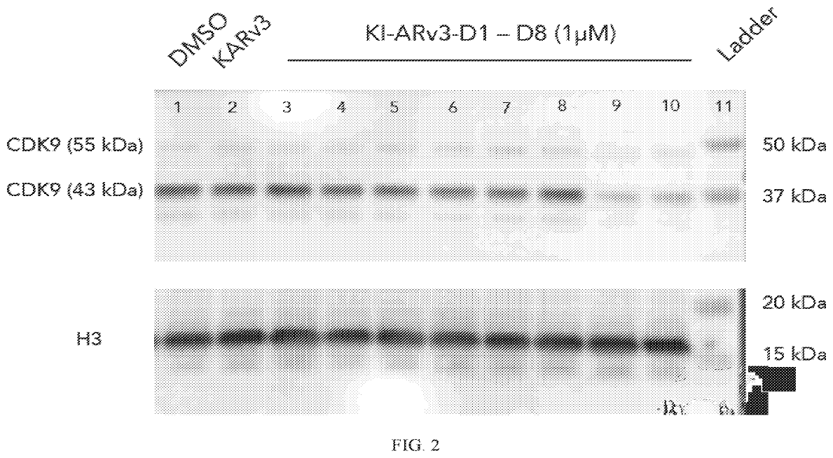

MOLT4 cells were treated with 1 μM of compounds KI-ARv3-D01 through -D08 for 6 hours and CDK9 protein levels were monitored per Western Blot (FIG. 2). Compounds KI-ARv3-D07 and KI-ARv3-D08 (FIG. 2, lane 9 and 10) reduced CDK9 protein levels by approximately 80% compared to DMSO as well as parent compound (KI-ARv3) treatment.

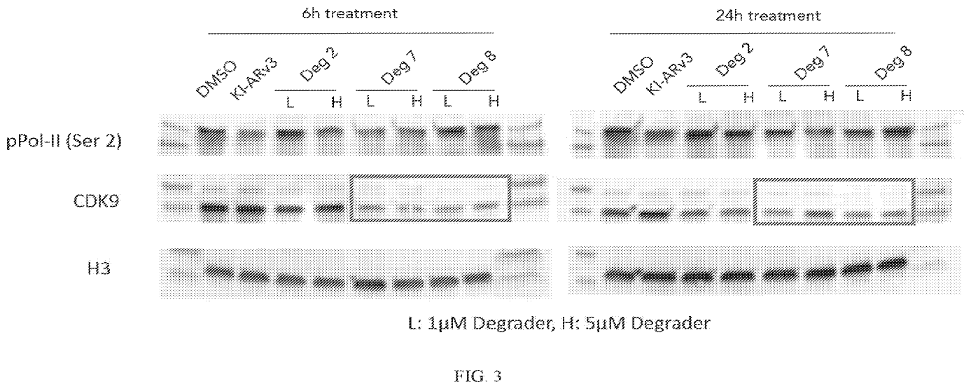

Compounds KI-ARv3-D07 and KI-ARv3-D08 were evaluated in subsequent studies investigating different cell lines as well as different treatment times. Efficiencies were measured by monitoring their impact on CDK9 itself as well as downstream targets of CDK9 phosphorylation such as AR and Pol II. MOLT4 cells were exposed to low dose (1 μM) and high dose (5 μM) of compounds KI-ARv3-D02 (control), -D07, and -D08 for 6 hours and 24 hours, respectively (FIG. 3). Compared to DMSO treatment, CDK9 degradation was observed for both KI-ARv3-D07 and -D08 after 6 hours, while KI-ARv3 treatment alone did not induce degradation. Incubation for 24 hours led to significant degradation of CDK9 at low doses (1 μM) of all compounds. CDK9 levels were reduced by KI-ARv3-D07 and -D08. However, at high dose (5 μM) the commonly occurring “hook effect” was observed for KI-ARv3-D07, but not for the other compounds, suggesting alternate pharmacokinetic behavior or different mode of action (MoA). This effect is commonly observed upon saturation of both targets (ligase and protein of interest) and describes the concentration of degrader at which both protein of interest and E3 ubiquitin ligase are saturated in a univalent fashion, thus inhibiting bivalent bridging of CDK9 and CRBN in this case.

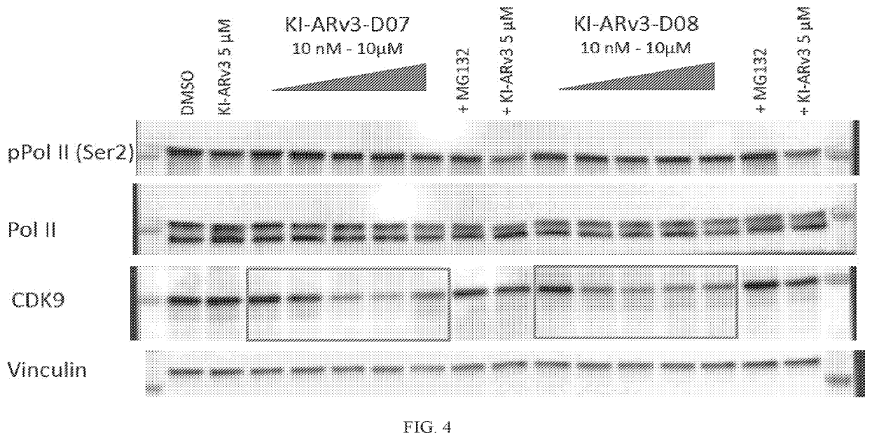

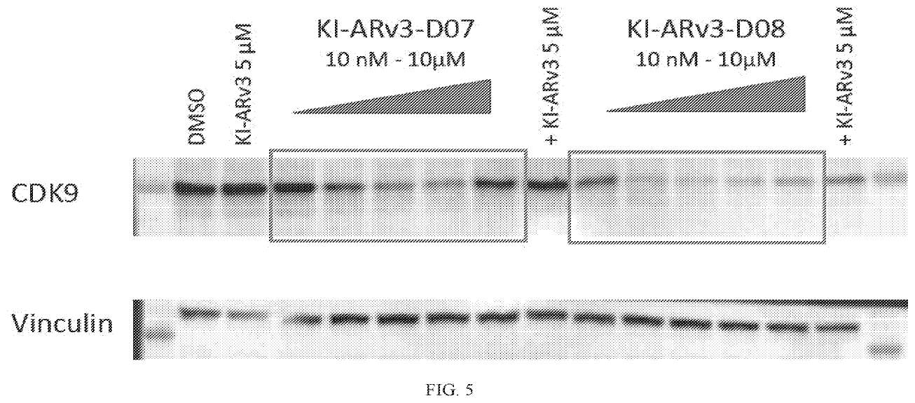

A dose range of KI-ARv3-D07 and KI-ARv3-D08 (10 nM-10 μM) was evaluated in MOLT4 cells to gauge the minimal concentration at which the compounds show maximal efficacy (FIGS. 4 and 5). After 6 hours of treatment a concentration dependent decrease of CDK9 levels starting at 100 nM of each compound was observed. In addition, CDK9 levels were rescued by cotreatment of each compound at 1 μM concentration with proteasome inhibitor MG132 (10 μM) or parent compound KI-ARv3 (5 μM). These results suggest that compounds of Formula (I) facilitate CDK9 degradation through the proteasomal pathway and through direct interaction with CDK9.

Treatment with the compounds for 24 hours gave a similar reduction of CDK9 protein levels. However, subtle differences in the MoA of KI-ARv3-D07 and KI-ARv3-D08 were demonstrated as well. Degradation of CDK9 was not demonstrated by applying 10 μM of KI-ARv3-D07 indicating that the “hook effect” is observed through univalent binding of CDK9 and E3 ubiquitin ligase CRBN. On the contrary, KI-ARv3-D08 facilitated stable CDK9 degradation (by approx. 80%) across the whole range of applied concentrations. Differences in the rigidity of the utilized linker (flexible carbon chain vs rigid biphenyl) as well as different pharmacokinetic properties of these compounds may account for this difference.

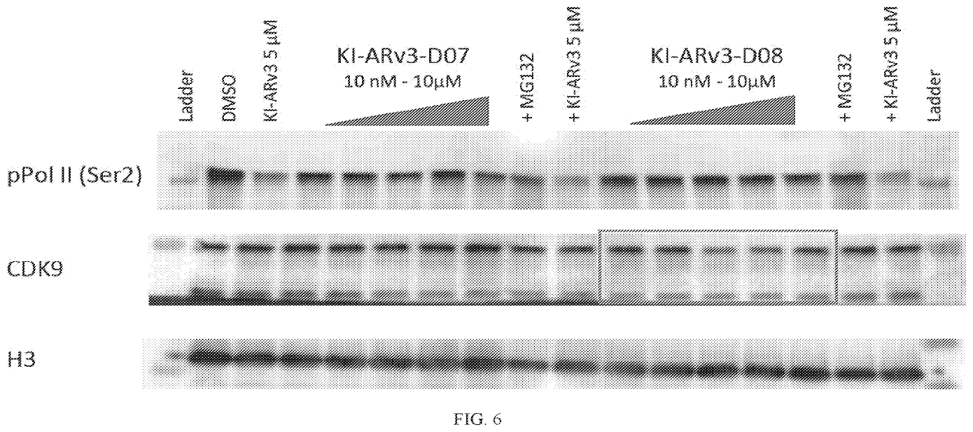

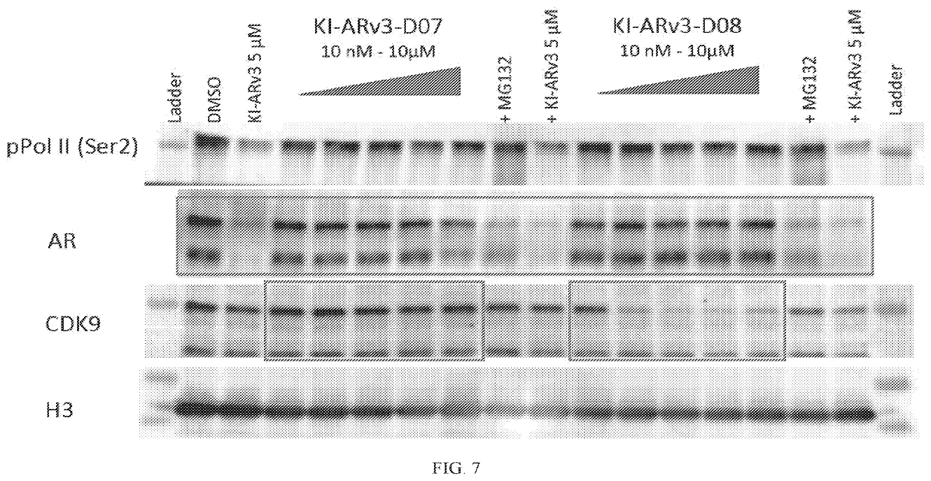

Compounds were evaluated in ARv7 positive 22Rv1 cells in order to assess their potential to degrade CDK9 and its associated downstream phosphorylation targets Pol II as well as AR and related splice variants (FIGS. 6 and 7). After short exposure (6 hours) to KI-ARv3-D07 and KI-ARv3-D08 no significant reduction of CDK9 levels was observed for KI-ARv3-D07 across the whole range of applied compound concentrations (10 nM-10 μM). For KI-ARv3-D08, however, subtle reduction in CDK9 protein levels starting at 500 nM compound concentration was observed. This effect was rescued by cotreatment with MG132 (10 μM) or parent compound KI-ARv3 (5 μM) using 1 μM KI-ARv3-D08 concentration.

Surprisingly, KI-ARv3-D07 (at any applied concentration 10 nM-10 μM) was also not able to facilitate degradation of CDK9 after prolonged exposure (24 hours) in 22Rv1 cells (FIG. 7), representing the first time that lineage specific degradation (MOLT4 vs 22Rv1) was demonstrated for a chimeric degrader. However, KI-ARv3-D08 induced significant CDK9 degradation in 22Rv1 cells after 24 hours of treatment starting at 100 nM compound concentration as it was observed in MOLT4. CDK9 protein level rescue using MG132 and KI-ARv3 was demonstrated, but protein levels were not rescued to parental levels, most likely due to the intrinsic impact of the parent compound KI-ARv3 as well as MG132 on the viability of 22Rv1 cells.

While treatment with 5 μM of the parent compound KI-ARv3 reduced AR, AR splice variant, and Pol II levels, treatment with KI-ARv3-D08 did exclusively impact CDK9 protein levels with no observed effect on global AR and Pol II protein levels (FIG. 7).

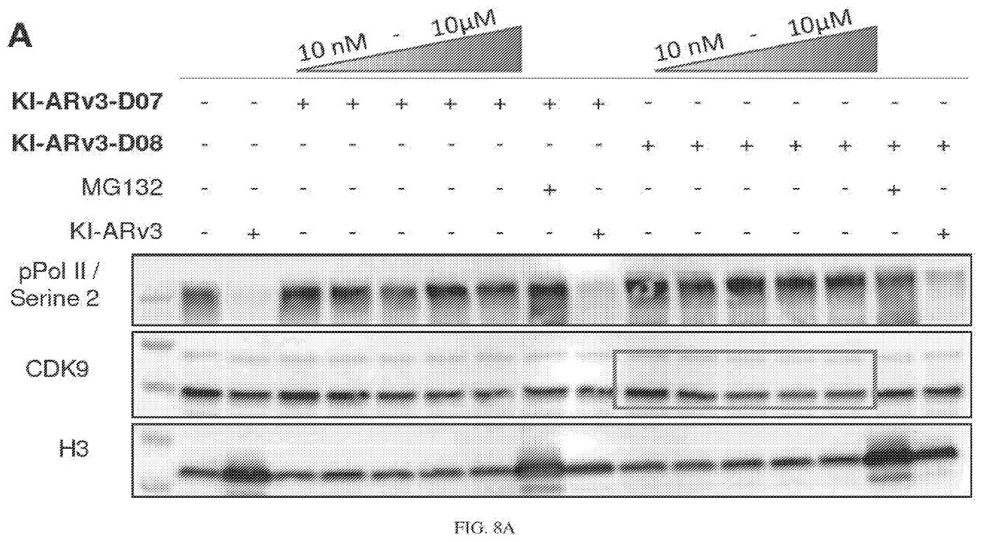

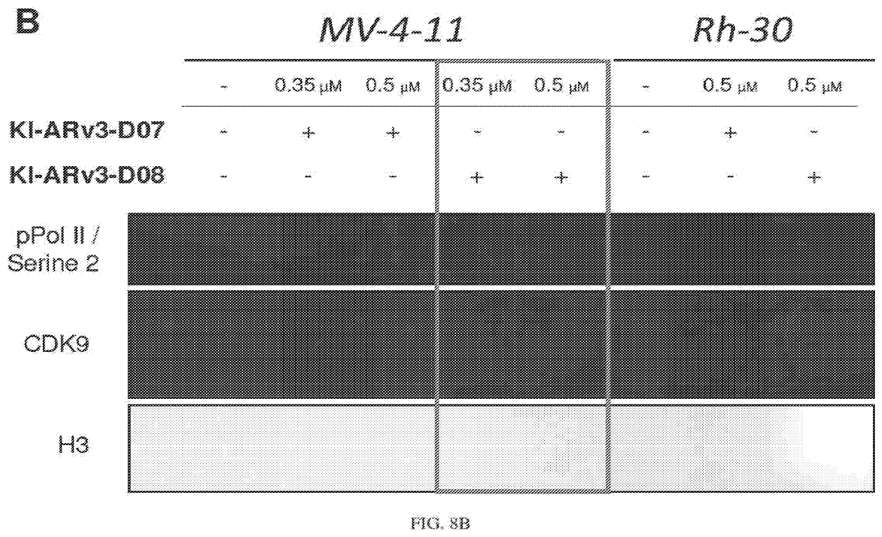

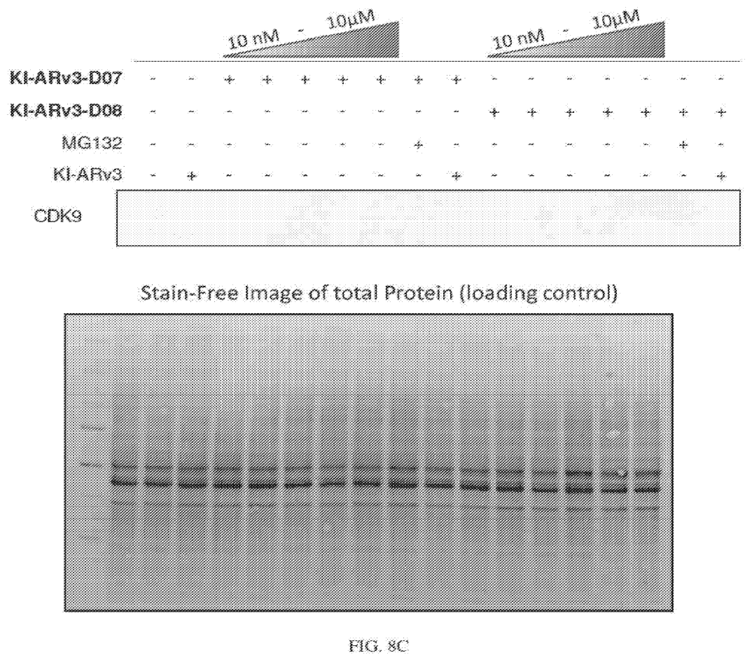

The effect of KI-ARv3-D07 and -08 on impacting CDK9 levels in the additional cell lineages MV-4-11 (leukemia cell line) and the Rh-30 (rhabdomyosarcoma cell line) at 6 h and 8 h treatment times (FIG. 8) was also evaluated. Western blotting illustrated that the biphenyl linker equipped chimera KI-ARv3-D08 reduced CDK9 levels by approximately 60% in MV-4-11 at 6 h and 8 h treatment time starting at 350 nM compound concentration. Rescue experiments using proteasome inhibitor MG132 demonstrated that this effect was reversible and proteasome dependent (FIG. 8A). KI-ARv3-D07 (C9-linker) did not lead to significant changes in CDK9 levels (FIGS. 8A and 8B). In Rh-30 no changes in CDK9 levels were detected at any compound concentration (10 nM, 100 nM, 500 nM, 1 μM, 10 μM) at either 6 h or 8 h treatment time (FIGS. 8B and 8C). These results support a lineage specific mode of action of the KI-ARv-03 based degraders.

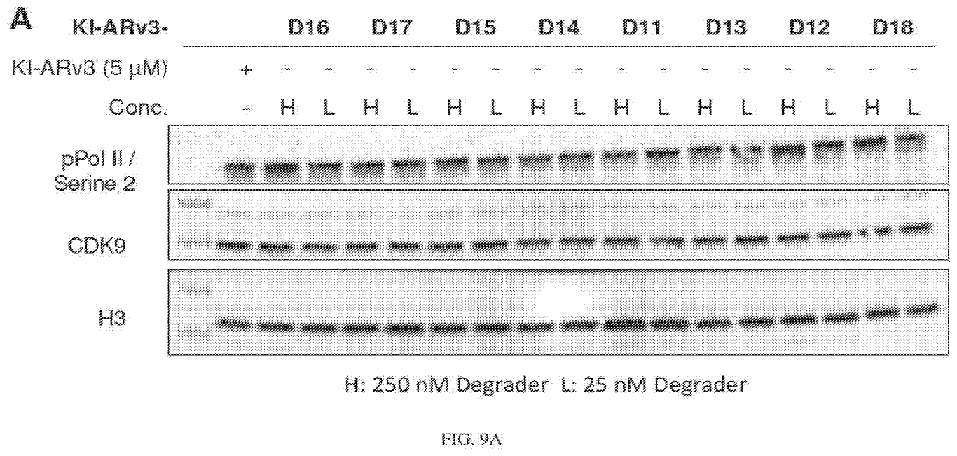

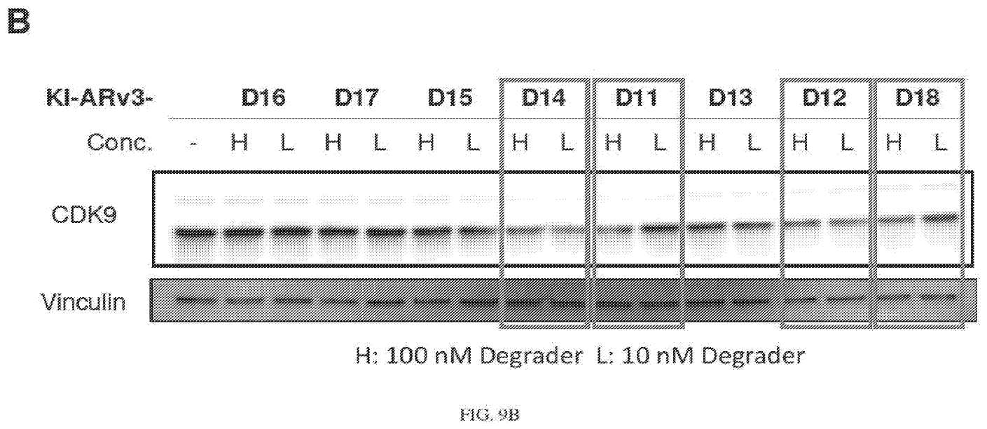

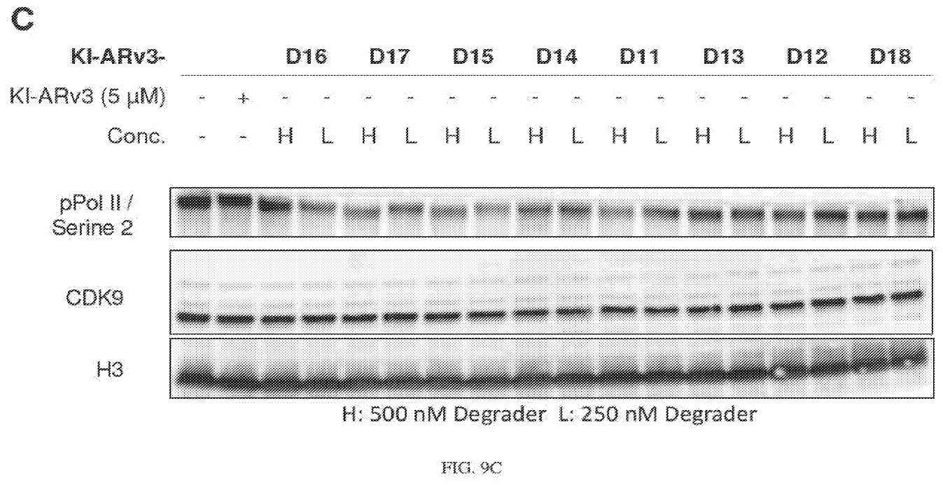

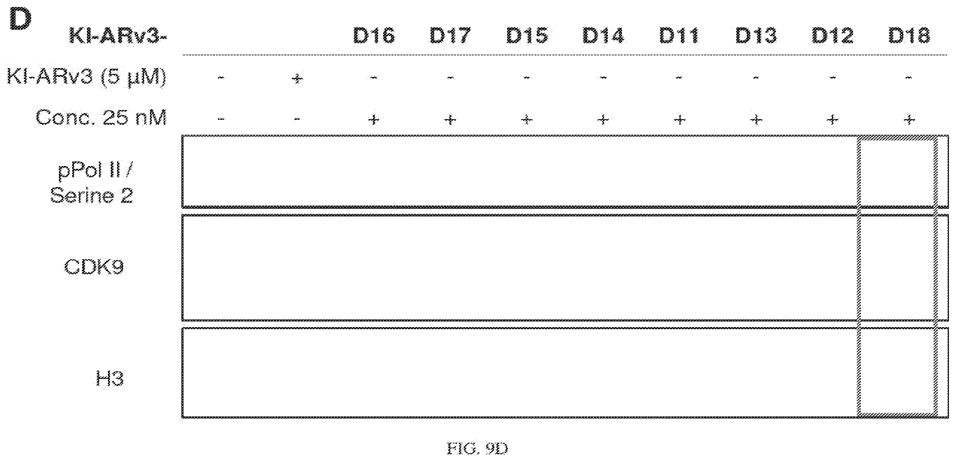

The VHL-based chimeras KI-ARv3-D11 through -D18 were evaluated in treatment studies using 22Rv1 and MOLT-4 cell lines (FIG. 9). Compounds which contain PEG-based and biphenyl linkers (KI-ARv3-D11/D12/D14/D18) lead to a decrease in CDK9 levels of up to 80% at 10 nM and/or 100 nM concentrations in MOLT-4 at 7 h treatment time (FIG. 9B).

VHL-based degraders illustrate a different lineage specificity as compared to pomalidomide based degraders. While none of the KI-ARv3-D11 through -D18 chimeras impact CDK9 levels at concentrations of 250 nM and 500 nM (FIG. 9C), KI-ARv3-D18 leads to a reduction of CDK9 levels of approximately 80% at lower concentrations (25 nM, FIG. 9D).

Recent studies suggest that the introduction of hydrophobic bi-aryl extensions to certain CDK targeting pharmacophores results in the generation of a neohydrophobic surface around the lip of their active site which can result in the recruitment of the CUL4-RBX1-DDB1 ligase core through DDB1 ultimately facilitating ubiquitination of the kinase itself or their associated cyclins. In comparison to traditional PROTACs and IMiD based degraders these molecules act as molecular glue degraders which do not feature chemical linkers. Compounds 4 and 5 (KI-ARv-03-D09 and -D10) incorporate such bi-aryl extensions and were therefore tested for their ability to degrade CDK9 and/or its associated cyclin T. In addition, the corresponding molecules KB-0742-D01 and -D02 which comprise the KB-0472 warhead and bi-aryl extensions were evaluated.

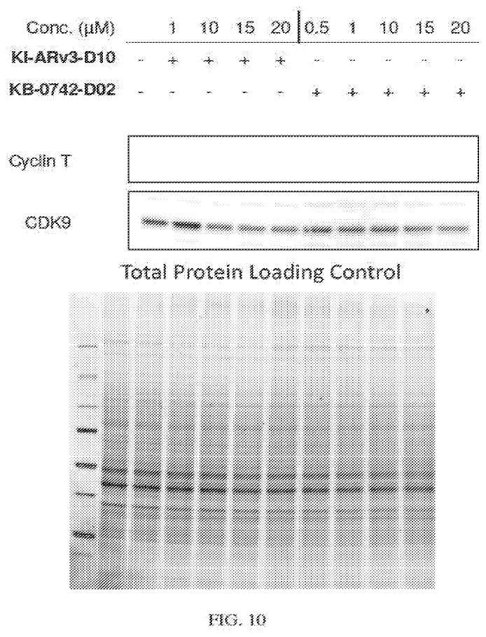

KI-ARv3-D10 and KB-0742-D02 (lacking E3 ligase targeting moieties) led to a reduction in protein levels of both Cyclin T and CDK9 in MOLT-4 leukemia cells at 10 μM after 8 h treatment time (FIG. 10). These results suggest that KI-ARv3-D10 and KB-0742-D02 act through a different mode of action or degradation pathway (e.g., molecular glue degraders or through hydrophobic tagging) ultimately also initiating the delivery of the target protein to the proteasome for degradation.

### Equivalents and Scope

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. A compound of Formula (I): 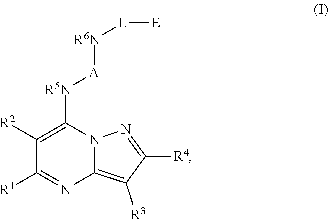
or a pharmaceutically acceptable salt thereof, wherein:
each of R¹, R², R³, and R⁴ is independently hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, substituted or unsubstituted heteroalkyl, —N(R^{A})₂, —OR^{A}, —SR^{A}, —C(═O)OR^{A}, —C(═O)N(R^{A})₂, —NR^{A}C(═O)R^{A}, —C(═O)R^{A}, —NR^{A}C(═O)OR^{A}, —NR^{A}C(═O)N(R^{A})₂, —OC(═O)R^{A}, —OC(═O)OR^{A}, —OC(═O)N(R^{A})₂, —S(O)₂N(R^{A})₂, or —NR^{A}S(O)₂R^{A};
each of R⁵ and R⁶ is independently hydrogen, substituted or unsubstituted alkyl, —C(═O)R^{A}, or a nitrogen protecting group;
A is substituted or unsubstituted carbocyclylene, or substituted or unsubstituted heterocyclylene;
E is an E3 ligase binding moiety;
L is a bond, substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, —S(O)₂NR^{A}—, —NR^{A}S(O)₂—, or a combination thereof; and
each occurrence of R^{A} is, independently, hydrogen, substituted or unsubstituted acyl, substituted or unsubstituted alkyl, substituted or unsubstituted alkenyl, substituted or unsubstituted alkynyl, substituted or unsubstituted heteroalkyl, substituted or unsubstituted carbocyclyl, substituted or unsubstituted heterocyclyl, substituted or unsubstituted aryl, substituted or unsubstituted heteroaryl, an oxygen protecting group when attached to an oxygen atom, or a nitrogen protecting group when attached to a nitrogen atom, or two R^{A} groups are joined to form a substituted or unsubstituted heterocyclic ring.

2. The compound of claim 1, or a pharmaceutically acceptable salt thereof, wherein:
each of R¹, R², R³, and R⁴ is independently hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, or substituted or unsubstituted heteroalkyl.

3. The compound of claim 1 or 2, or a pharmaceutically acceptable salt thereof, wherein:
each of R¹, R², R³, and R⁴ is independently hydrogen, halogen, or substituted or unsubstituted alkyl.

4. The compound of any one of claims 1-3, or a pharmaceutically acceptable salt thereof, wherein:
each of R², R³, and R⁴ is hydrogen; and
R¹ is hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, or substituted or unsubstituted heteroalkyl.

5. The compound of any one of claims 1-4, or a pharmaceutically acceptable salt thereof, wherein:
each of R², R³, and R⁴ is hydrogen; and R¹ is hydrogen, halogen, or substituted or unsubstituted alkyl.

6. The compound of any one of claims 1-5, or a pharmaceutically acceptable salt thereof, wherein:
each of R², R³, and R⁴ is hydrogen; and R¹ is substituted or unsubstituted alkyl.

7. The compound of any one of claims 1-6, or a pharmaceutically acceptable salt thereof, wherein:
each of R⁵ and R⁶ is independently hydrogen or substituted or unsubstituted alkyl.

8. The compound of any one of claims 1-7, or a pharmaceutically acceptable salt thereof, wherein each of R⁵ and R⁶ is hydrogen.

9. The compound of any one of claims 1-8, or a pharmaceutically acceptable salt thereof, wherein A is substituted or unsubstituted carbocyclylene.

10. The compound of any one of claims 1-9, or a pharmaceutically acceptable salt thereof, wherein A is substituted or unsubstituted C₃₋₆ cycloalkylene.

11. The compound of any one of claims 1-10, or a pharmaceutically acceptable salt thereof, wherein A is substituted or unsubstituted C₅₋₆ cycloalkylene.

12. The compound of any one of claims 1-11, or a pharmaceutically acceptable salt thereof, wherein A is substituted or unsubstituted cyclopentylene.

13. The compound of any one of claims 1-12, or a pharmaceutically acceptable salt thereof, wherein A is

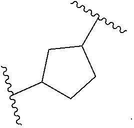 [SMILES: *C1CCC(*)C1]

14. The compound of any one of claims 1-13, or a pharmaceutically acceptable salt thereof, wherein A is

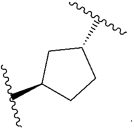 [SMILES: *[C@@H]1CC[C@@H](*)C1]

15. The compound of any one of claims 1-13, or a pharmaceutically acceptable salt thereof, wherein A is

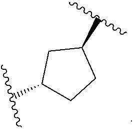 [SMILES: *[C@H]1CC[C@H](*)C1]

16. The compound of any one of claims 1-15, or a pharmaceutically acceptable salt thereof, wherein L is substituted or unsubstituted alkylene, substituted or unsubstituted alkenylene, substituted or unsubstituted alkynylene, substituted or unsubstituted heteroalkylene, substituted or unsubstituted heteroalkenylene, substituted or unsubstituted heteroalkynylene, substituted or unsubstituted arylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}, —NR^{A}C(═O)O—, —NR^{A}C(═O)N(R^{A})—, —OC(═O)—, —OC(═O)O—, —OC(═O)N(R^{A})—, —S(O)₂NR^{A}—, —NR^{A}S(O)₂—, or a combination thereof.

17. The compound of any one of claims 1-16, or a pharmaceutically acceptable salt thereof, wherein L is substituted or unsubstituted alkylene, substituted or unsubstituted alkynylene, substituted or unsubstituted heteroalkylene, substituted or unsubstituted arylene, —O—, —C(═O)—, or a combination thereof.

18. The compound of any one of claims 1-17, or a pharmaceutically acceptable salt thereof, wherein L is substituted or unsubstituted alkylene, substituted or unsubstituted heteroalkylene, substituted or unsubstituted arylene, —C(═O)—, or a combination thereof.

19. The compound of any one of claims 1-18, or a pharmaceutically acceptable salt thereof, wherein:
L is 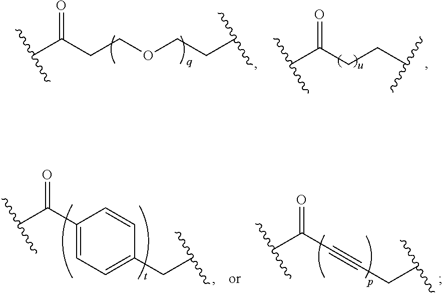
q is 1-12;
u is 1-12;
t is 1-3; and
p is 1-3.

20. The compound of any one of claims 1-19, or a pharmaceutically acceptable salt thereof, wherein:
L is 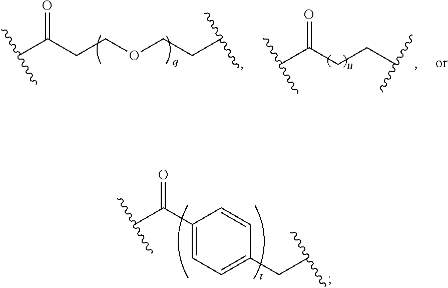
q is 1-12;
u is 1-12; and
t is 1-3.

21. The compound of any one of claims 1-20, or a pharmaceutically acceptable salt thereof, wherein:
L is 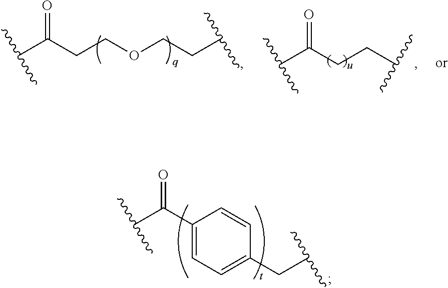
q is 1-4;
u is 1-8; and
t is 1-2.

22. The compound of any one of claims 1-21, or a pharmaceutically acceptable salt thereof, wherein:
L is 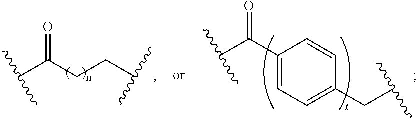
u is 1-8; and
t is 1-2.

23. The compound of any one of claims 1-19, or a pharmaceutically acceptable salt thereof, wherein:
L is

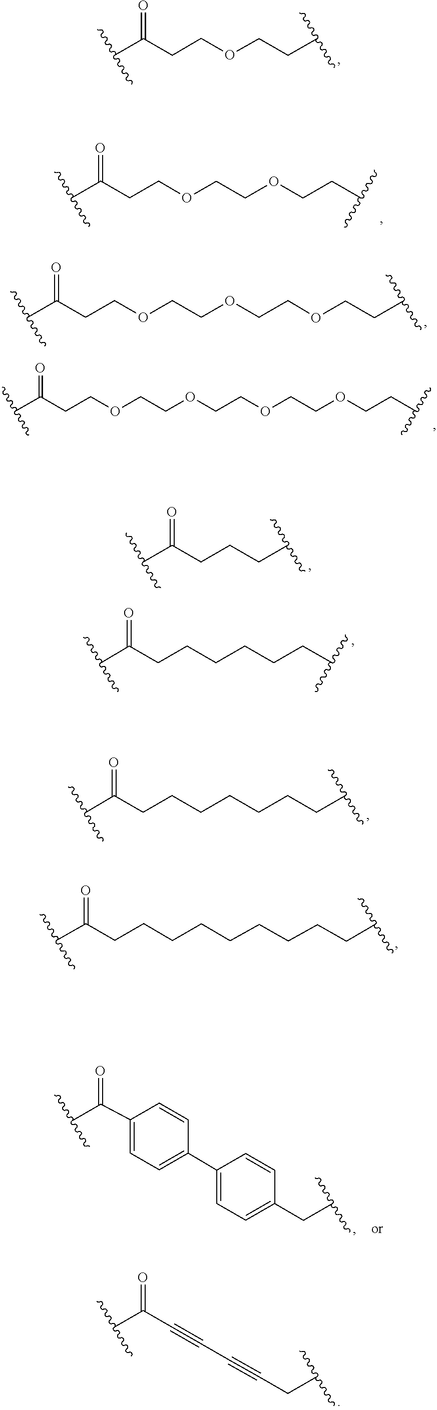

24. The compound of any one of claims 1-19, or a pharmaceutically acceptable salt thereof, wherein:
L is

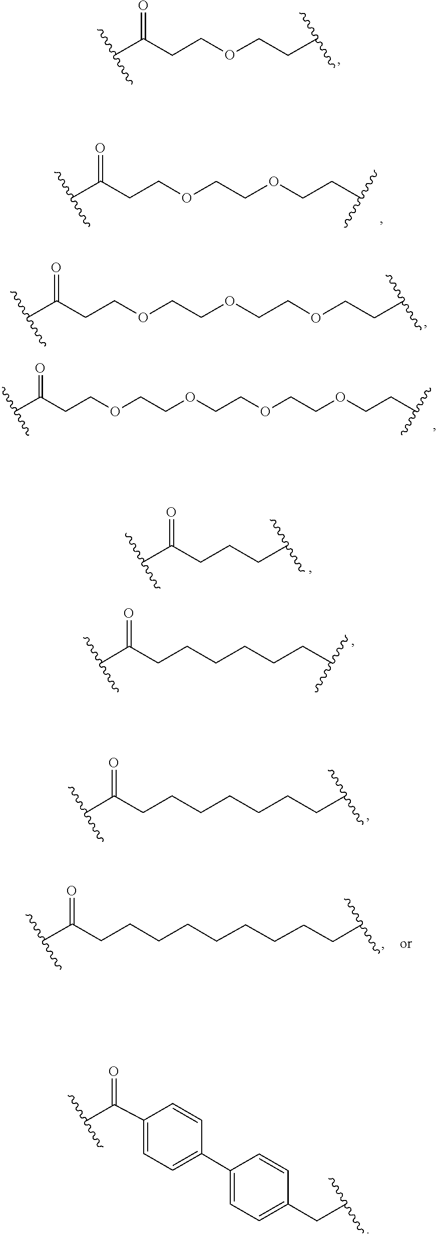

25. The compound of any one of claims 1-24, or a pharmaceutically acceptable salt thereof, wherein:
L is

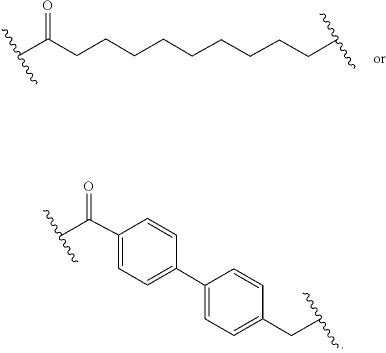 [SMILES: *CCCCCCCCCC(*)=O ; *Cc1ccc(-c2ccc(C(*)=O)cc2)cc1]

26. The compound of any one of claims 1-25, or a pharmaceutically acceptable salt thereof, wherein E is a cereblon E3 ubiquitin ligase binding moiety or a VHL E3 ubiquitin ligase binding moiety.

27. The compound of any one of claims 1-26, or a pharmaceutically acceptable salt thereof, wherein E is of Formula (E-I): 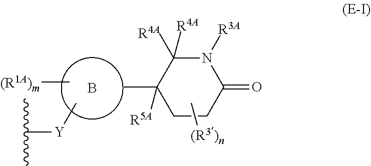
wherein:
B is a substituted or unsubstituted monocyclic, bicyclic, or tricyclic fused ring system;
Y is —(CH₂)ₖ—, —(CH₂)ₖ—O—, —O(CH₂)ₖ—, —NR^{B}(CH₂)ₖ—, —(CH₂)ₖ—NR^{B}—, —(CH₂)ₖ—(C═O)NR^{B}—, —O(CH₂)ₖ—(C═O)NR^{B}—, —O(CH₂)ₖ—NR^{B}(C═O)—, —NR^{B}(C═O)—(CH₂)ₖ—O—, —NR^{B}(CH₂)ₖ—NR^{B}(C═O)—, or —(CH₂)ₖ—NR^{B}(C═O)—;
each R^{B} is, independently, hydrogen, or substituted or unsubstituted alkyl;
each R^{1A} is, independently, halogen, OH, C₁-C₆ alkyl, or C₁-C₆ alkoxy;
each R^{3A} is, independently, hydrogen or C₁-C₃ alkyl;
each R^{3′} is, independently, C₁-C₃ alkyl;
each R^{4A} is, independently, hydrogen or C₁-C₃ alkyl; or two R^{4A}, together with the carbon atom to which they are attached, form a C(O), C₃-C₆ carbocycle, or a 4-6-membered heterocycle comprising 1 or 2 heteroatoms selected from N and O;
R^{5A} is hydrogen, C₁-C₃ alkyl, F, or Cl;
k is 0, 1, 2, 3, 4, 5, or 6;
m is 0, 1, 2 or 3; and
n is 0, 1, or 2.

28. The compound of any one of claims 1-27, or a pharmaceutically acceptable salt thereof, wherein E is of Formula (E-I-a): 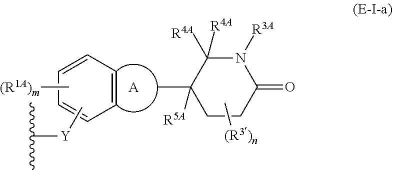
wherein:
A is a substituted or unsubstituted heterocyclyl, or substituted or unsubstituted heteroaryl ring;
Y is —(CH₂)ₖ—, —(CH₂)ₖ—O—, —O(CH₂)ₖ—, —NR^{B}(CH₂)ₖ—, —(CH₂)ₖ—NR^{B}—, —(CH₂)ₖ—(C═O)NR^{B}—, —O(CH₂)ₖ—(C═O)NR^{B}—, —O(CH₂)ₖ—NR^{B}(C═O)—, —NR^{B}(C═O)—(CH₂)ₖ—O—, —NR^{B}(CH₂)ₖ—NR^{B}(C═O)—, or —(CH₂)ₖ—NR^{B}(C═O)—;
each R^{B} is, independently, hydrogen, or substituted or unsubstituted alkyl;
each R^{1A} is, independently, halogen, OH, C₁-C₆ alkyl, or C₁-C₆ alkoxy;
each R^{3A} is, independently, hydrogen or C₁-C₃ alkyl;
each R^{3′} is, independently, C₁-C₃ alkyl;
each R^{4A} is, independently, hydrogen or C₁-C₃ alkyl; or two R^{4A}, together with the carbon atom to which they are attached, form a C(O), C₃-C₆ carbocycle, or a 4-6-membered heterocycle comprising 1 or 2 heteroatoms selected from N and O;
R^{5A} is hydrogen, C₁-C₃ alkyl, F, or Cl;
k is 0, 1, 2, 3, 4, 5, or 6;
m is 0, 1, 2 or 3; and
n is 0, 1, or 2.

29. The compound of any one of claims 1-28, or a pharmaceutically acceptable salt thereof, wherein E is of Formula (E-I-b): 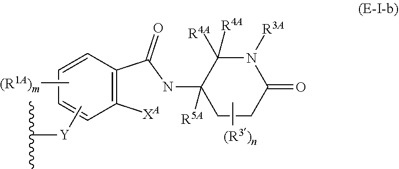
wherein:
Y is —(CH₂)ₖ—, —(CH₂)ₖ—O—, —O(CH₂)ₖ—, —NR^{B}(CH₂)ₖ—, —(CH₂)ₖ—NR^{B}—, —(CH₂)ₖ—(C═O)NR^{B}—, —O(CH₂)ₖ—(C═O)NR^{B}—, —O(CH₂)ₖ—NR^{B}(C═O)—, —NR^{B}(C═O)—(CH₂)ₖ—O—, —NR^{B}(CH₂)ₖ—NR^{B}(C═O)—, or —(CH₂)ₖ—NR^{B}(C═O)—;
X^{A} is C(O) or C(R^{3A})₂;
each R^{B} is, independently, hydrogen, or substituted or unsubstituted alkyl;
each R^{1A} is, independently, halogen, OH, C₁-C₆ alkyl, or C₁-C₆ alkoxy;
each R^{3A} is, independently, hydrogen, or C₁-C₃ alkyl;
each R^{3′} is, independently, C₁-C₃ alkyl;
each R^{4A} is, independently, H or C₁-C₃ alkyl; or two R^{4A}, together with the carbon atom to which they are attached, form a C(O), C₃-C₆ carbocycle, or a 4-, 5-, or 6-membered heterocycle comprising 1 or 2 heteroatoms selected from N and O;
R^{5A} is hydrogen, C₁-C₃ alkyl, F, or Cl;
k is 0, 1, 2, 3, 4, 5, or 6;
m is 0, 1, 2 or 3; and
n is 0, 1, or 2.

30. The compound of claim 29, or a pharmaceutically acceptable salt thereof, wherein E is of Formula (E-I-c):

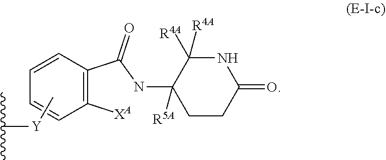

31. The compound of any one of claims 1-26, or a pharmaceutically acceptable salt thereof, wherein E is of Formula (E-III): 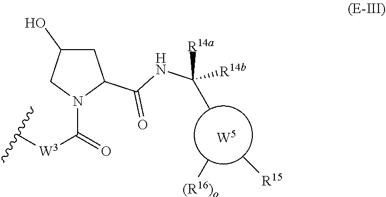
wherein:
W³ is substituted or unsubstituted arylene, substituted or unsubstituted heteroarylene, or 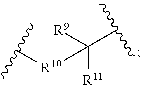
R⁹ and R¹¹ are independently hydrogen, substituted or unsubstituted alkyl, substituted or unsubstituted cycloalkyl, substituted or unsubstituted hydroxyalkyl, substituted or unsubstituted heteroaryl, or haloalkyl; or R⁹, R¹¹, and the carbon atom to which they are attached form a substituted or unsubstituted cycloalkyl;
R¹⁰ is —O—, —NH—, substituted or unsubstituted heterocyclylene, substituted or unsubstituted heteroarylene, or substituted or unsubstituted arylene;
R^{14a} and R^{14b} are each independently hydrogen, haloalkyl, or substituted or unsubstituted alkyl;
W⁵ is aryl or heteroaryl;
R₁₅ is hydrogen, halogen, CN, OH, NO₂, —NR^{14a}R^{14b}, OR^{14a}, CONR^{14a}R^{14b}, NR^{14a}COR^{14b}, SO₂NR^{14a}R^{14b}, NR^{14a}SO₂R^{14b}, substituted or unsubstituted alkyl, haloalkyl, haloalkoxy, substituted or unsubstituted aryl, substituted or unsubstituted heteroaryl, substituted or unsubstituted cycloalkyl, or substituted or unsubstituted heterocyclyl;
each R¹⁶ is independently halo, substituted or unsubstituted alkyl, haloalkyl, hydroxy, or haloalkoxy; or 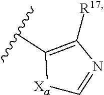
wherein R¹⁷ is hydrogen, halogen, substituted or unsubstituted C₃₋₆ cycloalkyl, substituted or unsubstituted C₁₋₆ alkyl, substituted or unsubstituted C₁₋₆ alkenyl, or C₁₋₆ haloalkyl; Xₐ is S or O; and
o is 0, 1, 2, 3, or 4.

32. The compound of any one of claims 1-26 or 31, or a pharmaceutically acceptable salt thereof, wherein E is of Formula (E-III-a):

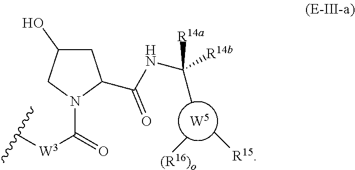

33. The compound of any one of claims 1-26, 31, or 32, or a pharmaceutically acceptable salt thereof, wherein E is of Formula (E-III-b):

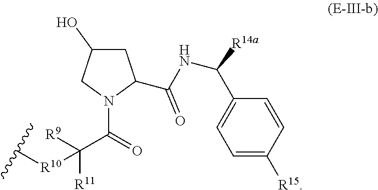

34. The compound of any one of claims 1-26 or 31-33, or a pharmaceutically acceptable salt thereof, wherein E is of Formula (E-III-d-1):

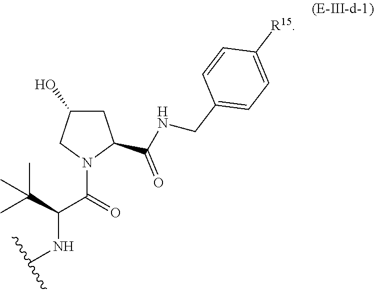

35. The compound of any one of claims 1-26, or a pharmaceutically acceptable salt thereof, wherein E is of formula:

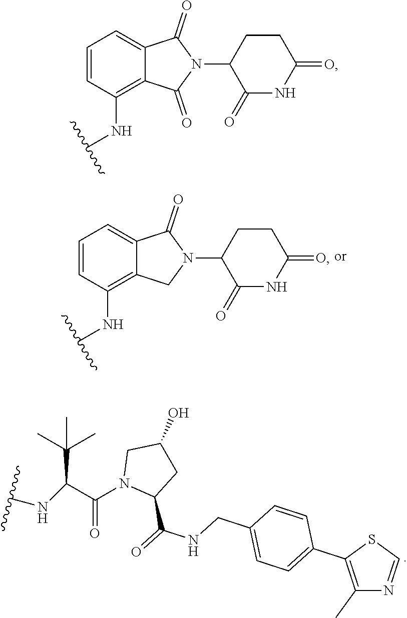 [SMILES: *Nc1cccc2c1C(=O)N(C1CCC(=O)NC1=O)C2=O ; *Nc1cccc2c1CN(C1CCC(=O)NC1=O)C2=O ; *N[C@H](C(=O)N1C[C@H](O)C[C@H]1C(=O)NCc1ccc(-c2scnc2C)cc1)C(C)(C)C]

36. The compound of any one of claims 1-30, or a pharmaceutically acceptable salt thereof, wherein E is of the formula:

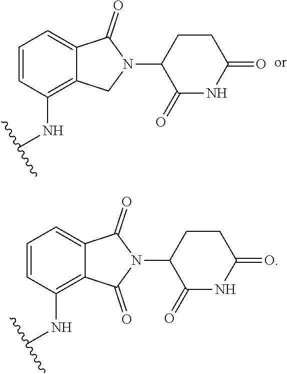 [SMILES: *Nc1cccc2c1CN(C1CCC(=O)NC1=O)C2=O ; *Nc1cccc2c1C(=O)N(C1CCC(=O)NC1=O)C2=O]

37. The compound of any one of claims 1-30, or a pharmaceutically acceptable salt thereof, wherein E is of the formula:

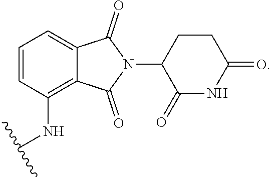 [SMILES: *Nc1cccc2c1C(=O)N(C1CCC(=O)NC1=O)C2=O]

38. The compound of any one of claims 1-26, or a pharmaceutically acceptable salt thereof, wherein E is of the formula:

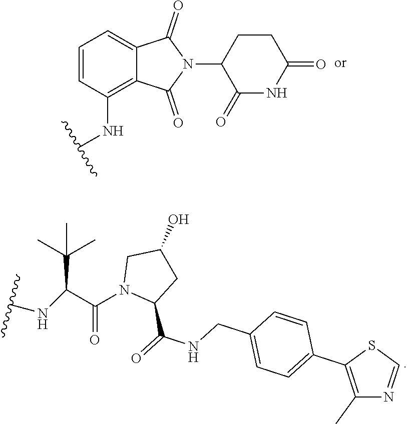 [SMILES: *Nc1cccc2c1C(=O)N(C1CCC(=O)NC1=O)C2=O ; *N[C@H](C(=O)N1C[C@H](O)C[C@H]1C(=O)NCc1ccc(-c2scnc2C)cc1)C(C)(C)C]

39. The compound of any one of claims 1-26 or 31-35, or a pharmaceutically acceptable salt thereof, wherein E is of the formula:

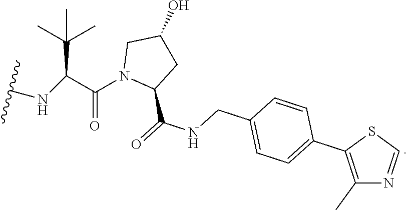 [SMILES: *N[C@H](C(=O)N1C[C@H](O)C[C@H]1C(=O)NCc1ccc(-c2scnc2C)cc1)C(C)(C)C]

40. The compound of any one of claims 1-39, wherein the compound is of Formula (I-a): 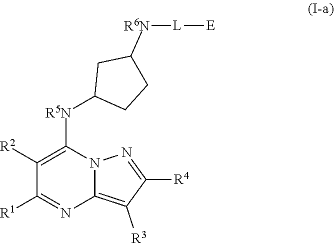
or a pharmaceutically acceptable salt thereof.

41. The compound of any one of claims 1-40, wherein the compound is of Formula (I-b): 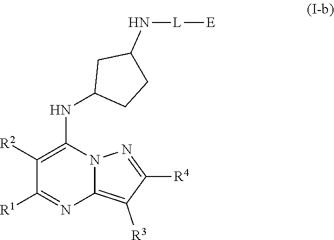
or a pharmaceutically acceptable salt thereof.

42. The compound of any one of claims 1-41, wherein the compound is of Formula (I-c): 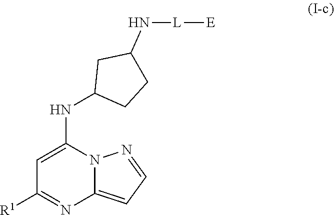
or a pharmaceutically acceptable salt thereof.

43. The compound of any one of claims 1-42, wherein the compound is of Formula (I-d): 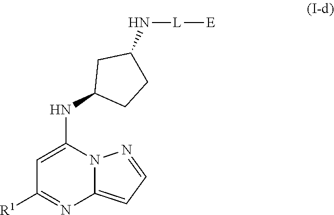
or a pharmaceutically acceptable salt thereof.

44. The compound of any one of claims 1-42, wherein the compound is of Formula (I-d-1): 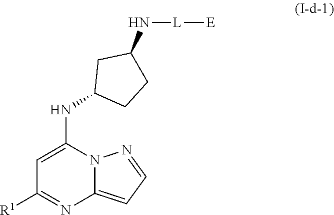
or a pharmaceutically acceptable salt thereof.

45. The compound of any one of claims 1-42, wherein the compound is of Formula (I-e): 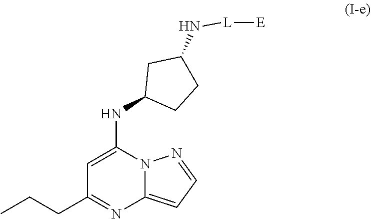
or a pharmaceutically acceptable salt thereof.

46. The compound of any one of claims 1-42, wherein the compound is of Formula (I-e-1): 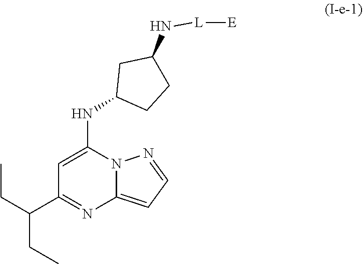
or a pharmaceutically acceptable salt thereof.

47. The compound of any one of claims 1-42, wherein the compound is of Formula (I-f): 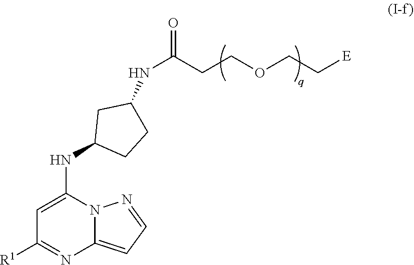
or a pharmaceutically acceptable salt thereof, wherein q is 1-12.

48. The compound of any one of claims 1-42, wherein the compound is of Formula (I-f-2): 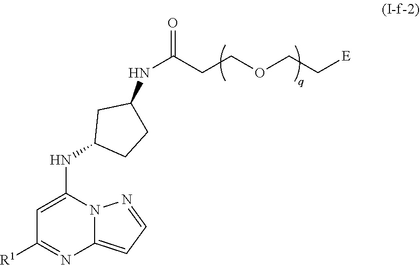
or a pharmaceutically acceptable salt thereof, wherein q is 1-12.

49. The compound of any one of claims 1-42, wherein the compound is of Formula (I-g): 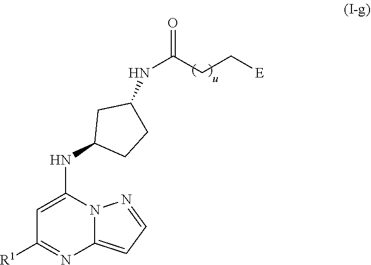
or a pharmaceutically acceptable salt thereof, wherein u is 1-8.

50. The compound of any one of claims 1-42, wherein the compound is of Formula (I-g-2): 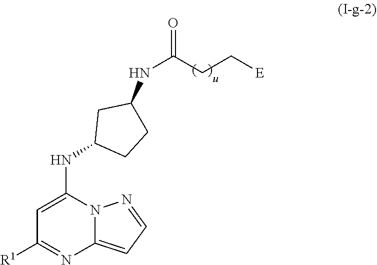
or a pharmaceutically acceptable salt thereof, wherein u is 1-8.

51. The compound of any one of claims 1-42, wherein the compound is of Formula (I-h): 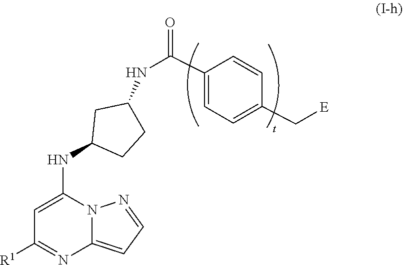
or a pharmaceutically acceptable salt thereof, wherein t is 1 or 2.

52. The compound of any one of claims 1-42, wherein the compound is of Formula (I-h-2): 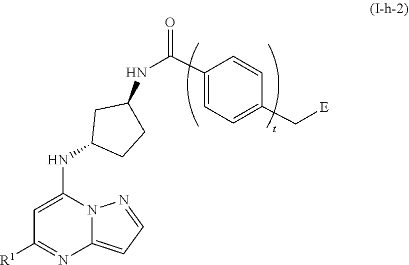
or a pharmaceutically acceptable salt thereof, wherein t is 1 or 2.

53. The compound of any one of claims 1-42, wherein the compound is of Formula (I-i): 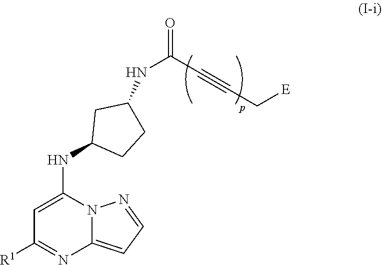
or a pharmaceutically acceptable salt thereof, wherein p is 1 or 2.

54. The compound of any one of claims 1-42, wherein the compound is of Formula (I-i-2): 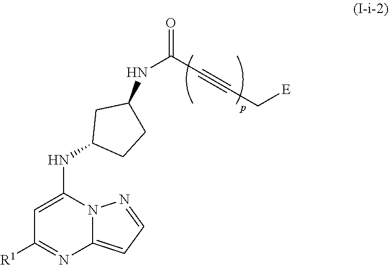
or a pharmaceutically acceptable salt thereof, wherein p is 1 or 2.

55. The compound of any one of claims 1-42, wherein the compound is of Formula (I-j): 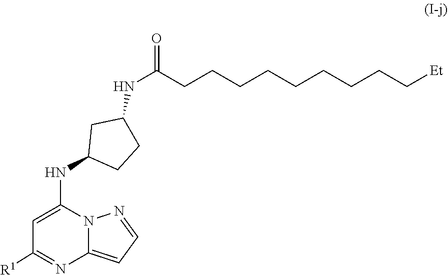
or a pharmaceutically acceptable salt thereof.

56. The compound of any one of claims 1-42, wherein the compound is of Formula (I-j-2): 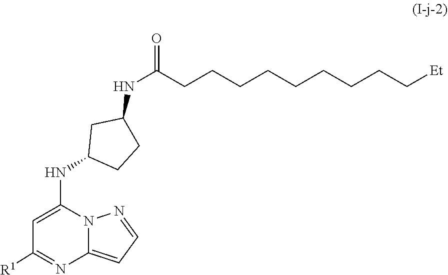
or a pharmaceutically acceptable salt thereof.

57. The compound of any one of claims 1-42, wherein the compound is of Formula (I-k): 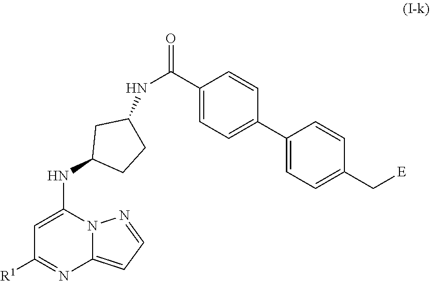
or a pharmaceutically acceptable salt thereof.

58. The compound of any one of claims 1-42, wherein the compound is of Formula (I-k-2): 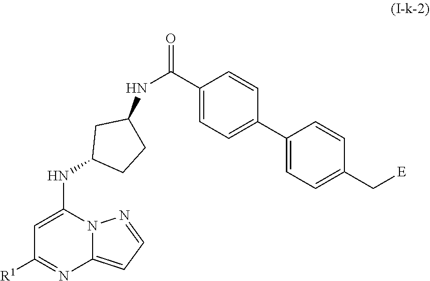
or a pharmaceutically acceptable salt thereof.

59. The compound of claim 1, wherein the compound is 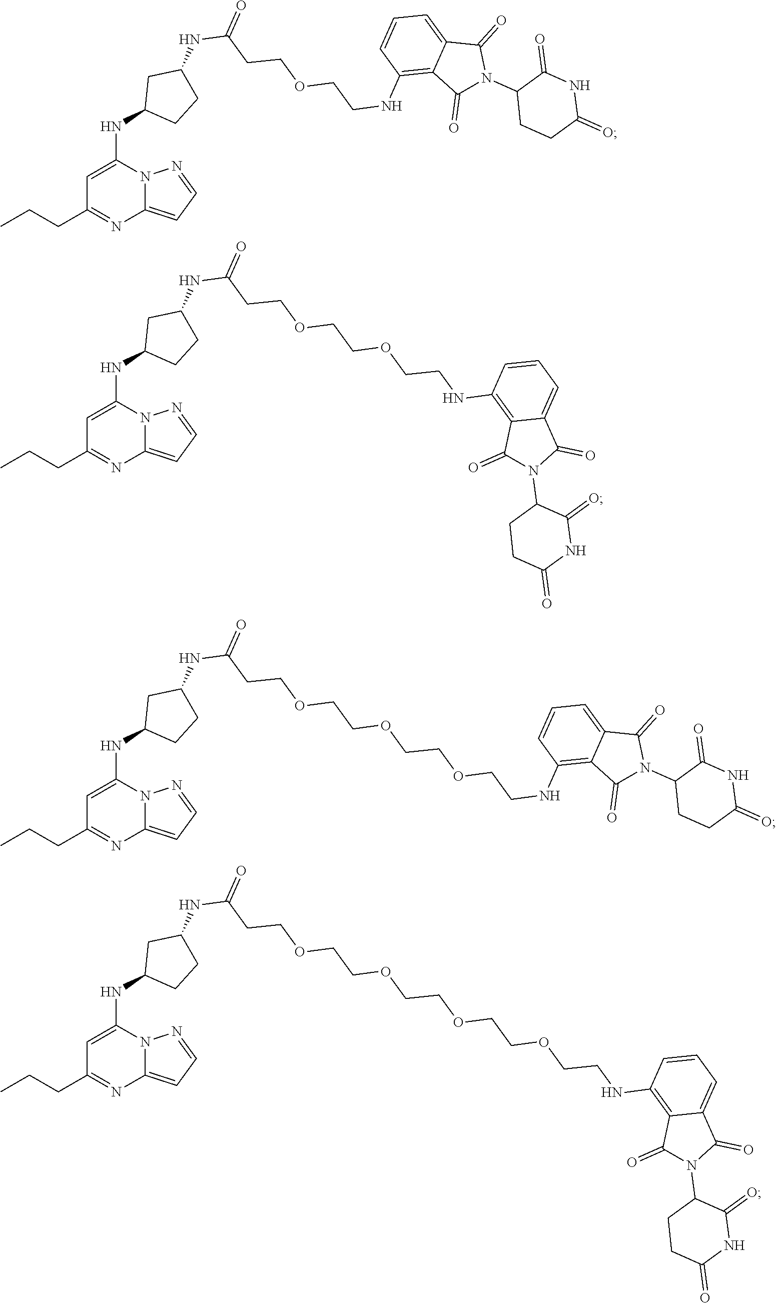 [SMILES: CCCc1cc(N[C@@H]2CC[C@@H](NC(=O)CCOCCNc3cccc4c3C(=O)N(C3CCC(=O)NC3=O)C4=O)C2)n2nccc2n1 ; CCCc1cc(N[C@@H]2CC[C@@H](NC(=O)CCOCCOCCNc3cccc4c3C(=O)N(C3CCC(=O)NC3=O)C4=O)C2)n2nccc2n1 ; CCCc1cc(N[C@@H]2CC[C@@H](NC(=O)CCOCCOCCOCCNc3cccc4c3C(=O)N(C3CCC(=O)NC3=O)C4=O)C2)n2nccc2n1 ; CCCc1cc(N[C@@H]2CC[C@@H](NC(=O)CCOCCOCCOCCOCCNc3cccc4c3C(=O)N(C3CCC(=O)NC3=O)C4=O)C2)n2nccc2n1] 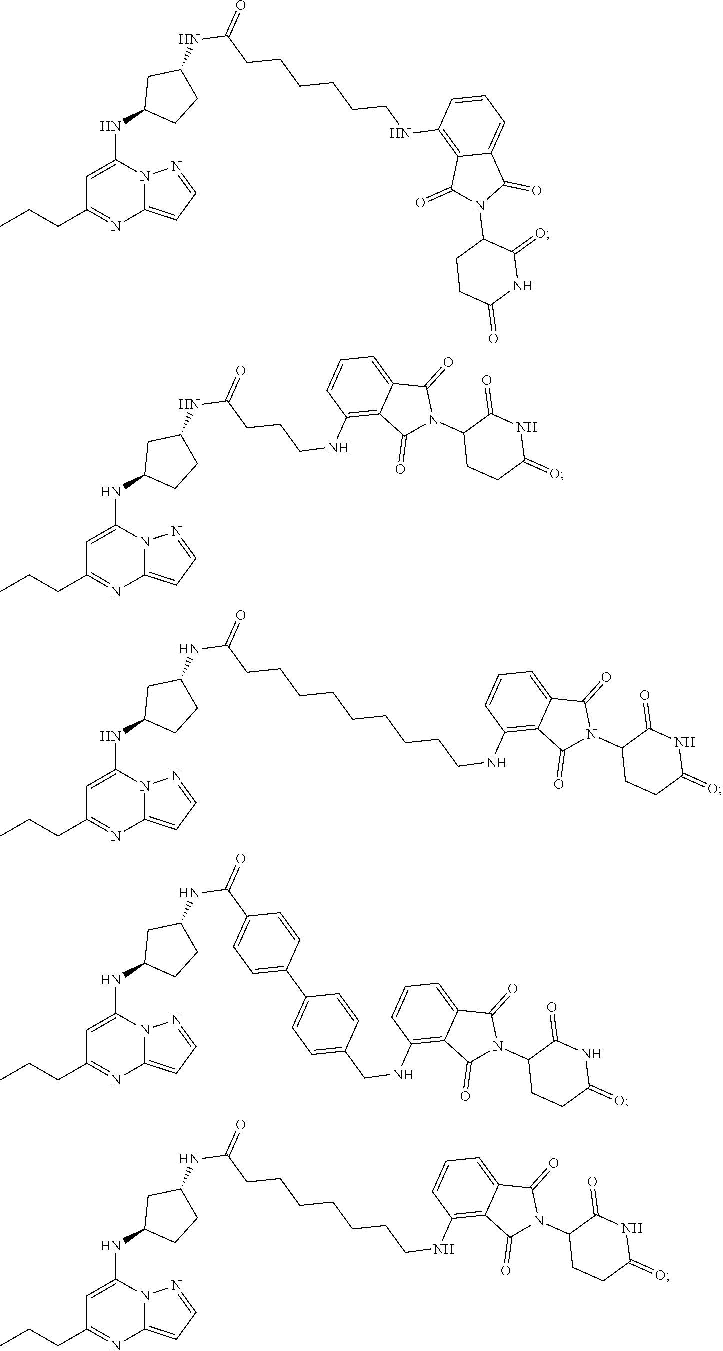 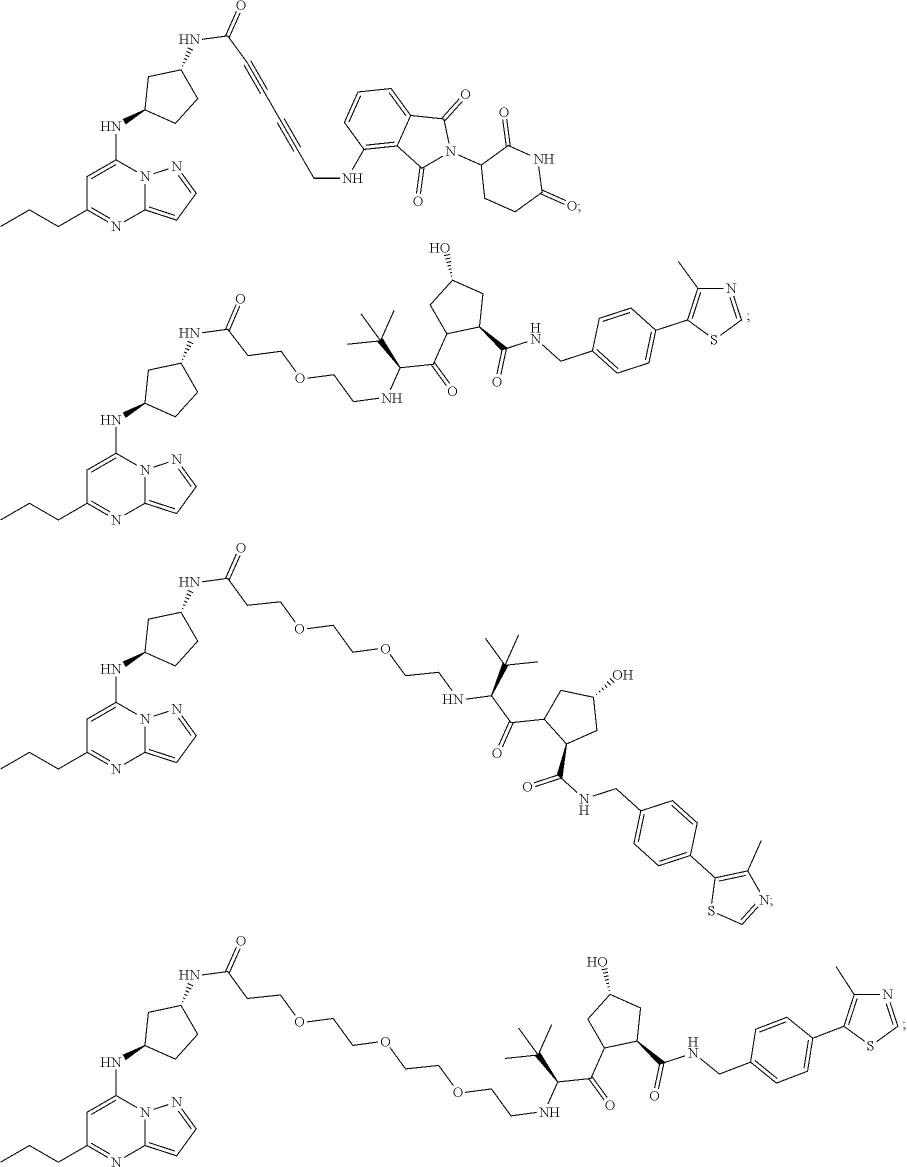 [SMILES: CCCc1cc(N[C@@H]2CC[C@@H](NC(=O)C#CC#CCNc3cccc4c3C(=O)N(C3CCC(=O)NC3=O)C4=O)C2)n2nccc2n1 ; CCCc1cc(N[C@@H]2CC[C@@H](NC(=O)CCOCCN[C@H](C(=O)C3C[C@H](O)C[C@H]3C(=O)NCc3ccc(-c4scnc4C)cc3)C(C)(C)C)C2)n2nccc2n1 ; CCCc1cc(N[C@@H]2CC[C@@H](NC(=O)CCOCCOCCN[C@H](C(=O)C3C[C@H](O)C[C@H]3C(=O)NCc3ccc(-c4scnc4C)cc3)C(C)(C)C)C2)n2nccc2n1 ; CCCc1cc(N[C@@H]2CC[C@@H](NC(=O)CCOCCOCCOCCN[C@H](C(=O)C3C[C@H](O)C[C@H]3C(=O)NCc3ccc(-c4scnc4C)cc3)C(C)(C)C)C2)n2nccc2n1] 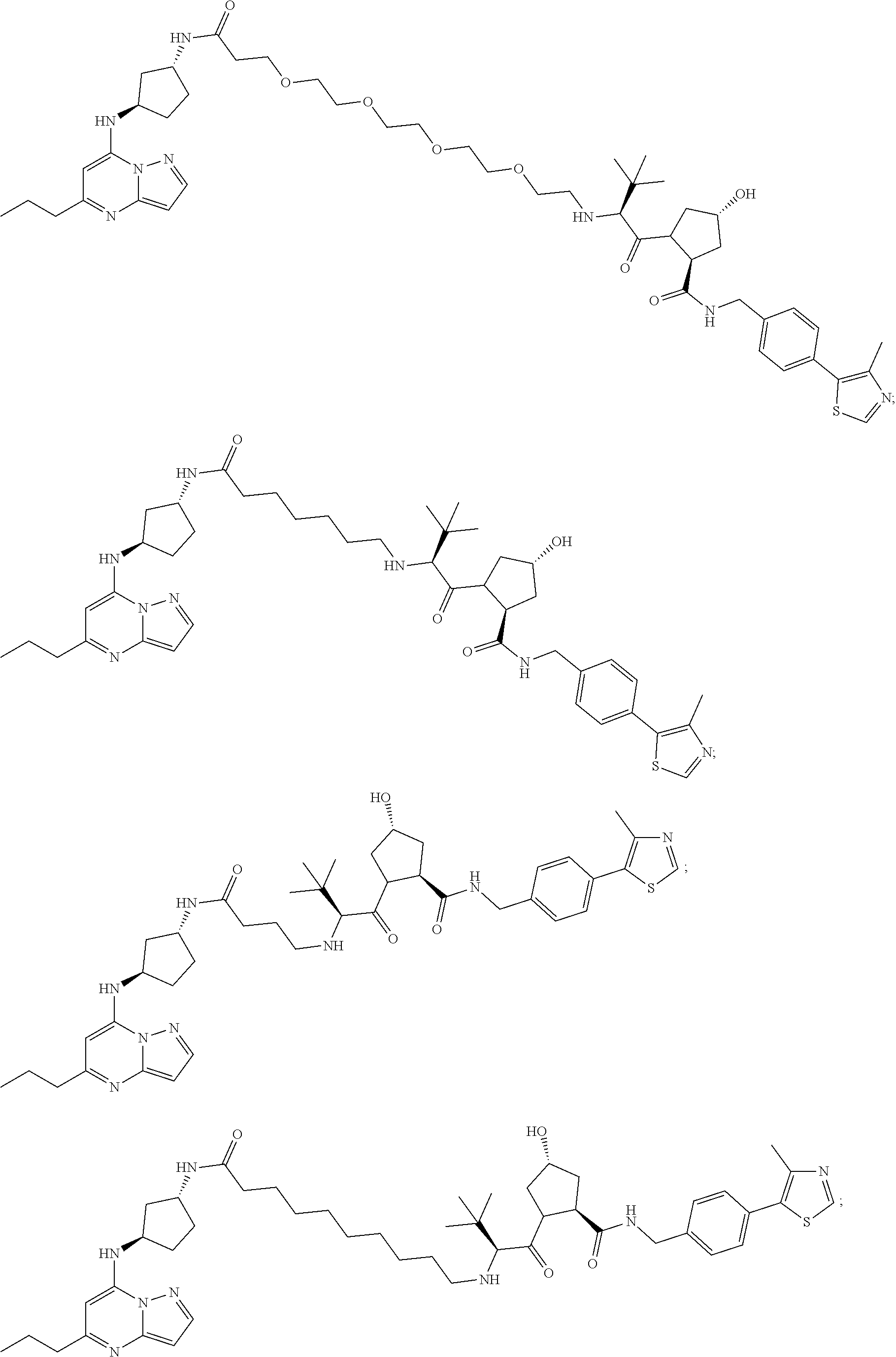 [SMILES: CCCc1cc(N[C@@H]2CC[C@@H](NC(=O)CCOCCOCCOCCOCCN[C@H](C(=O)C3C[C@H](O)C[C@H]3C(=O)NCc3ccc(-c4scnc4C)cc3)C(C)(C)C)C2)n2nccc2n1 ; CCCc1cc(N[C@@H]2CC[C@@H](NC(=O)CCCCCCN[C@H](C(=O)C3C[C@H](O)C[C@H]3C(=O)NCc3ccc(-c4scnc4C)cc3)C(C)(C)C)C2)n2nccc2n1 ; CCCc1cc(N[C@@H]2CC[C@@H](NC(=O)CCCN[C@H](C(=O)C3C[C@H](O)C[C@H]3C(=O)NCc3ccc(-c4scnc4C)cc3)C(C)(C)C)C2)n2nccc2n1 ; CCCc1cc(N[C@@H]2CC[C@@H](NC(=O)CCCCCCCCCN[C@H](C(=O)C3C[C@H](O)C[C@H]3C(=O)NCc3ccc(-c4scnc4C)cc3)C(C)(C)C)C2)n2nccc2n1] 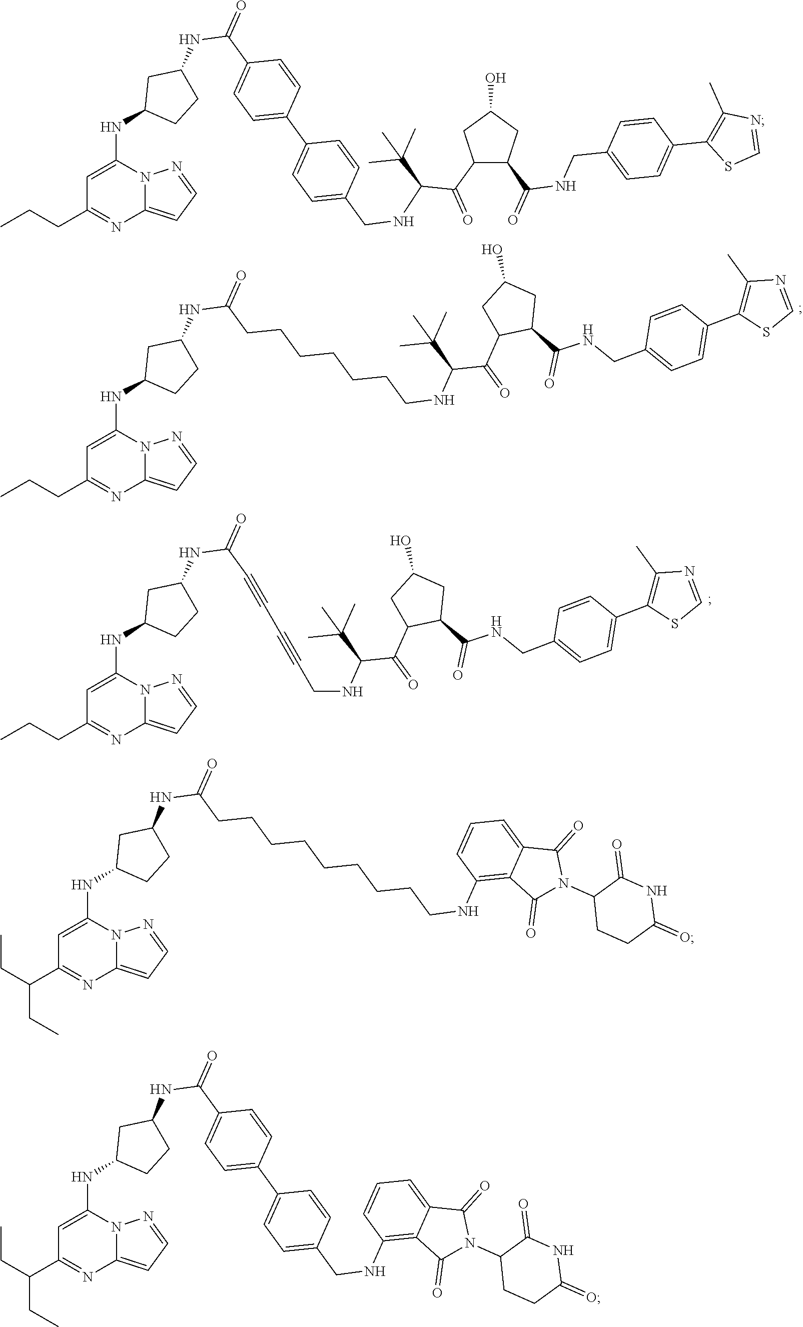
or a pharmaceutically acceptable salt thereof.

60. A compound of the formula: 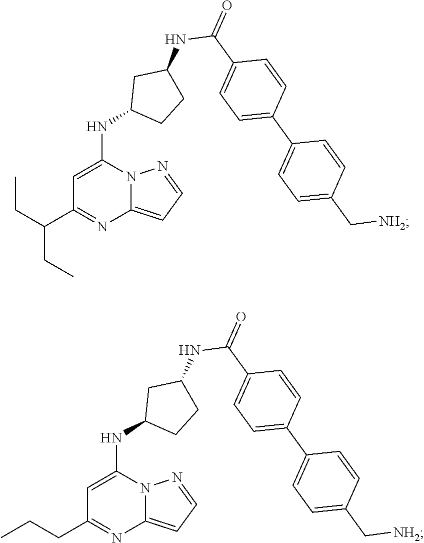 [SMILES: CCC(CC)c1cc(N[C@H]2CC[C@H](NC(=O)c3ccc(-c4ccc(CN)cc4)cc3)C2)n2nccc2n1 ; CCCc1cc(N[C@@H]2CC[C@@H](NC(=O)c3ccc(-c4ccc(CN)cc4)cc3)C2)n2nccc2n1]
or a pharmaceutically acceptable salt thereof.

61. A pharmaceutical composition comprising a compound of any one of claims 1-60, or a pharmaceutically acceptable salt thereof, and a pharmaceutically acceptable excipient.

62. A method of treating cancer in a subject in need thereof, the method comprising administering a compound of any one of claims 1-60, or a pharmaceutically acceptable salt thereof, or a pharmaceutical composition of claim 61 to the subject.

63. The method of claim 62, wherein the cancer is a solid tumor or a hematological cancer.

64. The method of claim 62 or 63, wherein the cancer is a leukemia or a lymphoma.

65. The method of any one of claims 62-64, wherein the cancer is acute myeloid leukemia (AML) or acute lymphoblastic leukemia (ALL).

66. The method of claim 62 or 63, wherein the cancer is hepatocellular carcinoma, prostate cancer, or neuroblastoma.

67. A method of promoting the degradation of cyclin-dependent kinase 9 (CDK9), the method comprising contacting CDK9 with a compound of any one of claims 1-49, or a pharmaceutically acceptable salt thereof, or a pharmaceutical composition of claim 50.

68. The method of claim 67, wherein the degradation is in a cell.

69. The method of claim 67 or 68, wherein the degradation is in a subject.

70. The method of claim 67 or 68, wherein the degradation is in a biological sample.

71. A method of promoting the ubiquitination of cyclin-dependent kinase 9 (CDK9) by an E3 ubiquitin ligase, the method comprising contacting CDK9 with a compound of any one of claims 1-60, or a pharmaceutically acceptable salt thereof, or a pharmaceutical composition of claim 61.

72. The method of claim 71, wherein the ubiquitination is in a cell.

73. The method of claim 71 or 72, wherein the ubiquitination is in a subject.

74. The method of claim 71 or 72, wherein the ubiquitination is in a biological sample.

75. The method of any one of claims 71-74, wherein the E3 ubiquitin ligase is Cereblon or von Hippel-Lindau tumor suppressor (VHL).

76. A compound of any one of claims 1-60, or a pharmaceutically acceptable salt thereof, or a pharmaceutical composition of claim 61, for use in treating cancer in a subject in need thereof.

77. The compound or pharmaceutical composition of claim 76, wherein the cancer is a solid tumor or a hematological cancer.

78. The compound or pharmaceutical composition of claim 76 or 77, wherein the cancer is a leukemia or a lymphoma.

79. The compound or pharmaceutical composition of any of claims 76-78, wherein the cancer is acute myeloid leukemia (AML) or acute lymphoblastic leukemia (ALL).

80. The compound or pharmaceutical composition of claim 76 or 77, wherein the cancer is hepatocellular carcinoma, prostate cancer, or neuroblastoma.

81. A kit comprising a compound of any one of claims 1-60, or a pharmaceutically acceptable salt thereof, or a pharmaceutical composition of claim 61; and instructions for administering the compound, the pharmaceutically acceptable salt thereof, or the pharmaceutical composition to a subject.
